(19) United States
(12) Patent Application Publication
(10) Pub. No.: US 2009/0181963 A1
(43) Pub. Date: Jul. 16, 2009

(54) 3H-[1,2,3]TRIAZOLO[4,5-D]PYRIMIDINE COMPOUNDS, THEIR USE AS mTOR KINASE AND PI3 KINASE INHIBITORS, AND THEIR SYNTHESES

(75) Inventors: Christoph Martin Dehnhardt, New York, NY (US); Aranapakam Mudumbai Venkatesan, Rego Park, NY (US); Efren Guillermo Delos Santos, Nanuet, NY (US); Zecheng Chen, New City, NY (US); Osvaldo Dos Santos, Astoria, NY (US); Natasja Brooijmans, New York, NY (US); Arie Zask, New York, NY (US); Jeroen Cunera Verheijen, Highland Mills, NY (US); Semiramis Ayral-Kaloustian, Tarrytown, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/354,027

(22) Filed: Jan. 15, 2009

Related U.S. Application Data
(60) Provisional application No. 61/021,084, filed on Jan. 15, 2008.
(60) Provisional application No. 61/034,680, filed on Mar. 7, 2008.

(51) Int. Cl. A61K 31/5377; C07D 413/14; A61P 35/00

(52) U.S. Cl. 514/234.2; 544/118

(57) ABSTRACT

The invention relates to 3H-[1,2,3]triazolo[4,5-d]pyrimidine compounds of the Formula 1:

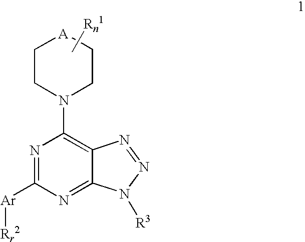

or a pharmaceutically acceptable salt thereof, wherein the constituent variables are as defined herein, compositions comprising the compounds, and methods for making and using the compounds.

### FIELD OF THE INVENTION

The invention relates to 3H-[1,2,3]triazolo[4,5-d]pyrimidine compounds, compositions comprising a 3H-[1,2,3]triazolo[4,5-d]pyrimidine compound, methods of synthesizing these compounds, and methods for treating PI3K-related diseases. The invention also relates to methods for treating mTOR-related diseases.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a&b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) has been reported to inhibit both PI3K, and PI3K, as well as the mTOR enzymes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) has been reported to inhibit PI3K_{α} and PI3K_{γ} but not the mTOR enzymes with an IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth which originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) has been reported to inhibit both PI3K_{α} and PI3K_{γ} as well as the mTOR enzymes with IC₅₀ values 4, 5, and “nanomolar”. Testing in human tumor xenograft models of cancer demonstrated activity against human tumor models of prostrate (PC-3) and glioblastoma (U-87) cancer (Verheijen, J. C. and Zask, A., Phosphatidylinositol 3-kinase (PI3K) inhibitors as anticancer drugs, Drugs Fut. 2007, 32 (6): 537-547).

The compound SF-1126 (a prodrug form of LY-294002, which is 2-(4-morpholinyl)-8-phenyl-4H-1-benzopyran-4-one) has been reported to be “a pan-PI3K inhibitor”. It is active in preclinical mouse cancer models of prostate, breast, ovarian, lung, multiple myeloma, and brain cancers. It began clinical trials in April, 2007 for the solid tumors endometrial, renal cell, breast, hormone refractory prostate and ovarian cancers. (Verheijen, J. C. and Zask, A., Phosphatidylinositol 3-kinase (PI3K) inhibitors as anticancer drugs, Drugs Fut. 2007, 32 (6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure), which were reported to be potentially useful as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to (Verheijen, J.C. and Zask, A., Phosphatidylinositol 3-kinase (PI3K) inhibitors as anticancer drugs, Drugs Fut. 2007, 32 (6): 537-547),
Although it seems clear that inhibition of the a isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. Everolimus is in a phase II clinical study for patients with Stage 1V Malignant Melanoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

In view of the foregoing information, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides a compound of the Formula 1:

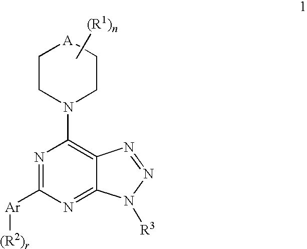

or a pharmaceutically acceptable salt thereof, wherein the constituent variables are as defined below.

In other aspects, the invention provides pharmaceutical compositions comprising compounds or pharmaceutically acceptable salts of compounds of the present formula 1.

In one aspect, the compounds or pharmaceutically acceptable salts thereof of the present formula 1 are useful as mTOR inhibitors.

In one aspect, the compounds or pharmaceutically acceptable salts thereof of the present formula 1 are useful as PI3K inhibitors.

In one aspect, the invention provides methods for treating an mTOR-related disorder, comprising administering to a mammal in need thereof, the compounds or pharmaceutically acceptable salts of compounds of the present formula 1 in an amount effective to treat an mTOR-related disorder.

In one aspect, the invention provides methods for treating a PI3K-related disorder, comprising administering to a mammal in need thereof the compounds or pharmaceutically acceptable salts of compounds of the present formula 1 in an amount effective to treat a PI3K-related disorder.

In other aspects, the invention provides further methods of synthesizing the compounds or pharmaceutically acceptable salts of compounds of the present formula 1.

### DETAILED DESCRIPTION OF THE INVENTION

In one aspect, the invention provides a compound of the Formula 1:

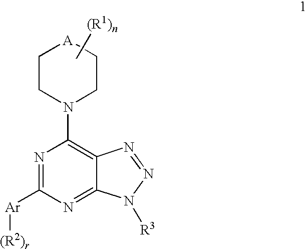

or a pharmaceutically acceptable salt thereof, wherein

### A is —O—, —CH₂O—, or —S(O)ₘ—;

m is 0, 1, or 2;
Ar is phenyl, naphthyl, or a nitrogen-containing mono- or bicyclic heteroaryl;
R¹ is independently C₁-C₆alkyl, C₆-C₁₄aryl, C₁-C₉heteroaryl, C₂-C₆alkenyl, C₂-C₆alkynyl, or C₃-C₈cycloalkyl;
or two R¹ groups on the same carbon atom, when taken together with the carbon to which they are attached, optionally form a carbonyl (C═O) group;
n is 0, 1, 2, or 3;
R² is independently halogen; C₁-C₆alkyl; C₂-C₆alkenyl; C₁-C₆alkoxy; C₂-C₆alkynyl; C₃-C₈cycloalkyl; C₆-C₁₄aryl; C₁-C₉heteroaryl; hydroxyl; C₁-C₆hydroxylalkyl-; —NR⁴R⁵; —NO₂; —CHO; —CN; —C(O)NR⁴R⁵; R⁶C(O)NH—; —CO₂H; —CF₃; —OCF₃; R⁴R⁵NC(O)NH—; or R⁶OC(O)NH—;
r is 0, 1, 2, 3, 4, or 5;
R⁴ and R⁵ are each independently H; (C₁-C₆alkoxy)carbonyl; C₁-C₆alkyl; C₆-C₁₄aryl, optionally substituted with R⁷R⁸NC(O)—, R⁷R⁸NC(O)NH—, CO₂H, —CONH₂, —CN, —NO₂, R⁷R⁸N—, R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, C₁-C₉heterocyclyl-O—, C₁-C₆alkyl, C₁-C₆alkoxy, C₁-C₆hydroxylalkyl-, C₁-C₉heterocycle, wherein the ring portion of the C₁-C₉heterocycle group is optionally substituted by C₁-C₆alkyl, halogen, NH₂—C₁-C₆alkylene-, (C₁-C₆alkyl)-NH—C₁-C₆alkylene-, (C₁-C₆alkyl)(C₁-C₆alkyl)N—C₁-C₆alkylene-, or (C₁-C₆alkoxy)carbonyl; C₁-C₉heteroaryl, optionally substituted by R⁷R⁸NC(O)—, R⁷R⁸NC(O)NH—, CO₂H, —CONH₂, —CN, —NO₂, R⁷R⁸N, R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, C₁-C₉heterocyclyl-O—, C₁-C₆alkyl, C₁-C₆alkoxy, C₁-C₆hydroxylalkyl-, C₁-C₉heterocycle, wherein the ring portion of the C₁-C₉heterocycle group is optionally substituted by C₁-C₆alkyl, halogen, NH₂—C₁-C₆alkylene-, (C₁-C₆alkyl)-NH—C₁-C₆alkylene-, (C₁-C₆alkyl) (C₁-C₆alkyl)N—C₁-C₆alkylene-, or (C₁-C₆alkoxy)carbonyl-; C₁-C₉heterocycle optionally substituted by (C₆-C₁₄aryl)alkyl-OC(O)— or C₁-C₆alkyl; C₃-C₈cycloalkyl; heterocyclyl(C₁-C₆alkyl) optionally substituted with C₁-C₆alkyl; C₁-C₆alkyl-OC(O)N(C₁-C₃alkyl)C₁-C₆alkylene; NH₂—C₁-C₆alkylene-; (C₁-C₆alkyl)-NH—C₁-C₆alkylene-; or (C₁-C₆alkyl)(C₁-C₆alkyl)N—C₁-C₆alkylene-;
or R⁴ and R⁵ when taken together with the nitrogen to which they are attached optionally form a 3- to 7-membered nitrogen containing heterocycle wherein up to two of the carbon atoms of the heterocycle is optionally replaced with —N(H)—, —O—, or —S(O)ₚ—;
p is 0, 1 or 2;
R⁶ is C₁-C₆alkyl; C₆-C₁₄aryl; (C₆-C₁₄aryl)alkyl, optionally substituted by NH₂; or C₁-C₆ perfluoroalkyl-;
R⁷ and R⁸ are each independently H; C₁-C₆alkyl optionally substituted with C₁-C₆alkoxy; C₁-C₈acyl optionally substituted with NH₂, (C₁-C₆alkyl)amino, or di(C₁-C₆alkyl)amino; (C₁-C₆alkyl)SO₂— optionally substituted with NH₂, (C₁-C₆alkyl)amino, or di(C₁-C₆alkyl)amino; (C₁-C₆alkyl)SO— optionally substituted with NH₂, (C₁-C₆alkyl)amino, or di(C₁-C₆alkyl)amino; C₆-C₁₄aryl-; (C₆-C₁₄aryl)SO₂—; (C₆-C₁₄aryl)SO—; aryl(C₁-C₆alkyl) optionally substituted with C₁-C₆alkoxy, C₁-C₆alkyl, or halo; C₁-C₉heteroaryl; (C₁-C₉heteroaryl)SO₂—; (C₁-C₉heteroaryl)SO—; heterocyclylSO₂—; heterocyclylSO—; C₁-C₆hydroxylalkyl; heteroaryl(C₁-C₆alkyl) optionally substituted with C₁-C₆alkoxy, C₁-C₆alkyl, or halo; heterocyclyl(C₁-C₆alkyl) optionally substituted with C₁-C₆alkyl; C₁-C₉heterocycle optionally substituted by (C₆-C₁₄aryl)alkyl-OC(O)—; NH₂—C₁-C₆alkylene-; (C₁-C₆alkyl)-NH—C₁-C₆alkylene-; or (C₁-C₆alkyl) (C₁-C₆alkyl)N—C₁-C₆alkylene-;
or R⁷ and R⁸ when taken together with the nitrogen to which they are attached optionally form a 3- to 7-membered nitrogen containing heterocycle wherein up to two of the carbon atoms of the heterocycle are optionally replaced with —N(R⁹)—, —O—, or —S(O)_{q}—, and wherein the heterocycle is optionally substituted with from 1 to 3 substituents independently selected from C₁-C₆alkyl; (C₁-C₆alkyl)amino-, C₆-C₁₄aryl, di(C₁-C₆alkyl)amino-, H₂N—, C₁-C₉heteroaryl, and C₁-C₉heterocycle;
q is 0, 1 or 2;
R⁹ is H or C₁-C₆alkyl;

### R³ is:

(a) hydrogen;
(b) C₁-C₆alkyl optionally substituted with from 1 to 3 substituents independently selected from:
   (i) C₁-C₆alkoxy,
   (ii) NH₂,
   (iii) (C₁-C₆alkyl)amino,
   (iv) di(C₁-C₆alkyl)amino,
   (v) CO₂H,
   (vi) and (C₁-C₆alkoxy)carbonyl;
(c) carboxyamidoalkyl optionally substituted with a substituent selected from:
   (i) halogen,
   (ii) and di(C₁-C₆alkyl)amino;
(d) C₁-C₆ perfluoroalkyl-;
(e) C₃-C₈cycloalkyl;
(f) C₆-C₁₄aryl optionally substituted with a substituent selected from:
   (i) —O—C₁-C₆alkylene-NH₂,
   (ii) —COOH,
   (iii) C₁-C₆hydroxylalkyl,
   (iv) R¹⁰R¹¹NC(O)—,
   (v) and (C₁-C₆alkoxy)carbonyl;
(g) monocyclic C₁-C₆heterocycle optionally substituted with from 1 to 3 substituents independently selected from:
   (i) C₁-C₈acyl, wherein the C₁-C₈acyl is optionally substituted with a NH₂,
   (ii) C₁-C₆alkyl,
   (iii) heteroaryl(C₁-C₆alkyl) wherein the ring portion of the heteroaryl(C₁-C₆alkyl) group is optionally substituted with from 1 to 3 substituents independently selected from:
      A) C₁-C₆alkylC(O)NH—,
      B) halogen,
      C) NH₂,
      D) and C₁-C₆alkyl,
   (iv) heterocyclyl(C₁-C₆alkyl), wherein the ring portion of the heterocyclyl(C₁-C₆alkyl) group is optionally substituted by a (C₆-C₁₄aryl)alkyl,
   (v) (C₆-C₁₄aryl)alkyl, wherein the ring portion of the (C₆-C₁₄aryl)alkyl group is optionally substituted by 1 to 3 substituents independently selected from:
      A) halogen,
      B) C₁-C₆alkyl,
      C) di(C₁-C₆alkyl)amino-(C₁-C₆alkylene)-O—,
      D) and C₁-C₉heteroaryl;
   (vi) and (C₁-C₆alkoxy)carbonyl;
(h) heterocyclyl(C₁-C₆alkyl) optionally substituted with a substituent selected from:
   (i) C₁-C₆alkyl,
   (ii) C₃-C₈cycloalkyl,
   (iii) (C₁-C₆alkoxy)carbonyl,
   (iv) C₁-C₆alkylcarboxy,
   (v) (C₆-C₁₄aryl)alkyl wherein the ring portion of the (C₆-C₁₄aryl)alkyl group is optionally substituted by a:
      A) halogen,
      B) C₁-C₉heteroaryl,
      C) or di(C₁-C₆alkyl)amino-(C₁-C₆alkylene)-O—,
   (vi) heteroaryl(C₁-C₆alkyl) wherein the ring portion of the heteroaryl(C₁-C₆alkyl) group is optionally substituted by a halogen,
   (vii) and C₁-C₈acyl, wherein the C₁-C₈acyl is optionally substituted with from 1 to 3 independently selected halogens,
(i) (C₁-C₆alkyl)-C(O)—NH—(C₁-C₆alkylene)-;
(j) heteroaryl(C₁-C₆alkyl);
(k) (C₆-C₁₄aryl)alkyl wherein the ring portion of the (C₆-C₁₄aryl)alkyl group is optionally substituted by a:
   (i) ClC₆H₄C(O)NH—,
   (ii) (C₁-C₆alkoxy)carbonyl,
   (iii) CO₂H,
   (iv) or R¹⁰R¹¹NC(O);
(l) C₁-C₆hydroxylalkyl;
(m) or C₁-C₉heteroaryl;
   R¹⁰ and R¹¹ are each independently:
(a) H;
(b) C₁-C₆alkyl optionally substituted with a substituent selected from:
   (i) C₁-C₆alkylC(O)NH—,
   (ii) NH₂,
   (iii) (C₁-C₆alkyl)amino,
   (iv) or di(C₁-C₆alkyl)amino,
(c) C₃-C₈cycloalkyl;
(d) C₆-C₁₄aryl optionally substituted with a substituent selected from:
   (v) halogen,
   (vi) and monocyclic C₁-C₆heterocycle wherein the monocyclic C₁-C₆heterocycle is optionally substituted with (C₁-C₆alkoxy)carbonyl;
(n) C₁-C₉heteroaryl;
(o) heteroaryl(C₁-C₆alkyl);
(p) heterocyclyl(C₁-C₆alkyl);
(q) (C₆-C₁₄aryl)alkyl, wherein the chain portion of the (C₆-C₁₄aryl)alkyl group is optionally substituted by a hydroxyl;
(r) or monocyclic C₁-C₆heterocycle optionally substituted with a (C₁-C₆alkoxy)carbonyl;
   or R¹⁰ and R¹¹ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

R⁴ and R⁵ are suitably each independently H; (C₁-C₆alkoxy)carbonyl; C₁-C₆alkyl; C₆-C₁₄aryl, optionally substituted with halogen, R⁷R⁸NC(O)—, CO₂H, —CONH₂, —CN, R⁷R⁸N, R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, heterocyclyl, heterocyclyl-O—, C₁-C₆alkyl, C₁-C₆alkoxy, C₁-C₆hydroxylalkyl-, C₁-C₉heterocycle, wherein the ring portion of the C₁-C₉heterocycle group in turn is optionally substituted by C₁-C₆alkyl, halogen, NH₂—C₁-C₆alkylene-, (C₁-C₆alkyl)-NH—C₁-C₆alkylene-, (C₁-C₆alkyl)(C₁-C₆alkyl)N—C₁-C₆alkylene-, or (C₁-C₆alkoxy)carbonyl; C₁-C₉heteroaryl, optionally substituted by C₁-C₆alkyl R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, heterocyclyl, or heterocyclyl-O—; C₁-C₉heterocycle optionally substituted by (C₆-C₁₄aryl)alkyl-OC(O)— or C₁-C₆alkyl; C₃-C₈cycloalkyl; heterocyclyl(C₁-C₆alkyl) optionally substituted with C₁-C₆alkyl; C₁-C₆alkyl-OC(O)N(C₁-C₃alkyl)C₁-C₆alkylene; NH₂—C₁-C₆alkylene-; (C₁-C₆alkyl)-NH—C₁-C₆alkylene-; or (C₁-C₆alkyl) (C₁-C₆alkyl)N—C₁-C₆alkylene-.

R⁷ and R⁸ are suitably each independently H; C₁-C₆alkyl; C₁-C₈acyl optionally substituted with NH₂, (C₁-C₆alkyl)amino, or di(C₁-C₆alkyl)amino; (C₁-C₆alkyl)SO₂— optionally substituted with NH₂, (C₁-C₆alkyl)amino, or di(C₁-C₆alkyl)amino; (C₁-C₆alkyl)SO— optionally substituted with NH₂, (C₁-C₆alkyl)amino, or di(C₁-C₆alkyl)amino; (C₆-C₁₄aryl)SO₂—; (C₆-C₁₄aryl)SO—; (C₁-C₉heteroaryl)SO₂—; (C₁-C₉heteroaryl)SO—; heterocyclylSO₂—; heterocyclylSO—; C₁-C₆hydroxylalkyl; heteroaryl(C₁-C₆alkyl) optionally substituted with C₁-C₆alkyl; heterocyclyl(C₁-C₆alkyl) optionally substituted with C₁-C₆alkyl; C₁-C₉heterocycle optionally substituted by (C₆-C₁₄aryl)alkyl-OC(O)—; NH₂—C₁-C₆alkylene-; (C₁-C₆alkyl)-NH—C₁-C₆alkylene-; or (C₁-C₆alkyl)(C₁-C₆alkyl)N—C₁-C₆alkylene-.

R⁷ and R⁸ when taken together with the nitrogen to which they are attached suitably form a 3- to 7-membered nitrogen containing heterocycle wherein up to two of the carbon atoms of the heterocycle are optionally replaced with —N(R⁹)—, —O—, or —S(O)_{q}—, and wherein the heterocycle is optionally substituted with from 1 to 3 substituents independently selected from C₁-C₆alkyl; C₆-C₁₄aryl, C₁-C₉heteroaryl, and C₁-C₉heterocycle.

In certain embodiments n is 0.

In certain embodiments A is —O—.

In certain embodiments r is 1.

Ar may suitably represent a nitrogen-containing monocyclic heteroaryl.

Ar may suitably represent pyridinyl.

Ar may represent 3-pyridinyl.

In certain embodiments Ar may represent phenyl.

Ar may suitably represent phenyl substituted in the 4-position by R², where R² may suitably be hydroxyl or

### —NHC(O)NR⁴R⁵.

R³ may suitably be C₁-C₆alkyl or ethyl.

In certain embodiments R⁴ is C₆-C₁₄aryl, optionally substituted with R⁷R⁸NC(O)—;

R⁴ may suitably be phenyl, substituted with R⁷R⁸NC(O)—, e.g. phenyl, substituted in the 4-position with R⁷R⁸NC(O)—.

In certain embodiments R⁵ is H.

In certain embodiments R⁷ is (C₁-C₆alkyl)(C₁-C₆alkyl)N—C₁-C₆alkylene-.

R⁷ may suitably be 2-(dimethylamino)ethyl.

In certain embodiments R⁸ is H.

In certain embodiments R⁷ and R⁸ taken together with the nitrogen to which they are attached form a 3- to 7-membered nitrogen containing heterocycle wherein up to two of the carbon atoms of the heterocycle are optionally replaced with —N(R⁹)—, —O—, or —S(O)_{q}—.

R⁷ and R⁸ may suitably betaken together with the nitrogen to which they are attached form a 6-membered nitrogen containing heterocycle wherein one of the carbon atoms of the heterocycle is replaced with —N(R⁹)—, e.g.

R⁷ and R⁸ taken together are 4-methylpiperazin-1-yl.
R⁹ may suitably be C₁-C₆ alkyl.

In certain embodiments R³ is a monocyclic C₁-C₆heterocycle optionally substituted with from 1 to 3 substituents independently selected from C₁-C₈acyl, C₁-C₆alkyl, heterocyclyl(C₁-C₆alkyl), wherein the ring portion of the heterocyclyl(C₁-C₆alkyl) group is optionally substituted by 1 to 3 substituents independently selected from halogen, —NH₂, —O(C₁-C₆alkyl), C₁-C₆alkyl, monocyclic C₁-C₆heterocycle, (C₆-C₁₄aryl)alkyl, and C₃-C₈cycloalkyl, (C₆-C₁₄aryl)alkyl, wherein the ring portion of the (C₆-C₁₄aryl)alkyl group is optionally substituted by 1 to 3 substituents independently selected from halogen, —NH₂, —O(C₁-C₆alkyl), C₁-C₆alkyl, monocyclic C₁-C₆heterocycle, and C₃-C₈cycloalkyl.

In particular embodiments R³ is a piperidinyl group optionally substituted with from 1 to 3 substituents independently selected from C₁-C₈acyl, C₁-C₆alkyl, heterocyclyl(C₁-C₆alkyl), wherein the ring portion of the heterocyclyl(C₁-C₆alkyl) group is optionally substituted by 1 to 3 substituents independently selected from halogen, —NH₂, —O(C₁-C₆alkyl), C₁-C₆alkyl, monocyclic C₁-C₆heterocycle, (C₆-C₁₄aryl)alkyl, and C₃-C₈cycloalkyl, (C₆-C₁₄aryl)alkyl, wherein the ring portion of the (C₆-C₁₄aryl)alkyl group is optionally substituted by 1 to 3 substituents independently selected from halogen, —NH₂, —O(C₁-C₆alkyl), C₁-C₆alkyl, monocyclic C₁-C₆heterocycle, and C₃-C₈cycloalkyl.

R³ may suitably be a piperidin-4-yl group optionally substituted with from 1 to 3 substituents independently selected from C₁-C₈acyl, C₁-C₆alkyl, heterocyclyl(C₁-C₆alkyl),

wherein the ring portion of the heterocyclyl(C₁-C₆alkyl) group is optionally substituted by 1 to 3 substituents independently selected from halogen, —NH₂, —O(C₁-C₆alkyl), C₁-C₆alkyl, monocyclic C₁-C₆heterocycle, (C₆-C₁₄aryl)alkyl, and C₃-C₈cycloalkyl, (C₆-C₁₄aryl)alkyl, wherein the ring portion of the (C₆-C₁₄aryl)alkyl group is optionally substituted by 1 to 3 substituents independently selected from halogen, —NH₂, —O(C₁-C₆alkyl), C₁-C₆alkyl, monocyclic C₁-C₆heterocycle, and C₃-C₈cycloalkyl.

R³ may suitably be a piperidinyl group substituted with from 1 to 3 substituents independently selected from heterocyclyl(C₁-C₆alkyl), wherein the ring portion of the heterocyclyl(C₁-C₆alkyl) group is optionally substituted by 1 to 3 substituents independently selected from halogen, and C₁-C₆alkyl, and (C₆-C₁₄aryl)alkyl wherein the ring portion of the (C₆-C₁₄aryl)alkyl group is optionally substituted by 1 to 3 halogens.

R³ may suitably be a piperidinyl group substituted with heterocyclyl(C₁-C₆alkyl),

wherein the ring portion of the heterocyclyl(C₁-C₆alkyl) group is optionally substituted by 1 to 3 substituents independently selected from halogen, —and C₁-C₆alkyl.

or R³ may suitably be a piperidinyl group substituted with (C₆-C₁₄aryl)alkyl wherein the ring portion of the (C₆-C₁₄aryl)alkyl group is optionally substituted by 1 to 3 halogens.

In one aspect, n is 0, A is —O—, r is 1, Ar is 3-pyridinyl, R² is hydroxyl, and R³ is a 4-piperidinyl group substituted with from 1 to 3 substituents independently selected from heterocyclyl(C₁-C₆alkyl), wherein the ring portion of the heterocyclyl(C₁-C₆alkyl) group is optionally substituted by 1 to 3 substituents independently selected from halogen, or C₁-C₆alkyl, and (C₆-C₁₄aryl)alkyl wherein the ring portion of the (C₆-C₁₄aryl)alkyl group is optionally substituted by 1 to 3 halogens.

In one aspect, n is 0, A is —O—, r is 1, Ar is 3-pyridinyl, R² is hydroxyl, and R³ is a 4-piperidinyl group substituted with pyridylmethyl, wherein the ring portion of the pyridylmethyl group is by halogen.

In one aspect, n is 0, A is —O—, r is 1, Ar is 3-pyridinyl, R² is hydroxyl, and R³ is a 4-piperidinyl group substituted with benzyl, wherein the ring portion of the benzyl group is optionally substituted by 1 to 3 halogens.

In one aspect, n is 0, A is —O—, r is 1, Ar is phenyl, R² is —NHC(O)NR⁴R⁵, R⁴ is C₆-C₁₄aryl, optionally substituted with R⁷R⁸NC(O)—, and R³ is C₁-C₆alkyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ is (C₁-C₆alkyl)(C₁-C₆alkyl)N—C₁-C₆alkylene-, R⁸ is H, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ is 2-(dimethylamino)ethyl, R⁸ is H, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 3- to 7-membered nitrogen containing heterocycle wherein up to two of the carbon atoms of the heterocycle optionally are replaced with —N(R⁹)—, —O—, or —S(O)_{q}—, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 6-membered nitrogen containing heterocycle wherein one of the carbon atoms of the heterocycle is replaced with —N(R⁹)—, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 6-membered nitrogen containing heterocycle wherein one of the carbon atoms of the heterocycle is replaced with —N(R⁹)—, R⁹ is C₁-C₆alkyl, R⁵ is H, and R³ is ethyl.

In one aspect, n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 6-membered nitrogen containing heterocycle wherein one of the carbon atoms of the heterocycle is replaced with —N(R⁹)—, R⁹ is methyl, R⁵ is H, and R³ is ethyl.

Illustrative compounds of Formula 1 include by the following compounds:
3-[3-(1-benzylpiperidin-4-yl)-7-morpholin-4-yl-3H-[1,2,3]triazolo[4,5-d]pyrimidin-5-yl]phenol;
5-[3-(1-benzylpiperidin-4-yl)-7-morpholin-4-yl-3H-[1,2,3]triazolo[4,5-d]pyrimidin-5-yl]pyrimidin-2-amine;
5-[3-(1-benzylpiperidin-4-yl)-7-morpholin-4-yl-3H-[1,2,3]triazolo[4,5-d]pyrimidin-5-yl]pyridin-3-ol;
1-{4-[3-(1-benzylpiperidin-4-yl)-7-morpholin-4-yl-3H-[1,2,3]triazolo[4,5-d]pyrimidin-5-yl]phenyl}-3-[2-(dimethylamino)ethyl]urea;
N-{4-[3-(1-benzylpiperidin-4-yl)-7-morpholin-4-yl-3H-[1,2,3]triazolo[4,5-d]pyrimidin-5-yl]phenyl}-2,2,2-trifluoroacetamide;
1-{4-[3-(1-benzylpiperidin-4-yl)-7-morpholin-4-yl-3H-[1,2,3]triazolo[4,5-d]pyrimidin-5-yl]phenyl}-3-methylurea;
N-{2-[5-(3-hydroxyphenyl)-7-morpholin-4-yl-3H-[1,2,3]triazolo[4,5-d]pyrimidin-3-yl]ethyl}acetamide;
N-(2-{5-[3-(hydroxymethyl)phenyl]-7-morpholin-4-yl-3H-[1,2,3]triazolo[4,5-d]pyrimidin-3-yl}ethyl)acetamide;
3-[7-morpholin-4-yl-3-(3-pyrrolidin-1-ylpropyl)-3H-[1,2,3]triazolo[4,5-d]pyrimidin-5-yl]phenol;
{3-[7-morpholin-4-yl-3-(3-pyrrolidin-1-ylpropyl)-3H-[1,2,3]triazolo[4,5-d]pyrimidin-5-yl]phenyl}methanol;
5-(1H-indazol-4-yl)-7-morpholin-4-yl-3-(3-pyrrolidin-1-ylpropyl)-3H-[1,2,3]triazolo[4,5-d]pyrimidine;
5-(7-morpholin-4-yl-3-piperidin-4-yl-3H-[1,2,3]triazolo[4,5-d]pyrimidin-5-yl)pyridin-3-ol;
5-{3-[1-(2-furylmethyl)piperidin-4-yl]-7-morpholin-4-yl-3H-[1,2,3]triazolo[4,5-d]pyrimidin-5-yl}pyridin-3-ol;
5-{3-[1-(4-fluorobenzyl)piperidin-4-yl]-7-morpholin-4-yl-3H-[1,2,3]triazolo[4,5-d]pyrimidin-5-yl}pyridin-3-ol;
5-(3-{1-[(6-bromopyridin-3-yl)methyl]piperidin-4-yl}-7-morpholin-4-yl-3H-[1,2,3]triazolo[4,5-d]pyrimidin-5-yl)pyridin-3-ol;
5-(3-{1-[(5-bromopyridin-3-yl)methyl]piperidin-4-yl}-7-morpholin-4-yl-3H-[1,2,3]triazolo[4,5-d]pyrimidin-5-yl)pyridin-3-ol;
5-[3-(1-{4-[3-(dimethylamino)propoxy]benzyl}piperidin-4-yl)-7-morpholin-4-yl-3H-[1,2,3]triazolo[4,5-d]pyrimidin-5-yl]pyridin-3-ol;
5-{3-[1-(3,4-difluorobenzyl)piperidin-4-yl]-7-morpholin-4-yl-3H-[1,2,3]triazolo[4,5-d]pyrimidin-5-yl}pyridin-3-ol;
5-(3-{1-[(1-methyl-1H-pyrrol-2-yl)methyl]piperidin-4-yl}-7-morpholin-4-yl-3H-[1,2,3]triazolo[4,5-d]pyrimidin-5-yl)pyridin-3-ol;
5-(3-{1-[(6-chloropyridin-3-yl)methyl]piperidin-4-yl}-7-morpholin-4-yl-3H-[1,2,3]triazolo[4,5-d]pyrimidin-5-yl)pyridin-3-ol;
5-(3-{1-[(5-methyl-2-thienyl)methyl]piperidin-4-yl}-7-morpholin-4-yl-3H-[1,2,3]triazolo[4,5-d]pyrimidin-5-yl)pyridin-3-ol;
5-[3-(1-methylpiperidin-4-yl)-7-morpholin-4-yl-3H-[1,2,3]triazolo[4,5-d]pyrimidin-5-yl]pyridin-3-ol;
5-{3-[1-(2,4-difluorobenzyl)piperidin-4-yl]-7-morpholin-4-yl-3H-[1,2,3]triazolo[4,5-d]pyrimidin-5-yl}pyridin-3-ol;
5-(3-{1-[(1-methyl-1H-imidazol-5-yl)methyl]piperidin-4-yl}-7-morpholin-4-yl-3H-[1,2,3]triazolo[4,5-d]pyrimidin-5-yl)pyridin-3-ol;
N-[3-({4-[5-(5-hydroxypyridin-3-yl)-7-morpholin-4-yl-3H-[1,2,3]triazolo[4,5-d]pyrimidin-3-yl]piperidin-1-yl}methyl)pyridin-2-yl]-2,2-dimethylpropanamide;
5-(3-{1-[(4,5-dimethyl-2-thienyl)methyl]piperidin-4-yl}-7-morpholin-4-yl-3H-[1,2,3]triazolo[4,5-d]pyrimidin-5-yl)pyridin-3-ol;
5-[3-(1-butylpiperidin-4-yl)-7-morpholin-4-yl-3H-[1,2,3]triazolo[4,5-d]pyrimidin-5-yl]pyridin-3-ol;
5-(3-{1-[(4-benzylpiperazin-1-yl)methyl]piperidin-4-yl}-7-morpholin-4-yl-3H-[1,2,3]triazolo[4,5-d]pyrimidin-5-yl)pyridin-3-ol;
5-{7-morpholin-4-yl-3-[1-(1H-pyrrol-2-ylmethyl)piperidin-4-yl]-3H-[1,2,3]triazolo[4,5-d]pyrimidin-5-yl}pyridin-3-ol;
5-(3-{1-[(1-methyl-1H-pyrazol-5-yl)methyl]piperidin-4-yl}-7-morpholin-4-yl-3H-[1,2,3]triazolo[4,5-d]pyrimidin-5-yl)pyridin-3-ol;
5-{7-morpholin-4-yl-3-[1-(4-pyridin-4-ylbenzyl)piperidin-4-yl]-3H-[1,2,3]triazolo[4,5-d]pyrimidin-5-yl}pyridin-3-ol;
4-(3-ethyl-7-morpholin-4-yl-3H-[1,2,3]triazolo[4,5-d]pyrimidin-5-yl)aniline;
1-[4-(3-ethyl-7-morpholin-4-yl-3H-[1,2,3]triazolo[4,5-d]pyrimidin-5-yl)phenyl]-3-pyridin-4-ylurea;
1-[2-(dimethylamino)ethyl]-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-(2-methylpyridin-4-yl)urea;
1-[4-(3-ethyl-7-morpholin-4-yl-3H-[1,2,3]triazolo[4,5-d]pyrimidin-5-yl)phenyl]-3-(4H-1,2,4-triazol-4-yl)urea;
1-[4-(3-ethyl-7-morpholin-4-yl-3H-[1,2,3]triazolo[4,5-d]pyrimidin-5-yl)phenyl]-3-(1,3-thiazol-2-yl)urea;
2-(4-aminophenyl)ethyl [4-(3-ethyl-7-morpholin-4-yl-3H-[1,2,3]triazolo[4,5-d]pyrimidin-5-yl)phenyl]carbamate;
1-[4-(3-ethyl-7-morpholin-4-yl-3H-[1,2,3]triazolo[4,5-d]pyrimidin-5-yl)phenyl]-3-pyridin-3-ylurea;
1-[4-(3-ethyl-7-morpholin-4-yl-3H-[1,2,3]triazolo[4,5-d]pyrimidin-5-yl)phenyl]-3-(2-thienyl)urea;
methyl 4-({[4-(3-ethyl-7-morpholin-4-yl-3H-[1,2,3]triazolo[4,5-d]pyrimidin-5-yl)phenyl]carbamoyl}amino)benzoate;
4-({[4-(3-ethyl-7-morpholin-4-yl-3H-[1,2,3]triazolo[4,5-d]pyrimidin-5-yl)phenyl]carbamoyl}amino)benzoic acid;
N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)benzamide;
1-[4-(3-ethyl-7-morpholin-4-yl-3H-[1,2,3]triazolo[4,5-d]pyrimidin-5-yl)phenyl]-3-{4-[(4-methylpiperazin-1-yl)carbonyl]phenyl}urea;
N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)-N-methylbenzamide;
4-({[4-(3-ethyl-7-morpholin-4-yl-3H-[1,2,3]triazolo[4,5-d]pyrimidin-5-yl)phenyl]carbamoyl}amino)-N-(2-hydroxyethyl)benzamide;
N-[3-(dimethylamino)propyl]-4-({[4-(3-ethyl-7-morpholin-4-yl-3H-[1,2,3]triazolo[4,5-d]pyrimidin-5-yl)phenyl]carbamoyl}amino)benzamide;
1-[4-(3-ethyl-7-morpholin-4-yl-3H-[1,2,3]triazolo[4,5-d]pyrimidin-5-yl)phenyl]-3-{4-[(4-morpholin-4-ylpiperidin-1-yl)carbonyl]phenyl}urea;
4-({[4-(3-ethyl-7-morpholin-4-yl-3H-[1,2,3]triazolo[4,5-d]pyrimidin-5-yl)phenyl]carbamoyl}amino)-N-[2-(4-methylpiperazin-1-yl)ethyl]benzamide;
1-[4-(1,4′-bipiperidin-1′-ylcarbonyl)phenyl]-3-[4-(3-ethyl-7-morpholin-4-yl-3H-[1,2,3]triazolo[4,5-d]pyrimidin-5-yl)phenyl]urea;
4-({[4-(3-ethyl-7-morpholin-4-yl-3H-[1,2,3]triazolo[4,5-d]pyrimidin-5-yl)phenyl]carbamoyl}amino)-N-(pyridin-4-ylmethyl)benzamide;
4-({[4-(3-ethyl-7-morpholin-4-yl-3H-[1,2,3]triazolo[4,5-d]pyrimidin-5-yl)phenyl]carbamoyl}amino)-N-methyl-N-[2-(methylamino)ethyl]benzamide;
4-({[4-(3-ethyl-7-morpholin-4-yl-3H-[1,2,3]triazolo[4,5-d]pyrimidin-5-yl)phenyl]carbamoyl}amino)-N-(2-morpholin-4-ylethyl)benzamide;
1-[4-(3-ethyl-7-morpholin-4-yl-3H-[1,2,3]triazolo[4,5-d]pyrimidin-5-yl)phenyl]-3-(4-{[(3R)-3-methylpiperazin-1-yl]carbonyl}phenyl)urea;
4-({[4-(3-ethyl-7-morpholin-4-yl-3H-[1,2,3]triazolo[4,5-d]pyrimidin-5-yl)phenyl]carbamoyl}amino)-N-[3-(4-methylpiperazin-1-yl)propyl]benzamide;
4-({[4-(3-ethyl-7-morpholin-4-yl-3H-[1,2,3]triazolo[4,5-d]pyrimidin-5-yl)phenyl]carbamoyl}amino)-N-(2-piperidin-1-ylethyl)benzamide;
1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-{4-[(4-pyridin-2-ylpiperazin-1-yl)carbonyl]phenyl}urea;
4-({[4-(3-ethyl-7-morpholin-4-yl-3H-[1,2,3]triazolo[4,5-d]pyrimidin-5-yl)phenyl]carbamoyl}amino)-N-[(1-ethylpyrrolidin-2-yl)methyl]benzamide;
4-({[4-(3-ethyl-7-morpholin-4-yl-3H-[1,2,3]triazolo[4,5-d]pyrimidin-5-yl)phenyl]carbamoyl}amino)benzamide;
4-({[4-(3-ethyl-7-morpholin-4-yl-3H-[1,2,3]triazolo[4,5-d]pyrimidin-5-yl)phenyl]carbamoyl}amino)-N,N-dimethylbenzamide;
N-butyl-4-({[4-(3-ethyl-7-morpholin-4-yl-3H-[1,2,3]triazolo[4,5-d]pyrimidin-5-yl)phenyl]carbamoyl}amino)benzamide;
4-({[4-(3-ethyl-7-morpholin-4-yl-3H-[1,2,3]triazolo[4,5-d]pyrimidin-5-yl)phenyl]carbamoyl}amino)-N-(2-pyridin-2-ylethyl)benzamide;
N-ethyl-4-({[4-(3-ethyl-7-morpholin-4-yl-3H-[1,2,3]triazolo[4,5-d]pyrimidin-5-yl)phenyl]carbamoyl}amino)benzamide; benzyl 4-({[4-(3-ethyl-7-morpholin-4-yl-3H-[1,2,3]triazolo[4,5-d]pyrimidin-5-yl)phenyl]carbamoyl}amino)piperidine-1-carboxylate;
1-[4-(3-ethyl-7-morpholin-4-yl-3H-[1,2,3]triazolo[4,5-d]pyrimidin-5-yl)phenyl]-3-piperidin-4-ylurea;
4-{3-ethyl-7-[(3S)-3-methylmorpholin-4-yl]-3H-[1,2,3]triazolo[4,5-d]pyrimidin-5-yl}aniline;
1-(4-{3-ethyl-7-[(3S)-3-methylmorpholin-4-yl]-3H-[1,2,3]triazolo[4,5-d]pyrimidin-5-yl}phenyl)-3-[4-(2-hydroxyethyl)phenyl]urea;
1-(4-{3-ethyl-7-[(3S)-3-methylmorpholin-4-yl]-3H-[1,2,3]triazolo[4,5-d]pyrimidin-5-yl}phenyl)-3-(2-thienyl)urea;
1-(4-{3-ethyl-7-[(3S)-3-methylmorpholin-4-yl]-3H-[1,2,3]triazolo[4,5-d]pyrimidin-5-yl}phenyl)-3-[4-(hydroxymethyl)phenyl]urea;
1-(4-{3-ethyl-7-[(3S)-3-methylmorpholin-4-yl]-3H-[1,2,3]triazolo[4,5-d]pyrimidin-5-yl}phenyl)-3-pyridin-4-ylurea;
1-(4-{3-ethyl-7-[(3S)-3-methylmorpholin-4-yl]-3H-[1,2,3]triazolo[4,5-d]pyrimidin-5-yl}phenyl)-3-pyridin-3-ylurea;
1-(4-{3-ethyl-7-[(3S)-3-methylmorpholin-4-yl]-3H-[1,2,3]triazolo[4,5-d]pyrimidin-5-yl}phenyl)-3-(4-methoxyphenyl)urea;
1-(4-{3-ethyl-7-[(3S)-3-methylmorpholin-4-yl]-3H-[1,2,3]triazolo[4,5-d]pyrimidin-5-yl}phenyl)-3-(4-fluorophenyl)urea;
1-(4-cyanophenyl)-3-(4-{3-ethyl-7-[(3S)-3-methylmorpholin-4-yl]-3H-[1,2,3]triazolo[4,5-d]pyrimidin-5-yl}phenyl)urea;
1-(4-{3-ethyl-7-[(3S)-3-methylmorpholin-4-yl]-3H-[1,2,3]triazolo[4,5-d]pyrimidin-5-yl}phenyl)-3-[4-(4-methylpiperazin-1-yl)phenyl]urea;
4-(3-cyclopropyl-7-morpholin-4-yl-3H-[1,2,3]triazolo[4,5-d]pyrimidin-5-yl)aniline;
1-[4-(3-cyclopropyl-7-morpholin-4-yl-3H-[1,2,3]triazolo[4,5-d]pyrimidin-5-yl)phenyl]-3-pyridin-4-ylurea;
1-[4-(3-cyclopropyl-7-morpholin-4-yl-3H-[1,2,3]triazolo[4,5-d]pyrimidin-5-yl)phenyl]-3-pyridin-4-ylurea;
1-[4-(3-cyclopropyl-7-morpholin-4-yl-3H-[1,2,3]triazolo[4,5-d]pyrimidin-5-yl)phenyl]-3-(2-thienyl)urea;
4-(3-isopropyl-7-morpholin-4-yl-3H-[1,2,3]triazolo[4,5-d]pyrimidin-5-yl)aniline;
1-[4-(3-isopropyl-7-morpholin-4-yl-3H-[1,2,3]triazolo[4,5-d]pyrimidin-5-yl)phenyl]-3-pyridin-4-ylurea;
1-[4-(3-isopropyl-7-morpholin-4-yl-3H-[1,2,3]triazolo[4,5-d]pyrimidin-5-yl)phenyl]-3-pyridin-3-ylurea;
1-[4-(hydroxymethyl)phenyl]-3-[4-(3-isopropyl-7-morpholin-4-yl-3H-[1,2,3]triazolo[4,5-d]pyrimidin-5-yl)phenyl]urea;
1-[4-(3-isopropyl-7-morpholin-4-yl-3H-[1,2,3]triazolo[4,5-d]pyrimidin-5-yl)phenyl]-3-(4-morpholin-4-ylphenyl)urea;
1-[4-(dimethylamino)phenyl]-3-[4-(3-isopropyl-7-morpholin-4-yl-3H-[1,2,3]triazolo[4,5-d]pyrimidin-5-yl)phenyl]urea;
1-(4-fluorophenyl)-3-[4-(3-isopropyl-7-morpholin-4-yl-3H-[1,2,3]triazolo[4,5-d]pyrimidin-5-yl)phenyl]urea;
1-[2-(dimethylamino)ethyl]-3-[4-(3-isopropyl-7-morpholin-4-yl-3H-[1,2,3]triazolo[4,5-d]pyrimidin-5-yl)phenyl]urea;
1-[4-(3-isopropyl-7-morpholin-4-yl-3H-[1,2,3]triazolo[4,5-d]pyrimidin-5-yl)phenyl]-3-(4-methoxyphenyl)urea;
1-[4-(3-isopropyl-7-morpholin-4-yl-3H-[1,2,3]triazolo[4,5-d]pyrimidin-5-yl)phenyl]-3-(4-methylphenyl)urea;
1-[4-(3-isopropyl-7-morpholin-4-yl-3H-[1,2,3]triazolo[4,5-d]pyrimidin-5-yl)phenyl]-3-methylurea;
1-[(1-ethylpyrrolidin-2-yl)methyl]-3-[4-(3-isopropyl-7-morpholin-4-yl-3H-[1,2,3]triazolo[4,5-d]pyrimidin-5-yl)phenyl]urea;
4-({[4-(3-isopropyl-7-morpholin-4-yl-3H-[1,2,3]triazolo[4,5-d]pyrimidin-5-yl)phenyl]carbamoyl}amino)benzamide;
1-[4-(3-isopropyl-7-morpholin-4-yl-3H-[1,2,3]triazolo[4,5-d]pyrimidin-5-yl)phenyl]-3-isoxazol-4-ylurea;
1-[4-(3-isopropyl-7-morpholin-4-yl-3H-[1,2,3]triazolo[4,5-d]pyrimidin-5-yl)phenyl]-3-(1H-pyrrol-3-yl)urea;
1-[4-(3-isopropyl-7-morpholin-4-yl-3H-[1,2,3]triazolo[4,5-d]pyrimidin-5-yl)phenyl]urea;
tert-butyl 4-{2-[5-(3-hydroxyphenyl)-7-morpholin-4-yl-3H-[1,2,3]triazolo[4,5-d]pyrimidin-3-yl]ethyl}piperazine-1-carboxylate;
3-[7-morpholin-4-yl-3-(2-piperazin-1-ylethyl)-3H-[1,2,3]triazolo[4,5-d]pyrimidin-5-yl]phenol;
3-{3-[2-(4-benzoylpiperazin-1-yl)ethyl]-7-morpholin-4-yl-3H-[1,2,3]triazolo[4,5-d]pyrimidin-5-yl}phenol;
3-{7-morpholin-4-yl-3-[2-(4-propionylpiperazin-1-yl)ethyl]-3H-[1,2,3]triazolo[4,5-d]pyrimidin-5-yl}phenol;
3-(3-{2-[4-(4-fluorobenzoyl)piperazin-1-yl]ethyl}-7-morpholin-4-yl-3H-[1,2,3]triazolo[4,5-d]pyrimidin-5-yl)phenol;
3-(3-{2-[4-(3,4-difluorobenzoyl)piperazin-1-yl]ethyl}-7-morpholin-4-yl-3H-[1,2,3]triazolo[4,5-d]pyrimidin-5-yl)phenol;
3-{3-[2-(4-isonicotinoylpiperazin-1-yl)ethyl]-7-morpholin-4-yl-3H-[1,2,3]triazolo[4,5-d]pyrimidin-5-yl}phenol;
3-(7-morpholin-4-yl-3-{2-[4-(phenylacetyl)piperazin-1-yl]ethyl}-3H-[1,2,3]triazolo[4,5-d]pyrimidin-5-yl)phenol;
3-{3-[2-(4-acetylpiperazin-1-yl)ethyl]-7-morpholin-4-yl-3H-[1,2,3]triazolo[4,5-d]pyrimidin-5-yl}phenol;
3-{3-[2-(4-cyclohexylpiperazin-1-yl)ethyl]-7-morpholin-4-yl-3H-[1,2,3]triazolo[4,5-d]pyrimidin-5-yl}phenol;
3-{3-[2-(4-butylpiperazin-1-yl)ethyl]-7-morpholin-4-yl-3H-[1,2,3]triazolo[4,5-d]pyrimidin-5-yl}phenol;
3-{3-[2-(4-isobutylpiperazin-1-yl)ethyl]-7-morpholin-4-yl-3H-[1,2,3]triazolo[4,5-d]pyrimidin-5-yl}phenol;
3-(3-{2-[4-(3-fluorobenzyl)piperazin-1-yl]ethyl}-7-morpholin-4-yl-3H-[1,2,3]triazolo[4,5-d]pyrimidin-5-yl)phenol;
3-{3-[2-(4-{4-[3-(dimethylamino)propoxy]benzyl}piperazin-1-yl)ethyl]-7-morpholin-4-yl-3H-[1,2,3]triazolo[4,5-d]pyrimidin-5-yl}phenol;
3-(7-morpholin-4-yl-3-{2-[4-(pyridin-3-ylmethyl)piperazin-1-yl]ethyl}-3H-[1,2,3]triazolo[4,5-d]pyrimidin-5-yl)phenol;
3-(7-morpholin-4-yl-3-{2-[4-(1H-pyrrol-2-ylmethyl)piperazin-1-yl]ethyl}-3H-[1,2,3]triazolo[4,5-d]pyrimidin-5-yl)phenol;
3-(3-{2-[4-(2-furylmethyl)piperazin-1-yl]ethyl}-7-morpholin-4-yl-3H-[1,2,3]triazolo[4,5-d]pyrimidin-5-yl)phenol;
3-{3-[2-(4-benzylpiperazin-1-yl)ethyl]-7-morpholin-4-yl-3H-[1,2,3]triazolo[4,5-d]pyrimidin-5-yl}phenol;
methyl 3-[5-(3-hydroxyphenyl)-7-morpholin-4-yl-3H-[1,2,3]triazolo[4,5-d]pyrimidin-3-yl]benzoate;
methyl 3-[5-(3-formylphenyl)-7-morpholin-4-yl-3H-[1,2,3]triazolo[4,5-d]pyrimidin-3-yl]benzoate;
[(7-morpholin-4-yl-3H-[1,2,3]triazolo[4,5-d]pyrimidine-3,5-diyl)di-3,1-phenylene]dimethanol;
3-[5-(3-hydroxyphenyl)-7-morpholin-4-yl-3H-[1,2,3]triazolo[4,5-d]pyrimidin-3-yl]benzoic acid;
3-[5-(3-hydroxyphenyl)-7-morpholin-4-yl-3H-[1,2,3]triazolo[4,5-d]pyrimidin-3-yl]benzamide;
3-(7-morpholin-4-yl-3-{3-[(4-pyrrolidin-1-ylpiperidin-1-yl)carbonyl]phenyl}-3H-[1,2,3]triazolo[4,5-d]pyrimidin-5-yl)phenol;
3-[5-(3-hydroxyphenyl)-7-morpholin-4-yl-3H-[1,2,3]triazolo[4,5-d]pyrimidin-3-yl]-N-methylbenzamide;
N-[2-(dimethylamino)ethyl]-3-[5-(3-hydroxyphenyl)-7-morpholin-4-yl-3H-[1,2,3]triazolo[4,5-d]pyrimidin-3-yl]benzamide;
3-(7-morpholin-4-yl-5-{4-[(phenylcarbamoyl)amino]phenyl}-3H-[1,2,3]triazolo[4,5-d]pyrimidin-3-yl)benzoic acid;
tert-butyl 3-[5-(3-hydroxyphenyl)-7-morpholin-4-yl-3H-[1,2,3]triazolo[4,5-d]pyrimidin-3-yl]azetidine-1-carboxylate;
3-(3-azetidin-3-yl-7-morpholin-4-yl-3H-[1,2,3]triazolo[4,5-d]pyrimidin-5-yl)phenol;
3-{3-[1-(2-aminobenzoyl)azetidin-3-yl]-7-morpholin-4-yl-3H-[1,2,3]triazolo[4,5-d]pyrimidin-5-yl}phenol;
3-[3-(1-benzylazetidin-3-yl)-7-morpholin-4-yl-3H-[1,2,3]triazolo[4,5-d]pyrimidin-5-yl]phenol;
3-(3-{1-[(6-fluoropyridin-3-yl)methyl]azetidin-3-yl}-7-morpholin-4-yl-3H-[1,2,3]triazolo[4,5-d]pyrimidin-5-yl)phenol;
(11bS)-11,11b-dimethyl-2,3,5,6,11,11b-hexahydro-1H-indolizino[8,7-b]indol-8-ol; diethyl 8-ethynyl-7-hydroxydibenzo[b,d]furan-3,4-dicarboxylate;
tert-butyl 3-(7-morpholin-4-yl-5-{4-[(phenylcarbamoyl)amino]phenyl}-3H-[1,2,3]triazolo[4,5-d]pyrimidin-3-yl)azetidine-1-carboxylate;
tert-butyl 3-(7-morpholin-4-yl-5-{4-[(2-thienylcarbamoyl)amino]phenyl}-3H-[1,2,3]triazolo[4,5-d]pyrimidin-3-yl)azetidine-1-carboxylate;
4-[7-morpholin-4-yl-3-(2,2,2-trifluoroethyl)-3H-[1,2,3]triazolo[4,5-d]pyrimidin-5-yl]aniline;
1-{4-[7-morpholin-4-yl-3-(2,2,2-trifluoroethyl)-3H-[1,2,3]triazolo[4,5-d]pyrimidin-5-yl]phenyl}-3-pyridin-4-ylurea;
1-{4-[7-morpholin-4-yl-3-(2,2,2-trifluoroethyl)-3H-[1,2,3]triazolo[4,5-d]pyrimidin-5-yl]phenyl}-3-pyridin-3-ylurea;
1-{4-[7-morpholin-4-yl-3-(2,2,2-trifluoroethyl)-3H-[1,2,3]triazolo[4,5-d]pyrimidin-5-yl]phenyl}-3-pyrimidin-5-ylurea;
1-[4-(dimethylamino)phenyl]-3-{4-[7-morpholin-4-yl-3-(2,2,2-trifluoroethyl)-3H-[1,2,3]triazolo[4,5-d]pyrimidin-5-yl]phenyl}urea;
1-[4-(2-hydroxyethyl)phenyl]-3-{4-[7-morpholin-4-yl-3-(2,2,2-trifluoroethyl)-3H-[1,2,3]triazolo[4,5-d]pyrimidin-5-yl]phenyl}urea;
tert-butyl methyl {2-[({4-[7-morpholin-4-yl-3-(2,2,2-trifluoroethyl)-3H-[1,2,3]triazolo[4,5-d]pyrimidin-5-yl]phenyl}carbamoyl)amino]ethyl}carbamate;
1-[2-(methylamino)ethyl]-3-{4-[7-morpholin-4-yl-3-(2,2,2-trifluoroethyl)-3H-[1,2,3]triazolo[4,5-d]pyrimidin-5-yl]phenyl}urea;
1-{4-[7-morpholin-4-yl-3-(2,2,2-trifluoroethyl)-3H-[1,2,3]triazolo[4,5-d]pyrimidin-5-yl]phenyl}-3-(2-thienyl)urea;
1-{4-[7-morpholin-4-yl-3-(2,2,2-trifluoroethyl)-3H-[1,2,3]triazolo[4,5-d]pyrimidin-5-yl]phenyl}-3-(3-thienyl)urea;
tert-butyl 4-[5-(3-hydroxyphenyl)-7-morpholin-4-yl-3H-[1,2,3]triazolo[4,5-d]pyrimidin-3-yl]piperidine-1-carboxylate;
3-(7-morpholin-4-yl-3-piperidin-4-yl-3H-[1,2,3]triazolo[4,5-d]pyrimidin-5-yl)phenol;
3-{7-morpholin-4-yl-3-[1-(1H-pyrrol-2-ylmethyl)piperidin-4-yl]-3H-[1,2,3]triazolo[4,5-d]pyrimidin-5-yl}phenol;
3-[3-(1-{4-[3-(dimethylamino)propoxy]benzyl}piperidin-4-yl)-7-morpholin-4-yl-3H-[1,2,3]triazolo[4,5-d]pyrimidin-5-yl]phenol;
3-{3-[1-(4-fluorobenzyl)piperidin-4-yl]-7-morpholin-4-yl-3H-[1,2,3]triazolo[4,5-d]pyrimidin-5-yl}phenol;
tert-butyl 4-[5-(2-aminopyrimidin-5-yl)-7-morpholin-4-yl-3H-[1,2,3]triazolo[4,5-d]pyrimidin-3-yl]piperidine-1-carboxylate;
3-{7-morpholin-4-yl-3-[1-(pyridin-2-ylmethyl)piperidin-4-yl]-3H-[1,2,3]triazolo[4,5-d]pyrimidin-5-yl}phenol;
tert-butyl 4-(7-morpholin-4-yl-5-{4-[(pyridin-3-ylcarbamoyl)amino]phenyl}-3H-[1,2,3]triazolo[4,5-d]pyrimidin-3-yl)piperidine-1-carboxylate;
tert-butyl 4-{5-[4-({[2-(dimethylamino)ethyl]carbamoyl}amino)phenyl]-7-morpholin-4-yl-3H-[1,2,3]triazolo[4,5-d]pyrimidin-3-yl}piperidine-1-carboxylate;
1-[2-(dimethylamino)ethyl]-3-[4-(7-morpholin-4-yl-3-piperidin-4-yl-3H-[1,2,3]triazolo[4,5-d]pyrimidin-5-yl)phenyl]urea;
1-[2-(dimethylamino)ethyl]-3-(4-{3-[1-(4-fluorobenzyl)piperidin-4-yl]-7-morpholin-4-yl-3H-[1,2,3]triazolo[4,5-d]pyrimidin-5-yl}phenyl)urea;
1-[2-(dimethylamino)ethyl]-3-(4-{7-morpholin-4-yl-3-[1-(pyridin-3-ylmethyl)piperidin-4-yl]-3H-[1,2,3]triazolo[4,5-d]pyrimidin-5-yl}phenyl)urea;
1-[4-(3-{1-[(6-bromopyridin-3-yl)methyl]piperidin-4-yl}-7-morpholin-4-yl-3H-[1,2,3]triazolo[4,5-d]pyrimidin-5-yl)phenyl]-3-[2-(dimethylamino)ethyl]urea;
1-(4-{3-[1-(4-chloro-2-fluorobenzyl)piperidin-4-yl]-7-morpholin-4-yl-3H-[1,2,3]triazolo[4,5-d]pyrimidin-5-yl}phenyl)-3-[2-(dimethylamino)ethyl]urea;
1-[2-(dimethylamino)ethyl]-3-[4-(3-{1-[(6-fluoropyridin-3-yl)methyl]piperidin-4-yl}-7-morpholin-4-yl-3H-[1,2,3]triazolo[4,5-d]pyrimidin-5-yl)phenyl]urea;
1-[2-(dimethylamino)ethyl]-3-[4-(3-{1-[(5-methyl-2-thienyl)methyl]piperidin-4-yl}-7-morpholin-4-yl-3H-[1,2,3]triazolo[4,5-d]pyrimidin-5-yl)phenyl]urea;
1-{4-[3-(1-butylpiperidin-4-yl)-7-morpholin-4-yl-3H-[1,2,3]triazolo[4,5-d]pyrimidin-5-yl]phenyl}-3-[2-(dimethylamino)ethyl]urea;
1-[2-(dimethylamino)ethyl]-3-(4-{7-morpholin-4-yl-3-[1-(4-pyridin-4-ylbenzyl)piperidin-4-yl]-3H-[1,2,3]triazolo[4,5-d]pyrimidin-5-yl}phenyl)urea;
1-[2-(dimethylamino)ethyl]-3-(4-{7-morpholin-4-yl-3-[1-(1H-pyrrol-2-ylmethyl)piperidin-4-yl]-3H-[1,2,3]triazolo[4,5-d]pyrimidin-5-yl}phenyl)urea;
1-[2-(dimethylamino)ethyl]-3-{4-[3-(1-{4-[3-(dimethylamino)propoxy]benzyl}piperidin-4-yl)-7-morpholin-4-yl-3H-[1,2,3]triazolo[4,5-d]pyrimidin-5-yl]phenyl}urea;
1-[4-(7-morpholin-4-yl-3-piperidin-4-yl-3H-[1,2,3]triazolo[4,5-d]pyrimidin-5-yl)phenyl]-3-pyridin-3-ylurea;
1-{4-[3-(1-methylpiperidin-4-yl)-7-morpholin-4-yl-3H-[1,2,3]triazolo[4,5-d]pyrimidin-5-yl]phenyl}-3-pyridin-3-ylurea;
tert-butyl 4-[5-(4-{[(4-fluorophenyl)carbamoyl]amino}phenyl)-7-morpholin-4-yl-3H-[1,2,3]triazolo[4,5-d]pyrimidin-3-yl]piperidine-1-carboxylate;
tert-butyl 4-(7-morpholin-4-yl-5-{4-[(pyridin-4-ylcarbamoyl)amino]phenyl}-3H-[1,2,3]triazolo[4,5-d]pyrimidin-3-yl)piperidine-1-carboxylate;
1-[4-(7-morpholin-4-yl-3-piperidin-4-yl-3H-[1,2,3]triazolo[4,5-d]pyrimidin-5-yl)phenyl]-3-pyridin-4-ylurea;
tert-butyl 4-(5-{4-[(methylcarbamoyl)amino]phenyl}-7-morpholin-4-yl-3H-[1,2,3]triazolo[4,5-d]pyrimidin-3-yl)piperidine-1-carboxylate;
tert-butyl 4-[5-(4-{[(methoxycarbonyl)carbamoyl]amino}phenyl)-7-morpholin-4-yl-3H-[1,2,3]triazolo[4,5-d]pyrimidin-3-yl]piperidine-1-carboxylate;
1-{4-[3-(1-benzylpiperidin-4-yl)-7-morpholin-4-yl-3H-[1,2,3]triazolo[4,5-d]pyrimidin-5-yl]phenyl}-3-(3-chlorophenyl)urea;
5-(3-{1-[(2-amino-1,3-thiazol-5-yl)methyl]piperidin-4-yl}-7-morpholin-4-yl-3H-[1,2,3]triazolo[4,5-d]pyrimidin-5-yl)pyridin-3-ol;
3-{3-[(1-ethylpyrrolidin-2-yl)methyl]-7-morpholin-4-yl-3H-[1,2,3]triazolo[4,5-d]pyrimidin-5-yl}phenol;
{5-[3-(1-benzylpiperidin-4-yl)-7-morpholin-4-yl-3H-[1,2,3]triazolo[4,5-d]pyrimidin-5-yl]pyridin-3-yl}methanol;
[5-(7-morpholin-4-yl-3-piperidin-4-yl-3H-[1,2,3]triazolo[4,5-d]pyrimidin-5-yl)pyridin-3-yl]methanol;
4-(3-ethyl-7-morpholin-4-yl-3H-[1,2,3]triazolo[4,5-d]pyrimidin-5-yl)-2-methoxyaniline;
[3-(7-morpholin-4-yl-3-piperidin-4-yl-3H-[1,2,3]triazolo[4,5-d]pyrimidin-5-yl)phenyl]methanol;
{3-[3-(1-benzylpiperidin-4-yl)-7-morpholin-4-yl-3H-[1,2,3]triazolo[4,5-d]pyrimidin-5-yl]phenyl}methanol;
4-[3-(2,2-dimethoxyethyl)-7-morpholin-4-yl-3H-[1,2,3]triazolo[4,5-d]pyrimidin-5-yl]aniline;
1-{4-[3-(2,2-dimethoxyethyl)-7-morpholin-4-yl-3H-[1,2,3]triazolo[4,5-d]pyrimidin-5-yl]phenyl}-3-(4-methylphenyl)urea;
1-{4-[3-(2,2-dimethoxyethyl)-7-morpholin-4-yl-3H-[1,2,3]triazolo[4,5-d]pyrimidin-5-yl]phenyl}-3-(4-fluorophenyl)urea;
1-{4-[3-(2,2-dimethoxyethyl)-7-morpholin-4-yl-3H-[1,2,3]triazolo[4,5-d]pyrimidin-5-yl]phenyl}-3-pyridin-3-ylurea;
4-[({4-[3-(2,2-dimethoxyethyl)-7-morpholin-4-yl-3H-[1,2,3]triazolo[4,5-d]pyrimidin-5-yl]phenyl}carbamoyl)amino]benzamide;
1-{4-[3-(2-hydroxyethyl)-7-morpholin-4-yl-3H-[1,2,3]triazolo[4,5-d]pyrimidin-5-yl]phenyl}-3-pyridin-4-ylurea;
1-{4-[3-(2-hydroxyethyl)-7-morpholin-4-yl-3H-[1,2,3]triazolo[4,5-d]pyrimidin-5-yl]phenyl}-3-pyridin-3-ylurea;
1-{4-[3-(2-hydroxyethyl)-7-morpholin-4-yl-3H-[1,2,3]triazolo[4,5-d]pyrimidin-5-yl]phenyl}-3-(4-methoxyphenyl)urea;
1-{4-[3-(2-hydroxyethyl)-7-morpholin-4-yl-3H-[1,2,3]triazolo[4,5-d]pyrimidin-5-yl]phenyl}-3-phenylurea;
tert-butyl 3-{[5-(4-aminophenyl)-7-morpholin-4-yl-3H-[1,2,3]triazolo[4,5-d]pyrimidin-3-yl]methyl}azetidine-1-carboxylate;
tert-butyl 3-[(7-morpholin-4-yl-5-{4-[(phenylcarbamoyl)amino]phenyl}-3H-[1,2,3]triazolo[4,5-d]pyrimidin-3-yl)methyl]azetidine-1-carboxylate;
1-{4-[3-(azetidin-3-ylmethyl)-7-morpholin-4-yl-3H-[1,2,3]triazolo[4,5-d]pyrimidin-5-yl]phenyl}-3-phenylurea;
1-(4-{3-[(1-benzoylazetidin-3-yl)methyl]-7-morpholin-4-yl-3H-[1,2,3]triazolo[4,5-d]pyrimidin-5-yl}phenyl)-3-phenylurea;
1-(4-{3-[(1-benzylazetidin-3-yl)methyl]-7-morpholin-4-yl-3H-[1,2,3]triazolo[4,5-d]pyrimidin-5-yl}phenyl)-3-phenylurea;
1-[4-(3-{[1-(4-fluorobenzyl)azetidin-3-yl]methyl}-7-morpholin-4-yl-3H-[1,2,3]triazolo[4,5-d]pyrimidin-5-yl)phenyl]-3-phenylurea;
1-[4-(7-morpholin-4-yl-3-{[1-(4-pyridin-4-ylbenzyl)azetidin-3-yl]methyl}-3H-[1,2,3]triazolo[4,5-d]pyrimidin-5-yl)phenyl]-3-phenylurea;
1-(4-{3-[(1-{4-[3-(dimethylamino)propoxy]benzyl}azetidin-3-yl)methyl]-7-morpholin-4-yl-3H-[1,2,3]triazolo[4,5-d]pyrimidin-5-yl}phenyl)-3-phenylurea;
3-[7-morpholin-4-yl-3-(2-piperidin-1-ylethyl)-3H-[1,2,3]triazolo[4,5-d]pyrimidin-5-yl]phenol;
3-[7-morpholin-4-yl-3-(2-pyridin-2-ylethyl)-3H-[1,2,3]triazolo[4,5-d]pyrimidin-5-yl]phenol;
4-chloro-N-(4-{[5-(3-hydroxyphenyl)-7-morpholin-4-yl-3H-[1,2,3]triazolo[4,5-d]pyrimidin-3-yl]methyl}phenyl)benzamide;
1-{4-[7-morpholin-4-yl-3-(tetrahydro-2H-pyran-4-yl)-3H-[1,2,3]triazolo[4,5-d]pyrimidin-5-yl]phenyl}-3-pyridin-4-ylurea;
1-[4-(3-methyl-7-morpholin-4-yl-3H-[1,2,3]triazolo[4,5-d]pyrimidin-5-yl)phenyl]-3-pyridin-3-ylurea;
1-[4-(3-methyl-7-morpholin-4-yl-3H-[1,2,3]triazolo[4,5-d]pyrimidin-5-yl)phenyl]-3-(2-thienyl)urea;
1-[4-(3-methyl-7-morpholin-4-yl-3H-[1,2,3]triazolo[4,5-d]pyrimidin-5-yl)phenyl]-3-(3-thienyl)urea;
3-{3-[4-(dimethylamino)butyl]-7-morpholin-4-yl-3H-[1,2,3]triazolo[4,5-d]pyrimidin-5-yl}phenol;
3-{3-[4-(methylamino)butyl]-7-morpholin-4-yl-3H-[1,2,3]triazolo[4,5-d]pyrimidin-5-yl}phenol;
3-[3-(4-aminobutyl)-7-morpholin-4-yl-3H-[1,2,3]triazolo[4,5-d]pyrimidin-5-yl]phenol;
3-[7-morpholin-4-yl-3-(4-pyrrolidin-1-ylbutyl)-3H-[1,2,3]triazolo[4,5-d]pyrimidin-5-yl]phenol;
3-{3-[4-(4-benzylpiperazin-1-yl)butyl]-7-morpholin-4-yl-3H-[1,2,3]triazolo[4,5-d]pyrimidin-5-yl}phenol;
4-{[5-(3-hydroxyphenyl)-7-morpholin-4-yl-3H-[1,2,3]triazolo[4,5-d]pyrimidin-3-yl]methyl}-N-methylbenzamide;
tert-butyl 4-[(4-{[5-(3-hydroxyphenyl)-7-morpholin-4-yl-3H-[1,2,3]triazolo[4,5-d]pyrimidin-3-yl]methyl}benzoyl)amino]piperidine-1-carboxylate;
tert-butyl [1-(4-{[5-(3-hydroxyphenyl)-7-morpholin-4-yl-3H-[1,2,3]triazolo[4,5-d]pyrimidin-3-yl]methyl}benzoyl)piperidin-4-yl]carbamate;
N-(2-acetamidoethyl)-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(3-pyrrolidin-1-ylpropyl)benzamide;
N-benzyl-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(2-pyrrolidin-1-ylethyl)benzamide;
N-[2-(dimethylamino)ethyl]-4-{[5-(3-hydroxyphenyl)-7-morpholin-4-yl-3H-[1,2,3]triazolo[4,5-d]pyrimidin-3-yl]methyl}benzamide;
N-[3-(dimethylamino)propyl]-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-pyridin-3-ylbenzamide;
N-(4-fluorophenyl)-4-{[5-(3-hydroxyphenyl)-7-morpholin-4-yl-3H-[1,2,3]triazolo[4,5-d]pyrimidin-3-yl]methyl}benzamide;
tert-butyl 4-{4-[(4-{[5-(3-hydroxyphenyl)-7-morpholin-4-yl-3H-[1,2,3]triazolo[4,5-d]pyrimidin-3-yl]methyl}benzoyl)amino]phenyl}piperazine-1-carboxylate;
N-ethyl-4-{[5-(3-hydroxyphenyl)-7-morpholin-4-yl-3H-[1,2,3]triazolo[4,5-d]pyrimidin-3-yl]methyl}benzamide;
N,N-diethyl-4-{[5-(3-hydroxyphenyl)-7-morpholin-4-yl-3H-[1,2,3]triazolo[4,5-d]pyrimidin-3-yl]methyl}benzamide;
N-cyclopropyl-4-{[5-(3-hydroxyphenyl)-7-morpholin-4-yl-3H-[1,2,3]triazolo[4,5-d]pyrimidin-3-yl]methyl}benzamide;
N-tert-butyl-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(2-phenylethyl)benzamide;
4-{[5-(3-hydroxyphenyl)-7-morpholin-4-yl-3H-[1,2,3]triazolo[4,5-d]pyrimidin-3-yl]methyl}-N-[(1S)-1-phenylethyl]benzamide;
4-{[5-(3-hydroxyphenyl)-7-morpholin-4-yl-3H-[1,2,3]triazolo[4,5-d]pyrimidin-3-yl]methyl}-N-[2-(1H-indol-3-yl)ethyl]benzamide;
N-(2-hydroxy-2-phenylethyl)-4-{[5-(3-hydroxyphenyl)-7-morpholin-4-yl-3H-[1,2,3]triazolo[4,5-d]pyrimidin-3-yl]methyl}benzamide;
3-{7-morpholin-4-yl-3-[4-(piperidin-1-ylcarbonyl)benzyl]-3H-[1,2,3]triazolo[4,5-d]pyrimidin-5-yl}phenol;
3-{7-morpholin-4-yl-3-[4-(pyrrolidin-1-ylcarbonyl)benzyl]-3H-[1,2,3]triazolo[4,5-d]pyrimidin-5-yl}phenol;
3-(7-morpholin-4-yl-3-{4-[(4-phenylpiperazin-1-yl)carbonyl]benzyl}-3H-[1,2,3]triazolo[4,5-d]pyrimidin-5-yl)phenol;
N-(2-furylmethyl)-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-[2-(1H-imidazol-5-yl)ethyl]benzamide;
tert-butyl {5-[3-(hydroxymethyl)phenyl]-7-morpholin-4-yl-3H-[1,2,3]triazolo[4,5-d]pyrimidin-3-yl}acetate;
tert-butyl [5-(3-hydroxyphenyl)-7-morpholin-4-yl-3H-[1,2,3]triazolo[4,5-d]pyrimidin-3-yl]acetate;
tert-butyl (7-morpholin-4-yl-5-{4-[(pyridin-4-ylcarbamoyl)amino]phenyl}-3H-[1,2,3]triazolo[4,5-d]pyrimidin-3-yl)acetate;
2-{5-[3-(hydroxymethyl)phenyl]-7-morpholin-4-yl-3H-[1,2,3]triazolo[4,5-d]pyrimidin-3-yl}-N-pyridin-3-ylacetamide;
2-{5-[3-(hydroxymethyl)phenyl]-7-morpholin-4-yl-3H-[1,2,3]triazolo[4,5-d]pyrimidin-3-yl}-N-methylacetamide;
2-{5-[3-(hydroxymethyl)phenyl]-7-morpholin-4-yl-3H-[1,2,3]triazolo[4,5-d]pyrimidin-3-yl}acetamide;
N-(4-fluorophenyl)-2-{5-[3-(hydroxymethyl)phenyl]-7-morpholin-4-yl-3H-[1,2,3]triazolo[4,5-d]pyrimidin-3-yl}acetamide;
N-[2-(dimethylamino)ethyl]-2-{5-[3-(hydroxymethyl)phenyl]-7-morpholin-4-yl-3H-[1,2,3]triazolo[4,5-d]pyrimidin-3-yl}acetamide;
{5-[3-(hydroxymethyl)phenyl]-7-morpholin-4-yl-3H-[1,2,3]triazolo[4,5-d]pyrimidin-3-yl}acetic acid;
methyl 4-{[5-(3-hydroxyphenyl)-7-morpholin-4-yl-3H-[1,2,3]triazolo[4,5-d]pyrimidin-3-yl]methyl}benzoate;
4-{[5-(3-hydroxyphenyl)-7-morpholin-4-yl-3H-[1,2,3]triazolo[4,5-d]pyrimidin-3-yl]methyl}benzoic acid;
methyl 4-({5-[3-(hydroxymethyl)phenyl]-7-morpholin-4-yl-3H-[1,2,3]triazolo[4,5-d]pyrimidin-3-yl}methyl)benzoate;
methyl 4-{[5-(3-fluoro-5-hydroxyphenyl)-7-morpholin-4-yl-3H-[1,2,3]triazolo[4,5-d]pyrimidin-3-yl]methyl}benzoate;
and [5-(3-hydroxyphenyl)-7-morpholin-4-yl-3H-[1,2,3]triazolo[4,5-d]pyrimidin-3-yl]acetic acid.

Illustrative compounds of Formula 1 include the following compounds:
1-{4-[(2,2-dimethylhydrazino)carbonyl]phenyl}-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-(4-nitrophenyl)urea;
1-(4-aminophenyl)-3-[4-(3-ethyl-7-morpholin-4-yl-3H-[1,2,3]triazolo[4,5-d]pyrimidin-5-yl)phenyl]urea;
N-[4-({[4-(3-ethyl-7-morpholin-4-yl-3H-[1,2,3]triazolo[4,5-d]pyrimidin-5-yl)phenyl]carbamoyl}amino)phenyl]-N2,N2-dimethylglycinamide;
3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoic acid;
4-[({4-[3-(3-carbamoylphenyl)-7-morpholin-4-yl-3H-[1,2,3]triazolo[4,5-d]pyrimidin-5-yl]phenyl}carbamoyl)amino]-N-[2-(dimethylamino)ethyl]benzamide;
1-[4-(3-ethyl-7-morpholin-4-yl-3H-[1,2,3]triazolo[4,5-d]pyrimidin-5-yl)phenyl]-3-{4-[(pyridin-4-ylmethyl)amino]phenyl}urea;
1-[4-(3-ethyl-7-morpholin-4-yl-3H-[1,2,3]triazolo[4,5-d]pyrimidin-5-yl)phenyl]-3-{4-[(pyridin-3-ylmethyl)amino]phenyl}urea;
1-[4-(3-ethyl-7-morpholin-4-yl-3H-[1,2,3]triazolo[4,5-d]pyrimidin-5-yl)phenyl]-3-(4-{[(6-fluoropyridin-3-yl)methyl]amino}phenyl)urea;
1-[4-(3-ethyl-7-morpholin-4-yl-3H-[1,2,3]triazolo[4,5-d]pyrimidin-5-yl)phenyl]-3-(4-{[(6-methoxypyridin-3-yl)methyl]amino}phenyl)urea;
N-[2-(dimethylamino)ethyl]-4-[({4-[3-(1-methylethyl)-7-morpholin-4-yl-3H-[1,2,3]triazolo[4,5-d]pyrimidin-5-yl]phenyl}carbamoyl)amino]benzamide;
1-{4-[3-(1-methylethyl)-7-morpholin-4-yl-3H-[1,2,3]triazolo[4,5-d]pyrimidin-5-yl]phenyl}-3-{4-[(4-methylpiperazin-1-yl)carbonyl]phenyl}urea;
1-[4-(3-ethyl-7-morpholin-4-yl-3H-[1,2,3]triazolo[4,5-d]pyrimidin-5-yl)phenyl]-3-{4-[(4-methylpiperazin-1-yl)methyl]phenyl}urea;
1-[4-(3-ethyl-7-morpholin-4-yl-3H-[1,2,3]triazolo[4,5-d]pyrimidin-5-yl)phenyl]-3-[4-(4-methylpiperazin-1-yl)phenyl]urea;
4-({[4-(3-ethyl-7-morpholin-4-yl-3H-[1,2,3]triazolo[4,5-d]pyrimidin-5-yl)phenyl]carbamoyl}amino)-N-pyridin-3-ylbenzamide;
N-[4-({[4-(3-ethyl-7-morpholin-4-yl-3H-[1,2,3]triazolo[4,5-d]pyrimidin-5-yl)phenyl]carbamoyl}amino)phenyl]-4-methylpiperazine-1-carboxamide;
N-[4-({[4-(3-ethyl-7-morpholin-4-yl-3H-[1,2,3]triazolo[4,5-d]pyrimidin-5-yl)phenyl]carbamoyl}amino)phenyl]pyridine-4-carboxamide;
N-[4-({[4-(3-ethyl-7-morpholin-4-yl-3H-[1,2,3]triazolo[4,5-d]pyrimidin-5-yl)phenyl]carbamoyl}amino)phenyl]morpholine-4-carboxamide;
3-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]benzamide;
1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]urea;
4-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]piperidine-1-carboxamide;
1-[4-(3-ethyl-7-morpholin-4-yl-3H-[1,2,3]triazolo[4,5-d]pyrimidin-5-yl)phenyl]-3-(4-{[(1-methylpiperidin-4-yl)carbamoyl]amino}phenyl)urea;
1-[4-(3-ethyl-7-morpholin-4-yl-3H-[1,2,3]triazolo[4,5-d]pyrimidin-5-yl)phenyl]-3-[4-({[2-(4-methylpiperazin-1-yl)ethyl]carbamoyl}amino)phenyl]urea;
N-[4-({[4-(3-ethyl-7-morpholin-4-yl-3H-[1,2,3]triazolo[4,5-d]pyrimidin-5-yl)phenyl]carbamoyl}amino)phenyl]-4-methyl-1,4-diazepane-1-carboxamide;
1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]-1-methylurea;
1-[4-(3-ethyl-7-morpholin-4-yl-3H-[1,2,3]triazolo[4,5-d]pyrimidin-5-yl)phenyl]-3-(4-{[(2-pyrrolidin-1-ylethyl)carbamoyl]amino}phenyl)urea;
N-[4-({[4-(3-ethyl-7-morpholin-4-yl-3H-[1,2,3]triazolo[4,5-d]pyrimidin-5-yl)phenyl]carbamoyl}amino)phenyl]-4-pyrrolidin-1-ylpiperidine-1-carboxamide;
1-[4-(3-ethyl-7-morpholin-4-yl-3H-[1,2,3]triazolo[4,5-d]pyrimidin-5-yl)phenyl]-3-(4-{[(pyridin-2-ylmethyl)carbamoyl]amino}phenyl)urea;
N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide;
4-ethyl-N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide;
1-[4-(3-ethyl-7-morpholin-4-yl-3H-[1,2,3]triazolo[4,5-d]pyrimidin-5-yl)phenyl]-3-(4-{[(2-methoxyethyl)carbamoyl]amino}phenyl)urea;
1-{4-[3-(1-methylethyl)-7-morpholin-4-yl-3H-[1,2,3]triazolo[4,5-d]pyrimidin-5-yl]phenyl}-3-[4-(4-methylpiperazin-1-yl)phenyl]urea;
1-{4-[3-(1-methylethyl)-7-morpholin-4-yl-3H-[1,2,3]triazolo[4,5-d]pyrimidin-5-yl]phenyl}-3-(4-nitrophenyl)urea;
N-[4-({[4-(3-isopropyl-7-morpholin-4-yl-3H-[1,2,3]triazolo[4,5-d]pyrimidin-5-yl)phenyl]carbamoyl}amino)phenyl]methanesulfonamide;
1-(4-aminophenyl)-3-[4-(3-isopropyl-7-morpholin-4-yl-3H-[1,2,3]triazolo[4,5-d]pyrimidin-5-yl)phenyl]urea;
1-(4-{[4-(dimethylamino)piperidin-1-yl]carbonyl}phenyl)-3-(4-{3-ethyl-7-[(3S)-3-methylmorpholin-4-yl]-3H-[1,2,3]triazolo[4,5-d]pyrimidin-5-yl}phenyl)urea;
1-(4-{3-ethyl-7-[(3S)-3-methylmorpholin-4-yl]-3H-[1,2,3]triazolo[4,5-d]pyrimidin-5-yl}phenyl)-3-{4-[(4-methylpiperazin-1-yl)carbonyl]phenyl}urea;
4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-pyrrolidin-1-ylethyl)benzamide;
4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-piperidin-1-ylethyl)benzamide;
N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-methylbenzamide;
N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}benzamide;
methyl 5-({[4-(3-ethyl-7-morpholin-4-yl-3H-[1,2,3]triazolo[4,5-d]pyrimidin-5-yl)phenyl]carbamoyl}amino)pyridine-2-carboxylate;
5-({[4-(3-ethyl-7-morpholin-4-yl-3H-[1,2,3]triazolo[4,5-d]pyrimidin-5-yl)phenyl]carbamoyl}amino)pyridine-2-carboxylic acid;
1-[4-(3-ethyl-7-morpholin-4-yl-3H-[1,2,3]triazolo[4,5-d]pyrimidin-5-yl)phenyl]-3-{6-[(4-methylpiperazin-1-yl)carbonyl]pyridin-3-yl}urea;
and N-[2-(dimethylamino)ethyl]-5-({[4-(3-ethyl-7-morpholin-4-yl-3H-[1,2,3]triazolo[4,5-d]pyrimidin-5-yl)phenyl]carbamoyl}amino)-N-methylpyridine-2-carboxamide.

Illustrative compounds of Formula 1 include the following compounds:
N-(2-(dimethylamino)ethyl)-N-methyl-4-(3-(4-(3-methyl-7-morpholino-3H-[1,2,3]triazolo[4,5-d]pyrimidin-5-yl)phenyl)ureido)benzamide;
N-(2-(dimethylamino)ethyl)-4-(3-(4-(3-methyl-7-morpholino-3H-[1,2,3]triazolo[4,5-d]pyrimidin-5-yl)phenyl)ureido)benzamide;
1-(4-(3-methyl-7-morpholino-3H-[1,2,3]triazolo[4,5-d]pyrimidin-5-yl)phenyl)-3-(4-(4-methylpiperazine-1-carbonyl)phenyl)urea;
1-(4-(4-(dimethylamino)piperidine-1-carbonyl)phenyl)-3-(4-(3-methyl-7-morpholino-3H-[1,2,3]triazolo[4,5-d]pyrimidin-5-yl)phenyl)urea;
N-(2-(dimethylamino)ethyl)-N-methyl-4-(3-(4-(7-morpholino-3-(2,2,2-trifluoroethyl)-3H-[1,2,3]triazolo[4,5-d]pyrimidin-5-yl)phenyl)ureido)benzamide;
N-(2-(dimethylamino)ethyl)-4-(3-(4-(7-morpholino-3-(2,2,2-trifluoroethyl)-3H-[1,2,3]triazolo[4,5-d]pyrimidin-5-yl)phenyl)ureido)benzamide;
1-(4-(4-methylpiperazine-1-carbonyl)phenyl)-3-(4-(7-morpholino-3-(2,2,2-trifluoroethyl)-3H-[1,2,3]triazolo[4,5-d]pyrimidin-5-yl)phenyl)urea;
and 1-(4-(4-(dimethylamino)piperidine-1-carbonyl)phenyl)-3-(4-(7-morpholino-3-(2,2,2-trifluoroethyl)-3H-[1,2,3]triazolo[4,5-d]pyrimidin-5-yl)phenyl)urea.

As some of the compounds of the present invention possess an asymmetric carbon atom in the morpholine ring, the present invention includes the racemate as well as the individual enantiomeric forms of the compounds of Formula 1 as described herein and in the claims. Mixtures of isomers of the compounds of the examples or chiral precursors thereof can be separated into individual isomers according to methods, which are known per se, e.g. fractional crystallization, adsorption chromatography or other suitable separation processes. Resulting racemates can be separated into antipodes in the usual manner after introduction of suitable salt-forming groupings, e.g. by forming a mixture of diastereosiomeric salts with optically active salt-forming agents, separating the mixture into diastereomeric salts and converting the separated salts into the free compounds. The enantiomeric forms may also be separated by fractionation through chiral high-pressure liquid chromatography columns.

The invention also includes pharmaceutical compositions comprising an effective amount of a 3H-[1,2,3]triazolo[4,5-d]pyrimidine compound of Formula 1 and a pharmaceutically acceptable carrier. The compound may be provided as a pharmaceutically acceptable prodrug, hydrated salt, such as a pharmaceutically acceptable salt, or mixtures thereof.

In another aspect, the invention provides methods of synthesizing compounds of the Formula 1 comprising: reacting a boronic acid of the formula (R²)ᵣ—Ar—B(OH)₂ with the 5-halo-3H-[1,2,3]triazolo[4,5-d]pyrimidine 2:

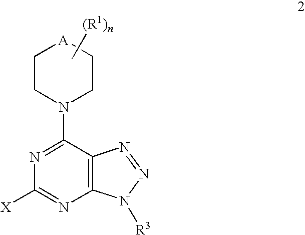

wherein X is halo and A, Ar, R¹, n, R², r, and R³, are as defined in Formula 1;

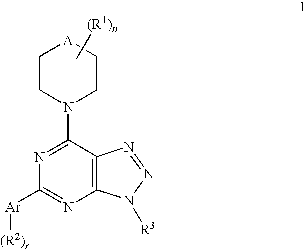

thereby producing the 3H-[1,2,3]triazolo[4,5-d]pyrimidine 1.

In one aspect, the invention provides methods of synthesizing compounds of the Formula 1 further comprising: (a) reacting the 2,4,6-trihalo-5-nitropyrimidine of Formula 3 with an amine 4 to substitute the halogen

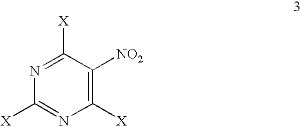

atom at position 4 of the pyrimidine

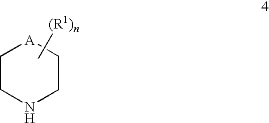

thereby producing 5:

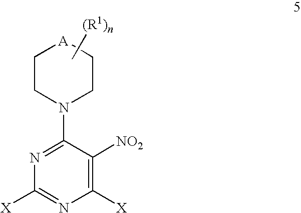

(b) reacting dihalo pyrimidine 5 with amine R³—NH₂ replacing the halogen atom at position 6 of the pyrimidine ring with radical R³—NH—;
c) reducing the product of the proceeding reaction to convert the nitro group at position 5 of the pyrimidine ring to an amino group without removing the halogen atom at position 2 of the pyrimidine;
d) diazotizing and cyclizing the diaminopyrimidine;

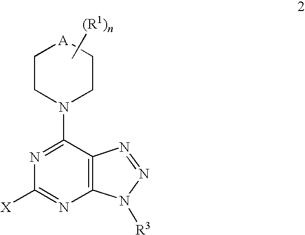

thereby producing 3H-[1,2,3]triazolo[4,5-d]pyrimidine 2.

Representative “pharmaceutically acceptable salts” include but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (4,4′-methylenebis-3-hydroxy-2-naphthoate, or embonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection with a 3H-[1,2,3]triazolo[4,5-d]pyrimidine compound of this invention is an amount effective for inhibiting mTOR or PI3K in a subject.

The following abbreviations are used herein and have the indicated definitions: ACN is acetonitrile, AcOH is acetic acid. ATP is adenosine triphosphate. BOC is t-butoxycarbonyl. Celite™ is flux-calcined diatomaceous earth. Celite™ is a registered trademark of World Minerals Inc. CHAPS is (3-[(3-cholamidopropyl)dimethylammonio]-1-propanesulfonic acid, DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation. EDCI is 3′-dimethylaminopropyl)carbodiimide or water-soluble carbodiimide, EDTA is ethylenediaminetetraacetic acid, ESI stands for Electrospray Ionization, EtOAc is ethyl acetate, and EtOH is ethanol. HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, MS is mass spectrometry, and NEt₃ is triethylamine. Ni(Ra) is Raney™ nickel, a sponge-metal catalyst produced when a block of nickel-aluminum alloy is treated with concentrated sodium hydroxide. Raney™ is a registered trademark of W. R. Grace and Company. NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

The following definitions are used in connection with the 3H-[1,2,3]triazolo[4,5-d]pyrimidine compounds of the present invention, unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like.

“Acyl” refers to a carbonyl group bonded to a moiety comprising from 1 to 8 carbon atoms in a straight, branched, or cyclic configuration or a combination thereof, attached to the parent structure through the carbonyl functionality. The moiety may be saturated or unsaturated, aliphatic or aromatic, and carbocyclic or heterocyclic. One or more carbons in the moiety may be replaced by oxygen, nitrogen (e.g., carboxyamido), or sulfur as long as the point of attachment to the parent remains at the carbonyl. Examples of C₁-C₈acyl include acetyl-, benzoyl-, nicotinoyl, propionyl-, isobutyryl-, oxalyl-, t-butoxycarbonyl-, benzyloxycarbonyl, morpholinylcarbonyl, and the like. An acyl group can be unsubstituted or substituted with one or more, e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, or C₃-C₈cycloalkyl.

“Alkenyl” refers to a straight or branched chain unsaturated hydrocarbon containing 2-10 carbon atoms and at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. An alkenyl group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more e.g. 1 to 3 of the following groups which may be the same or different: halogen, hydroxyl, C₁-C₆alkoxy, —NH₂, —NH(C₁-C₆alkyl), —N(C₁-C₆alkyl) (C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C 11-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“(Alkoxy)carbonyl” refers to the group alkyl-O—C(O)—. An (alkoxy)carbonyl group can be unsubstituted or substituted with one or more e.g. 1 to 3 of the following groups which may be the same or different: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂. Exemplary (C₁-C₆alkoxy)carbonyl groups include but are not limited to CH₃—O—C(O)—, CH₃CH₂—O—C(O)—, CH₃CH₂CH₂—O—C(O)—, (CH₃)₂CH—O—C(O)—, CH₃CH₂CH₂CH₂—O—C(O)—, and t-butoxycarbonyl.

“Alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 6 (inclusive) carbon atoms in it. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“(Alkyl)amido-” refers to a —C(O)NH— group in which the nitrogen atom of said group is attached to an alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, C(O)NHCH₂CH(CH₃)₂, —C(O)NHCH(CH₃)CH₂CH₃, —C(O)NH—C(CH₃)₃ and C(O)NHCH₂C(CH₃)₃.

“(Alkyl)amino-” refers to an —NH group, the nitrogen atom of said group being attached to an alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“Alkylcarboxy” refers to an alkyl group as defined above, attached to the parent structure through the oxygen atom of a carboxyl (C(O)—O—) functionality. Examples of C₁-C₆alkylcarboxy include acetoxy, ethylcarboxy, propylcarboxy, and isopentylcarboxy.

“(Alkyl)carboxyamido-” refers to a —NHC(O)— group in which the carbonyl carbon atom of said group is attached to an alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to the subsets of alkyl, alkenyl and alkynyl groups, as defined herein, including the same residues as alkyl, alkenyl, and alkynyl, but having two points of attachment within a chemical structure. Examples of C₁-C₆alkylene include methylene (—CH₂—), ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to groups of straight chain or branched chain with 1 to 6 carbon atoms, attached to the parent structure through a sulfur atom. Examples of a C₁-C₆alkylthio group include methylthio, ethylthio, n-propylthio, i-propylthio, n-butylthio, i-butylthio, s-butylthio, t-butylthio, n-pentylthio, and n-hexylthio.

“Alkynyl” refers to a straight or branched chain unsaturated hydrocarbon containing 2-10 carbon atoms, respectively, and at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. A alkynyl group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl)-group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Amino(alkyl)-” refers to an alkyl group, as defined above, wherein one or more of the alkyl group's hydrogen atoms has been replaced with —NH₂. Representative examples of an amino(C₁-C₆alkyl) group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂ (CH₂NH₂), —CH₂CH₂CH₂CH₂CH₂NH₂, and —CH₂CH₂CH(NH₂)CH₂CH₃. An amino(alkyl) group can be unsubstituted or substituted with one or two of the following groups C₁-C₆alkoxy, C₆-C₁₄aryl, C₁-C₉heteroaryl, C₃-C₈cycloalkyl, and C₁-C₆alkyl which may be the same or different.

“Aryl” refers to an aromatic hydrocarbon group. If not otherwise specified, in this specification the term aryl refers to a C₆-C₁₄aryl group. Examples of an C₆-C₁₄aryl group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl, groups. An aryl group can be unsubstituted or substituted with one or more e.g. 1 to 3 of the following groups which may be the same or different: C₁-C₆alkyl, C₃-C₈cycloalkyl, C₁-C₆ perfluoroalkyl-, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O) (C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with an C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more e.g. 1 to 3 of the following groups which may be the same or different: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“(Aryl)amino” refers to a radical of formula aryl-NH—, wherein “aryl” is as defined above. Examples of (C₆-C₁₄aryl)amino radicals include, but are not limited to, phenylamino (anilido), 1-naphthylamino, 2-naphthylamino and the like. An (aryl)amino group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, or C₃-C₈cycloalkyl.

“(Aryl)oxy” refers to the group Ar—O— where Ar is an aryl group, as defined above. Exemplary (C₆-C₁₄aryl)oxy groups include but are not limited to phenyloxy, α-naphthyloxy, and β-naphthyloxy. A (aryl)oxy group can be unsubstituted or substituted with one or more e.g. 1 to 3 of the following groups which may be the same or different: C₁-C₆alkyl, halo, haloalkyl-, hydroxyl, C₁-C₆hydroxylalkyl-, —NH₂, aminoalkyl-, -dialkylamino-, —COOH, —C(O)O—(C₁-C₆alkyl), —OC(O) (C₁-C₆alkyl), N-alkylamido-, —C(O)NH₂, (C₁-C₆alkyl)amido-, or —NO₂.

“Cycloalkyl” refers to a monocyclic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. A cycloalkyl can be unsubstituted or independently substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂. Additionally, each of any two hydrogen atoms on the same carbon atom of the cycloalkyl ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Bicyclic cycloalkyl” refers to a bicyclic, saturated hydrocarbon ring system containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamidoalkyl-” refers to a primary carboxyamide (—CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an alkylene group as defined above. Exemplary C₁-C₆-carboxyamidoalkyl-groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to non-aromatic, carbocyclic rings containing 3-10 carbon atoms with one or more carbon-to-carbon double bonds within the ring system. The “cycloalkenyl” may be a single ring or may be multi-ring. Multi-ring structures may be bridged or fused ring structures. A cycloalkenyl can be unsubstituted or independently substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆-carboxyamidoalkyl-, or —NO₂ Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom which has attached to it two alkyl groups, as defined above. Each alkyl group can be independently selected from the alkyl groups. Representative examples of an di(C₁-C₆alkyl)amino-group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ᵣ—. R is hydrogen, C₁-C₆alkyl, C₃-C₈cycloalkyl, C₆-C₁₄aryl, C₁-C₉heteroaryl, amino(C₁-C₆alkyl), or arylamino. Variable r is 0, 1, or 2.

“Halo” or “Halogen” is —F, —Cl, —Br or —I.

“Haloalkyl” refers to an alkyl group, as defined above, wherein one or more of the alkyl group's hydrogen atoms has been replaced with —F, —Cl, —Br, or —I. Each substitution can be independently selected from —F, —Cl, —Br, or —I. Representative examples of an C₁-C₆haloalkyl group include, but are not limited to —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂ CH(CI)CH₂CH₃, —CH(F)CH₂CH₃ and —C(CH₃)₂ (CH₂Cl).

“Heteroaryl” refers to 5-10-membered mono and bicyclic aromatic groups containing at least one heteroatom selected from oxygen, sulfur and nitrogen e.g. it can suitably contain 1 to 3 heteroatoms. Examples of monocyclic C₁-C₅heteroaryl radicals include, but are not limited to, pyrrolyl, thiazinyl, diazinyl, triazinyl, tetrazinyl, imidazolyl, tetrazolyl, isoxazolyl, furanyl, furazanyl, oxazolyl, thiazolyl, isothiazolyl, thiophenyl, pyrazolyl, triazolyl, pyrimidinyl, pyrazinyl, pyridazinyl, N-pyridyl, 2-pyridyl, 3-pyridyl and 4-pyridyl. Examples of C₁-C₉bicyclic heteroaryl radicals include but are not limited to, benzimidazolyl, indolyl, isoquinolinyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Heteroaryl(alkyl)” refers to an alkyl group, as defined above, wherein one or more of the alkyl group's hydrogen atoms has been replaced with a heteroaryl group as defined above. Heteroaryl(C₁-C₆alkyl) moieties include 2-pyridylmethyl, 2-thiophenylethyl, 3-pyridylpropyl, 2-quinolinylmethyl, 2-indolylmethyl, and the like. A heteroaryl(alkyl) group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

“(Heteroaryl)oxy” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more e.g. 1 to 3 of the following groups which may be the same or different: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

The term “heteroatom” refers to a sulfur, nitrogen, or oxygen atom.

“Heterocycle” refers to 3-10-membered mono and bicyclic groups containing at least one heteroatom selected from oxygen, sulfur and nitrogen e.g. it can suitably contain 1 to 3 heteroatoms. A heterocycle may be saturated or partially saturated. Exemplary C₁-C₉heterocycle groups include but are not limited to aziridine, oxirane, thiirane, pyrroline, pyrrolidine, dihydrofuran, tetrahydrofuran, dihydrothiophene, tetrahydrothiophene, dithiolane, piperidine, tetrahydropyran, pyran, thiane, thiine, piperazine, oxazine, thiazine, dithiane, dioxane, tetrahydroquinoline, and tetrahydroisoquinoline.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl) moieties include 1-piperazinylethyl, 4-morpholinylpropyl, 6-piperazinylhexyl, and the like. A heterocyclyl(alkyl) group can be unsubstituted or substituted with one or more e.g. 1 to 3 of the following groups which may be the same or different: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

“Hydroxylalkyl-” refers to an alkyl group, as defined above, wherein one or more of the alkyl group's hydrogen atoms has been replaced with hydroxyl groups. Examples of C₁-C₆hydroxylalkyl-moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to a straight or branched chain hydrocarbon, containing 3-6 carbon atoms, and at least one double bond, substituted on one or more sp³ carbon atom with a hydroxyl group. Examples of C₃-C₆hydroxylalkenyl-moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Monocyclic heterocycle” refers to a monocyclic cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a monocyclic C₁-C₆heterocycle group include, but are not limited to, piperidinyl, 1,2,5,6-tetrahydropyridinyl, piperazinyl, morpholinyl, oxazinyl, thiazinyl, pyrrolinyl, pyrrolidinyl, and homopiperidinyl. A monocyclic heterocycle group can be unsubstituted or substituted with one or more e.g. 1 to 3 of the following groups which may be the same or different: C₁-C₈acyl, C₁-C₆alkyl, heterocyclyl(C₁-C₆alkyl), (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Bicyclic heterocycle” refers to a bicyclic cycloalkyl or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a bicyclic C₁-C₉heterocycle group include, but are not limited to, indolinyl, tetrahydroisoquinolinyl, tetrahydroquinolinyl, and chromanyl. A bicyclic heterocycle group can be unsubstituted or substituted with one or more e.g. 1 to 3 of the following groups which may be the same or different: C₁-C₈acyl, C₁-C₆alkyl, heterocyclyl(C₁-C₆alkyl), (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Perfluoroalkyl-” refers to a straight or branched chain hydrocarbon having two or more fluorine atoms. Examples of a C₁-C₆ perfluoroalkyl-group include CF₃, CH₂CF₃, CF₂CF₃ and CH(CF₃)₂.

The term “optionally substituted”, unless otherwise specified, as used herein means that at least one hydrogen atom e.g. 1 to 3 atoms of the optionally substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1₁-C₆alkyl), —C(O)(C₁-C₆alkyl), C₆-C₁₄aryl, C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The 3H-[1,2,3]triazolo[4,5-d]pyrimidine compounds of the present invention exhibit an PI3K inhibitory activity and therefore, can be utilized in order to inhibit abnormal cell growth in which PI3K plays a role. Thus, the 3H-[1,2,3]triazolo[4,5-d]pyrimidine compounds are effective in the treatment of disorders with which abnormal cell growth actions of PI3K are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the 3H-[1,2,3]triazolo[4,5-d]pyrimidine compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The 3H-[1,2,3]triazolo[4,5-d]pyrimidine compounds of the present invention exhibit an mTOR inhibitory activity and therefore, can be utilized in order to inhibit abnormal cell growth in which mTOR plays a role. Thus, the 3H-[1,2,3]triazolo[4,5-d]pyrimidine compounds are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the 3H-[1,2,3]triazolo[4,5-d]pyrimidine compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

When administered to an animal, the compounds of the present invention or pharmaceutically acceptable salts thereof can be administered neat or as a component of a composition that comprises a pharmaceutically acceptable carrier or vehicle. A composition of the invention can be prepared using a method comprising admixing the compound of the present invention or pharmaceutically acceptable salt thereof and a physiologically acceptable carrier, excipient, or diluent. Admixing can be accomplished using methods well known in the art.

The present compositions, comprising compounds of the present invention or pharmaceutically acceptable salts thereof can be administered orally, or by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound of the present invention or pharmaceutically acceptable salt thereof into the bloodstream. The mode of administration is left to the discretion of the practitioner.

In one aspect, the compound of the present invention or pharmaceutically acceptable salt thereof is administered orally.

In another aspect, the compound of the present invention or pharmaceutically acceptable salt thereof is administered intravenously.

In another aspect, it can be desirable to administer the compound of the present invention or pharmaceutically acceptable salt thereof locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain aspects, it can be desirable to introduce the compound of the present invention or pharmaceutically acceptable salt thereof into the central nervous system, circulatory system or gastrointestinal tract by any suitable route, including intraventricular, intrathecal injection, paraspinal injection, epidural injection, enema, and by injection adjacent to the peripheral nerve. An intraventricular catheter, for example, can facilitate intraventricular injection attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain aspects, the compound of the present invention or pharmaceutically acceptable salt thereof can be formulated as a suppository, with traditional binders and excipients such as triglycerides.

In another aspect, compound of the present invention or pharmaceutically acceptable salt thereof can be delivered in a vesicle, in particular a liposome by methods known in the art.

In yet another aspect, the compound of the present invention or pharmaceutically acceptable salt thereof can be delivered in a controlled-release system or sustained-release system by methods known in the art. In one aspect, a pump can be used. In another aspect, polymeric materials can be used.

In yet another aspect, a controlled- or sustained-release system can be placed in proximity of a target of the compound of the present invention or a pharmaceutically acceptable salt thereof, e.g., the reproductive organs, thus requiring only a fraction of the systemic dose.

The present compositions can optionally comprise a suitable amount of a pharmaceutically acceptable excipient.

Such pharmaceutically acceptable excipients can be liquids, such as water and oils, including those of petroleum, animal, vegetable, or synthetic origin, such as peanut oil, soybean oil, mineral oil, sesame oil and the like. The excipients can be saline, gum acacia, gelatin, starch paste, talc, keratin, colloidal silica, urea and the like. In addition, auxiliary, stabilizing, thickening, lubricating, and coloring agents can be used. In one aspect, the excipients are sterile when administered to an animal. The excipient should be stable under the conditions of manufacture and storage and should be preserved against the contaminating action of microorganisms. Water is a particularly useful excipient in the practice of this invention where administration is performed intravenously. Saline solutions and aqueous dextrose and glycerol solutions can also be employed as liquid excipients, particularly for injectable solutions. Suitable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents known in the art.

Liquid carriers may be used in preparing solutions, suspensions, emulsions, syrups, and elixirs. The compound of the present invention or pharmaceutically acceptable salt thereof can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one aspect, the composition is in the form of a capsule.

In one aspect, the compound of the present invention or pharmaceutically acceptable salt thereof is formulated in accordance with known procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound of the present invention or pharmaceutically acceptable salt thereof. In tablets, the compound of the present invention or pharmaceutically acceptable salt thereof is mixed with a carrier having the necessary compression properties in suitable proportions and compacted in the shape and size desired. The powders and tablets can contain up to about 99% of the compound of the present invention or pharmaceutically acceptable salt thereof.

Capsules may contain mixtures of the compounds of the present invention or pharmaceutically acceptable salts thereof with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one aspect, the excipients are of pharmaceutical grade.

In another aspect, the compound of the present invention or pharmaceutically acceptable salt thereof can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the compound of the present invention or pharmaceutically acceptable salt thereof is to be administered by infusion, it can be dispensed, for example, with an infusion bottle containing sterile pharmaceutical grade water or saline. Where the compound of the present invention or pharmaceutically acceptable salt thereof is administered by injection, an ampoule of sterile water for injection or saline can be provided so that the ingredients can be mixed prior to administration.

In another aspect, the compound of the present invention or pharmaceutically acceptable salt thereof can be administered transdermally through the use of a transdermal patch. Transdermal administrations include administrations across the surface of the body and the inner linings of the bodily passages including epithelial and mucosal tissues. Such administrations can be carried out using the present compounds of the present invention or pharmaceutically acceptable salts thereof, in lotions, creams, foams, patches, suspensions, solutions, and suppositories (e.g., rectal or vaginal).

Transdermal administration can be accomplished through the use of a transdermal patch containing the compound of the present invention or pharmaceutically acceptable salt thereof and a carrier that is inert to the compound of the present invention or pharmaceutically acceptable salt thereof, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound of the present invention or pharmaceutically acceptable salt thereof into the blood stream, such as a semi-permeable membrane covering a reservoir containing the compound of the present invention or pharmaceutically acceptable salt thereof with or without a carrier, or a matrix containing the active ingredient.

The compounds of the present invention or pharmaceutically acceptable salts thereof may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound of the present invention or pharmaceutically acceptable salt thereof can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound of the present invention or a pharmaceutically acceptable salt thereof, and can thus reduce the occurrence of adverse side effects.

Controlled- or sustained-release compositions can initially release an amount of the compound of the present invention or pharmaceutically acceptable salt thereof that promptly produces the desired therapeutic or prophylactic effect, and gradually and continually release other amounts of the compound of the present invention or pharmaceutically acceptable salt thereof to maintain this level of therapeutic or prophylactic effect over an extended period of time.

In certain aspects, the present invention is directed to prodrugs of the compounds of the present invention or pharmaceutically acceptable salts of compounds of the present invention of the present invention. Various forms of prodrugs are known in the art.

The amount of the compound of the present invention or pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner.

The amount of the compound of the present invention or pharmaceutically acceptable salt thereof that is effective for treating or preventing an mTOR-related disorder or for treating or preventing a PI3K-related disorder will typically range from about 0.001 mg/kg to about 250 mg/kg of body weight per day, in one aspect, from about 1 mg/kg to about 250 mg/kg body weight per day, in another aspect, from about 1 mg/kg to about 50 mg/kg body weight per day, and in another aspect, from about 1 mg/kg to about 20 mg/kg of body weight per day.

In one aspect, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The present methods for treating or preventing an mTOR-related disorder, can further comprise administering another therapeutic agent to the animal being administered the compound of the present invention or pharmaceutically acceptable salt thereof. In one aspect, the other therapeutic agent is administered in an effective amount.

Effective amounts of other therapeutic agents to be administered simultaneously or sequentially with the present compound or pharmaceutically acceptable salt thereof are well known to those skilled in the art. However, it is well within the skilled artisan's purview to determine the other therapeutic agent's optimal effective amount range.

Suitable other therapeutic agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, hydroxyzine, glatiramer acetate, interferon beta-1a, interferon beta-1b, mitoxantrone, natalizumab, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one aspect, the compound of the present invention or pharmaceutically acceptable salt thereof is administered concurrently with another therapeutic agent.

In one aspect, a composition comprising an effective amount of the compound of the present invention or pharmaceutically acceptable salt thereof and an effective amount of another therapeutic agent within the same composition can be administered.

In another aspect, a composition comprising an effective amount of the compound of the present invention or a pharmaceutically acceptable salt of the compound of the present invention and a separate composition comprising an effective amount of another therapeutic agent can be concurrently administered. In another aspect, an effective amount of the compound of the present invention or a pharmaceutically acceptable salt thereof of the present invention administered prior to or subsequent to administration of an effective amount of another therapeutic agent.

In another aspect, a method of treating advanced renal cell carcinoma, comprising administering to a mammal in need thereof the compounds or a pharmaceutically acceptable salt thereof of the present formula 1 in an amount effective to treat advanced renal cell carcinoma.

In another aspect, a method of treating acute lymphoblastic leukemia, comprising administering to a mammal in need thereof the compounds or a pharmaceutically acceptable salt thereof of any of the present formula 1 in an amount effective to treat acute lymphoblastic leukemia.

In another aspect, a method of treating acute lymphoblastic leukemia, comprising administering to a mammal in need thereof the compounds or a pharmaceutically acceptable salt thereof of any of the present formula 1 in an amount effective to treat malignant melanoma.

In another aspect, a method of treating acute lymphoblastic leukemia, comprising administering to a mammal in need thereof the compounds or a pharmaceutically acceptable salt thereof of any of the present formula 1 in an amount effective to treat soft-tissue or bone sarcoma.

The general procedures used to synthesize the compounds of Formula 1 are described in Schemes 1-10 and are illustrated in the examples. Reasonable variations of the described procedures, which would be evident to one skilled in the art, are intended to be within the scope of the present invention:

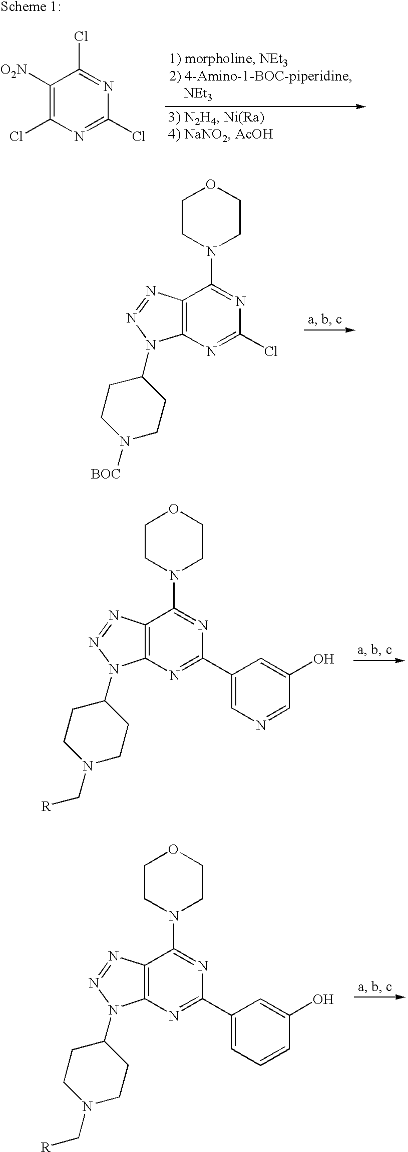

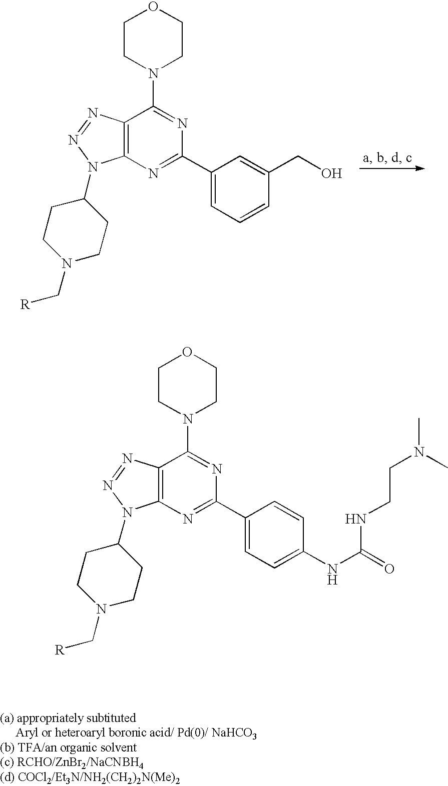

The key intermediate 3-(1-BOC-piperidin-4-yl)-5-chloro-7-morpholin-4-yl-3H-[1,2,3]triazolo[4,5-d]pyrimidine was made in four steps from the readily available 2,4,6-trichloropyrimidine. This BOC protected key intermediate could be coupled with a variety of functionalized boronic acids. Removal of the BOC protecting group, followed by reductive amination gave an array of piperidine compounds, elaborated on the 1-N atom.

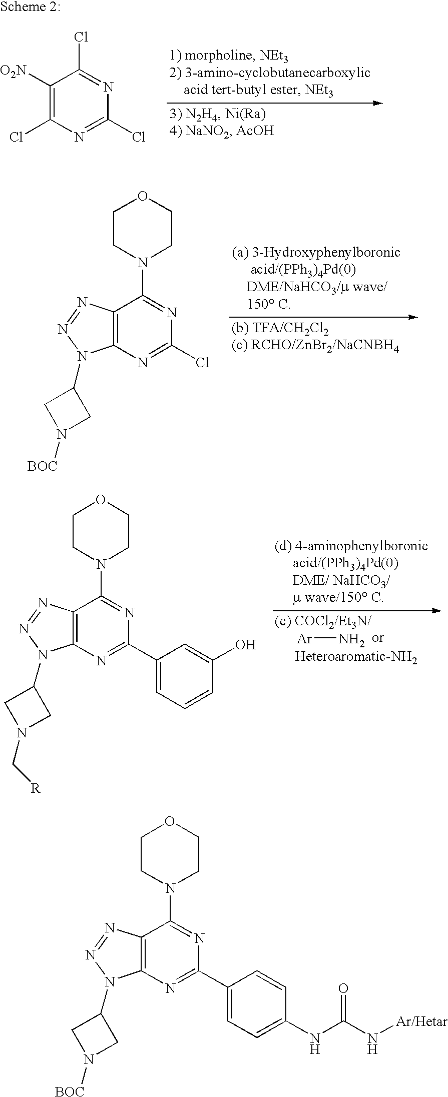

3-(5-Chloro-7-morpholin-4-yl-[1,2,3]triazolo[4,5-d]pyrimidin-3-yl)-azetidine-1-carboxylic acid tert-butyl ester was also made by a four-step process. The protected aziridine readily couples with 4-aminophenylboronic acid. Elaboration to a wide variety of urea compounds is done by phosgene mediated coupling with aromatic amines.

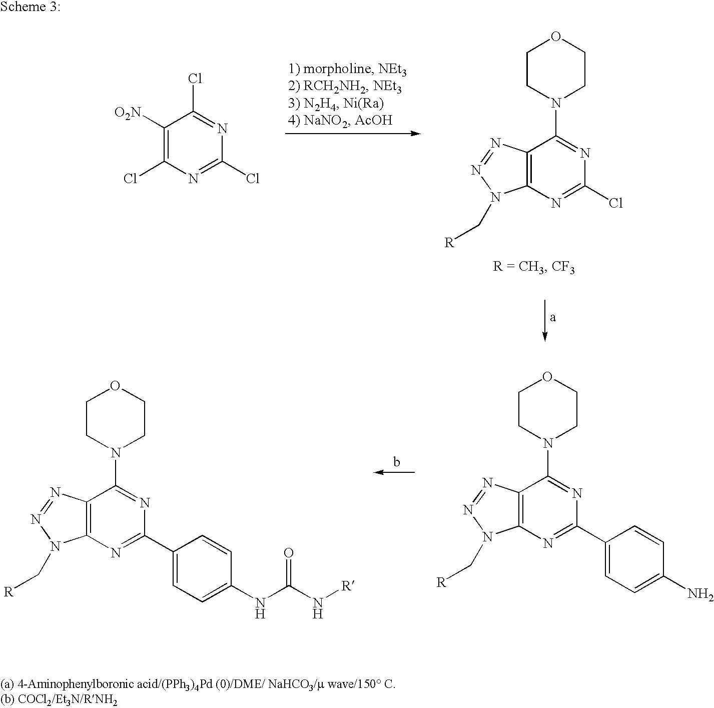

Simple 5-chloro-3-alkyl-7-morpholin-4-yl-3H-[1,2,3]triazolo[4,5-d]pyrimidine intermediate compounds were prepared using a four-step procedure. Suzuki coupling of these chlorinated intermediates with 4-aminophenylboronic acid gave an aniline intermediate. Elaboration to a wide variety of urea compounds is done by phosgene mediated coupling with aromatic amines.

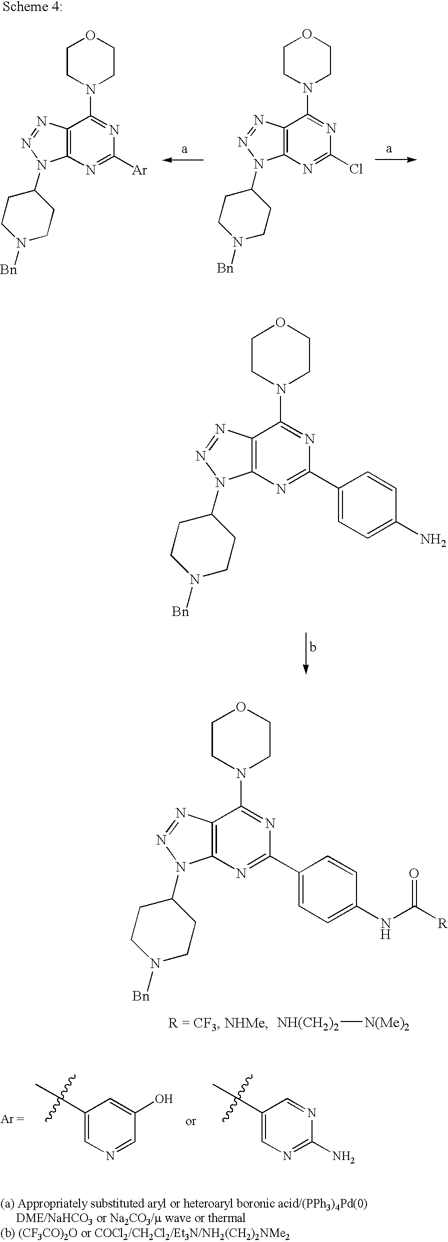

3-(1-Benzyl-piperidin-4-yl)-5-chloro-7-morpholin-4-yl-3H-[1,2,3]triazolo[4,5-d]pyrimidine smoothly underwent Suzuki coupling with a variety of aryl and heteroaryl boronic acids. Elaboration to a wide variety of urea compounds is done by phosgene mediated coupling with alkyl amines.

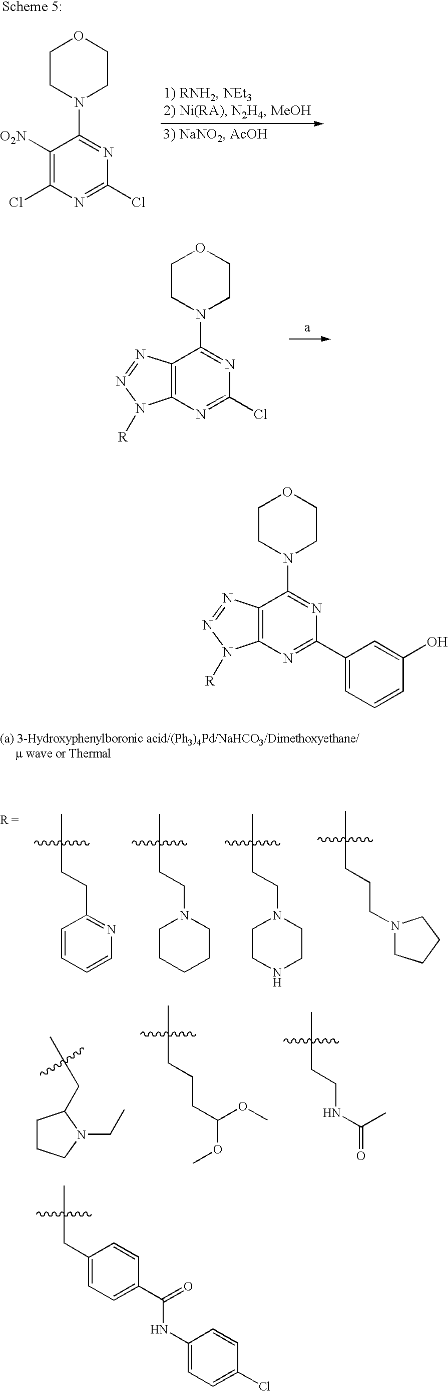

2,6-Dichloro-5-nitro-4-morpholino-pyrimidine, prepared as shown in Scheme 1, reacted with a wide variety of primary amines. Triazole formation, followed by Suzuki coupling with m-hydroxyphenylboronic acid gave the phenols shown above.

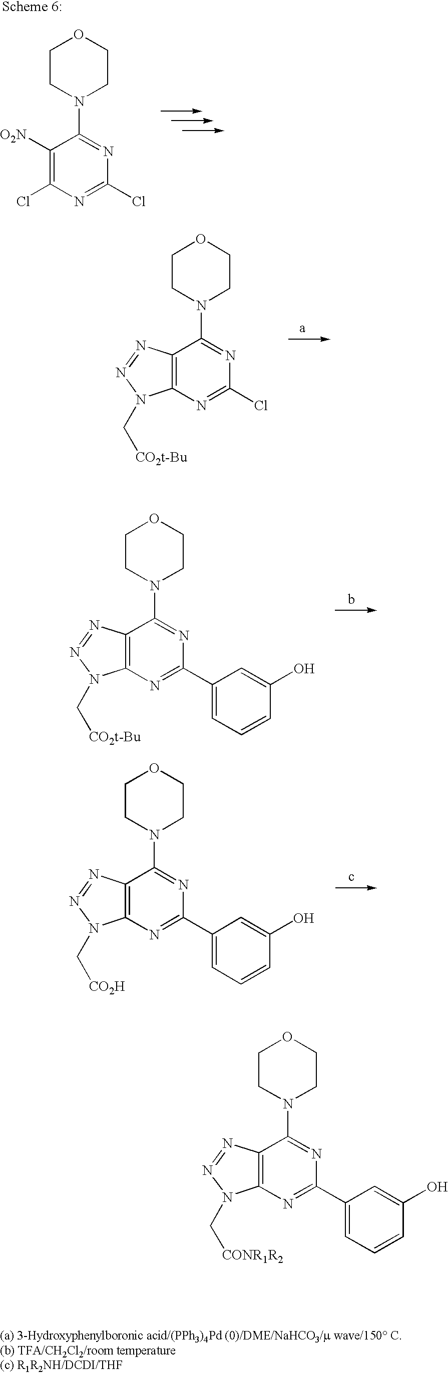

2,6-Dichloro-5-nitro-4-morpholino-pyrimidine, prepared as shown in Scheme 1, was converted to tert-butyl 2-(5-(3-hydroxyphenyl)-7-morpholino-3H-[1,2,3]triazolo[4,5-d]pyrimidin-3-yl)acetate. Suzuki coupling with m-hydroxypheneylboronic acid gave the tert-butyl ester shown. Removal of the ester group gave an acetic acid, which was converted to a variety of amides.

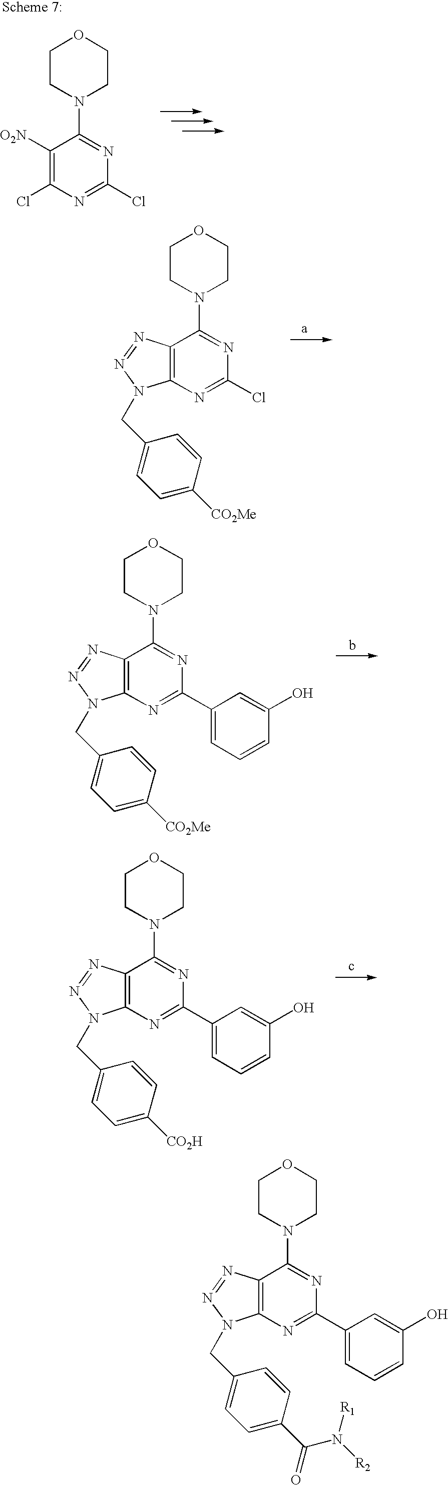

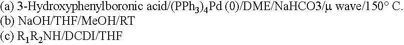

2,6-Dichloro-5-nitro-4-morpholino-pyrimidine, prepared as shown in Scheme 1, was converted to methyl 4-((5-chloro-7-morpholino-3H-[1,2,3]triazolo[4,5-d]pyrimidin-3-yl)methyl)benzoate. Suzuki coupling with m-hydroxypheneylboronic acid gave the methyl ester shown. Removal of the ester group gave a benzoic acid, which was converted to a variety of amides.

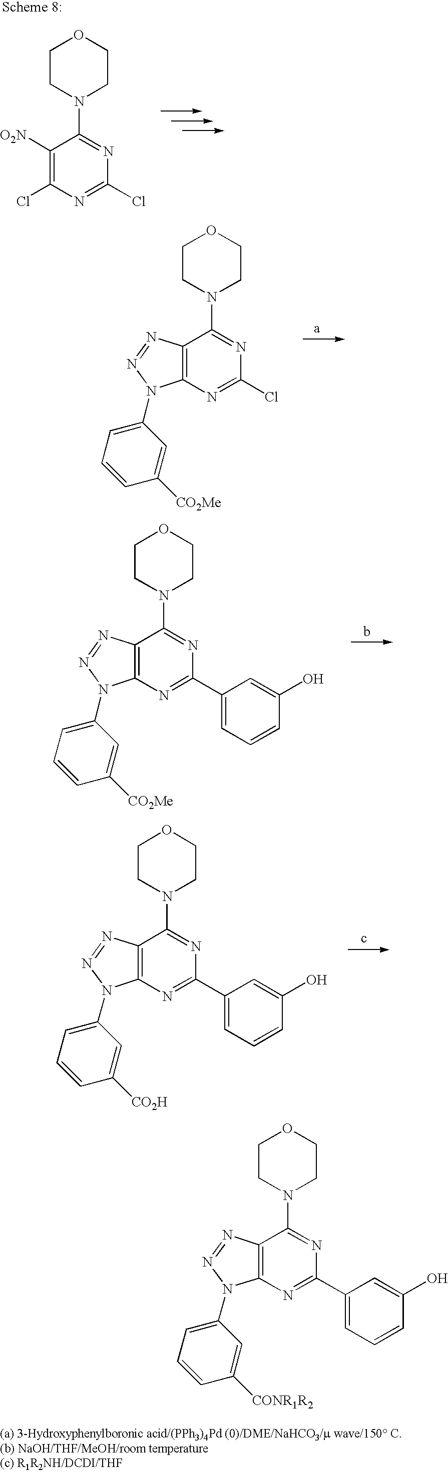

2,6-Dichloro-5-nitro-4-morpholino-pyrimidine, prepared as shown in Scheme 1, was converted to methyl 3-((5-chloro-7-morpholino-3H-[1,2,3]triazolo[4,5-d]pyrimidin-3-yl)methyl)benzoate. Suzuki coupling with m-hydroxypheneylboronic acid gave the methyl ester shown. Removal of the ester group gave a benzoic acid, which was converted to a variety of amides.

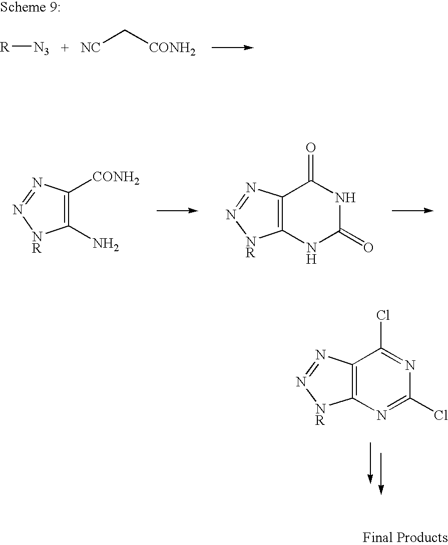

As an alternative synthesis, the triazole ring could be constructed first and the pyrimidine ring annealed to it. 5-Amino-1-substituted-1H-1,2,3-triazole-4-carboxamide compounds could be made from substituted azide compounds and 2-cyanoacetamide. Reaction with urea would give the 3-substituted-3H-[1,2,3]triazolo[4,5-d]pyrimidine-5,7(4H,6H)-dione shown. Treatment with POCl₃ would give the key intermediate 5,7-dichloro-3-substituted-3H-[1,2,3]triazolo[4,5-d]pyrimidine. Reaction with an amine 4 and Suzuki coupling with a boronic acid of the formula (R²)ᵣ-Ar—B(OH)₂ would give a variety of final products of formula 1.

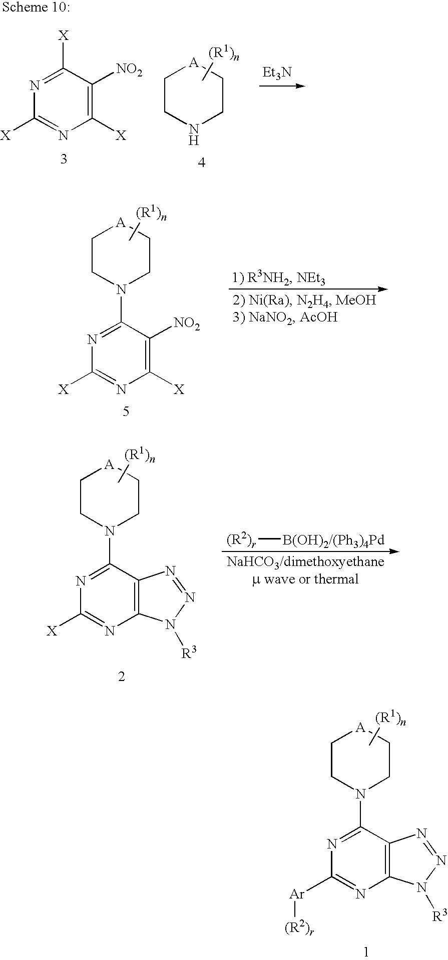

A general synthesis of 1 starts with the readily available 2,4,6-halo-5-nitropyrimidine compounds 3. Reaction with amine 4 followed by annulation of the triazole ring gave the 3H-[1,2,3]triazolo[4,5-d]pyrimidine 2. Suzuki coupling with a boronic acid of the formula (R²)ᵣ-Ar—B(OH)₂ gave a variety of final products of formula 1.

### EXAMPLES

The following procedures were used to synthesize the 3H-[1,2,3]triazolo[4,5-d]pyrimidine compounds in the Examples that follow.

### Experimental Procedures

Preparation of 2,6-dichloro-5-nitro-4-morpholino-pyrimidine. To a solution of 2,4,6-trichloronitropyrimidine (6.20 g, 27.2 mmol) in CH₂Cl₂ (170 mL) at 0° C. was added a solution of morpholine (2.34 g, 27.2 mmol) and NEt₃ (2.74 g, 27.2 mmol) in CH₂Cl₂ (70 mL) over a period of 1 hr. The reaction mixture was stirred for another 1 hr at 0° C. and allowed to warm to 20° C. and stirred for 12 hours to drive the reaction to competition. For purification, silica gel (20 g) was added to the reaction mixture and the solvent was removed so that product was adsorbed on the silica gel. The material was purified by flash chromatography using CH₂Cl₂ eluent the product was obtained as yellow solid after concentration. Yield: 6.90 g, 91%. MS (ESI) m/z 279.

### Procedure 1

### Step 1

Synthesis of 2-chloro-6-alkylamino-5-nitro-4-morpholino-pyrimidines with primary amines. To an appropriately substituted CH₂Cl₂ solution of the primary amine (1 eq) in CH₂Cl₂ (170 mL) at 0° C. was added a solution of 2,6-dichloro-5-nitro-4-morpholino-pyrimidine (2.34 g, 27.2 mmol) and NEt₃ (2.74 g, 27.2 mmol) dissolved in CH₂Cl₂ (70 mL) over a period of 1 hr. The reaction mixture was stirred for another 1 hr at 0° C. and allowed to warm to 20° C. and stirred for 1-4 hours to drive the reaction to completion. The product was purified by SiO₂ column chromatography by eluting it with CH₂Cl₂. Yellow solid (73-91% yield).

### Step 2

Reduction of 2-chloro-6-alkylamino-5-nitro-4-morpholino-pyrimidine. In a three-necked flask was suspended under nitrogen atmosphere (2-chloro-6-morpholin-4-yl-5-nitro-pyrimidin-4-yl)-alkyl-amines (1.0 mmol) and Raney™ nickel (850 mg) in methanol (30 mL). To the stirring reaction mixture was added slowly hydrazine (0.3 mL, 9 mmol, 9 eq) and the stirring was continued for 0.5 hours to drive the reduction to completion. The reaction mixture was filtered over Celite™ and the filtrate was evaporated and purified by flash purified by chromatography using CH₂Cl₂/MeOH/NH₃ (10:1:0.1) to obtain the product (73-100% yield) as off-white solid.

### Step 3

Synthesis of 8-aza-9-alkyl-2-chloro-6-morpholino-purines. To a stirred solution of N⁴-alkyl-2-chloro-6-morpholin-4-yl-pyrimidine-4,5-diamine (1 mmol) in acetic acid/water (1:1, 4 mL) at 0° C. was added aqueous (0.5 N) NaNO₂-solution (4 mL, 2 mmol, 2 eq) and the reaction mixture was allowed to stir for 2 hours. The off-white solid was collected by filtration and dried in vacuum to give the 8-aza-9-alkyl-2-chloro-6-morpholino-purines (64-95% yield).

### Procedure 2

Preparation of 8-aza-9-alkyl-2-(aryl/heteroaryl)-6-morpholino-purines. To a microwave processing tube was added dimethoxyethane (1.6 mL), aqueous Na₂CO₃ (2 M solution, 0.4 mL, 0.8 mmol, 2 eq), (Ph₃P)₄Pd (46 mg, 0.08 mmol), and the appropriately substituted boronic acid or ester (0.75 mmol, 2 eq) and the 8-aza-9-alkyl-2-chloro-6-morpholino-purines (0.38 mmol) and the vessel was sealed. The mixture was heated to 140° C. for 45 minutes. The solvents were removed on a rotary evaporator and the crude compound was purified by silica gel chromatography (CH₂Cl₂/MeOH/NH₃) to give the product as a off-white solid (45-76% yield).

### Preparation of 3-(1-Benzyl-piperidin-4-yl)-5-chloro-7-morpholin-4-yl-3H-[1,2,3]triazolo[4,5-d]pyrimidine

### Step 1

(1-Benzyl-piperidin-4-yl)-(2-chloro-6-morpholin-4-yl-5-nitro-pyrimidin-4-yl)-amine was prepared from 2,6-dichloro-5-nitro-4-morpholino-pyrimidine (1.5 g, 6.58 mmol) and 4-amino-1-benzylpiperidine (1.25 g, 6.58 mmol) following procedure 1 (step 1) to give the final product (2.0 g, 70% yield); MS (ESI) m/z 433.1.

### Step 2

N⁴-(1-Benzyl-piperidin-4-yl)-2-chloro-6-morpholin-4-yl-pyrimidine-4,5-diamine was prepared by reduction of (1-benzyl-piperidin-4-yl)-(2-chloro-6-morpholin-4-yl-5-nitro-pyrimidin-4-yl)-amine (1.0 g, 2.3 mmol) following procedure 1 (step 2) to give the final product (900 mg, 97% yield); MS (ESI) m/z 403.1.

### Step 3

3-(1-Benzyl-piperidin-4-yl)-5-chloro-7-morpholin-4-yl-3H-[1,2,3]triazolo[4,5-d]pyrimidine was prepared from N⁴-(1-benzyl-piperidin-4-yl)-2-chloro-6-morpholin-4-yl-pyrimidine-4,5-diamine (500 mg, 1.24 mmol) and aqueous (0.5N) NaNO₂ solution (5 mL, 2.5 mmol) following procedure 1 (step 3) to give the final product (510 mg, 100% yield); MS (ESI) m/z 414.2.

### Preparation of 5-Chloro-3-ethyl-7-morpholin-4-yl-3H-[1,2,3]triazolo[4,5-d]pyrimidine

### Step 1

(2-Chloro-6-morpholin-4-yl-5-nitro-pyrimidin-4-yl)ethylamine was prepared from 2,6-dichloro-5-nitro-4-morpholino-pyrimidine (2.0 g, 7.17 mmol) and ethylamine (2 molar solution in THF, 3.94 mL, 7.89 mmol) following procedure 1 (step 1) to give the final product (2.1 g, 100% yield); MS (ESI) m/z 288.

### Step 2

2-Chloro-N-4-ethyl-6-morpholin-4-yl-pyrimidine-4,5-diamine was prepared by the reduction of (2-chloro-6-morpholin-4-yl-5-nitro-pyrimidin-4-yl)ethylamine (600 mg, 2.08 mmol) following procedure 1 (step 2) to give the final product (374 mg, 70% yield); MS (ESI) m/z 258.

### Step 3

5-Chloro-3-ethyl-7-morpholin-4-yl-3H-[1,2,3]triazolo[4,5-d]pyrimidine was prepared from (2-chloro-6-morpholin-4-yl-5-nitro-pyrimidin-4-yl)-ethyl-amine (374 mg, 1.45 mmol) and aqueous (0.5N) NaNO₂ solution (3.75 mL, 1.88 mmol) following procedure 1 (step 3) to give the final product (250 mg, 64% yield); MS (ESI) m/z 269.

### Example 1

Preparation of 3-[3-(1-benzylpiperidin-4-yl)-7-morpholin-4-yl-3H-[1,2,3]triazolo[4,5-d]pyrimidin-5-yl]phenol. 3-[3-(1-benzylpiperidin-4-yl)-7-morpholin-4-yl-3H-[1,2,3]triazolo[4,5-d]pyrimidin-5-yl]phenol was prepared from 3-(1-benzyl-piperidin-4-yl)-5-chloro-7-morpholin-4-yl-3H-[1,2,3]triazolo[4,5-d]pyrimidine (100 mg, 0.24 mmol) and 3-hydroxyphenylboronic acid (60 mg, 0.36 mmol) following procedure 2 to give the titled product (70 mg, 61% yield). MS (ESI) m/z 472.

### Example 2

Preparation of 5-[3-(1-benzylpiperidin-4-yl)-7-morpholin-4-yl-3H-[1,2,3]triazolo[4,5-d]pyrimidin-5-yl]pyrimidin-2-amine. 1-benzylpiperidin-4-yl)-7-morpholin-4-yl-3H-[1,2,3]triazolo[4,5-d]pyrimidin-5-yl]pyrimidin-2-amine was prepared from 3-(1-benzyl-piperidin-4-yl)-5-chloro-7-morpholin-4-yl-3H-[1,2,3]triazolo[4,5-d]pyrimidine (100 mg, 0.24 mmol) and 2-aminopyrimidine-4-boronic acid (66 mg, 0.48 mmol) following procedure 2 to give the titled product (52 mg, 46% yield); MS (ESI) m/z 473.

### Example 3

Preparation of 5-[3-(1-benzylpiperidin-4-yl)-7-morpholin-4-yl-3H-[1,2,3]triazolo[4,5-d]pyrimidin-5-yl]pyridin-3-ol. 1-benzylpiperidin-4-yl)-7-morpholin-4-yl-3H-[1,2,3]triazolo[4,5-d]pyrimidin-5-yl]pyrimidin-2-amine was prepared from 3-(1-benzyl-piperidin-4-yl)-5-chloro-7-morpholin-4-yl-3H-[1,2,3]triazolo[4,5-d]pyrimidine (160 mg, 0.38 mmol) and 3-methoxymethoxy-5-(4,4,5,5-tetramethyl-[1,3,2]dioxaborolan-2-yl)-pyridine (151 mg, 0.57 mmol) following procedure 2 to give the intermediate 3-(1-benzyl-piperidin-4-yl)-5-(5-methoxymethoxy-pyridin-3-yl)-7-morpholin-4-yl-3H-[1,2,3]triazolo[4,5-d]pyrimidine. The 3-(1-benzyl-piperidin-4-yl)-5-(5-methoxymethoxy-pyridin-3-yl)-7-morpholin-4-yl-3H-[1,2,3]triazolo[4,5-d]pyrimidine was dissolved in conc. HCl (1 mL) and methanol (4 mL) and heated to reflux for 1 hr. The reaction mixture was cooled to 0° C. for 15 minutes and the titled product was collected by filtration (56 mg, 44% yield); MS (ESI) m/z 473.

Preparation of 4-[3-(1-Benzyl-piperidin-4-yl)-7-morpholin-4-yl-3H-[1,2,3]triazolo[4,5-d]pyrimidin-5-yl]-phenylamine. 4-[3-(1-Benzyl-piperidin-4-yl)-7-morpholin-4-yl-3H-[1,2,3]triazolo[4,5-d]pyrimidin-5-yl]-phenylamine was prepared from 3-(1-benzyl-piperidin-4-yl)-5-chloro-7-morpholin-4-yl-3H-[1,2,3]triazolo[4,5-d]pyrimidine (160 mg, 0.38 mmol) and 4-(4,4,5,5-tetramethyl-[1,3,2]dioxaborolan-2-yl)-phenylamine (152 mg, 0.69 mmol) following procedure 2 to give the titled product (180 mg, 100% yield); MS (ESI) m/z 471.3.

### Examples 4 and 5

Preparation of 1-{4-[3-(1-benzylpiperidin-4-yl)-7-morpholin-4-yl-3H-[1,2,3]triazolo[4,5-d]pyrimidin-5-yl]phenyl}-3-[2-(dimethylamino)ethyl]urea and N-{4-[3-(1-benzylpiperidin-4-yl)-7-morpholin-4-yl-3H-[1,2,3]triazolo[4,5-d]pyrimidin-5-yl]phenyl}-2,2,2-trifluoroacetamide. To a stirred solution of triphosgene (72 mg, 0.24 mmol) in CHCl₃ (2 mL) was added 4-[3-(1-benzyl-piperidin-4-yl)-7-morpholin-4-yl-3H-[1,2,3]triazolo[4,5-d]pyrimidin-5-yl]-phenylamine (TFA-salt, 100 mg, 0.14 mmol) at 0° C. The reaction mixture was stirred for 30 min. N,N-dimethylethylenediamine (100 mg, 1.13 mmol) and NEt₃ (36 mg, 0.36 mmol) in CHCl₃ (1 mL) was added and the reaction mixture was stirred for additional 1 hr. The solvents were removed on a rotary evaporator and the crude mixture was purified by semi-prep-HPLC (TFA-method) to give 1-{4-[3-(1-benzylpiperidin-4-yl)-7-morpholin-4-yl-3H-[1,2,3]triazolo[4,5-d]pyrimidin-5-yl]phenyl}-3-[2-(dimethylamino)ethyl]urea (33 mg, 29% yield) MS (ESI) m/z 585.3 and N-{4-[3-(1-benzylpiperidin-4-yl)-7-morpholin-4-yl-3H-[1,2,3]triazolo[4,5-d]pyrimidin-5-yl]phenyl}-2,2,2-trifluoroacetamide (33 mg, 39% yield) MS (ESI) m/z 567.2.

### Example 6

Preparation of 1-{4-[3-(1-benzylpiperidin-4-yl)-7-morpholin-4-yl-3H-[1,2,3]triazolo[4,5-d]pyrimidin-5-yl]phenyl}-3-methylurea. To a stirred solution of triphosgene (113 mg, 0.38 mmol) in CHCl₃ (3 mL) was added 4-[3-(1-benzyl-piperidin-4-yl)-7-morpholin-4-yl-3H-[1,2,3]triazolo[4,5-d]pyrimidin-5-yl]-phenylamine (141 mg, 0.3 mmol) at 0° C. The reaction mixture was stirred for 30 min. methylamine (2M in THF, 2 mL, 4 mmol) and the reaction mixture was stirred for additional 1 hr. The solvents were removed on a rotary evaporator and the crude mixture was purified by semi-prep-HPLC (TFA-method) to give 1-{4-[3-(1-benzylpiperidin-4-yl)-7-morpholin-4-yl-3H-[1,2,3]triazolo[4,5-d]pyrimidin-5-yl]phenyl}-3-methylurea (69 mg, 35% yield) MS (ESI) m/z 528.3.

### Preparation of N-[2-(5-Chloro-7-morpholin-4-yl-[1,2,3]triazolo[4,5-d]pyrimidin-3-yl)-ethyl]-acetamide

### Step 1

N-[2-(2-Chloro-6-morpholin-4-yl-5-nitro-pyrimidin-4-ylamino)-ethyl]-acetamide was prepared from 2,6-dichloro-5-nitro-4-morpholino-pyrimidine (500 mg, 1.8 mmol) and N-acetylethylendiamine (184 mg, 1.8 mmol) following procedure 1 (step 1) to give the final product (550 mg, 89% yield). MS (ESI) m/z 345.1.

### Step 2

N-[2-(5-Amino-2-chloro-6-morpholin-4-yl-pyrimidin-4-ylamino)-ethyl]-acetamide was prepared by reduction of N-[2-(2-chloro-6-morpholin-4-yl-5-nitro-pyrimidin-4-ylamino)-ethyl]-acetamide (550 mg, 1.59 mmol) following procedure 1 (step 2) to give the final product (500 mg, 100% yield). MS (ESI) m/z 315.1.

### Step 3

N-[2-(5-Chloro-7-morpholin-4-yl-[1,2,3]triazolo[4,5-d]pyrimidin-3-yl)-ethyl]-acetamide was prepared from N-[2-(5-amino-2-chloro-6-morpholin-4-yl-pyrimidin-4-ylamino)-ethyl]-acetamide (500 mg, 1.24 mmol) and aqueous (0.5N) NaNO₂ solution (5 mL, 2.5 mmol) following procedure 1 (step 3) to give the final product (300 mg, 58% yield). MS (ESI) m/z 326.

### Example 7

Preparation of N-{2-[5-(3-hydroxyphenyl)-7-morpholin-4-yl-3H-[1,2,3]triazolo[4,5-d]pyrimidin-3-yl]ethyl}acetamide. N-{2-[5-(3-hydroxyphenyl)-7-morpholin-4-yl-3H-[1,2,3]triazolo[4,5-d]pyrimidin-3-yl]ethyl}acetamide was prepared from N-[2-(5-chloro-7-morpholin-4-yl-[1,2,3]triazolo[4,5-d]pyrimidin-3-yl)-ethyl]-acetamide (150 mg, 0.5 mmol) and 3-hydroxyphenyl-boronic acid (138 mg, 1.0 mmol) following procedure 2 to give the final product (56 mg, 29% yield); MS (ESI) m/z 384.

### Example 8

Preparation of N-(2-{5-[3-(hydroxymethyl)phenyl]-7-morpholin-4-yl-3H-[1,2,3]triazolo[4,5-d]pyrimidin-3-yl}ethyl)acetamide. N-(2-{5-[3-(hydroxymethyl)phenyl]-7-morpholin-4-yl-3H-[1,2,3]triazolo[4,5-d]pyrimidin-3-yl}ethyl)acetamide was prepared from N-[2-(5-chloro-7-morpholin-4-yl-[1,2,3]triazolo[4,5-d]pyrimidin-3-yl)-ethyl]-acetamide (150 mg, 0.5 mmol) and 3-(hydroxymethyl)-phenyl boronic acid (151 mg, 1.0 mmol) following procedure 2 to give the final product (52 mg, 26% yield); MS (ESI) m/z 398.

### Preparation of 5-Chloro-7-morpholin-4-yl-3-(3-pyrrolidin-1-yl-propyl)-3H-[1,2,3]triazolo[4,5-d]pyrimidine

### Step 1

(2-Chloro-6-morpholin-4-yl-5-nitro-pyrimidin-4-yl)-(3-pyrrolidin-1-yl-propyl)-amine was prepared from 2,6-dichloro-5-nitro-4-morpholino-pyrimidine (550 mg, 1.96 mmol) and 1-aminopropyl-pyrrolidine (301 mg, 2.35 mmol) following procedure 1 (step 1) to give the final product (500 mg, 69% yield); MS (ESI) m/z 371.

### Step 2

2-Chloro-6-morpholin-4-yl-N⁴-(3-pyrrolidin-1-yl-propyl)-pyrimidine-4,5-diamine was prepared by the reduction of (2-chloro-6-morpholin-4-yl-5-nitro-pyrimidin-4-yl)-(3-pyrrolidin-1-yl-propyl)-amine (500 mg, 1.34 mmol) following procedure 1 (step 2) to give the final product (350 mg, 76% yield); MS (ESI) m/z 341.

### Step 3

5-Chloro-7-morpholin-4-yl-3-(3-pyrrolidin-1-yl-propyl)-3H-[1,2,3]triazolo[4,5-d]pyrimidine was prepared from 2-chloro-6-morpholin-4-yl-N⁴-(3-pyrrolidin-1-yl-propyl)-pyrimidine-4,5-diamine (350 mg, 1.02 mmol) and aqueous (0.5N) NaNO₂ solution (3.5 mL, 1.75 mmol) following procedure 1 (step 3) to give the final product (150 mg, 42% yield); MS (ESI) m/z 352.

### Example 9

Preparation of 3-[7-morpholin-4-yl-3-(3-pyrrolidin-1-ylpropyl)-3H-[1,2,3]triazolo[4,5-d]pyrimidin-5-yl]phenol. 3-[7-Morpholin-4-yl-3-(3-pyrrolidin-1-ylpropyl)-3H-[1,2,3]triazolo[4,5-d]pyrimidin-5-yl]phenol was prepared from 5-chloro-7-morpholin-4-yl-3-(3-pyrrolidin-1-yl-propyl)-3H-[1,2,3]triazolo[4,5-d]pyrimidine (50 mg, 0.14 mmol) and 3-hydroxyphenyl boronic acid (39 mg, 0.28 mmol) following procedure 2 to give the final product (34 mg, 58% yield); MS (ESI) m/z 410.

### Example 10

Preparation of {3-[7-morpholin-4-yl-3-(3-pyrrolidin-1-ylpropyl)-3H-[1,2,3]triazolo[4,5-d]pyrimidin-5-yl]phenyl}methanol was prepared from 5-chloro-7-morpholin-4-yl-3-(3-pyrrolidin-1-yl-propyl)-3H-[1,2,3]triazolo[4,5-d]pyrimidine (50 mg, 0.14 mmol) and 3-(hydroxymethyl)phenyl boronic acid (43 mg, 0.28 mmol) following procedure 2 to give the final product (34 mg, 57% yield); MS (ESI) m/z 424.

### Example 11

Preparation of 5-(1H-indazol-4-yl)-7-morpholin-4-yl-3-(3-pyrrolidin-1-ylpropyl)-3H-[1,2,3]triazolo[4,5-d]pyrimidine was prepared from 5-chloro-7-morpholin-4-yl-3-(3-pyrrolidin-1-yl-propyl)-3H-[1,2,3]triazolo[4,5-d]pyrimidine (50 mg, 0.14 mmol) and 4-(4,4,5,5-tetramethyl-[1,3,2]dioxaborolan-2-yl)-1H-indazole (68 mg, 0.28 mmol) following procedure 2 to give the final product (18 mg, 29% yield); MS (ESI) m/z 434.

### Preparation of 3-(1-Boc-piperidin-4-yl)-5-chloro-7-morpholin-4-yl-3H-[1,2,3]triazolo[4,5-d]pyrimidine

### Step 1

(1-Boc-piperidin-4-yl)-(2-chloro-6-morpholin-4-yl-5-nitro-pyrimidin-4-yl)-amine was prepared from 2,6-dichloro-5-nitro-4-morpholino-pyrimidine (2.0 g, 7.17 mmol) and 4-amino-1-BOC-piperidine (1.43 g, 7.17 mmol) following procedure 1 (step 1) to give the final product (3.1 g, 99% yield); MS (ESI) m/z 443.2.

### Step 2

N⁴-(1-BOC-piperidin-4-yl)-2-chloro-6-morpholin-4-yl-pyrimidine-4,5-diamine was prepared by reduction of (1-BOC-piperidin-4-yl)-(2-chloro-6-morpholin-4-yl-5-nitro-pyrimidin-4-yl)-amine (3.13 g, 7.08 mmol) following procedure 1 (step 2) to give the final product (2.8 g, 96% yield); MS (ESI) m/z 413.2.

### Step 3

3-(1-Boc-piperidin-4-yl)-5-chloro-7-morpholin-4-yl-3H-[1,2,3]triazolo[4,5-d]pyrimidine was prepared from N⁴-(1-BOC-piperidin-4-yl)-2-chloro-6-morpholin-4-yl-pyrimidine-4,5-diamine (2.8 g, 6.79 mmol) and aqueous (0.5N) NaNO₂ solution (24 mL, 12 mmol) following procedure 1 (step 3) to give the final product (2.1 g, 73% yield). MS (ESI) m/z 424.2.

Preparation of 4-[5-(5-Methoxymethoxy-pyridin-3-yl)-7-morpholin-4-yl-[1,2,3]triazolo[4,5-d]pyrimidin-3-yl]-piperidine-1-carboxylic acid tert-butyl ester was prepared from 3-(1-BOC-piperidin-4-yl)-5-chloro-7-morpholin-4-yl-3H-[1,2,3]triazolo[4,5-d]pyrimidine (1.0 g, 2.35 mmol) and 3-methoxymethoxy-5-(4,4,5,5-tetramethyl-[1,3,2]dioxaborolan-2-yl)-pyridine (1.24 g, 4.7 mmol) following procedure 2 to give the titled product (1.3 g, 100%).

### Example 12

Preparation 5-(7-morpholin-4-yl-3-piperidin-4-yl-3H-[1,2,3]triazolo[4,5-d]pyrimidin-5-yl)pyridin-3-ol. 3-(1-Boc-piperidin-4-yl)-5-(5-methoxymethoxy-pyridin-3-yl)-7-morpholin-4-yl-3H-[1,2,3]triazolo[4,5-d]pyrimidine was dissolved CHCl₃ (15 mL) and TFA (5 mL) and stirred for 16 hours at 25° C., than the solvents were removed under reduced pressure and the residue was dissolved in conc. HCl (10 mL) and methanol (50 mL) and heated to reflux for 1 hr. The reaction mixture was cooled to 0° C. for 15 minutes and the titled compound was collected by filtration (56 mg, 44% yield); MS (ESI) m/z 383.3.

### Example 13

Preparation of 5-{3-[1-(2-furylmethyl)piperidin-4-yl]-7-morpholin-4-yl-3H-[1,2,3]triazolo[4,5-d]pyrimidin-5-yl}pyridin-3-ol. 5-(7-morpholin-4-yl-3-piperidin-4-yl-3H-[1,2,3]triazolo[4,5-d]pyrimidin-5-yl)pyridin-3-ol (17 mg, 0.044 mmol) was dissolved in methanol (1 mL) and 2-furalaldehyde (20 mg, 0.2 mmol), NaBH₃CN (10 mg, 0.088 mmol, 1 eq) and ZnCl₂ (10 mg, 0.044 mmol) was added. The suspension was stirred for 24 hours and the solvents were removed in vacuo. The crude product was dissolved in DMSO (2 mL), filtered and purified by semi-prep-HPLC using ACN/water/TFA as mobile phase. After unifying the product fraction and solvent removal, the product was obtained as a white solid. Yield: 16 mg, 35%; MS (ESI) m/z 463.4.

### Example 14

Preparation of 5-{3-[1-(4-fluorobenzyl)piperidin-4-yl]-7-morpholin-4-yl-3H-[1,2,3]triazolo[4,5-d]pyrimidin-5-yl}pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 4-fluorobenzaldehyde (20 mg, 0.16 mmol), NaBH₃CN (10 mg, 0.088 mmol), and ZnCl₂ (10 mg, 0.044 mmol) as described in example 13 to give the product (15 mg, 31% yield); MS (ESI) m/z 491.2.

### Example 15

Preparation of 5-(3-{1-[(6-bromopyridin-3-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 6-bromonicotinaldehyde (20 mg, 0.11 mmol), NaBH₃CN (10 mg, 0.088 mmol), and ZnCl₂ (10 mg, 0.044 mmol) as described in example 13 to give the product. Yield: 22 mg, 43%; MS (ESI) m/z 552.

### Example 16

Preparation of 5-(3-{1-[(5-bromopyridin-3-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 5-bromopicolinaldehyde (20 mg, 0.11 mmol), NaBH₃CN (10 mg, 0.088 mmol), and ZnCl₂ (10 mg, 0.044 mmol) as described in example 13 to give the product (20 mg, 38% yield); MS (ESI) m/z 552.

### Example 17

Preparation of 5-[3-(1-{4-[3-(dimethylamino)propoxy]benzyl}piperidin-4-yl)-7-morpholin-4-yl-3H-[1,2,3]triazolo[4,5-d]pyrimidin-5-yl]pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 4-(3-dimethylamino-propoxy)-benzaldehyde (20 mg, 0.10 mmol), NaBH₃CN (10 mg, 0.088 mmol), and ZnCl₂ (10 mg, 0.044 mmol) as described in example 13 to give the product (14 mg, 27% yield); MS (ESI) m/z 573.3.

### Example 18

Preparation of 5-{3-[1-(3,4-difluorobenzyl)piperidin-4-yl]-7-morpholin-4-yl-3H-[1,2,3]triazolo[4,5-d]pyrimidin-5-yl}pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 3,4-difluorobenzaldehyde (20 mg, 0.14 mmol), NaBH₃CN (10 mg, 0.088 mmol), and ZnCl₂ (10 mg, 0.044 mmol) as described in example 13 to give the product (15 mg, 31% yield); MS (ESI) m/z 508.

### Example 19

Preparation of 5-(3-{1-[(1-methyl-1H-pyrrol-2-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (20 mg, 0.052 mmol), 1-methylpyrrole-2-carbaldehyde (20 mg, 0.18 mmol), NaBH₃CN (20 mg, 0.18 mmol), and ZnCl₂ (20 mg, 0.18 mmol) as described in example 13 to give the product (18 mg, 46% yield); MS (ESI) m/z 475.2.

### Example 20

Preparation of 5-(3-{1-[(6-chloropyridin-3-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (20 mg, 0.052 mmol), 6-chloronicotinoylaldehyde (20 mg, 0.14 mmol), NaBH₃CN (20 mg, 0.18 mmol), and ZnCl₂ (20 mg, 0.18 mmol) as described in example 13 to give the product (29 mg, 71% yield); MS (ESI) m/z 508.2.

### Example 21

Preparation of 5-(3-{1-[(5-methyl-2-thienyl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (20 mg, 0.052 mmol), 5-methylthiophencarbaldehyde (20 mg, 0.14 mmol), NaBH₃CN (20 mg, 0.18 mmol), and ZnCl₂ (20 mg, 0.18 mmol) as described in example 13 to give the product (22 mg, 56% yield); MS (ESI) m/z 493.2.

### Example 22

Preparation of 5-[3-(1-methylpiperidin-4-yl)-7-morpholin-4-yl-3H-[1,2,3]triazolo[4,5-d]pyrimidin-5-yl]pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), aqueous (37%)-formaldehyde solution (20 mg, 0.24 mmol), NaBH₃CN (20 mg, 0.18 mmol), and ZnCl₂ (20 mg, 0.18 mmol) as described in example 13 to give the product (14 mg, 35% yield), MS (ESI) m/z 397.2.

### Example 23

Preparation of 5-{3-[1-(2,4-difluorobenzyl)piperidin-4-yl]-7-morpholin-4-yl-3H-[1,2,3]triazolo[4,5-d]pyrimidin-5-yl}pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 2,4-difluorobenazaldehyde (20 mg, 0.14 mmol), NaBH₃CN (20 mg, 0.18 mmol), and ZnCl₂ (20 mg, 0.18 mmol) as described in example 13 to give the product (15 mg, 32% yield). MS (ESI) m/z 509.2.

### Example 24

Preparation of 5-(3-{1-[(1-methyl-1H-imidazol-5-yl)methyl]piperidin-4-yl}-7-morpholin-4-yl-3H-[1,2,3]triazolo[4,5-d]pyrimidin-5-yl)pyridin-3-ol. was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 1-methyl-imidazol-5-carbaldehyde (20 mg, 0.18 mmol), NaBH₃CN (20 mg, 0.18 mmol), and ZnCl₂ (20 mg, 0.18 mmol) as described in example 13 to give the product (14 mg, 31% yield); MS (ESI) m/z 477.2.

### Example 25

Preparation of N-[3-({4-[5-(5-hydroxypyridin-3-yl)-7-morpholin-4-yl-3H-[1,2,3]triazolo[4,5-d]pyrimidin-3-yl]piperidin-1-yl}methyl)pyridin-2-yl]-2,2-dimethylpropanamide was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), N-(3-formyl-pyridin-2-yl)-2,2-dimethyl-propionamide (20 mg, 0.1 mmol), NaBH₃CN (20 mg, 0.18 mmol), and ZnCl₂ (20 mg, 0.18 mmol) as described in example 13 to give the product (5 mg, 10% yield). MS (ESI) m/z 573.2.

### Example 26

Preparation of 5-(3-{1-[(4,5-dimethyl-2-thienyl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 4,5-dimethylthiophencarbaldehyde (20 mg, 0.1 mmol), NaBH₃CN (20 mg, 0.14 mmol), and ZnCl₂ (20 mg, 0.18 mmol) as described in example 13 to give the product (10 mg, 20% yield); MS (ESI) m/z 507.2.

### Example 27

Preparation of 5-[3-(1-butylpiperidin-4-yl)-7-morpholin-4-yl-3H-[1,2,3]triazolo[4,5-d]pyrimidin-5-yl]pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), butyraldehyde (20 mg, 0.1 mmol), NaBH₃CN (20 mg, 0.36 mmol), and ZnCl₂ (20 mg, 0.18 mmol) as described in example 13 to give the product (11 mg, 26% yield); MS (ESI) m/z 439.2.

### Example 28

Preparation of 5-(3-{1-[(4-benzylpiperazin-1-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 4-benzyl-piperazine-1-carbaldehyde (20 mg, 0.1 mmol), NaBH₃CN (20 mg, 0.36 mmol), and ZnCl₂ (20 mg, 0.18 mmol) as described in example 13 to give the product (15 mg, 28% yield); MS (ESI) m/z 571.

### Example 29

Preparation of 5-{7-morpholin-4-yl-3-[1-(1H-pyrrol-2-ylmethyl)piperidin-4-yl]-3H-[1,2,3]triazolo[4,5-d]pyrimidin-5-yl}pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), pyrrole-2-carbaldehyde (20 mg, 0.21 mmol), NaBH₃CN (20 mg, 0.36 mmol), and ZnCl₂ (20 mg, 0.18 mmol) as described in example 13 to give the titled product (9 mg, 20% yield), MS (ESI) m/z 462.

### Example 30

Preparation of 5-(3-{1-[(1-methyl-1H-pyrazol-5-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 1-methylpyrrazole-6-carbaldehyde (20 mg, 0.18 mmol), NaBH₃CN (20 mg, 0.36 mmol), and ZnCl₂ (20 mg, 0.18 mmol) as described in example 13 to give the product (16 mg, 33% yield); MS (ESI) m/z 477.2.

### Example 31

Preparation of 5-{7-morpholin-4-yl-3-[1-(4-pyridin-4-ylbenzyl)piperidin-4-yl]-3H-[1,2,3]triazolo[4,5-d]pyrimidin-5-yl}pyridin-3-ol was prepared from 5-(7-morpholin-4-yl-3-piperidin-4-yl-3H-[1,2,3]triazolo[4,5-d]pyrimidin-5-yl)pyridin-3-ol (25 mg, 0.066 mmol), 1-4-pyridin-4-yl-benzaldehyde (20 mg, 0.18 mmol), NaBH₃CN (20 mg, 0.36 mmol), and ZnCl₂ (20 mg, 0.18 mmol) as described in example 13 to give the product (16 mg, 33% yield). MS (ESI) m/z 550.2.

### Example 32

Preparation of 4-(3-Ethyl-7-morpholin-4-yl-3H-[1,2,3]triazolo[4,5-d]pyrimidin-5-yl)-phenylamine was prepared from 5-chloro-3-ethyl-7-morpholin-4-yl-3H-[1,2,3]triazolo[4,5-d]pyrimidine (1.45 g, 5.40 mmol) 4-(4,4,5,5-tetramethyl-[1,3,2]dioxaborolan-2-yl)-phenylamine (1.53 g, 7.03 mmol) following procedure 2 to give the titled product (1.63 g, 92% yield). MS (ESI) m/z 326.

### Example 33

Preparation of 1-[4-(3-ethyl-7-morpholin-4-yl-3H-[1,2,3]triazolo[4,5-d]pyrimidin-5-yl)phenyl]-3-pyridin-4-ylurea. To a stirred solution of triphosgene (68 mg, 0.23 mmol) in CH₂Cl₂ (5 mL) was added 4-(3-ethyl-7-morpholin-4-yl-3H-[1,2,3]triazolo[4,5-d]pyrimidin-5-yl)-phenylamine (100 mg, 0.46 mmol) at 0° C. The reaction mixture was stirred for 15 min and 4-aminopyridine (40 mg, 0.46 mmol) and NEt₃ (64 μL, 0.46 mmol) was added and the reaction mixture was stirred for additional 1 hr. The solvents were removed on a rotary evaporator and the crude mixture was purified by semi-prep-HPLC (NH₃-method) to give 1-[4-(3-ethyl-7-morpholin-4-yl-3H-[1,2,3]triazolo[4,5-d]pyrimidin-5-yl)phenyl]-3-pyridin-4-ylurea (22 mg, 11% yield) MS (ESI) m/z 446.

### Example 34

Preparation of 1-[2-(dimethylamino)ethyl]-3-[4-(3-ethyl-7-morpholin-4-yl-3H-[1,2,3]triazolo[4,5-d]pyrimidin-5-yl)phenyl]urea. To a stirred solution of triphosgene (90 mg, 0.31 mmol) in CHCl₃ (1 mL) was added 4-(3-ethyl-7-morpholin-4-yl-3H-[1,2,3]triazolo[4,5-d]pyrimidin-5-yl)-phenylamine (100 mg, 0.31 mmol) at 0° C. The reaction mixture was stirred for 15 min and N,N-dimethylethylenediamine (82 mg, 0.93 mmol) and NEt₃ (42 μL, 0.31 mmol) was added and the reaction mixture was stirred for additional 1 hr. The solvents were removed on a rotary evaporator and the crude mixture was purified by semi-prep-HPLC (NH₃-method) to give 1-[2-(dimethylamino)ethyl]-3-[4-(3-ethyl-7-morpholin-4-yl-3H-[1,2,3]triazolo[4,5-d]pyrimidin-5-yl)phenyl]urea (13 mg, 10% yield) MS (ESI) m/z 440.

### Example 35

Preparation of 1-[4-(3-ethyl-7-morpholin-4-yl-3H-[1,2,3]triazolo[4,5-d]pyrimidin-5-yl)phenyl]-3-(2-methylpyridin-4-yl)urea. The title compound was prepared as described in the example above using triphosgene (74 mg, 0.25 mmol), 4-(3-ethyl-7-morpholin-4-yl-3H-[1,2,3]triazolo[4,5-d]pyrimidin-5-yl)-phenylamine (100 mg, 0.31 mmol), 4-amino-2-methylpyridine (100 mg, 0.93 mmol) and NEt₃ (430 μL, 0.44 mmol) in CH₂Cl₂ (3 mL) to give 1-[4-(3-ethyl-7-morpholin-4-yl-3H-[1,2,3]triazolo[4,5-d]pyrimidin-5-yl)phenyl]-3-(2-methylpyridin-4-yl)urea (13 mg, 9% yield) MS (ESI) m/z 460.

### Example 36

Preparation of 1-[4-(3-ethyl-7-morpholin-4-yl-3H-[1,2,3]triazolo[4,5-d]pyrimidin-5-yl)phenyl]-3-(4H-1,2,4-triazol-4-yl)urea. The compound was prepared as described in the example above using triphosgene (69 mg, 0.23 mmol), 4-(3-ethyl-7-morpholin-4-yl-3H-[1,2,3]triazolo[4,5-d]pyrimidin-5-yl)-phenylamine (150 mg, 0.46 mmol), 4-amino-1,2,4-triazole (116 mg, 1.38 mmol) and NEt₃ (193 μL, 1.38 mmol) in CH₂Cl₂ (3 mL) to give 1-[4-(3-ethyl-7-morpholin-4-yl-3H-[1,2,3]triazolo[4,5-d]pyrimidin-5-yl)phenyl]-3-(4H-1,2,4-triazol-4-yl)urea (43 mg, 42% yield), MS (ESI) m/z 436.4.

### Example 37

Preparation of 1-[4-(3-ethyl-7-morpholin-4-yl-3H-[1,2,3]triazolo[4,5-d]pyrimidin-5-yl)phenyl]-3-(1,3-thiazol-2-yl)urea. The compound was prepared as described in the example above using triphosgene (46 mg, 0.15 mmol), 4-(3-ethyl-7-morpholin-4-yl-3H-[1,2,3]triazolo[4,5-d]pyrimidin-5-yl)-phenylamine (100 mg, 0.31 mmol), 2-amino-thiazole (93 mg, 0.93 mmol) and NEt₃ (129 μL, 0.93 mmol) in CH₂Cl₂ (2 mL) to give 1-[4-(3-ethyl-7-morpholin-4-yl-3H-[1,2,3]triazolo[4,5-d]pyrimidin-5-yl)phenyl]-3-(1,3-thiazol-2-yl)urea (48 mg, 34% yield). MS (ESI) m/z 452.3.

### Example 38

Preparation of 2-(4-aminophenyl)ethyl [4-(3-ethyl-7-morpholin-4-yl-3H-[1,2,3]triazolo[4,5-d]pyrimidin-5-yl)phenyl]carbamate. The compound was prepared as described in the example above using triphosgene (73 mg, 0.25 mmol), 4-(3-ethyl-7-morpholin-4-yl-3H-[1,2,3]triazolo[4,5-d]pyrimidin-5-yl)-phenylamine (80 mg, 0.25 mmol), 4-aminophenethyl alcohol (101 mg, 0.73 mmol) and NEt₃ (102 μL, 0.73 mmol) in CH₂Cl₂ (2 mL) to give 2-(4-aminophenyl)ethyl [4-(3-ethyl-7-morpholin-4-yl-3H-[1,2,3]triazolo[4,5-d]pyrimidin-5-yl)phenyl]carbamate (15 mg, 12% yield), MS (ESI) m/z 489.5.

### Example 39

Preparation of 1-[4-(3-ethyl-7-morpholin-4-yl-3H-[1,2,3]triazolo[4,5-d]pyrimidin-5-yl)phenyl]-3-pyridin-3-ylurea. To a stirred solution of 4-(3-ethyl-7-morpholin-4-yl-3H-[1,2,3]triazolo[4,5-d]pyrimidin-5-yl)-phenylamine (150 mg, 0.46 mmol) in anhydrous CHCl₃ (2 mL) was added pyridine-3-isocyanate (83 mg, 0.69 mmol) and NEt₃ (97 μL, 0.69 mmol). The mixture was stirred for 18 hours and the solvents were removed in vacuo to obtain the crude product, which was purified by semi-prep-HPLC (NH₃-method), to give 1-[4-(3-ethyl-7-morpholin-4-yl-3H-[1,2,3]triazolo[4,5-d]pyrimidin-5-yl)phenyl]-3-pyridin-3-ylurea as off white solid (55 mg, 26% yield), MS (ESI) m/z 446.4.

### Example 40

Preparation of 1-[4-(3-ethyl-7-morpholin-4-yl-3H-[1,2,3]triazolo[4,5-d]pyrimidin-5-yl)phenyl]-3-(2-thienyl)urea. To a stirred solution of 4-(3-ethyl-7-morpholin-4-yl-3H-[1,2,3]triazolo[4,5-d]pyrimidin-5-yl)-phenylamine (150 mg, 0.46 mmol) in anhydrous CHCl₃ (2 mL) was added thienyl-2-isocyanate (87 mg, 0.69 mmol) and NEt₃ (97 μL, 0.69 mmol). The mixture was stirred for 18 hours and the solvents were removed in vacuo to obtain the crude product, which was purified by semi-prep-HPLC (NH₃-method), to give 1-[4-(3-ethyl-7-morpholin-4-yl-3H-[1,2,3]triazolo[4,5-d]pyrimidin-5-yl)phenyl]-3-(2-thienyl)urea as off white solid (90 mg, 43% yield, MS (ESI) m/z 451.4.

### Example 41

Preparation of methyl 4-({[4-(3-ethyl-7-morpholin-4-yl-3H-[1,2,3]triazolo[4,5-d]pyrimidin-5-yl)phenyl]carbamoyl}amino)benzoate. To a stirred solution of 4-(3-ethyl-7-morpholin-4-yl-3H-[1,2,3]triazolo[4,5-d]pyrimidin-5-yl)-phenylamine (3.26 g, 10.0 mmol) in anhydrous CH₂Cl₂ (50 μL) was added a solution of methyl-4-isocyanatobenzoate (2.12 g, 12.0 mmol) in CH₂Cl₂ (50 mL). The mixture was stirred for 8 hours and the solid was collected by filtration. The filter cake was washed with hexane (10 mL) and dried in a vacuum oven to give the product as off white solid (3.54 g, 71% yield). MS (ESI) m/z 503.3.

### Example 42

Preparation of 4-({[4-(3-ethyl-7-morpholin-4-yl-3H-[1,2,3]triazolo[4,5-d]pyrimidin-5-yl)phenyl]carbamoyl}amino)benzoic acid. In a one-neck flask equipped with reflux condenser were suspended methyl 4-({[4-(3-ethyl-7-morpholin-4-yl-3H-[1,2,3]triazolo[4,5-d]pyrimidin-5-yl)phenyl]carbamoyl}amino)benzoate (3.54 g, 7.1 mmol) in THF (20 mL), methanol (5 mL) and NaOH (5N, 5 mL, 25 mmol). The mixture was heated at reflux for 2 hours and cooled to 0° C. and acidified (pH<1) with HCl (6N). During the acidification a white solid was formed, which was collected by filtration. The filter cake was washed with water (10 mL) and dried in a vacuum oven to give the product as off-white solid (3.34 g, 98% yield). MS (ESI) m/z 489.3

### Example 43

Preparation of N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (200 mg, 0.40 mmol), N,N-dimethylethylendiamine (87 μL, 0.8 mmol) and NEt₃ (112 μL, 0.8 mmol), HOBT (110 mg, 0.8 mmol) and EDCI (154 mg, 0.8 mmol) in anhydrous THF (3 mL) to give N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)benzamide as freebase. The free base was treated with MeOH/HCl to form N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)benzamide-HCl (89 mg, 37% yield). MS (ESI) m/z 559.3.

### Example 44

Preparation of 1-[4-(3-ethyl-7-morpholin-4-yl-3H-[1,2,3]triazolo[4,5-d]pyrimidin-5-yl)phenyl]-3-{4-[(4-methylpiperazin-1-yl)carbonyl]phenyl}urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (100 mg, 0.2 mmol), 1-methylpiperazine (40 mg, 0.4 mmol) and NEt₃ (56 μL, 0.4 mmol), HOBT (55 mg, 0.4 mmol) and EDCI (77 mg, 0.4 mmol) in anhydrous THF (2 mL) to give 1-[4-(3-ethyl-7-morpholin-4-yl-3H-[1,2,3]triazolo[4,5-d]pyrimidin-5-yl)phenyl]-3-{4-[(4-methylpiperazin-1-yl)carbonyl]phenyl}urea as freebase. The free base was treated with MeOH/HCl to form 1-[4-(3-ethyl-7-morpholin-4-yl-3H-[1,2,3]triazolo[4,5-d]pyrimidin-5-yl)phenyl]-3-{4-[(4-methylpiperazin-1-yl)carbonyl]phenyl}urea —HCl (67 mg, 55% yield). MS (ESI) m/z 571.3.

### Example 45

Preparation of N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)-N-methylbenzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (100 mg, 0.2 mmol), trimethylethylenediamine (41 mg, 0.4 mmol) and NEt₃ (56 μL, 0.4 mmol), HOBT (55 mg, 0.4 mmol) and EDCI (77 mg, 0.4 mmol) in anhydrous THF (2 mL) to give N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)-N-methylbenzamide as a free base. The free base was treated with MeOH/HCl to form N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)-N-methylbenzamide-HCl (50 mg, 41% yield). MS (ESI) m/z 573.3.

### Example 46

Preparation of 4-({[4-(3-ethyl-7-morpholin-4-yl-3H-[1,2,3]triazolo[4,5-d]pyrimidin-5-yl)phenyl]carbamoyl}amino)-N-(2-hydroxyethyl)benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (100 mg, 0.2 mmol), ethanolamine (24 mg, 0.4 mmol) and NEt₃ (56 μL, 0.4 mmol), HOBT (55 mg, 0.4 mmol) and EDCI (77 mg, 0.4 mmol) in anhydrous THF (2 mL) to give 4-({[4-(3-ethyl-7-morpholin-4-yl-3H-[1,2,3]triazolo[4,5-d]pyrimidin-5-yl)phenyl]carbamoyl}amino)-N-(2-hydroxyethyl)benzamide as freebase. The free base was treated with MeOH/HCl to form 4-({[4-(3-ethyl-7-morpholin-4-yl-3H-[1,2,3]triazolo[4,5-d]pyrimidin-5-yl)phenyl]carbamoyl}amino)-N-(2-hydroxyethyl)benzamide —HCl (83 mg, 78% yield). MS (ESI) m/z 532.3.

### Example 47

Preparation of N-[3-(dimethylamino)propyl]-4-({[4-(3-ethyl-7-morpholin-4-yl-3H-[1,2,3]triazolo[4,5-d]pyrimidin-5-yl)phenyl]carbamoyl}amino)benzamide. The compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (100 mg, 0.2 mmol), N,N-dimethylpropyldiamine (40 mg, 0.4 mmol) and NEt₃ (56 μL, 0.4 mmol), HOBT (55 mg, 0.4 mmol) and EDCI (77 mg, 0.4 mmol) in anhydrous THF (2 mL) to give N-[3-(dimethylamino)propyl]-4-({[4-(3-ethyl-7-morpholin-4-yl-3H-[1,2,3]triazolo[4,5-d]pyrimidin-5-yl)phenyl]carbamoyl}amino)benzamide as the free base. The free base was treated with MeOH/HCl to form N-[3-(dimethylamino)propyl]-4-({[4-(3-ethyl-7-morpholin-4-yl-3H-[1,2,3]triazolo[4,5-d]pyrimidin-5-yl)phenyl]carbamoyl}amino)benzamide—HCl (39 mg, 32% yield). MS (ESI) m/z 573.4.

### Example 48

Preparation of 1-[4-(3-ethyl-7-morpholin-4-yl-3H-[1,2,3]triazolo[4,5-d]pyrimidin-5-yl)phenyl]-3-{4-[(4-morpholin-4-ylpiperidin-1-yl)carbonyl]phenyl}urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (100 mg, 0.2 mmol), 4-morpholinopiperidine (68 mg, 0.4 mmol) and NEt₃ (56 μL, 0.4 mmol), HOBT (55 mg, 0.4 mmol) and EDCI (77 mg, 0.4 mmol) in anhydrous THF (2 mL) to give 1-[4-(3-ethyl-7-morpholin-4-yl-3H-[1,2,3]triazolo[4,5-d]pyrimidin-5-yl)phenyl]-3-{4-[(4-morpholin-4-ylpiperidin-1-yl)carbonyl]phenyl}urea as the free base. The free base was treated with MeOH/HCl to form 1-[4-(3-ethyl-7-morpholin-4-yl-3H-[1,2,3]triazolo[4,5-d]pyrimidin-5-yl)phenyl]-3-{4-[(4-morpholin-4-ylpiperidin-1-yl)carbonyl]phenyl}urea-HCl (54 mg, 39% yield), MS (ESI) m/z 641.3.

### Example 49

Preparation of 4-({[4-(3-ethyl-7-morpholin-4-yl-3H-[1,2,3]triazolo[4,5-d]pyrimidin-5-yl)phenyl]carbamoyl}amino)-N-[2-(4-methylpiperazin-1-yl)ethyl]benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 4-methylpiperazinylethanamine (20 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-[2-(4-methylpiperazin-1-yl)ethyl]benzamide (34 mg, 55% yield). MS (ESI) m/z 614.3.

### Example 50

Preparation of 1-[4-(1,4′-bipiperidin-1′-ylcarbonyl)phenyl]-3-[4-(3-ethyl-7-morpholin-4-yl-3H-[1,2,3]triazolo[4,5-d]pyrimidin-5-yl)phenyl]urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 4-piperidinopiperidine (34 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 1-[4-(1,4′-bipiperidin-1′-ylcarbonyl)phenyl]-3-[4-(3-ethyl-7-morpholin-4-yl-3H-[1,2,3]triazolo[4,5-d]pyrimidin-5-yl)phenyl]urea (45 mg, 71% yield). MS (ESI) m/z 639.3.

### Example 51

Preparation of 4-({[4-(3-ethyl-7-morpholin-4-yl-3H-[1,2,3]triazolo[4,5-d]pyrimidin-5-yl)phenyl]carbamoyl}amino)-N-(pyridin-4-ylmethyl)benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 4-aminomethylpyridine (22 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-(pyridin-4-ylmethyl)benzamide (20 mg, 34% yield). MS (ESI) m/z 579.3.

### Example 52

Preparation of 4-({[4-(3-ethyl-7-morpholin-4-yl-3H-[1,2,3]triazolo[4,5-d]pyrimidin-5-yl)phenyl]carbamoyl}amino)-N-methyl-N-[2-(methylamino)ethyl]benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), N,N′-dimethylethylendiamine (19 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-methyl-N-[2-(methylamino)ethyl]benzamide (5 mg, 9% yield). MS (ESI) m/z 559.3.

### Example 53

Preparation of 4-({[4-(3-ethyl-7-morpholin-4-yl-3H-[1,2,3]triazolo[4,5-d]pyrimidin-5-yl)phenyl]carbamoyl}amino)-N-(2-morpholin-4-ylethyl)benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 2-(4-morpholinyl)ethanamine (26 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-(2-morpholin-4-ylethyl)benzamide (30 mg, 49% yield). MS (ESI) m/z 601.3.

### Example 54

Preparation of 1-[4-(3-ethyl-7-morpholin-4-yl-3H-[1,2,3]triazolo[4,5-d]pyrimidin-5-yl)phenyl]-3-(4-{[(3R)-3-methylpiperazin-1-yl]carbonyl}phenyl)urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), (R)-2-methylpiperazine (20 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 1-[4-(3-ethyl-7-morpholin-4-yl-3H-[1,2,3]triazolo[4,5-d]pyrimidin-5-yl)phenyl]-3-(4-{[(3R)-3-methylpiperazin-1-yl]carbonyl}phenyl)urea (35 mg, 61% yield). MS (ESI) m/z 571.3.

### Example 55

Preparation of 4-({[4-(3-ethyl-7-morpholin-4-yl-3H-[1,2,3]triazolo[4,5-d]pyrimidin-5-yl)phenyl]carbamoyl}amino)-N-[3-(4-methylpiperazin-1-yl)propyl]benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 3-aminopropyl-(4-methylpiperazine, 32 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-[3-(4-methylpiperazin-1-yl)propyl]benzamide (46 mg, 74% yield). MS (ESI) m/z 628.3.

### Example 56

Preparation of 4-({[4-(3-ethyl-7-morpholin-4-yl-3H-[1,2,3]triazolo[4,5-d]pyrimidin-5-yl)phenyl]carbamoyl}amino)-N-(2-piperidin-1-ylethyl)benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 1-aminoethylpiperidine (25 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 4-({[4-(3-ethyl-7-morpholin-4-yl-3H-[1,2,3]triazolo[4,5-d]pyrimidin-5-yl)phenyl]carbamoyl}amino)-N-(2-piperidin-1-ylethyl)benzamide (53 mg, 89% yield). MS (ESI) m/z 599.4.

### Example 57

Preparation of 1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 2,2-dimethylpiprazine (23 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea (23 mg, 40% yield). MS (ESI) m/z 585.

### Example 58

Preparation of 1-[4-(3-ethyl-7-morpholin-4-yl-3H-[1,2,3]triazolo[4,5-d]pyrimidin-5-yl)phenyl]-3-{4-[(4-pyridin-2-ylpiperazin-1-yl)carbonyl]phenyl}urea. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 1-(2-pyridyl)-piperazine (33 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea (31 mg, 48% yield). MS (ESI) m/z 634.

### Example 59

Preparation of 4-({[4-(3-ethyl-7-morpholin-4-yl-3H-[1,2,3]triazolo[4,5-d]pyrimidin-5-yl)phenyl]carbamoyl}amino)-N-[(1-ethylpyrrolidin-2-yl)methyl]benzamide. The title compound was prepared as described in the example above using 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.1 mmol), 2-aminomethyl-1-ethylpyrrolidine (25 mg, 0.2 mmol) and NEt₃ (30 μL, 0.2 mmol), HOBT (30 mg, 0.2 mmol) and EDCI (40 mg, 0.2 mmol) in anhydrous THF (1 mL) to give 1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea (23 mg, 40% yield). MS (ESI) m/z 599.

### Example 60

Preparation of 4-({[4-(3-ethyl-7-morpholin-4-yl-3H-[1,2,3]triazolo[4,5-d]pyrimidin-5-yl)phenyl]carbamoyl}amino)benzamide. A solution of 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.102 mmol), Hunig's base (79 mg, 0.612 mmol), HBTU (116 mg, 0.306 mmol) in NMP (1 mL) was stirred for 1 hr at room temperature and NH₃ (0.5N in dioxane, 306 μL, 0.15 mmol) was added. The stirring was continued overnight. The solvents were removed in a nitrogen stream and the crude mixture was purified by semi-prep-HPLC (TFA-method) to give the product a white solid (6 mg, 11% yield). MS (ESI) m/z 488.

### Example 61

Preparation of 4-({[4-(3-ethyl-7-morpholin-4-yl-3H-[1,2,3]triazolo[4,5-d]pyrimidin-5-yl)phenyl]carbamoyl}amino)-N,N-dimethylbenzamide. A solution of 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.102 mmol), Hunig's base (79 mg, 0.612 mmol), HBTU (116 mg, 0.306 mmol) in NMP (1 mL) was stirred for 1 hr at room temperature and HNMe₂ (2M in THF, 77 μL, 0.15 mmol) was added. The stirring was continued overnight. The solvents were removed in a nitrogen stream and the crude mixture was purified by semi-prep-HPLC (TFA-method) to give the product a white solid (9 mg, 17% yield). MS (ESI) m/z 516.3.

### Example 62

Preparation of N-butyl-4-({[4-(3-ethyl-7-morpholin-4-yl-3H-[1,2,3]triazolo[4,5-d]pyrimidin-5-yl)phenyl]carbamoyl}amino)benzamide. A solution of 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.102 mmol), Hunig's base (79 mg, 0.612 mmol), HBTU (116 mg, 0.306 mmol) in NMP (1 mL) was stirred for 1 hr at room temperature and n-butylamine (14 mg, 0.15 mmol) was added. The stirring was continued overnight. The solvents were removed in a nitrogen stream and the crude mixture was purified by semi-prep-HPLC (TFA-method) to give the product a white solid (30 mg, 54% yield). MS (ESI) m/z 544.3.

### Example 63

Preparation of 4-({[4-(3-ethyl-7-morpholin-4-yl-3H-[1,2,3]triazolo[4,5-d]pyrimidin-5-yl)phenyl]carbamoyl}amino)-N-(2-pyridin-2-ylethyl)benzamide. A solution of 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.102 mmol), Hunig's base (79 mg, 0.612 mmol), HBTU (116 mg, 0.306 mmol) in NMP (1 mL) was stirred for 1 hr at room temperature and 2-(2-aminoethyl)pyridine (19 mg, 0.15 mmol) was added. The stirring was continued overnight. The solvents were removed in a nitrogen stream and the crude mixture was purified by semi-prep-HPLC (TFA-method) to give the product a white solid (44 mg, 61% yield). MS (ESI) m/z 593.3.

### Example 64

Preparation N-ethyl-4-({[4-(3-ethyl-7-morpholin-4-yl-3H-[1,2,3]triazolo[4,5-d]pyrimidin-5-yl)phenyl]carbamoyl}amino)benzamide. A solution of 4-({[4-(3-ethyl-7-morpholin-4-yl-3H-[1,2,3]triazolo[4,5-d]pyrimidin-5-yl)phenyl]carbamoyl}amino)benzoic acid (50 mg, 0.102 mmol), Hunig's base (79 mg, 0.612 mmol), HBTU (116 mg, 0.306 mmol) in NMP (1 mL) was stirred for 1 hr at room temperature and ethylamine (2M in THF, 77 μL, 0.15 mmol) was added. The stirring was continued overnight. The solvents were removed in a nitrogen stream and the crude mixture was purified by semi-prep-HPLC (TFA-method) to give the product a white solid (16 mg, 30% yield). MS (ESI) m/z 516.2.

### Example 65

Preparation of benzyl 4-({[4-(3-ethyl-7-morpholin-4-yl-3H-[1,2,3]triazolo[4,5-d]pyrimidin-5-yl)phenyl]carbamoyl}amino)piperidine-1-carboxylate. To a stirred solution of 4-(3-ethyl-7-morpholin-4-yl-3H-[1,2,3]triazolo[4,5-d]pyrimidin-5-yl)-phenylamine (150 mg, 0.46 mmol) in anhydrous CHCl₃ (2 mL) was added of benzyl-4-isocyanatopiperidinecarboxylate (180 mg, 0.69 mmol) and NEt₃ (92 μL, 0.69 mmol). The mixture was stirred for 8 hours and the solvent was removed on a rotary evaporator. The crude material was purified by flash chromatography with CHCl₃/MeOH (10:1) as eluent to give the benzyl 4-({[4-(3-ethyl-7-morpholin-4-yl-3H-[1,2,3]triazolo[4,5-d]pyrimidin-5-yl)phenyl]carbamoyl}amino)piperidine-1-carboxylate as white solid (95 mg, 35%% yield). MS (ESI) m/z 586.5.

### Example 66

Preparation of 1-[4-(3-ethyl-7-morpholin-4-yl-3H-[1,2,3]triazolo[4,5-d]pyrimidin-5-yl)phenyl]-3-piperidin-4-ylurea. Benzyl 4-({[4-(3-ethyl-7-morpholin-4-yl-3H-[1,2,3]triazolo[4,5-d]pyrimidin-5-yl)phenyl]carbamoyl}amino)piperidine-1-carboxylate (120 mg, 0.21 mmol) and Pd/C (10%, wet, 80 mg) were suspended in ethanol (20 mL) and 1 drop conc. HCl was added. The mixture was hydrogenated (at 1 atm pressure) for 3 h. After completion, the catalyst was removed by filtration over Celite™ and the solvents were removed in vacuo to obtain the crude product, which was purified by semi-prep-HPLC (TFA-method), to give (25 mg 26% yield) of 1-{4-[4-morpholin-4-yl-6-(tetrahydro-pyran-4-yl)-[1,3,5]triazin-2-yl]-phenyl}-3-pyridin-4-yl-urea. MS (ESI) m/z 452.4.

### Preparation of (S)-4-(5-chloro-3-ethyl-3H-[1,2,3]triazolo[4,5-d]pyrimidin-7-yl)-3-methylmorpholine

Preparation of (S)-4-(2,6-dichloro-5-nitropyrimidin-4-yl)-3-methylmorpholine. To a solution of 2,4,6-trichloro-5-nitropyrimidine (1.98 g, 8.68 mmol) in CHCl₃ (50 mL) was added a solution of 3(S)-3-methylmorpholine (877 mg, 8.67 mmol) and Et₃N (1.21 mL, 8.67 mmol) in CHCl₃ (25 mL) at 0° C. and stirred for 5 min. at room temperature. Evaporated the solvent and purified by silica gel chromatography, Hex:EtOAc (3:1) to give the product as a yellow oil (2.48 g, 98% yield). MS (ESI) m/z 293.1.

### Step 1

(S)-2-chloro-N-ethyl-6-(3-methylmorpholino)-5-nitropyrimidin-4-amine was prepared from (S)-4-(2,6-dichloro-5-nitropyrimidin-4-yl)-3-methylmorpholine (2.3 g, 7.8 mmol) ethylamine and Et₃N (1.48 mL, 10.6 mmol) according to procedure 1 (step 1) to give the product as a yellow oil (2.3 g, 97% yield). MS (ESI) m/z 302.1.

### Step 2

(S)-2-chloro-N⁴-ethyl-6-(3-methylmorpholino)pyrimidine-4,5-diamine was prepared from (S)-4-(2,6-dichloro-5-nitropyrimidin-4-yl)-3-methylmorpholine (2.1 g, 6.96 mmol) with Raney™ nickel (5.25 g) and hydrazine (1.05 g) according to procedure 1 (step 2) to give the product as dark brown solid (1.35 g, 71% yield). MS (ESI) m/z 272.2.

### Step 3

(S)-4-(5-chloro-3-ethyl-3H-[1,2,3]triazolo[4,5-d]pyrimidin-7-yl)-3-methylmorpholine was prepared from (S)-2-chloro-N⁴-ethyl-6-(3-methylmorpholino)pyrimidine-4,5-diamine (1.2 g, 4.42 mmol), H₂O (12 mL) and AcOH (12 mL) according to procedure 2 (step 3) to give the product as a brown oil (1.2 g, 96% yield). MS (ESI) m/z 283.2.

### Example 67

Preparation of (S)-4-(3-ethyl-7-(3-methylmorpholino)-3H-[1,2,3]triazolo[4,5-d]pyrimidin-5-yl)aniline. (S)-4-(3-ethyl-7-(3-methylmorpholino)-3H-[1,2,3]triazolo[4,5-d]pyrimidin-5-yl)aniline was prepared from (S)-4-(5-chloro-3-ethyl-3H-[1,2,3]triazolo[4,5-d]pyrimidin-7-yl)-3-methylmorpholine (1.45 g, 5.40 mmol) and 4-(4,4,5,5-tetramethyl-[1,3,2]dioxaborolan-2-yl)-phenylamine (1.53 g, 7.03 mmol) following procedure 2 to give the product as a white solid (650 mg, 54% yield). MS (ESI) m/z 340.3.

### Example 68

Preparation of (S)-1-(4-(3-ethyl-7-(3-methylmorpholino)-3H-[1,2,3]triazolo[4,5-d]pyrimidin-5-yl)phenyl)-3-(4-(2-hydroxyethyl)phenyl)urea. To a solution of triphosgene (44 mg, 0.148 mmol) in CH₂Cl₂ (0.75 mL) was added (S)-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then a solution of 2-(4-aminophenyl)ethanol (61 mg, 0.44 mmol), Et₃N (62 μL, 0.44 mmol) in CH₂Cl₂ (1 mL) was added and the mixture was stirred overnight. The solvents were removed in a nitrogen stream and the residue was purified by HPLC to give the product (4.8 mg, 6% yield). MS (ESI) m/z 503.2.

### Example 69

Preparation of (S)-1-(4-(3-ethyl-7-(3-methylmorpholino)-3H-[1,2,3]triazolo[4,5-d]pyrimidin-5-yl)phenyl)-3-(thiophen-2-yl)urea. To a solution of (S)-4-(3-ethyl-7-(3-methylmorpholino)-3H-[1,2,3]triazolo[4,5-d]pyrimidin-5-yl)aniline (75 mg, 0.221 mmol) in CHCl₃ (1 mL) was added Et₃N (46 μL, 0.332 mmol) then 2-thienyl isocyanate (42 mg, 0.332 mmol). The mixture was stirred overnight and the solvent was evaporated and purified by HPLC to give the product as a tan solid (48 mg, 47% yield). MS (ESI) m/z 465.2.

### Example 70

Preparation of (S)-1-(4-(3-ethyl-7-(3-methylmorpholino)-3H-[1,2,3]triazolo[4,5-d]pyrimidin-5-yl)phenyl)-3-(4-(hydroxymethyl)phenyl)urea. To a solution of triphosgene (44 mg, 0.148 mmol) in CH₂Cl₂ (0.75 mL) was added (S)-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then a solution of (4-aminophenyl)methanol (54 mg, 0.44 mmol) and Et₃N (62 μL, 0.44 mmol) in CH₂Cl₂ (1 mL) were added and the mixture was stirred overnight. The solvents were removed in a nitrogen stream, and the residue was purified by HPLC to give the product (2.8 mg, 4% yield). MS (ESI) m/z 489.2.

### Example 71

Preparation of (S)-1-(4-(3-ethyl-7-(3-methylmorpholino)-3H-[1,2,3]triazolo[4,5-d]pyrimidin-5-yl)phenyl)-3-(pyridin-4-yl)urea. To a solution of triphosgene (44 mg, 0.148 mmol) in CH₂Cl₂ (0.75 mL) was added (S)-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then pyridin-4-amine (42 mg, 0.44 mmol) and Et₃N (62 μL, 0.44 mmol) in CH₂Cl₂ (1 mL) were added and the mixture was stirred overnight. The solvents were removed in a nitrogen stream, and the residue was purified by HPLC to give the product (32.4 mg, 38% yield). MS (ESI) m/z 460.2.

### Example 72

Preparation of (S)-1-(4-(3-ethyl-7-(3-methylmorpholino)-3H-[1,2,3]triazolo[4,5-d]pyrimidin-5-yl)phenyl)-3-(pyridin-3-yl)urea. To a solution of triphosgene (44 mg, 0.148 mmol) in CH₂Cl₂ (0.75 mL) was added (S )-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then pyridin-3-amine (42 mg, 0.44 mmol) and Et₃N (62 μL, 0.44 mmol) in CH₂Cl₂ (1 mL) were added and the mixture was stirred overnight. The solvents were removed in a nitrogen stream, and the residue was purified by HPLC to give the product (30.2 mg, 36% yield). MS (ESI) m/z 460.2.

### Example 73

Preparation of (S)-1-(4-(3-ethyl-7-(3-methylmorpholino)-3H-[1,2,3]triazolo[4,5-d]pyrimidin-5-yl)phenyl)-3-(4-methoxyphenyl)urea. To a solution of triphosgene (44 mg, 0.148 mmol) in CH₂Cl₂ (0.75 mL) was added (S)-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then 4-methoxyaniline (54 mg, 0.44 mmol), Et₃N (62 μL, 0.44 mmol) in CH₂Cl₂ (1 mL) were added and the mixture was stirred overnight. The solvents were removed in a nitrogen stream, and the residue was purified by HPLC to give the product (24.6 mg, 34% yield). MS (ESI) m/z 489.2.

### Example 74

Preparation of (S)-1-(4-(3-ethyl-7-(3-methylmorpholino)-3H-[1,2,3]triazolo[4,5-d]pyrimidin-5-yl)phenyl)-3-(4-fluorophenyl)urea. To a solution of triphosgene (44 mg, 0.148 mmol) in CH₂Cl₂ (0.75 mL) was added (S )-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then 4-fluoroaniline (49 mg, 0.44 mmol), Et₃N (62 μL, 0.44 mmol) in CH₂Cl₂ (1 mL) were added and the mixture was stirred overnight. The solvents were removed in a nitrogen stream, and the residue was purified by HPLC to give the product (29.8 mg, 43% yield). MS (ESI) m/z 477.2.

### Example 75

Preparation of (S)-1-(4-cyanophenyl)-3-(4-(3-ethyl-7-(3-methylmorpholino)-3H-[1,2,3]triazolo[4,5-d]pyrimidin-5-yl)phenyl)urea. To a solution of triphosgene (44 mg, 0.148 mmol) in CH₂Cl₂ (0.75 mL) was added (S )-4-(3-ethyl-7-(3-methylmorpholino)-3H-[1,2,3]triazolo[4,5-d]pyrimidin-5-yl)aniline (50 mg, 0.147 mmol) and the mixture was stirred for 30 min. Then 4-aminobenzonitrile (52 mg, 0.44 mmol), Et₃N (62 μL, 0.44 mmol) in CH₂Cl₂ (1 mL) were added, and the mixture was stirred overnight. The solvents were removed in a nitrogen stream, and the residue was purified by HPLC to give the product (15.2 mg, 21% yield). MS (ESI) m/z 484.2.

### Example 76

Preparation of (S)-1-(4-(3-ethyl-7-(3-methylmorpholino)-3H-[1,2,3]triazolo[4,5-d]pyrimidin-5-yl)phenyl)-3-(4-(4-methylpiperazin-1-yl)phenyl)urea. To a solution of triphosgene (44 mg, 0.148 mmol) in CH₂Cl₂ (0.75 mL) was added (S)-4-(3-ethyl-7-(3-methylmorpholino)-3H-[1,2,3]triazolo[4,5-d]pyrimidin-5-yl)aniline (50 mg, 0.147 mmol) and the mixture was stirred 30 minutes. Then 4-(4-methylpiperazin-1-yl)aniline (84 mg, 0.44 mmol) and Et₃N (62 μL, 0.44 mmol) in CH₂Cl₂ (1 mL) were added, and the mixture was stirred overnight. The solvents were removed in a nitrogen stream, and the residue was purified by HPLC to give the product (29.2 mg, 30% yield). MS (ESI) m/z 557.3.

### Example 77

Preparation of 4-(3-cyclopropyl-7-morpholino-3H-[1,2,3]triazolo[4,5-d]pyrimidin-5-yl)aniline was prepared from 4-(5-chloro-3-cyclopropyl-3H-[1,2,3]triazolo[4,5-d]pyrimidin-7-yl)morpholine (600 mg., 2.14 mmol) and 4-(4,4,5,5-tetramethyl-1,3,2-dioxaborolan-2-yl)aniline following procedure 2 to give the product as a off white solid (700 mg, 97% yield). MS (ESI) m/z 338.3.

### Example 78

Preparation of 1-(4-(3-cyclopropyl-7-morpholino-3H-[1,2,3]triazolo[4,5-d]pyrimidin-5-yl)phenyl)-3-(pyridin-4-yl)urea. To a solution of triphosgene (66 mg, 0.223 mmol) in CH₂Cl₂ (1 mL) wad added 4-(3-cyclopropyl-7-morpholino-3H-[1,2,3]triazolo[4,5-d]pyrimidin-5-yl)aniline (150 mg, 0.445 mmol) and the mixture was stirred for 30 minutes. Then, pyridin-4-amine (126 mg, 1.34 mmol) and Et₃N (187 μL, 1.34 mmol) in CH₂Cl₂ (1.5 mL) were added and the mixture was stirred overnight. The solvents were removed in a nitrogen stream, and the residue was purified by HPLC to give the product a white solid (120 mg, 59% yield). MS (ESI) m/z 458.3.

### Example 79

Preparation of 1-(4-(3-cyclopropyl-7-morpholino-3H-[1,2,3]triazolo[4,5-d]pyrimidin-5-yl)phenyl)-3-(pyridin-3-yl)urea. To a solution of 4-(3-cyclopropyl-7-morpholino-3H-[1,2,3]triazolo[4,5-d]pyrimidin-5-yl)aniline in CHCl₃ (2 mL) was added Et₃N (93 μL, 0.668 mmol) and 3-isocyanatopyridine (80 mg, 0.668 mmol). The mixture was stirred overnight and the solvent was evaporated and purified by HPLC to give the product as a white solid (112 mg, 55% yield). MS (ESI) m/z 458.3.

### Example 80

Preparation of 1-(4-(3-cyclopropyl-7-morpholino-3H-[1,2,3]triazolo[4,5-d]pyrimidin-5-yl)phenyl)-3-(thiophen-2-yl)urea. To a solution of 4-(3-cyclopropyl-7-morpholino-3H-[1,2,3]triazolo[4,5-d]pyrimidin-5-yl)aniline (75 mg, 0.222 mmol) in CHCl₃ (1 mL) was added Et₃N (46 μL, 0.0.333 mmol) and 2-isocyanatothiophene (42 mg, 0.333 mmol) The mixture was stirred overnight and the solvent was evaporated and purified by HPLC to give the product as a white solid (51 mg, 50% yield). MS (ESI) m/z 463.3.

### Preparation of 5-Chloro-3-isopropyl-7-morpholin-4-yl-3H-[1,2,3]triazolo[4,5-d]pyrimidine

### Step 1

(2-Chloro-6-morpholin-4-yl-5-nitro-pyrimidin-4-yl)-isopropyl-amine was prepared from 2,6-dichloro-5-nitro-4-morpholino-pyrimidine (2.0 g, 7.19 mmol) and isopropylamine (424 mg, 7.19 mmol) following procedure 1 (step 1) to give the final product (2.2 g, 100% yield); MS (ESI) m/z 302.

### Step 2

2-Chloro-N-4-isopropyl-6-morpholin-4-yl-pyrimidine-4,5-diamine was prepared by the reduction of (2-chloro-6-morpholin-4-yl-5-nitro-pyrimidin-4-yl)-isopropyl-amine (2.2 g, 7.03 mmol) following procedure 1 (step 2) to give the crude product (2.2 g, 100% yield); MS (ESI) m/z 272.

### Step 3

5-Chloro-3-isopropyl-7-morpholin-4-yl-3H-[1,2,3]triazolo[4,5-d]pyrimidine was prepared from (2-chloro-6-morpholin-4-yl-5-nitro-pyrimidin-4-yl)-isopropyl-amine (2.2 g, 7.03 mmol) and aqueous (0.5N) NaNO₂ solution (22 mL, 11 mmol) following procedure 1 (step 3) to give the final product (1.5 g, 74% yield); MS (ESI) m/z 283.

### Example 81

Preparation of 4-(3-isopropyl-7-morpholin-4-yl-3H-[1,2,3]triazolo[4,5-d]pyrimidin-5-yl)-phenylamine was prepared from 5-chloro-3-isopropyl-7-morpholin-4-yl-3H-[1,2,3]triazolo[4,5-d]pyrimidine (1.50 g, 5.3 mmol) 4-(4,4,5,5-tetramethyl-[1,3,2]dioxaborolan-2-yl)-phenylamine (1.74 g, 7.97 mmol) following procedure 2 to give the titled product (1.22 g, 74% yield). MS (ESI) m/z 340.

### Example 82

Preparation of 1-[4-(3-isopropyl-7-morpholin-4-yl-3H-[1,2,3]triazolo[4,5-d]pyrimidin-5-yl)-phenyl]-3-pyridin-4-yl-urea. To a stirred solution of triphosgene (39 mg, 0.13 mmol) in CH₂Cl₂ (1 mL) was added 4-(3-isopropyl-7-morpholin-4-yl-3H-[1,2,3]triazolo[4,5-d]pyrimidin-5-yl)-phenylamine (50 mg, 0.14 mmol) at 25° C. The reaction mixture was stirred for 15 min and 4-aminopyridine (42 mg, 0.44 mmol) and NEt₃ (62 μL, 0.44 mmol) was added and the reaction mixture was stirred for additional 1 hr. The solvents were removed in a nitrogen stream and the crude mixture was purified by semi-prep-HPLC (TFA-method) to give 1-[4-(3-isopropyl-7-morpholin-4-yl-3H-[1,2,3]triazolo[4,5-d]pyrimidin-5-yl)-phenyl]-3-pyridin-4-yl-urea (22 mg, 57% yield) MS (ESI) m/z 460.

### Example 83

Preparation of 1-[4-(3-isopropyl-7-morpholin-4-yl-3H-[1,2,3]triazolo[4,5-d]pyrimidin-5-yl)-phenyl]-3-pyridin-3-yl-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 3-aminopyridine (42 mg, 0.44 mmol) and NEt₃ (62 μL, 0.44 mmol) in CH₂Cl₂ (1 mL) to give 1-[4-(3-isopropyl-7-morpholin-4-yl-3H-[1,2,3]triazolo[4,5-d]pyrimidin-5-yl)-phenyl]-3-pyridin-3-yl-urea (18 mg, 47% yield). MS (ESI) m/z 460.

### Example 84

Preparation of 1-(4-Hydroxymethyl-phenyl)-3-[4-(3-isopropyl-7-morpholin-4-yl-3H-[1,2,3]triazolo[4,5-d]pyrimidin-5-yl)-phenyl]-urea. The compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 4-aminobenzylalcohol (54 mg, 0.44 mmol) and NEt₃ (62 μL, 0.44 mmol) in CH₂Cl₂ (1 mL) to give 1-[4-(3-isopropyl-7-morpholin-4-yl-3H-[1,2,3]triazolo[4,5-d]pyrimidin-5-yl)-phenyl]-3-pyridin-3-yl-urea (18 mg, 47% yield). MS (ESI) m/z 489.

### Example 85

Preparation of 1-[4-(3-Isopropyl-7-morpholin-4-yl-3H-[1,2,3]triazolo[4,5-d]pyrimidin-5-yl)-phenyl]-3-(4-morpholin-4-yl-phenyl)-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 4-morpholinylaniline (79 mg, 0.44 mmol) and NEt₃ (62 μL, 0.44 mmol) in CH₂Cl₂ (1 mL) to give 1-[4-(3-isopropyl-7-morpholin-4-yl-3H-[1,2,3]triazolo[4,5-d]pyrimidin-5-yl)-phenyl]-3-(4-morpholin-4-yl-phenyl)-urea (14 mg, 31% yield). MS (ESI) m/z 544.

### Example 86

Preparation of 1-(4-dimethylamino-phenyl)-3-[4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 4-N,N-dimethylaniline (60 mg, 0.44 mmol) and NEt₃ (62 μL, 0.44 mmol) in CH₂Cl₂ (1 mL) to give 1-(4-dimethylamino-phenyl)-3-[4-(3-isopropyl-7-morpholin-4-yl-3H-[1,2,3]triazolo[4,5-d]pyrimidin-5-yl)-phenyl]-urea (26 mg, 63% yield). MS (ESI) m/z 502.

### Example 87

Preparation of 1-(4-fluoro-phenyl)-3-[4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 4-fluoroaniline (49 mg, 0.44 mmol) and NEt₃ (62 μL, 0.44 mmol) in CH₂Cl₂ (1 mL) to give 1-(4-fluoro-phenyl)-3-[4-(3-isopropyl-7-morpholin-4-yl-3H-[1,2,3]triazolo[4,5-d]pyrimidin-5-yl)-phenyl]-urea (4 mg, 13% yield). MS (ESI) m/z 477.

### Example 88

Preparation of 1-[2-(dimethylamino)ethyl]-3-[4-(3-isopropyl-7-morpholin-4-yl-3H-[1,2,3]triazolo[4,5-d]pyrimidin-5-yl)phenyl]urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), N,N-dimethylethylendiamine (40 mg, 0.44 mmol) and NEt₃ (62 μL, 0.44 mmol) in CH₂Cl₂ (1 mL) to give 1-[2-(dimethylamino)ethyl]-3-[4-(3-isopropyl-7-morpholin-4-yl-3H-[1,2,3]triazolo[4,5-d]pyrimidin-5-yl)phenyl]urea (13 mg, 33% yield). MS (ESI) m/z 454.

### Example 89

Preparation of 1-(4-methoxy-phenyl)-3-[4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), p-anisidine (54 mg, 0.44 mmol) and NEt₃ (62 μL, 0.44 mmol) in CH₂Cl₂ (1 mL) to give 1-(4-methoxy-phenyl)-3-[4-(3-isopropyl-7-morpholin-4-yl-3H-[1,2,3]triazolo[4,5-d]pyrimidin-5-yl)-phenyl]-urea (5 mg, 14% yield). MS (ESI) m/z 489.

### Example 90

Preparation of 1-(4-methyl-phenyl)-3-[4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), p-toludine (54 mg, 0.44 mmol) and NEt₃ (62 μL, 0.44 mmol) in CH₂Cl₂ (1 mL) to give 1-(4-methyl-phenyl)-3-[4-(3-isopropyl-7-morpholin-4-yl-3H-[1,2,3]triazolo[4,5-d]pyrimidin-5-yl)-phenyl]-urea (6 mg, 19% yield). MS (ESI) m/z 473.

### Example 91

Preparation of 1-[4-(3-isopropyl-7-morpholin-4-yl-3H-[1,2,3]triazolo[4,5-d]pyrimidin-5-yl)-phenyl]-3-methyl-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), methylamine (2M solution in THF, 1 mL, 1 mmol) and NEt₃ (62 μL, 0.44 mmol) in CH₂Cl₂ (1 mL) to give 1-[4-(3-isopropyl-7-morpholin-4-yl-3H-[1,2,3]triazolo[4,5-d]pyrimidin-5-yl)-phenyl]-3-methyl-urea (21 mg, 79% yield). MS (ESI) m/z 397.

### Example 92

Preparation of 1-(1-ethyl-pyrrolidin-2-ylmethyl)-3-[4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), 2-aminomethyl-1-ethyl-pyrrolidine (56 mg, 0.44 mmol) and NEt₃ (62 μL, 0.44 mmol) in CH₂Cl₂ (1 mL) to give 1-(1-ethyl-pyrrolidin-2-ylmethyl)-3-[4-(3-isopropyl-7-morpholin-4-yl-3H-[1,2,3]triazolo[4,5-d]pyrimidin-5-yl)-phenyl]-urea (24 mg, 60% yield). MS (ESI) m/z 494.

### Example 93

Preparation of 4-{3-[4-(3-isopropyl-7-morpholin-4-yl-3H-[1,2,3]triazolo[4,5-d]pyrimidin-5-yl)-phenyl]-ureido}-benzamide. The title compound was prepared as described in the example above using triphosgene (100 mg, 0.33 mmol), 4-(3-isopropyl-7-morpholin-4-yl-3H-[1,2,3]triazolo[4,5-d]pyrimidin-5-yl)-phenylamine (140 mg, 0.41 mmol), 4-aminobenzamide (163 mg, 1.2 mmol) and NEt₃ (567 μL, 4.1 mmol) in CH₂Cl₂ (5 mL) to give 4-{3-[4-(3-isopropyl-7-morpholin-4-yl-3H-[1,2,3]triazolo[4,5-d]pyrimidin-5-yl)-phenyl]-ureido}-benzamide (68 mg, 33% yield), MS (ESI) m/z 502.

### Example 94

Preparation of 1-[4-(3-isopropyl-7-morpholin-4-yl-3H-[1,2,3]triazolo[4,5-d]pyrimidin-5-yl)-phenyl]-3-isoxazol-4-yl-urea. The title compound was prepared as described in the example above using triphosgene (100 mg, 0.33 mmol), 4-(3-isopropyl-7-morpholin-4-yl-3H-[1,2,3]triazolo[4,5-d]pyrimidin-5-yl)-phenylamine (125 mg, 0.37 mmol), isoxazol-4-ylamine (120 mg, 1.42 mmol) and NEt₃ (567 μL, 4.1 mmol) in CH₂Cl₂ (5 mL) to give 1-[4-(3-isopropyl-7-morpholin-4-yl-3H-[1,2,3]triazolo[4,5-d]pyrimidin-5-yl)-phenyl]-3-isoxazol-4-yl-urea (45 mg, 27% yield), MS (ESI) m/z 450.

### Example 95

Preparation of 1-[4-(3-isopropyl-7-morpholin-4-yl-3H-[1,2,3]triazolo[4,5-d]pyrimidin-5-yl)-phenyl]-3-(1H-pyrrol-3-yl)-urea. The title compound was prepared as described in the example above using triphosgene (58 mg, 0.20 mmol), 4-(3-isopropyl-7-morpholin-4-yl-3H-[1,2,3]triazolo[4,5-d]pyrimidin-5-yl)-phenylamine (66 mg, 0.20 mmol), 1H pyrrol-3-ylamine (120 mg, 1.42 mmol) and NEt₃ (567 μL, 4.1 mmol) in CH₂Cl₂ (5 mL) to give 1-[4-(3-isopropyl-7-morpholin-4-yl-3H-[1,2,3]triazolo[4,5-d]pyrimidin-5-yl)-phenyl]-3-(1H-pyrrol-3-yl)-urea (27 mg, 30% yield), MS (ESI) m/z 448.

### Example 96

Preparation of [4-(3-isopropyl-7-morpholin-4-yl-3H-[1,2,3]triazolo[4,5-d]pyrimidin-5-yl)-phenyl]-urea. The title compound was prepared as described in the example above using triphosgene (39 mg, 0.13 mmol), 4-(3-isopropyl-7-morpholin-4-yl-3H-[1,2,3]triazolo[4,5-d]pyrimidin-5-yl)-phenylamine (50 mg, 0.14 mmol), NH₄Cl (49 mg, 1 mmol) and NEt₃ (62 μL, 0.44 mmol) in CH₂Cl₂ (1 mL) to give [4-(3-isopropyl-7-morpholin-4-yl-3H-[1,2,3]triazolo[4,5-d]pyrimidin-5-yl)-phenyl]-urea (23 mg, 43% yield). MS (ESI) m/z 383.

Preparation of 4-[2-(5-Chloro-7-morpholin-4-yl-[1,2,3]triazolo[4,5-d]pyrimidin-3-yl)-ethyl]-piperazine-1-carboxylic acid tert-butyl ester.

### Step 1

4-[2-(2-Chloro-6-morpholin-4-yl-5-nitro-pyrimidin-4-ylamino)-ethyl]-piperazine-1-carboxylic acid tert-butyl ester was prepared from 2,6-dichloro-5-nitro-4-morpholino-pyrimidine (606 mg, 2.18 mmol) and 4-(2-amino-ethyl)-piperazine-1-carboxylic acid tert-butyl ester (500 mg, 2.18 mmol) following procedure 1 (step 1) to give the final product (1.0 g, 100% yield); MS (ESI) m/z 472.

### Step 2

4-[2-(5-Amino-2-chloro-6-morpholin-4-yl-pyrimidin-4-ylamino)-ethyl]-piperazine-1-carboxylic acid tert-butyl ester was prepared by the reduction of 4-[2-(2-chloro-6-morpholin-4-yl-5-nitro-pyrimidin-4-ylamino)-ethyl]-piperazine-1-carboxylic acid tert-butyl ester (1.03 g, 2.18 mmol) following procedure 1 (step 2) to give the final product (800 mg, 83% yield); MS (ESI) m/z 442.

### Step 3

4-[2-(5-Chloro-7-morpholin-4-yl-[1,2,3]triazolo[4,5-d]pyrimidin-3-yl)-ethyl]-piperazine-1-carboxylic acid tert-butyl ester was prepared from 4-[2-(5-amino-2-chloro-6-morpholin-4-yl-pyrimidin-4-ylamino)-ethyl]-piperazine-1-carboxylic acid tert-butyl ester (800 mg, 1.81 mmol) and aqueous (0.5N) NaNO₂ solution (18 mL, 9 mmol) following procedure 1 (step 3) to give the final product (700 mg, 85% yield); MS (ESI) m/z 453.

### Example 97

Preparation of tert-butyl 4-{2-[5-(3-hydroxyphenyl)-7-morpholin-4-yl-3H-[1,2,3]triazolo[4,5-d]pyrimidin-3-yl]ethyl}piperazine-1-carboxylate was prepared from 4-[2-(5-chloro-7-morpholin-4-yl-[1,2,3]triazolo[4,5-d]pyrimidin-3-yl)-ethyl]-piperazine-1-carboxylic acid tert-butyl ester (300 mg, 0.66 mmol) and (3-hydroxyphenyl)-boronic acid (182 mg, 1.32 mmol) following procedure 2 to give the off-white product (336 mg, 100% yield). MS (ESI) m/z 511.

### Example 98

Preparation of 3-[7-morpholin-4-yl-3-(2-piperazin-1-ylethyl)-3H-[1,2,3]triazolo[4,5-d]pyrimidin-5-yl]phenol. tert-Butyl 4-{2-[5-(3-hydroxyphenyl)-7-morpholin-4-yl-3H-[1,2,3]triazolo[4,5-d]pyrimidin-3-yl]ethyl}piperazine-1-carboxylate (500 mg, 0.18 mmol) was dissolved in CHCl₃/TFA (4:1, 20 mL) and stirred for 4 hours at 25° C., than the solvents were removed on a rotary evaporator and the crude mixture was purified by semi-prep-HPLC (NH₃-method) to give (310 mg, 76% yield). MS (ESI) m/z 411.

### Example 99

Preparation of 3-{3-[2-(4-benzoylpiperazin-1-yl)ethyl]-7-morpholin-4-yl-3H-[1,2,3]triazolo[4,5-d]pyrimidin-5-yl}phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added benzoyl chloride (10 μL). The solvents were removed in a nitrogen stream and the crude mixture was purified by semi-prep-HPLC (NH₃-method) to give the product a white solid (12 mg, 37%). MS (ESI) m/z 515.

### Example 100

Preparation of 3-{7-morpholin-4-yl-3-[2-(4-propionylpiperazin-1-yl)ethyl]-3H-[1,2,3]triazolo[4,5-d]pyrimidin-5-yl}phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added propionyl chloride (10 μL). The solvents were removed in a nitrogen stream and the crude mixture was purified by semi-prep-HPLC (NH₃-method) to give the product a white solid (8 mg, 27%). MS (ESI) m/z 467.

### Example 101

Preparation of 3-(3-{2-[4-(4-fluorobenzoyl)piperazin-1-yl]ethyl}-7-morpholin-4-yl-3H-[1,2,3]triazolo[4,5-d]pyrimidin-5-yl)phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added 4-fluorobenzoylchloride (10 μL). The solvents were removed in a nitrogen stream and the crude mixture was purified by semi-prep-HPLC (NH₃-method) to give the product a white solid (7 mg, 19%). MS (ESI) m/z 533.

### Example 102

Preparation of 3-(3-{2-[4-(3,4-difluorobenzoyl)piperazin-1-yl]ethyl}-7-morpholin-4-yl-3H-[1,2,3]triazolo[4,5-d]pyrimidin-5-yl)phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added 3,4-difluorobenzoylchloride (10 μL). The solvents were removed in a nitrogen stream and the crude mixture was purified by semi-prep-HPLC (NH₃-method) to give the product a white solid (10 mg, 28%). MS (ESI) m/z 551.

### Example 103

Preparation of 3-{3-[2-(4-isonicotinoylpiperazin-1-yl)ethyl]-7-morpholin-4-yl-3H-[1,2,3]triazolo[4,5-d]pyrimidin-5-yl}phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added isonicotinoyl chloride (10 μL). The solvents were removed in a nitrogen stream and the crude mixture was purified by semi-prep-HPLC (NH₃-method) to give the product a white solid (8 mg, 23%). MS (ESI) m/z 516.

### Example 104

Preparation of 3-(7-morpholin-4-yl-3-{2-[4-(phenylacetyl)piperazin-1-yl]ethyl}-3H-[1,2,3]triazolo[4,5-d]pyrimidin-5-yl)phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added phenylacetyl chloride (10 μL). The solvents were removed in a nitrogen stream and the crude mixture was purified by semi-prep-HPLC (NH₃-method) to give the product a white solid (7 mg, 22%). MS (ESI) m/z 529.

### Example 105

Preparation of 3-{3-[2-(4-acetylpiperazin-1-yl)ethyl]-7-morpholin-4-yl-3H-[1,2,3]triazolo[4,5-d]pyrimidin-5-yl}phenol. To a stirred solution of 3-[7-morpholin-4-yl-3-(2-piperazin-1-ylethyl)-3H-[1,2,3]triazolo[4,5-d]pyrimidin-5-yl]phenol (26 mg, 0.06 mmol) and NEt₃ (10 μL, 0.07 mmol) in THF (1 mL) was added acetyl chloride (10 μL). The solvents were removed in a nitrogen stream and the crude mixture was purified by semi-prep-HPLC (NH₃-method) to give the product a white solid (13 mg, 45%). MS (ESI) m/z 453.

### Example 106

Preparation of 3-{3-[2-(4-cyclohexylpiperazin-1-yl)ethyl]-7-morpholin-4-yl-3H-[1,2,3]triazolo[4,5-d]pyrimidin-5-yl}phenol. 3-[7-Morpholin-4-yl-3-(2-piperazin-1-ylethyl)-3H-[1,2,3]triazolo[4,5-d]pyrimidin-5-yl]phenol (15 mg, 0.04 mmol) was dissolved in methanol (1 mL) and cyclohexanone (15 μL, 0.2 mmol), NaBH₃CN (15 mg, 0.23 mmol) and ZnCl₂ (15 mg, 0.11 mmol) was added. The suspension was stirred for 24 hours and the solvents were removed in vacuo. The crude product was dissolved in DMSO (2 mL), filtered, and purified by semi-prep-HPLC using ACN/water/NH₃ as mobile phase. After combining the product fractions and removing solvent, the product was obtained as a white solid. (8 mg, 42%). MS (ESI) m/z 493.4.

### Example 107

Preparation of 3-{3-[2-(4-butylpiperazin-1-yl)ethyl]-7-morpholin-4-yl-3H-[1,2,3]triazolo[4,5-d]pyrimidin-5-yl}phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), butyraldehyde (15 μL), NaBH₃CN (15 mg, 0.23 mmol), and ZnCl₂ (15 mg, 0.11 mmol) as described in the example above to give the product (10 mg, 58% yield). MS (ESI) m/z 467.

### Example 108

Preparation of 3-{3-[2-(4-isobutylpiperazin-1-yl)ethyl]-7-morpholin-4-yl-3H-[1,2,3]triazolo[4,5-d]pyrimidin-5-yl}phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), isobutyraldehyde (15 μL), NaBH₃CN (15 mg, 0.23 mmol), and ZnCl₂ (15 mg, 0.11 mmol) as described in the example above to give the product (10 mg, 58% yield). MS (ESI) m/z 467.

### Example 109

Preparation of 3-(3-{2-[4-(3-fluorobenzyl)piperazin-1-yl]ethyl}-7-morpholin-4-yl-3H-[1,2,3]triazolo[4,5-d]pyrimidin-5-yl)phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), 3-fluorobenzaldehyde (15 μL), NaBH₃CN (15 mg, 0.23 mmol), and ZnCl₂ (15 mg, 0.11 mmol) as described in the example above to give the product (9 mg, 48% yield). MS (ESI) m/z 519.

### Example 110

Preparation of 3-{3-[2-(4-{4-[3-(dimethylamino)propoxy]benzyl}piperazin-1-yl)ethyl]-7-morpholin-4-yl-3H-[1,2,3]triazolo[4,5-d]pyrimidin-5-yl}phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), 4-(3-dimethylaminopropoxy)-benzaldehyde (15 μL), NaBH₃CN (15 mg, 0.23 mmol), and ZnCl₂ (15 mg, 0.11 mmol) as described in the example above to give the product (10 mg, 47% yield); MS (ESI) m/z 602.

### Example 111

Preparation of 3-(7-morpholin-4-yl-3-{2-[4-(pyridin-3-ylmethyl)piperazin-1-yl]ethyl}-3H-[1,2,3]triazolo[4,5-d]pyrimidin-5-yl)phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), 3-pyridinecarbaldehyde (15 μL), NaBH₃CN (15 mg, 0.23 mmol), and ZnCl₂ (15 mg, 0.11 mmol) as described in the example above to give the product (9 mg, 51% yield); MS (ESI) m/z 502.

### Example 112

Preparation of 3-(7-morpholin-4-yl-3-{2-[4-(1H-pyrrol-2-ylmethyl)piperazin-1-yl]ethyl}-3H-[1,2,3]triazolo[4,5-d]pyrimidin-5-yl)phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), 2-pyrrolcarbaldehyde (15 mg), NaBH₃CN (15 mg, 0.23 mmol), and ZnCl₂ (15 mg, 0.11 mmol) as described in the example above to give the product (10 mg, 57% yield); MS (ESI) m/z 490.

### Example 113

Preparation of 3-(3-{2-[4-(2-furylmethyl)piperazin-1-yl]ethyl}-7-morpholin-4-yl-3H-[1,2,3]triazolo[4,5-d]pyrimidin-5-yl)phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), furylaldehyde (15 mg), NaBH₃CN (15 mg, 0.23 mmol), and ZnCl₂ (15 mg, 0.11 mmol) as described in the example above to give the product (16 mg, 92% yield); MS (ESI) m/z 491.

### Example 114

Preparation of 3-{3-[2-(4-benzylpiperazin-1-yl)ethyl]-7-morpholin-4-yl-3H-[1,2,3]triazolo[4,5-d]pyrimidin-5-yl}phenol was prepared from 3-[7-morpholin-4-yl-3-(2-piperazin-1-ylethyl)-3H-[1,2,3]triazolo[4,5-d]pyrimidin-5-yl]phenol (15 mg, 0.04 mmol), benzaldehyde (15 μL), NaBH₃CN (15 mg, 0.23 mmol), and ZnCl₂ (15 mg, 0.11 mmol) as described in the example above to give the product (8 mg, 46% yield); MS (ESI) m/z 501.

### 3-(5-Chloro-7-morpholin-4-yl-[1,2,3]triazolo[4,5-d]pyrimidin-3-yl)-benzoic Acid Methyl Ester

### Step 1

3-(2-Chloro-6-morpholin-4-yl-5-nitro-pyrimidin-4-ylamino)-benzoic acid methyl ester was prepared from 2,6-dichloro-5-nitro-4-morpholino-pyrimidine (2.0 g, 7.17 mmol) and methyl-4-amino-benzoate (1.09 mL, 7.19 mmol) following procedure 1 (step 1) to give the final product (2.04 g, 71% yield); MS (ESI) m/z 394.

### Step 2

3-(5-Amino-2-chloro-6-morpholin-4-yl-pyrimidin-4-ylamino)-benzoic acid methyl ester was prepared by the reduction of 3-(2-chloro-6-morpholin-4-yl-5-nitro-pyrimidin-4-ylamino)-benzoic acid methyl ester (2.4 g, 6.13 mmol) following procedure 1 (step 2) to give the final product (2.4 g, 100% yield); MS (ESI) m/z 364.

### Step 3

3-(5-chloro-7-morpholin-4-yl-[1,2,3]triazolo[4,5-d]pyrimidin-3-yl)-benzoic acid methyl ester was prepared from 3-(5-amino-2-chloro-6-morpholin-4-yl-pyrimidin-4-ylamino)-benzoic acid methyl ester (2.4 g, 6.13 mmol) and aqueous (0.5N) NaNO₂ solution (26 mL, 13 mmol) following procedure 1 (step 3) to give the final product (1.3 g, 70% yield); MS (ESI) m/z 375.

### Example 115

Preparation of methyl 3-[5-(3-hydroxyphenyl)-7-morpholin-4-yl-3H-[1,2,3]triazolo[4,5-d]pyrimidin-3-yl]benzoate was prepared from 3-(5-chloro-7-morpholin-4-yl-[1,2,3]triazolo[4,5-d]pyrimidin-3-yl)-benzoic acid methyl ester (250 mg, 0.66 mmol) and 3-hydroxyphenylboronic acid (184 mg, 0.99 mmol) following procedure 2 to give the titled product (220 mg, 77% yield). MS (ESI) m/z 433.3.

Intermediate

Preparation of methyl 3-[5-(4-aminophenyl)-7-morpholin-4-yl-3H-[1,2,3]triazolo[4,5-d]pyrimidin-3-yl]benzoate was prepared from 3-(5-chloro-7-morpholin-4-yl-[1,2,3]triazolo[4,5-d]pyrimidin-3-yl)-benzoic acid methyl ester (1.3 g, 3.5 mmol) and 4-(4,4,5,5-tetramethyl-[1,3,2]dioxaborolan-2-yl)-phenylamine (1.15 g, 5.25 mmol) following procedure 2 to give the titled product (1.1 g, 73% yield). MS (ESI) m/z 432.2.

### Example 116

Preparation of methyl 3-[5-(3-formylphenyl)-7-morpholin-4-yl-3H-[1,2,3]triazolo[4,5-d]pyrimidin-3-yl]benzoate was prepared from 3-(5-chloro-7-morpholin-4-yl-[1,2,3]triazolo[4,5-d]pyrimidin-3-yl)-benzoic acid methyl ester (375 mg, 1 mmol) and 3-formylphenylboronic acid (300 mg, 2 mmol) following procedure 2 to give the titled product (400 mg, 90% yield). MS (ESI) m/z 445.4.

### Example 117

Preparation of [(7-morpholin-4-yl-3H-[1,2,3]triazolo[4,5-d]pyrimidine-3,5-diyl)di-3,1-phenylene]dimethanol. 3-[5-(3-formylphenyl)-7-morpholin-4-yl-3H-[1,2,3]triazolo[4,5-d]pyrimidin-3-yl]benzoate (100 mg, 0.22 mmol) was suspended in anhydrous THF (2 mL) and cooled to 0° C. LAH (2M solution in THF, 110 mL, 0.22 mmol) was added slowly and the mixture was stirred for 2 hours. After the reaction was completed, THF/water (9: 1, 100 mL) and NaOH (1N, 100 mL) was added, the solid was filtered off. The filtrate was evaporated and the crude compound was purified by preparative HPLC using ACN/water/TFA-gradients as eluent to give the product as white solid (10 mg, 35%), MS (ESI) m/z 419.

### Example 118

Preparation of 3-[5-(3-hydroxyphenyl)-7-morpholin-4-yl-3H-[1,2,3]triazolo[4,5-d]pyrimidin-3-yl]benzoic acid. In a one-neck flask equipped with reflux condenser were suspended 3-[5-(3-hydroxyphenyl)-7-morpholin-4-yl-3H-[1,2,3]triazolo[4,5-d]pyrimidin-3-yl]benzoate (60 mg, 7.1 mmol) in THF (4 mL) and NaOH (2.5N, 4 mL, 10 mmol). The mixture was heated at reflux for 1 hours and cooled to 0° C. and acidified (pH<1) with HCl (6N). During the acidification a white solid was formed, which was collected by filtration. The filter cake was washed with water (0.1 mL) and dried in a vacuum oven to give the product as white solid (16 mg, 27% yield), MS (ESI) m/z 417.2.

### Example 119

Preparation of 3-[5-(3-hydroxyphenyl)-7-morpholin-4-yl-3H-[1,2,3]triazolo[4,5-d]pyrimidin-3-yl]benzamide. In a one-neck flask, equipped with a stirring bar, under nitrogen atmosphere, was suspended 3-[5-(3-hydroxyphenyl)-7-morpholin-4-yl-3H-[1,2,3]triazolo[4,5-d]pyrimidin-3-yl]benzoic acid (80 mg, 0.2 mmol) in CHCl₃ (1 mL). The mixture was stirred at 25° C. and COCl₂ (2M in CH₂Cl₂, 0.3 mL, 0.6 mmol) and one drop of DMF were added. After 30 minutes NH₃ (2M solution in THF, 0.6 mL, 1.2 mmol) was added and the reaction mixture was stirred for additional 1 hr. The solvents were removed in a nitrogen stream and the crude mixture was purified by semi-prep-HPLC (TFA-method) to give 3-[5-(3-hydroxyphenyl)-7-morpholin-4-yl-3H-[1,2,3]triazolo[4,5-d]pyrimidin-3-yl]benzamide (12 mg, 14% yield) MS (ESI) m/z 418.2.

### Example 120

Preparation of 3-(7-morpholin-4-yl-3-{3-[(4-pyrrolidin-1-ylpiperidin-1-yl)carbonyl]phenyl}-3H-[1,2,3]triazolo[4,5-d]pyrimidin-5-yl)phenol. To a stirred suspension 4-({[4-(3-ethyl-7-morpholin-4-yl-3H-[1,2,3]triazolo[4,5-d]pyrimidin-5-yl)phenyl]carbamoyl}amino)benzoic acid (10 mg, 0.02 mmol,) HOBT (10 mg, 0.08 mmol), 4-(1-pyrrolidinyl)-piperidine (20 mg, 0.13 mmol) and NEt₃ (10 μL, 0.08 mmol) was added EDCI (10 mg, 0.05 mmol) and the mixture allowed to stir overnight. The solvents were removed in a nitrogen stream and the crude mixture was purified by semi-prep-HPLC (TFA-method) to give the product as a white solid (5 mg, 34%). MS (ESI) m/z 555.

### Example 121

Preparation of 3-[5-(3-hydroxyphenyl)-7-morpholin-4-yl-3H-[1,2,3]triazolo[4,5-d]pyrimidin-3-yl]-N-methylbenzamide. To a stirred suspension 4-({[4-(3-ethyl-7-morpholin-4-yl-3H-[1,2,3]triazolo[4,5-d]pyrimidin-5-yl)phenyl]carbamoyl}amino)benzoic acid (10 mg, 0.02 mmol,) HOBT (10 mg, 0.08 mmol), MeNH₂ (2M solution in THF, 50 μL, 0.1 mmol) and NEt₃ (10 μL, 0.08 mmol) was added EDCI (10 mg, 0.05 mmol) and the mixture allowed to stir overnight. The solvents were removed in a nitrogen stream and the crude mixture was purified by semi-prep-HPLC (TFA-method) to give the product as a white solid (6 mg, 56%). MS (ESI) m/z 432.

### Example 122

Preparation of N-[2-(dimethylamino)ethyl]-3-[5-(3-hydroxyphenyl)-7-morpholin-4-yl-3H-[1,2,3]triazolo[4,5-d]pyrimidin-3-yl]benzamide. To a stirred suspension 4-({[4-(3-ethyl-7-morpholin-4-yl-3H-[1,2,3]triazolo[4,5-d]pyrimidin-5-yl)phenyl]carbamoyl}amino)benzoic acid (10 mg, 0.02 mmol,) HOBT (10 mg, 0.08 mmol), N,N-dimethylethylenediamine (10 mg, 0.1 mmol) and NEt₃ (10 μL, 0.08 mmol) was added EDCI (10 mg, 0.05 mmol) and the mixture allowed to stir overnight. The solvents were removed in a nitrogen stream and the crude mixture was purified by semi-prep-HPLC (TFA-method) to give the product as a white solid (6 mg, 45%). MS (ESI) m/z 489.

### Example 123

Preparation of 3-(7-morpholin-4-yl-5-{4-[(phenylcarbamoyl)amino]phenyl}-3H-[1,2,3]triazolo[4,5-d]pyrimidin-3-yl)benzoic acid. To a stirred solution of methyl 3-[5-(4-aminophenyl)-7-morpholin-4-yl-3H-[1,2,3]triazolo[4,5-d]pyrimidin-3-yl]benzoate (240 mg, 0.55 mmol) in anhydrous CH₂Cl₂ (30 mL) was added 4-phenylisocyanate (340 mg, 2.86 mmol) and DMAP (20 mg, 0.16 mmol). The mixture was stirred for 8 hours and the solid was collected by filtration. The filter cake was washed with hexane (10 mL). The obtained solid was placed in a one-neck flask equipped with reflux condenser and THF (4 mL) and NaOH (2.5N, 4 mL, 10 mmol) were added. The mixture was heated at reflux for 1 hours and cooled to 0° C. and acidified (pH<1) with HCl (6N). During the acidification a white solid was formed, which was collected by filtration. The filter cake was washed with water (0.1 mL) and dried in a vacuum oven to give the product as white solid (120 mg, 49% yield), (ESI) m/z 432.2.

### 3-(5-Chloro-7-morpholin-4-yl-[1,2,3]triazolo[4,5-d]pyrimidin-3-yl)-azetidine-1-carboxylic Acid Tert-Butyl Ester

### Step 1

3-(2-Chloro-6-morpholin-4-yl-5-nitro-pyrimidin-4-ylamino)-azetidine-1-carboxylic acid tert-butyl ester was prepared from 2,6-dichloro-5-nitro-4-morpholino-pyrimidine (1.62 g, 5.8 mmol) and 3-amino-cyclobutanecarboxylic acid tert-butyl ester (1 g, 5.8 mmol) following procedure 1 (step 1) to give the final product (2.0 g, 83% yield); MS (ESI) m/z 415.

### Step 2

3-(5-Amino-2-chloro-6-morpholin-4-yl-pyrimidin-4-ylamino)-azetidine-1-carboxylic acid tert-butyl ester was prepared by the reduction of 3-(2-chloro-6-morpholin-4-yl-5-nitro-pyrimidin-4-ylamino)-azetidine-1-carboxylic acid tert-butyl ester (800 mg, 1.93 mmol) following procedure 1 (step 2) to give the final product (740 g, 100% yield); MS (ESI) m/z 385.

### Step 3

3-(5-Chloro-7-morpholin-4-yl-[1,2,3]triazolo[4,5-d]pyrimidin-3-yl)-azetidine-1-carboxylic acid tert-butyl ester was prepared from 3-(5-amino-2-chloro-6-morpholin-4-yl-pyrimidin-4-ylamino)-azetidine-1-carboxylic acid tert-butyl ester (740 mg, 1.93 mmol) and aqueous (0.5N) NaNO₂ solution (8 mL, 13 mmol) following procedure 1 (step 3) to give the final product (600 mg, 78% yield); MS (ESI) m/z 396.

### Example 124

Preparation of tert-butyl 3-[5-(3-hydroxyphenyl)-7-morpholin-4-yl-3H-[1,2,3]triazolo[4,5-d]pyrimidin-3-yl]azetidine-1-carboxylate was prepared from 3-(5-chloro-7-morpholin-4-yl-[1,2,3]triazolo[4,5-d]pyrimidin-3-yl)-azetidine-1-carboxylic acid tert-butyl ester (180 mg, 0.66 mmol) and 3-hydroxyphenylboronic acid (125 mg, 0.9 mmol) following procedure 2 to give the titled product (180 mg, 88% yield). MS (ESI) m/z 454.4.

### Example 125

Preparation of 3-(3-azetidin-3-yl-7-morpholin-4-yl-3H-[1,2,3]triazolo[4,5-d]pyrimidin-5-yl)phenol. tert-butyl 3-[5-(3-hydroxyphenyl)-7-morpholin-4-yl-3H-[1,2,3]triazolo[4,5-d]pyrimidin-3-yl]azetidine-1-carboxylate (180 mg, 0.4 mmol). was dissolved

CHCl₃/TFA (2:1, 6 mL) and stirred for 4 hours at 25° C., than the solvents were removed on a rotary evaporator and the crude mixture was purified by semi-prep-HPLC (NH₃-method) to give (80 mg, 55% yield). MS (ESI) m/z 354.4.

### Example 126

Preparation of (2-amino-phenyl)-{3-[5-(3-hydroxy-phenyl)-7-morpholin-4-yl-[1,2,3]triazolo[4,5-d]pyrimidin-3-yl]-azetidin-1-yl}-methanone. To a stirred solution of 3-(3-Azetidin-3-yl-7-morpholin-4-yl-3H-[1,2,3]triazolo[4,5-d]pyrimidin-5-yl)-phenol (300 mg, 0.85 mmol) and NEt₃ (177 μL, 1.27 mmol) in CHCl₃ (4 mL) was added 2-nitrobenzoychloride (236 mg, 1.27 ml). The solvents were removed in a nitrogen stream and the crude mixture was dissolved in methanol (40 ml), Ni(Ra)™ (1 g,) and hydrazine (200 μL) were added. The suspension was stirred for 15 minutes and the catalyst was removed by filtration with Celite™. The volatiles were removed on a rotary evaporator and the crude mixture was purified by semi-prep-HPLC (NH₃-method) to give (2-amino-phenyl)-{3-[5-(3-hydroxy-phenyl)-7-morpholin-4-yl-[1,2,3]triazolo[4,5-d]pyrimidin-3-yl]-azetidin-1-yl}-methadone (168 mg, 42% yield). MS (ESI) m/z 473.2.

### Example 127

Preparation of 3-[3-(1-benzylazetidin-3-yl)-7-morpholin-4-yl-3H-[1,2,3]triazolo[4,5-d]pyrimidin-5-yl]phenol. 3-(3-Azetidin-3-yl-7-morpholin-4-yl-3H-[1,2,3]triazolo[4,5-d]pyrimidin-5-yl)phenol (20 mg, 0.06 mmol) was dissolved in methanol (1 mL) and benzaldehyde (15 μL, 0.1 mmol), NaBH₃CN (15 mg, 0.23 mmol) and ZnCl₂ (15 mg, 0.11 mmol) was added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions and removal of solvent, the product was obtained as a white solid. (13 mg, 52%). MS (ESI) m/z 444.

### Example 128

Preparation of 3-(3-{1-[(6-fluoropyridin-3-yl)methyl]azetidin-3-yl}-7-morpholin-4-yl-3H-[1,2,3]triazolo[4,5-d]pyrimidin-5-yl)phenol. 3-(3-Azetidin-3-yl-7-morpholin-4-yl-3H-[1,2,3]triazolo[4,5-d]pyrimidin-5-yl)phenol (20 mg, 0.06 mmol) was dissolved in methanol (1 mL) and 6-fluoronicotinaldehyde (13 mg, 0.1 mmol), NaBH₃CN (15 mg, 0.23 mmol) and ZnCl₂ (15 mg, 0.11 mmol) was added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions and solvent removal, the product was obtained as a white solid. (14 mg, 54%). MS (ESI) m/z 463.

### Example 129

Preparation of tert-butyl 3-(7-morpholin-4-yl-5-{4-[(pyridin-4-ylcarbamoyl)amino]phenyl}-3H-[1,2,3]triazolo[4,5-d]pyrimidin-3-yl)azetidine-1-carboxylate. To a stirred solution of triphosgene (20 mg, 0.07 mmol) in CHCl₃ (1 mL) was added 3-[5-(4-amino-phenyl)-7-morpholin-4-yl-[1,2,3]triazolo[4,5-d]pyrimidin-3-yl]-azetidine-1-carboxylic acid tert-butyl ester (30 mg, 0.07 mmol) at 0° C. The reaction mixture was stirred for 15 min and 4-aminopyridine (50 mg, 0.46 mmol) and NEt₃ (64 μL, 0.46 mmol) was added and the reaction mixture was stirred for additional 1 hr. The solvents were removed on a rotary evaporator and the crude mixture was purified by semi-prep-HPLC (TFA-method) to give tert-butyl 3-(7-morpholin-4-yl-5-{4-[(pyridin-4-ylcarbamoyl)amino]phenyl}-3H-[1,2,3]triazolo[4,5-d]pyrimidin-3-yl)azetidine-1-carboxylate (7 mg, 16% yield) MS (ESI) m/z 573.

### Example 130

Preparation of tert-butyl 3-(7-morpholin-4-yl-5-{4-[(pyridin-3-ylcarbamoyl)amino]phenyl}-3H-[1,2,3]triazolo[4,5-d]pyrimidin-3-yl)azetidine-1-carboxylate. To a stirred solution of triphosgene (20 mg, 0.07 mmol) in CHCl₃ (1 mL) was added 3-[5-(4-amino-phenyl)-7-morpholin-4-yl-[1,2,3]triazolo[4,5-d]pyrimidin-3-yl]-azetidine-1-carboxylic acid tert-butyl ester (30 mg, 0.07 mmol) at 0° C. The reaction mixture was stirred for 15 min and 3-aminopyridine (50 mg, 0.46 mmol) and NEt₃ (64 μL, 0.46 mmol) was added and the reaction mixture was stirred for additional 1 hr. The solvents were removed on a rotary evaporator and the crude mixture was purified by semi-prep-HPLC (TFA-method) to give tert-butyl 3-(7-morpholin-4-yl-5-{4-[(pyridin-4-ylcarbamoyl)amino]phenyl}-3H-[1,2,3]triazolo[4,5-d]pyrimidin-3-yl)azetidine-1-carboxylate (6 mg, 14% yield) MS (ESI) m/z 573.

### Example 131

Preparation of tert-butyl 3-(7-morpholin-4-yl-5-{4-[(phenylcarbamoyl)amino]phenyl}-3H-[1,2,3]triazolo[4,5-d]pyrimidin-3-yl)azetidine-1-carboxylate. To a stirred solution of methyl 3-[5-(4-amino-phenyl)-7-morpholin-4-yl-[1,2,3]triazolo[4,5-d]pyrimidin-3-yl]-azetidine-1-carboxylic acid tert-butyl ester (30 mg, 0.07 mmol) in anhydrous CH₂Cl₂ (1 mL) was added 4-phenylisocyanate (18 mg, 0.15 mmol). The mixture was stirred for 8 hours and the solvents were removed on a rotary evaporator and the crude mixture was purified by semi-prep-HPLC (TFA-method) to give tert-butyl 3-(7-morpholin-4-yl-5-{4-[(phenylcarbamoyl)amino]phenyl}-3H-[1,2,3]triazolo[4,5-d]pyrimidin-3-yl)azetidine-1-carboxylate (18 mg, 55% yield) MS (ESI) m/z 496.

### Example 132

Preparation of tert-butyl 3-(7-morpholin-4-yl-5-{4-[(2-thienylcarbamoyl)amino]phenyl}-3H-[1,2,3]triazolo[4,5-d]pyrimidin-3-yl)azetidine-1-carboxylate. To a stirred solution of methyl 3-[5-(4-amino-phenyl)-7-morpholin-4-yl-[1,2,3]triazolo[4,5-d]pyrimidin-3-yl]-azetidine-1-carboxylic acid tert-butyl ester (30 mg, 0.07 mmol) in anhydrous CH₂Cl₂ (1 mL) was added 2-thienyllisocyanate (8 mg, 0.07 mmol). The mixture was stirred for 8 hours and the solvents were removed on a rotary evaporator and the crude mixture was purified by semi-prep-HPLC (TFA-method) to give tert-butyl thienylcarbamoyl)amino]phenyl}-3H-[1,2,3]triazolo[4,5-d]pyrimidin-3-yl)azetidine-1-carboxylate (7 mg, 21% yield) MS (ESI) m/z 502.

### Preparation of 5-Chloro-7-morpholin-4-yl-3-(2,2,2-trifluoro-ethyl)-3H-[1,2,3]triazolo[4,5-d]pyrimidine

### Step 1

(2-Chloro-6-morpholin-4-yl-5-nitro-pyrimidin-4-yl)-(2,2,2-trifluoro-ethyl)-amine was prepared from 2,6-dichloro-5-nitro-4-morpholino-pyrimidine (1.0 g, 3.58 mmol) and 2,2,2-trifluoroethylamine (3.94 mg, 3.94 mmol) following procedure 1 (step 1) to give the final product (700 mg, 57% yield); MS (ESI) m/z 341.

### Step 2

2-Chloro-6-morpholin-4-yl-N-4-(2,2,2-trifluoro-ethyl)-pyrimidine-4,5-diamine was prepared by the reduction of (2-chloro-6-morpholin-4-yl-5-nitro-pyrimidin-4-yl)-(2,2,2-trifluoroethyl)-amine (700 mg, 2.05 mmol) following procedure 1 (step 2) to give the final product (600 mg, 94% yield); MS (ESI) m/z 312.

### Step 3

5-Chloro-7-morpholin-4-yl-3-(2,2,2-trifluoro-ethyl)-3H-[1,2,3]triazolo[4,5-d]pyrimidine was prepared from (2-chloro-6-morpholin-4-yl-N⁴-(2,2,2-trifluoro-ethyl)-pyrimidine-4,5-diamine (600 mg, 1.93 mmol) and aqueous (0.5N) NaNO₂ solution (6 mL, 3.0 mmol) following procedure 1 (step 3) to give the final product (430 mg, 68% yield); MS (ESI) m/z 323.

### Example 133

Preparation of 4-[7-morpholin-4-yl-3-(2,2,2-trifluoroethyl)-3H-[1,2,3]triazolo[4,5-d]pyrimidin-5-yl]aniline was prepared from 5-chloro-7-morpholin-4-yl-3-(2,2,2-trifluoroethyl)-3H-[1,2,3]triazolo[4,5-d]pyrimidine (400 mg, 1.23 mmol) and 4-(4,4,5,5-tetramethyl-[1,3,2]dioxaborolan-2-yl)-phenylamine (500 mg, 2.28 mmol) following procedure 2 to give the titled product (244 mg, 50% yield). MS (ESI) m/z 380.2.

### Example 134

Preparation of 1-{4-[7-morpholin-4-yl-3-(2,2,2-trifluoroethyl)-3H-[1,2,3]triazolo[4,5-d]pyrimidin-5-yl]phenyl}-3-pyridin-4-ylurea. To a stirred solution of triphosgene (189 mg, 0.64 mmol) in CHCl₃ (15 mL) was added 4-[7-morpholin-4-yl-3-(2,2,2-trifluoroethyl)-3H-[1,2,3]triazolo[4,5-d]pyrimidin-5-yl]aniline (240 mg, 0.64 mmol) at 0° C. The reaction mixture was stirred for 15 min and 4-aminopyridine (94 mg, 1 mmol) and NEt₃ (200 μL, 1.44 mmol) was added and the reaction mixture was stirred for additional 1 hr. The solvents were removed on a rotary evaporator and the crude mixture was purified by semi-prep-HPLC (NH₃-method) to give 1-{4-[7-morpholin-4-yl-3-(2,2,2-trifluoroethyl)-3H-[1,2,3]triazolo[4,5-d]pyrimidin-5-yl]phenyl}-3-pyridin-4-ylurea (93 mg, 29% yield) MS (ESI) m/z 500.

### Example 135

Preparation of 1-{4-[7-morpholin-4-yl-3-(2,2,2-trifluoroethyl)-3H-[1,2,3]triazolo[4,5-d]pyrimidin-5-yl]phenyl}-3-pyridin-3-ylurea. To a stirred solution of triphosgene (94 mg, 0.32 mmol) in CHCl₃ (7 mL) was added 4-[7-morpholin-4-yl-3-(2,2,2-trifluoroethyl)-3H-[1,2,3]triazolo[4,5-d]pyrimidin-5-yl]aniline (120 mg, 0.32 mmol) at 0° C. The reaction mixture was stirred for 15 min and 3-aminopyridine (94 mg, 1 mmol) and NEt₃ (100 μL, 0.77 mmol) was added and the reaction mixture was stirred for additional 1 hr. The solvents were removed on a rotary evaporator and the crude mixture was purified by semi-prep-HPLC (NH₃-method) to give 1-{4-[7-morpholin-4-yl-3-(2,2,2-trifluoroethyl)-3H-[1,2,3]triazolo[4,5-d]pyrimidin-5-yl]phenyl}-3-pyridin-3-ylurea (15 mg, 10% yield) MS (ESI) m/z 500.

### Example 136

Preparation of 1-{4-[7-morpholin-4-yl-3-(2,2,2-trifluoroethyl)-3H-[1,2,3]triazolo[4,5-d]pyrimidin-5-yl]phenyl}-3-pyrimidin-5-ylurea. To a stirred solution of triphosgene (29 mg, 0.1 mmol) in CHCl₃ (7 mL) was added 4-[7-morpholin-4-yl-3-(2,2,2-trifluoroethyl)-3H-[1,2,3]triazolo[4,5-d]pyrimidin-5-yl]aniline (75 mg, 0.2 mmol) at 0° C. The reaction mixture was stirred for 15 min and 5-aminopyrimidine (57 mg, 0.6 mmol) and NEt₃ (83 μL, 0.6 mmol) was added and the reaction mixture was stirred for 1 hr. The solvents were removed on a rotary evaporator and the crude mixture was purified by semi-prep-HPLC (NH₃-method) to give 1-{4-[7-morpholin-4-yl-3-(2,2,2-trifluoroethyl)-3H-[1,2,3]triazolo[4,5-d]pyrimidin-5-yl]phenyl}-3-pyrimidin-5-ylurea (19 mg, 19% yield) MS (ESI) m/z 501.3.

### Example 137

Preparation of 1-[4-(dimethylamino)phenyl]-3-{4-[7-morpholin-4-yl-3-(2,2,2-trifluoroethyl)-3H-[1,2,3]triazolo[4,5-d]pyrimidin-5-yl]phenyl}urea. To a stirred solution of triphosgene (59 mg, 0.2 mmol) in CHCl₃ (7 mL) was added 4-[7-morpholin-4-yl-3-(2,2,2-trifluoroethyl)-3H-[1,2,3]triazolo[4,5-d]pyrimidin-5-yl]aniline (75 mg, 0.2 mmol) at 0° C. The reaction mixture was stirred for 15 min and N,N-dimethylphenylenediamine (81 mg, 0.6 mmol) and NEt₃ (83 μL, 0.6 mmol) was added and the reaction mixture was stirred for 1 hr. The solvents were removed on a rotary evaporator and the crude mixture was purified by semi-prep-HPLC (NH₃-method) to give 1-[4-(dimethylamino)phenyl]-3-{4-[7-morpholin-4-yl-3-(2,2,2-trifluoroethyl)-3H-[1,2,3]triazolo[4,5-d]pyrimidin-5-yl]phenyl}urea (17 mg, 16% yield) MS (ESI) m/z 542.2.

### Example 138

Preparation of 1-[4-(2-hydroxyethyl)phenyl]-3-{4-[7-morpholin-4-yl-3-(2,2,2-trifluoroethyl)-3H-[1,2,3]triazolo[4,5-d]pyrimidin-5-yl]phenyl}urea. To a stirred solution of triphosgene (59 mg, 0.2 mmol) in CHCl₃ (7 mL) was added 4-[7-morpholin-4-yl-3-(2,2,2-trifluoroethyl)-3H-[1,2,3]triazolo[4,5-d]pyrimidin-5-yl]aniline (75 mg, 0.2 mmol) at 0° C. The reaction mixture was stirred for 15 min and 4-amino-phenethyl alcohol (82 mg, 0.6 mmol) and NEt₃ (83 μL, 0.6 mmol) was added and the reaction mixture was stirred for 1 hr. The solvents were removed on a rotary evaporator and the crude mixture was purified by semi-prep-HPLC (NH₃-method) to give 1-[4-(2-hydroxyethyl)phenyl]-3-{4-[7-morpholin-4-yl-3-(2,2,2-trifluoroethyl)-3H-[1,2,3]triazolo[4,5-d]pyrimidin-5-yl]phenyl}urea (28 mg, 26% yield) MS (ESI) m/z 542.2.

### Example 139

Preparation of tert-butyl methyl{2-[({4-[7-morpholin-4-yl-3-(2,2,2-trifluoroethyl)-3H-[1,2,3]triazolo[4,5-d]pyrimidin-5-yl]phenyl}carbamoyl)amino]ethyl}carbamate. To a stirred solution of triphosgene (59 mg, 0.2 mmol) in CHCl₃ (7 mL) was added 4-[7-morpholin-4-yl-3-(2,2,2-trifluoroethyl)-3H-[1,2,3]triazolo[4,5-d]pyrimidin-5-yl]aniline (150 mg, 0.4 mmol) at 0° C. The reaction mixture was stirred for 15 min and (2-methylamino-ethyl)-carbamic acid tert-butyl ester (207 mg, 1.2 mmol) and NEt₃ (165 μL, 1.2 mmol) was added and the reaction mixture was stirred for 1 hr. The solvents were removed on a rotary evaporator and the crude mixture was purified by semi-prep-HPLC (NH₃-method) to give tert-butyl methyl{2-[({4-[7-morpholin-4-yl-3-(2,2,2-trifluoroethyl)-3H-[1,2,3]triazolo[4,5-d]pyrimidin-5-yl]phenyl}carbamoyl)amino]ethyl}carbamate (112 mg, 48% yield) MS (ESI) m/z 580.4.

### Example 140

Preparation of 1-[2-(methylamino)ethyl]-3-{4-[7-morpholin-4-yl-3-(2,2,2-trifluoroethyl)-3H-[1,2,3]triazolo[4,5-d]pyrimidin-5-yl]phenyl}urea. tert-Butyl methyl{2-[({4-[7-morpholin-4-yl-3-(2,2,2-trifluoroethyl)-3H-[1,2,3]triazolo[4,5-d]pyrimidin-5-yl]phenyl}carbamoyl)amino]ethyl}carbamate (92 mg, 0.16 mmol) was dissolved CH₂Cl₂ (2 mL) and TFA (123 μL, 1.59 mmol) was added and the mixture was stirred for 4 hours at 25° C., than the solvents were removed on a rotary evaporator and the crude mixture was purified by semi-prep-HPLC (TFA-method) to give (62 mg, 65% yield). MS (ESI) m/z 480.3.

### Example 141

Preparation of 1-{4-[7-morpholin-4-yl-3-(2,2,2-trifluoroethyl)-3H-[1,2,3]triazolo[4,5-d]pyrimidin-5-yl]phenyl}-3-(2-thienyl)urea. To a stirred solution of 4-[7-morpholin-4-yl-3-(2,2,2-trifluoroethyl)-3H-[1,2,3]triazolo[4,5-d]pyrimidin-5-yl]aniline (75 mg, 0.2 mmol) in anhydrous CHCl₃ (1 mL) was added thienyl-2-isocyanate (37 mg, 0.3 mmol) and NEt₃ (41 μL, 0.3 mmol). The mixture was stirred for 18 hours and the solvents were removed in vacuo to obtain the crude product, which was purified by semi-prep-HPLC (NH₃-method), to give 1-{4-[7-morpholin-4-yl-3-(2,2,2-trifluoroethyl)-3H-[1,2,3]triazolo[4,5-d]pyrimidin-5-yl]phenyl}-3-(2-thienyl)urea (41 mg, 41% yield MS (ESI) m/z 505.3.

### Example 142

Preparation of 1-{4-[7-morpholin-4-yl-3-(2,2,2-trifluoroethyl)-3H-[1,2,3]triazolo[4,5-d]pyrimidin-5-yl]phenyl}-3-(3-thienyl)urea. To a stirred solution of 4-[7-morpholin-4-yl-3-(2,2,2-trifluoroethyl)-3H-[1,2,3]triazolo[4,5-d]pyrimidin-5-yl]aniline (75 mg, 0.2 mmol) in anhydrous CHCl₃ (1 mL) was added thienyl-3-isocyanate (37 mg, 0.3 mmol) and NEt₃ (41 μL, 0.3 mmol). The mixture was stirred for 18 hours and the solvents were removed in vacuo to obtain the crude product, which was purified by semi-prep-HPLC (NH₃-method), to give 1-{4-[7-morpholin-4-yl-3-(2,2,2-trifluoroethyl)-3H-[1,2,3]triazolo[4,5-d]pyrimidin-5-yl]phenyl}-3-(2-thienyl)urea (51 mg, 51% yield MS (ESI) m/z 505.3.

### Preparation of Tert-Butyl 4-(5-chloro-7-morpholino-3H-[1,2,3]triazolo[4,5-d]pyrimidin-3-yl)piperidine-1-carboxylate

### Step 1

tert-butyl 4-(2-chloro-6-morpholino-5-nitropyrimidin-4-ylamino)piperidine-1-carboxylate was prepared from 5-nitro-4-morpholino-pyrimidine (2.3 g, 7.8 mmol) ethylamine and Et₃N (1.48 mL, 10.6 mmol) according to procedure 1 (step 1) to give the product as a yellow oil (2.3 g, 97% yield). MS (ESI) m/z 443.9.

### Step 2

tert-Butyl 4-(5-amino-2-chloro-6-morpholinopyrimidin-4-ylamino)piperidine-1-carboxylate was prepared by reduction of tert-butyl 4-(2-chloro-6-morpholino-5-nitropyrimidin-4-ylamino)piperidine-1-carboxylate (2.2 g, 4.97 mmol) in MeOH (220 mL) with Raney™ nickel (5.5 g) and hydrazine (1.1 g) following procedure 1 (step 2) to give the product as dark solid (1.28 g, 62% yield). MS (ESI) m/z 413.9.

### Step 3

tert-Butyl 4-(5-chloro-7-morpholino-3H-[1,2,3]triazolo[4,5-d]pyrimidin-3-yl)piperidine-1-carboxylate was prepared from tert-butyl 4-(5-amino-2-chloro-6-morpholinopyrimidin-4-ylamino)piperidine-1-carboxylate (1.2 g, 2.91 mmol) and aqueous (0.5N) NaNO₂ solution (12 mL, 9 mmol) following procedure 1 (step 3 to give the product as a white solid (1.2 g, 97% yield). MS (ESI) m/z 424.9.

### Example 143

Preparation of tert-butyl 4-[5-(3-hydroxyphenyl)-7-morpholin-4-yl-3H-[1,2,3]triazolo[4,5-d]pyrimidin-3-yl]piperidine-1-carboxylate. A mixture of 3-(1-BOC-piperidin-4-yl)-5-chloro-7-morpholin-4-yl-3H-[1,2,3]triazolo[4,5-d]pyrimidine (0.40 g, 0.94 mmol), DME (50 mL), aqueous Na₂CO₃ (2M, 2 mL, 4 mmol), Pd(Ph₃P)₄ (30 mg, 0.03 mmol), 3-hydroxyphenyl boronic acid (196 mg, 1.4 mmol) was heated at reflux for 16 hours. The mixture was cooled to room temperature and the solvent was evaporated. The residue was dissolved again in methylene chloride and filtered though Celite™. Extraction with methylene chloride/water, dried with MgSO₄. The solvent was removed and the residue was purified by chromatography on silica column eluting with methylene chloride/EtOAc (5:1) to give 0.37 g (82% yield) of the product. MS (ESI) m/z 482.

### Example 144

Preparation of 3-(7-morpholin-4-yl-3-piperidin-4-yl-3H-[1,2,3]triazolo[4,5-d]pyrimidin-5-yl)phenol. tert-Butyl 4-[5-(3-hydroxyphenyl)-7-morpholin-4-yl-3H-[1,2,3]triazolo[4,5-d]pyrimidin-3-yl]piperidine-1-carboxylate (370 mg, 0.77 mmol). was dissolved CHCl₃/TFA (4:1, 20 mL) and stirred for 4 hours at 25° C., then the solvents were removed on a rotary evaporator and the crude mixture was purified by semi-prep-HPLC (TFA-method) to give (244 mg, 83% yield). MS (ESI) m/z 382.

### Example 145

Preparation of 3-{7-morpholin-4-yl-3-[1-(1H-pyrrol-2-ylmethyl)piperidin-4-yl]-3H-[1,2,3]triazolo[4,5-d]pyrimidin-5-yl}phenol. To a solution of 3-(7-morpholin-4-yl-3-piperidin-4-yl-3H-[1,2,3]triazolo[4,5-d]pyrimidin-5-yl)phenol (100 mg, 0.24 mmol) in methanol (1 mL) was added 1H-pyrrole-2-carbaldehyde (37 mg, 0.39 mmol), silica supported NaBH₃CN (700 mg) and ZnCl₂ (72 mg, 0.53 mmol) was added. The suspension was stirred for 12 hours and was filtered. The filter cake was washed THF (5 mL) and the filtrate was evaporated. The crude product was dissolved in DMSO (2 mL), filtered and purified by semi-prep-HPLC using ACN/water/TFA as mobile phase. After combining the product fractions and solvent removal, the product was obtained as a white solid. (35 mg, 29%). MS (ESI) m/z 493.4.

### Example 146

Preparation of 3-[3-(1-{4-[3-(dimethylamino)propoxy]benzyl}piperidin-4-yl)-7-morpholin-4-yl-3H-[1,2,3]triazolo[4,5-d]pyrimidin-5-yl]phenol. To a solution of 3-(7-morpholin-4-yl-3-piperidin-4-yl-3H-[1,2,3]triazolo[4,5-d]pyrimidin-5-yl)phenol (100 mg, 0.24 mmol) in methanol (1 mL) was added 4-(3-dimethylaminopropoxy)-benzaldehyde (87 mg, 0.39 mmol), silica supported NaBH₃CN (700 mg) and ZnCl₂ (72 mg, 0.53 mmol) was added. The suspension was stirred for 12 hours and was filtered. The filter cake was washed THF (5 mL) and the filtrate was evaporated. The crude product was dissolved in DMSO (2 mL), filtered and purified by semi-prep-HPLC using ACN/water/TFA as mobile phase. After combining the product fractions and solvent removal, the product was obtained as a white solid. (30 mg, 20%). MS (ESI) m/z 573.5.

### Example 147

Preparation of 3-{3-[1-(4-fluorobenzyl)piperidin-4-yl]-7-morpholin-4-yl-3H-[1,2,3]triazolo[4,5-d]pyrimidin-5-yl}phenol. To a solution of 3-(7-morpholin-4-yl-3-piperidin-4-yl-3H-[1,2,3]triazolo[4,5-d]pyrimidin-5-yl)phenol (100 mg, 0.24 mmol) in methanol (1 mL) was added 4-fluorobenzaldehyde (49 mg, 0.39 mmol), silica supported NaBH₃CN (700 mg) and ZnCl₂ (72 mg, 0.53 mmol) was added. The suspension was stirred for 12 hours and was filtered. The filter cake was washed THF (5 mL) and the filtrate was evaporated. The crude product was dissolved in DMSO (2 mL), filtered and purified by semi-prep-HPLC using ACN/water/TFA as mobile phase. After unifying the product fraction and solvent removal, the product was obtained as a white solid. (31 mg, 24%). MS (ESI) m/z 490.4.

### Example 148

Preparation of tert-butyl 4-[5-(2-aminopyrimidin-5-yl)-7-morpholin-4-yl-3H-[1,2,3]triazolo[4,5-d]pyrimidin-3-yl]piperidine-1-carboxylate. A mixture of 3-(1-BOC-piperidin-4-yl)-5-chloro-7-morpholin-4-yl-3H-[1,2,3]triazolo[4,5-d]pyrimidine (0.49 g, 1.2 mmol), DME (50 mL), aqueous Na₂CO₃ (2M, 2 mL, 4 mmol), Pd(Ph₃P)₄ (30 mg, 0.03 mmol), 2-aminopyrimidine-4-boronic acid pinacol ester (196 mg, 1.4 mmol) was heated at reflux for 16 hours. The mixture was cooled to room temperature and the solvent was evaporated. The residue was dissolved again in methylene chloride, filtered though Celite™, extracted with methylene chloride/water, and dried with MgSO₄. The solvent was removed and the residue was purified by chromatography on silica column eluting with methylene chloride/EtOAc (5:1) to give (430 mg 78% yield) of the product. MS (ESI) m/z 483.

### Example 149

Preparation of 3-{7-morpholin-4-yl-3-[1-(pyridin-2-ylmethyl)piperidin-4-yl]-3H-[1,2,3]triazolo[4,5-d]pyrimidin-5-yl}phenol. To a solution of 3-(7-morpholin-4-yl-3-piperidin-4-yl-3H-[1,2,3]triazolo[4,5-d]pyrimidin-5-yl)phenol (100 mg, 0.24 mmol) in methanol (1 mL) was added picolinealdehyde (41 mg, 0.39 mmol), silica supported NaBH₃CN (700 mg) and ZnCl₂ (72 mg, 0.53 mmol) was added. The suspension was stirred for 12 hours and was filtered. The filter cake was washed THF (5 mL) and the filtrate was evaporated. The crude product was dissolved in DMSO (2 mL), filtered and purified by semi-prep-HPLC using ACN/water/TFA as mobile phase. After combining the product fractions and solvent removal, the product was obtained as a white solid. (36 mg, 29%). MS (ESI) m/z 473.4.

### Example 150

Preparation of tert-butyl 4-(7-morpholin-4-yl-5-{4-[(pyridin-3-ylcarbamoyl)amino]phenyl}-3H-[1,2,3]triazolo[4,5-d]pyrimidin-3-yl)piperidine-1-carboxylate. To a stirred solution of tert-butyl 4-[5-(4-aminophenyl)-7-morpholin-4-yl-3H-[1,2,3]triazolo[4,5-d]pyrimidin-3-yl]piperidine-1-carboxylate (100 mg, 0.22 mmol) in anhydrous CH₂Cl₂ (15 mL) was added of 3-isocyanatopyridine (37 mg, 0.33 mmol) and a catalytic amount of DMAP (2 mg). The mixture was stirred for 16 hours. Afterwards, the solvents were removed in a nitrogen stream and the crude mixture was dissolved in DMSO (2 mL) and was purified by semi-prep-HPLC (TFA-method) to give the product as a white solid (31 mg, 25%). MS (ESI) m/z 601.

### Example 151

Preparation of tert-butyl 4-(5-(4-(3-(2-(dimethylamino)ethyl)ureido)phenyl)-7-morpholino-3H-[1,2,3]triazolo[4,5-d]pyrimidin-3-yl)piperidine-1-carboxylate. To a solution of triphosgene (334 mg, 1.13 mmol) in CH₂Cl₂ (4 mL) was added tert-butyl 4-(5-(4-aminophenyl)-7-morpholino-3H-[1,2,3]triazolo[4,5-d]pyrimidin-3-yl)piperidine-1-carboxylate (1.08 g, 2.25 mmol) and the mixture was stirred for 30 minutes. Then, N,N-dimethylethylenediamine (595 mg, 6.75 mmol)), Et₃N (941 μL, 6.75 mmol) in CH₂Cl₂ (6 mL) were added, and the mixture was stirred overnight. The solvents were removed in a nitrogen stream, and the residue was purified by HPLC to give the product as off-white solid (860 mg, 64% yield). MS (ESI) m/z 595.4.

### Example 152

Preparation of 1-(2-(dimethylamino)ethyl)-3-(4-(7-morpholino-3-(piperidin-4-yl)-3H-[1,2,3]triazolo[4,5-d]pyrimidin-5-yl)phenyl)urea. To tert-butyl 4-(5-(4-(3-(2-(dimethylamino)ethyl)ureido)phenyl)-7-morpholino-3H-[1,2,3]triazolo[4,5-d]pyrimidin-3-yl)piperidine-1-carboxylate (420 mg, 0.706 mmol)) in CH₂Cl₂ (5 mL) was added TFA (844 μL, 10.96 mmol) at 0° C. and the mixture was stirred for 3 hours at 25° C. The reaction mixture was neutralized with NH₄OH, the solvent was removed under reduced pressure. The residue was purified by HPLC to give the product as a white solid (250 mg, 72% yield). MS (ESI) m/z 495.4.

### Example 153

Preparation of 1-(2-(dimethylamino)ethyl)-3-(4-(3-(1-(4-fluorobenzyl)piperidin-4-yl)-7-morpholino-3H-[1,2,3]triazolo[4,5-d]pyrimidin-5-yl)phenyl)urea. To 1-(2-(dimethylamino)ethyl)-3-(4-(7-morpholino-3-(piperidin-4-yl)-3H-[1,2,3]triazolo[4,5-d]pyrimidin-5-yl)phenyl)urea (40 mg, 0.081 mmol) and THF (1 mL) was added 4-fluorobenzaldehyde (13 mg, 0.105 mmol) followed by NaBH(OAc)₃ (26 mg, 0.122 mmol) and then AcOH (6 μL, 0.105 mmol)). The mixture was stirred overnight, concentrated and purified by HPLC to give the product (8.1 mg, 17% yield). MS (ESI) m/z 603.3.

### Example 154

Preparation of 1-(2-(dimethylamino)ethyl)-3-(4-(7-morpholino-3-(1-(pyridin-3-ylmethyl)piperidin-4-yl)-3H-[1,2,3]triazolo[4,5-d]pyrimidin-5-yl)phenyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (40 mg, 0.081 mmol) in THF (1 mL) was reacted according to the procedure above with nicotinaldehyde (11 mg, 0.105 mmol), NaBH(OAc)₃ (26 mg, 0.122 mmol) and AcOH (6 μL, 0.105 mmol)) to give the product (18.8 mg, 40% yield). MS (ESI) m/z 586.3.

### Example 155

Preparation of 1-(4-(3-(1-((6-bromopyridin-3-yl)methyl)piperidin-4-yl)-7-morpholino-3H-[1,2,3]triazolo[4,5-d]pyrimidin-5-yl)phenyl)-3-(2-(dimethylamino)ethyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (40 mg, 0.081 mmol) in THF (1 mL) was reacted according the procedure above with 6-bromonicotinaldehyde (20 mg, 0.105 mmol), NaBH(OAc)₃ (26 mg, 0.122 mmol) and AcOH (6 μL, 0.105 mmol)) to give the product (15 mg, 28% yield). MS (ESI) m/z 664.3.

### Example 156

Preparation of 1-(4-(3-(1-(4-chloro-2-fluorobenzyl)piperidin-4-yl)-7-morpholino-3H-[1,2,3]triazolo[4,5-d]pyrimidin-5-yl)phenyl)-3-(2-(dimethylamino)ethyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (40 mg, 0.081 mmol) in THF (1 mL) was reacted according to the procedure above with 4-chloro-2-fluorobenzaldehyde (17 mg, 0.105 mmol), NaBH(OAc)₃ (26 mg, 0.122 mmol) and AcOH (6 μL, 0.105 mmol)) to give the product (33.9 mg, 66% yield). MS (ESI) m/z 637.3.

### Example 157

Preparation of 1-(2-(dimethylamino)ethyl)-3-(4-(3-(1-((6-fluoropyridin-3-yl)methyl)piperidin-4-yl)-7-morpholino-3H-[1,2,3]triazolo[4,5-d]pyrimidin-5-yl)phenyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with 6-fluoronicotinaldehyde (8.3 mg, 0.066 mmol), NaBH(OAc)₃ (16 mg, 0.076 mmol) and AcOH (4 μL, 0.066 mmol)) to give the product (17 mg, 55% yield). MS (ESI) m/z 604.5.

### Example 158

Preparation of 1-(2-(dimethylamino)ethyl)-3-(4-(3-(1-((5-methylthiophen-2-yl)methyl)piperidin-4-yl)-7-morpholino-3H-[1,2,3]triazolo[4,5-d]pyrimidin-5-yl)phenyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with 5-methylthiophene-2-carbaldehyde (8.3 mg, 0.066 mmol), NaBH(OAc)₃ (16 mg, 0.076 mmol) and AcOH (4 μL, 0.066 mmol)) to give the product (16 mg, 52% yield). MS (ESI) m/z 605.3.

### Example 159

Preparation of 1-(4-(3-(1-butylpiperidin-4-yl)-7-morpholino-3H-[1,2,3]triazolo[4,5-d]pyrimidin-5-yl)phenyl)-3-(2-(dimethylamino)ethyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with butyraldehyde (4.8 mg, 0.066 mmol), NaBH(OAc)₃ (16 mg, 0.076 mmol) and AcOH (4 μL, 0.066 mmol)) to give the product (6.8 mg, 24% yield). MS (ESI) m/z 551.3.

### Example 160

Preparation of 1-(2-(dimethylamino)ethyl)-3-(4-(7-morpholino-3-(1-(4-(pyridin-4-yl)benzyl)piperidin-4-yl)-3H-[1,2,3]triazolo[4,5-d]pyrimidin-5-yl)phenyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with 4-(pyridin-4-yl)benzaldehyde (12.1 mg, 0.066 mmol), NaBH(OAc)₃ (16 mg, 0.076 mmol) and AcOH (4 μL, 0.066 mmol)) to give the product (11.1 mg, 33% yield). MS (ESI) m/z 662.4.

### Example 161

Preparation of 1-(4-(3-(1-((1H-pyrrol-2-yl)methyl)piperidin-4-yl)-7-morpholino-3H-[1,2,3]triazolo[4,5-d]pyrimidin-5-yl)phenyl)-3-(2-(dimethylamino)ethyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with 1H-pyrrole-2-carbaldehyde (6.3 mg, 0.066 mmol), NaBH(OAc)₃ (16 mg, 0.076 mmol) and AcOH (4 μL, 0.066 mmol)) to give the product (14.4 mg, 49% yield). MS (ESI) m/z 574.3.

### Example 162

Preparation of 1-(2-(dimethylamino)ethyl)-3-(4-(3-(1-(4-(3-(dimethylamino)propoxy)benzyl)piperidin-4-yl)-7-morpholino-3H-[1,2,3]triazolo[4,5-d]pyrimidin-5-yl)phenyl)urea. 1-(2-(Dimethylamino)ethyl)-3-(4-(7-morpholino-3-(piperidin-4-yl)-3H-[1,2,3]triazolo[4,5-d]pyrimidin-5-yl)phenyl)urea (25 mg, 0.051 mmol) in THF (1 mL) was reacted according to the example above with 4-(3-(dimethylamino)propoxy)benzaldehyde (13.7 mg, 0.066 mmol), NaBH(OAc)₃ (16 mg, 0.076 mmol) and AcOH (4 μL, 0.066 mmol)) to give the product (5.2 mg, 15% yield). MS (ESI) m/z 686.5.

### Example 163

Preparation of 1-[4-(7-morpholin-4-yl-3-piperidin-4-yl-3H-[1,2,3]triazolo[4,5-d]pyrimidin-5-yl)phenyl]-3-pyridin-3-ylurea. tert-Butyl 4-(7-morpholin-4-yl-5-{4-[(pyridin-3-ylcarbamoyl)amino]phenyl}-3H-[1,2,3]triazolo[4,5-d]pyrimidin-3-yl)piperidine-1-carboxylate (250 mg, 0.41 mmol). was dissolved CHCl₃/TFA (4:1, 20 mL) and stirred for 4 hours at 25° C., then the solvents were removed on a rotary evaporator and the crude mixture was purified by semi-prep-HPLC (TFA-method) to give 1-[4-(7-morpholin-4-yl-3-piperidin-4-yl-3H-[1,2,3]triazolo[4,5-d]pyrimidin-5-yl)phenyl]-3-pyridin-3-ylurea (110 mg, 55% yield). MS (ESI) m/z 501.5.

### Example 164

Preparation of 1-{4-[3-(1-methylpiperidin-4-yl)-7-morpholin-4-yl-3H-[1,2,3]triazolo[4,5-d]pyrimidin-5-yl]phenyl}-3-pyridin-3-ylurea was prepared from 1-[4-(7-morpholin-4-yl-3-piperidin-4-yl-3H-[1,2,3]triazolo[4,5-d]pyrimidin-5-yl)phenyl]-3-pyridin-3-ylurea (150 mg, 2.3 mmol), formaldehyde (37% aqueous solution, 150 mg, 1.85 mmol), NaBH₃CN (150 mg, 2.3 mmol), and ZnCl₂ (200 mg, 1.5 mmol) as described in the example above to give the product (29 mg, 29% yield); MS (ESI) m/z 515.5.

### Example 165

Preparation of tert-butyl 4-[5-(4-{[(4-fluorophenyl)carbamoyl]amino}phenyl)-7-morpholin-4-yl-3H-[1,2,3]triazolo[4,5-d]pyrimidin-3-yl]piperidine-1-carboxylate. To a stirred solution of triphosgene (250 mg, 0.84 mmol) in CH₂Cl₂ (40 mL) was added tert-butyl 4-[5-(4-aminophenyl)-7-morpholin-4-yl-3H-[1,2,3]triazolo[4,5-d]pyrimidin-3-yl]piperidine-1-carboxylate (182 mg, 0.37 mmol) at 25° C. The reaction mixture was stirred for 15 min and 4-fluoroaniline (100 mg, 0.90 mmol) and NEt₃ (909 mg, 9.0 mmol) was added and the reaction mixture was stirred for additional 1 hr. The solvents were removed in a nitrogen stream and the crude mixture was purified by semi-prep-HPLC (TFA-method) to give tert-butyl 4-[5-(4-{[(4-fluorophenyl)carbamoyl]amino}phenyl)-7-morpholin-4-yl-3H-[1,2,3]triazolo[4,5-d]pyrimidin-3-yl]piperidine-1-carboxylate (40 mg, 17% yield) MS (ESI) m/z 618.5.

### Example 166

Preparation of tert-butyl 4-(7-morpholin-4-yl-5-{4-[(pyridin-4-ylcarbamoyl)amino]phenyl}-3H-[1,2,3]triazolo[4,5-d]pyrimidin-3-yl)piperidine-1-carboxylate. To a stirred solution of triphosgene (250 mg, 0.84 mmol) in CH₂Cl₂ (40 mL) was added tert-butyl 4-[5-(4-aminophenyl)-7-morpholin-4-yl-3H-[1,2,3]triazolo[4,5-d]pyrimidin-3-yl]piperidine-1-carboxylate (500 mg, 1.02 mmol) at 25° C. The reaction mixture was stirred for 15 min and 4-aminopyridine (270 mg, 3.0 mmol) and NEt₃ (909 mg, 9.0 mmol) was added and the reaction mixture was stirred for additional 1 hr. The solvents were removed in a nitrogen stream and the crude mixture was purified by semi-prep-HPLC (TFA-method) to give tert-butyl

4-(7-morpholin-4-yl-5-{4-[(pyridin-4-ylcarbamoyl)amino]phenyl}-3H-[1,2,3]triazolo[4,5-d]pyrimidin-3-yl)piperidine-1-carboxylate (130 mg, 21% yield) MS (ESI) m/z 601.5.

### Example 167

Preparation of 1-[4-(7-morpholin-4-yl-3-piperidin-4-yl-3H-[1,2,3]triazolo[4,5-d]pyrimidin-5-yl)phenyl]-3-pyridin-4-ylurea. tert-Butyl 4-(7-morpholin-4-yl-5-{4-[(pyridin-4-ylcarbamoyl)amino]phenyl}-3H-[1,2,3]triazolo[4,5-d]pyrimidin-3-yl)piperidine-1-carboxylate (100 mg, 0.17 mmol). was dissolved CH₂Cl₂/TFA (4:1, 20 mL) and stirred for 4 hours at 25° C., than the solvents were removed on a rotary evaporator and the crude mixture was purified by semi-prep-HPLC (NH₃-method) to give 1-[4-(7-morpholin-4-yl-3-piperidin-4-yl-3H-[1,2,3]triazolo[4,5-d]pyrimidin-5-yl)phenyl]-3-pyridin-4-ylurea (38 mg, 45% yield). MS (ESI) m/z 501.5.

### Example 168

Preparation of tert-butyl 4-(5-{4-[(methylcarbamoyl)amino]phenyl}-7-morpholin-4-yl-3H-[1,2,3]triazolo[4,5-d]pyrimidin-3-yl)piperidine-1-carboxylate. To a stirred solution of triphosgene (250 mg, 0.84 mmol) in CH₂Cl₂ (40 mL) was added tert-butyl 4-[5-(4-aminophenyl)-7-morpholin-4-yl-3H-[1,2,3]triazolo[4,5-d]pyrimidin-3-yl]piperidine-1-carboxylate (200 mg, 0.4 mmol) at 25° C. The reaction mixture was stirred for 15 min and NH₂Me (2M solution in THF, 1.2 mL, 2.4 mmol) and NEt₃ (909 mg, 9.0 mmol) was added and the reaction mixture was stirred for additional 1 hr. The solvents were removed in a nitrogen stream and the crude mixture was purified by semi-prep-HPLC (TFA-method) to give tert-butyl 4-(5-{4-[(methylcarbamoyl)amino]phenyl}-7-morpholin-4-yl-3H-[1,2,3]triazolo[4,5-d]pyrimidin-3-yl)piperidine-1-carboxylate (35 mg, 16% yield) MS (ESI) m/z 538.5.

### Example 169

Preparation of tert-butyl 4-[5-(4-{[(methoxycarbonyl)carbamoyl]amino}phenyl)-7-morpholin-4-yl-3H-[1,2,3]triazolo[4,5-d]pyrimidin-3-yl]piperidine-1-carboxylate. To a stirred solution of tert-butyl 4-[5-(4-aminophenyl)-7-morpholin-4-yl-3H-[1,2,3]triazolo[4,5-d]pyrimidin-3-yl]piperidine-1-carboxylate (100 mg, 0.22 mmol) in anhydrous CH₂Cl₂ (12 mL) was added methyl isocyanatoformate (37 mg, 0.33 mmol) in a catalytic amount of DMAP (2 mg). The mixture was stirred for 16 hours. Afterwards, the solvents were removed in a nitrogen stream and the crude mixture was dissolved in DMSO (2 mL) and was purified by semi-prep-HPLC (TFA-method) to give the tert-butyl 4-[5-(4-{[(methoxycarbonyl)carbamoyl]amino}phenyl)-7-morpholin-4-yl-3H-[1,2,3]triazolo[4,5-d]pyrimidin-3-yl]piperidine-1-carboxylate as a white solid (30 mg, 23%).MS (ESI) m/z 582.6.

### Example 170

Preparation of 1-{4-[3-(1-benzylpiperidin-4-yl)-7-morpholin-4-yl-3H-[1,2,3]triazolo[4,5-d]pyrimidin-5-yl]phenyl}-3-(3-chlorophenyl)urea. To a stirred solution of triphosgene (90 mg, 0.30 mmol) in CHCl₃ (10 mL) was added 4-[3-(1-benzyl-piperidin-4-yl)-7-morpholin-4-yl-3H-[1,2,3]triazolo[4,5-d]pyrimidin-5-yl]-phenylamine (140 mg, 0.30 mmol) at 0° C. The reaction mixture was stirred for 15 min and 3-chloroaniline (113 mg, 0.89 mmol) and NEt₃ (450 mg, 0.45 mmol) was added and the reaction mixture was stirred for additional 1 hr. The solvents were removed on a rotary evaporator and the crude mixture was purified by semi-prep-HPLC (TFA-method) to give 1-{4-[3-(1-benzylpiperidin-4-yl)-7-morpholin-4-yl-3H-[1,2,3]triazolo[4,5-d]pyrimidin-5-yl]phenyl}-3-(3-chlorophenyl)urea (15 mg, 7% yield) MS (ESI) m/z 625.2.

### Example 171

Preparation of 5-(3-{1-[(2-amino-1,3-thiazol-5-yl)methyl]piperidin-4-yl}-7-morpholin-4-yl-3H-[1,2,3]triazolo[4,5-d]pyrimidin-5-yl)pyridin-3-ol was prepared from 5-(7-morpholin-4-yl-3-piperidin-4-yl-3H-[1,2,3]triazolo[4,5-d]pyrimidin-5-yl)pyridin-3-ol (17 mg, 0.044 mmol), 2-amino-thiazole-5-carbaldehyde (21 mg, 0.16 mmol), NaBH₃CN (10 mg, 0.088 mmol), and ZnCl₂ (10 mg, 0.044 mmol) as described in example 13 to give the product (9 mg, 18% yield). MS (ESI) m/z 495.5.

### Example 172

Preparation of 3-{3-[(1-ethylpyrrolidin-2-yl)methyl]-7-morpholin-4-yl-3H-[1,2,3]triazolo[4,5-d]pyrimidin-5-yl}phenol was prepared from 5-chloro-3-(2-ethyl-pyrrolidin-1-ylmethyl)-7-morpholin-4-yl-3H-[1,2,3]triazolo[4,5-d]pyrimidine (130 mg, 0.37 mmol) and 3-hydroxyphenylboronic acid (102 mg, 0.74 mmol) following procedure 2 to give the titled product (63 mg, 41% yield). MS (ESI) m/z 410.

### Example 173

Preparation of {5-[3-(1-benzylpiperidin-4-yl)-7-morpholin-4-yl-3H-[1,2,3]triazolo[4,5-d]pyrimidin-5-yl]pyridin-3-yl}methanol was prepared from 3-(1-benzylpiperidin-4-yl)-5-chloro-7-morpholin-4-yl-3H-[1,2,3]triazolo[4,5-d]pyrimidine (200 mg, 0.48 mmol) and 5-(4,4,5,5-tetramethyl-[1,3,2]dioxaborolan-2-yl)-pyridine-3-carbaldehyde (370 mg, 1.58 mmol) following procedure 2 to give the intermediate 5-[3-(1-benzyl-piperidin-4-yl)-7-morpholin-4-yl-3H-[1,2,3]triazolo[4,5-d]pyrimidin-5-yl]-pyridine-3-carbaldehyde (140 mg). The 5-[3-(1-benzyl-piperidin-4-yl)-7-morpholin-4-yl-3H-[1,2,3]triazolo[4,5-d]pyrimidin-5-yl]-pyridine-3-carbaldehyde (140 mg, 0.28 mmol) was dissolved in methanol (5 mL) and NaBH4 (60 mg, 1.57 mmol)was added. The reaction mixture was stirred for 2 hours at 25° C. and water (0.5 mL) was added and stirring was continued for another 30 minutes. The solid was filtered off. The filtrate was evaporated and the residue was dissolved in DMSO and purified by preparative HPLC using ACN/water/TFA-gradients as eluent to give the product as white solid (32 mg, 24%), MS (ESI) m/z 487.4.

### Example 174

Preparation of [5-(7-morpholin-4-yl-3-piperidin-4-yl-3H-[1,2,3]triazolo[4,5-d]pyrimidin-5-yl)pyridin-3-yl]methanol was prepared from 3-(1-BOC-piperidin-4-yl)-5-chloro-7-morpholin-4-yl-3H-[1,2,3]triazolo[4,5-d]pyrimidine (350 mg, 0.71 mmol) and 5-(4,4,5,5-tetramethyl-[1,3,2]dioxaborolan-2-yl)-pyridine-3-carbaldehyde (700 mg, 3 mmol) following procedure 2 to give the intermediate 5-[3-(1-BOC-piperidin-4-yl)-7-morpholin-4-yl-3H-[1,2,3]triazolo[4,5-d]pyrimidin-5-yl]-pyridine-3-carbaldehyde (300 mg). The 5-[3-(1-BOC-piperidin-4-yl)-7-morpholin-4-yl-3H-[1,2,3]triazolo[4,5-d]pyrimidin-5-yl]-pyridine-3-carbaldehyde (140 mg, 0.28 mmol) was dissolved CHCl₃/TFA (4:1, 5 mL) and stirred for 4 hours at 25° C., than the solvents were removed on a rotary evaporator. The residue was dissolved in methanol (5 mL) and NaBH₄ (60 mg, 1.57 mmol) was added. The reaction mixture was stirred for 2 hours at 25° C. and water (0.5 mL) was added and stirring was continued for another 30 minutes. The solid was filtered off. The filtrate was evaporated and the residue was dissolved in DMSO and purified by preparative HPLC using ACN/water/NH₃-gradients as eluent to give the product as white solid (3 mg, 1%), MS (ESI) m/z 397.

### Example 175

Preparation of 4-(3-ethyl-7-morpholin-4-yl-3H-[1,2,3]triazolo[4,5-d]pyrimidin-5-yl)-2-methoxyaniline was prepared from 5-chloro-3-ethyl-7-morpholin-4-yl-3H-[1,2,3]triazolo[4,5-d]pyrimidine (600 mg, 2.23 mmol) and 2-methoxy-4-(4,4,5,5-tetramethyl-[1,3,2]dioxaborolan-2-yl)-phenylamine (986 mg, 4.5 mmol) following procedure 2 to give the intermediate [4-(3-ethyl-7-morpholin-4-yl-3H-[1,2,3]triazolo[4,5-d]pyrimidin-5-yl)-2-methoxy-phenyl]-carbamic acid tert-butyl ester (800 mg, 79% yield) MS (ESI) m/z 456.

[4-(3-Ethyl-7-morpholin-4-yl-3H-[1,2,3]triazolo[4,5-d]pyrimidin-5-yl)-2-methoxyphenyl]-carbamic acid tert-butyl ester (400 mg, 0.88 mmol) was dissolved CHCl₃/TFA (1:1, 5 mL) and stirred for 4 hours at 25° C., than the CHCL₃ (100 mL) were added and the organic layer were extracted with sat NaHCO₃-solution (10 mL) and brine (10 mL) and the combined organic layers were dried over MgSO₄. Filtration and solvent removal on a rotary evaporator gave the off-white product (300 mg, 91% yield). MS (ESI) m/z 356.

### Example 176

### Preparation of [3-(7-morpholin-4-yl-3-piperidin-4-yl-3H-[1,2,3]triazolo[4,5-d]pyrimidin-5-yl)phenyl]methanol Intermediate

tert-butyl 4-{5-[3-(hydroxymethyl)phenyl]-7-morpholin-4-yl-3H-[1,2,3]triazolo[4,5-d]pyrimidin-3-yl}piperidine-1-carboxylate was prepared from 3-(1-BOC-piperidin-4-yl)-5-chloro-7-morpholin-4-yl-3H-[1,2,3]triazolo[4,5-d]pyrimidine (500 mg, 1.18 mmol) and 3-hydroxymethylphenylboronic acid (269 mg, 1.77 mmol) following procedure 2 to give the titled product (510 mg, 87% yield).MS (ESI) m/z 496.4 tert-butyl 4-{5-[3-(hydroxymethyl)phenyl]-7-morpholin-4-yl-3H-[1,2,3]triazolo[4,5-d]pyrimidin-3-yl}piperidine-1-carboxylate (480 mg, 0.97 mmol). was dissolved CH₂Cl₂ (5 mL) and TFA (745 μL, 9.67 mmol) was added and the mixture was stirred for 4 hours at 25° C., than the mixture was made basic with NaOH (1N). The organic layer was separated and dried over Na₂SO₄. The crude material was purified by flash chromatography to give the product (106 mg, 28% yield). MS (ESI) m/z 396.4.

### Example 177

Preparation of {3-[3-(1-benzylpiperidin-4-yl)-7-morpholin-4-yl-3H-[1,2,3]triazolo[4,5-d]pyrimidin-5-yl]phenyl}methanol. [3-(7-Morpholin-4-yl-3-piperidin-4-yl-3H-[1,2,3]triazolo[4,5-d]pyrimidin-5-yl)phenyl]methanol (40 mg, 0.08 mmol) was dissolved in THF (2 mL), benzaldehyde (45 μL, 0.43 mmol), NaBH(OAc)₃ (105 mg, 0.49 mmol) and AcOH (84 μg, 0.43 mmol) were added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions, and solvent removal, the product was obtained as a white solid. (23 mg, 47%), MS (ESI) m/z 486.4.

### Preparation of 5-Chloro-3-(2,2-dimethoxy-ethyl)-7-morpholin-4-yl-3H-[1,2,3]triazolo[4,5-d]pyrimidine

### Step 1

(2-Chloro-6-morpholin-4-yl-5-nitro-pyrimidin-4-yl)-(2,2-dimethoxy-ethyl)-amine was prepared from 2,6-dichloro-5-nitro-4-morpholino-pyrimidine (800 g, 2.86 mmol) and aminoacetaldehyde dimethylacetal (300 mg, 2.86 mmol) following procedure 1 (step 1) to give the final product (1.0 g, 100% yield); MS (ESI) m/z 348.

### Step 2

2-Chloro-N-4-(2,2-dimethoxy-ethyl)-6-morpholin-4-yl-pyrimidine-4,5-diamine was prepared by the reduction of 2-chloro-6-morpholin-4-yl-5-nitro-pyrimidin-4-yl)-(2,2-dimethoxyethyl)-amine (1 g, 2.86 mmol) following procedure 1 (step 2) to give the final product (730 mg, 74% yield); MS (ESI) m/z 318.

### Step 3

5-Chloro-3-(2,2-dimethoxy-ethyl)-7-morpholin-4-yl-3H-[1,2,3]triazolo[4,5-d]pyrimidine was prepared from 2-chloro-N⁴-(2,2-dimethoxy-ethyl)-6-morpholin-4-yl-pyrimidine-4,5-diamine (730 mg, 2.23 mmol) and aqueous (0.5N) NaNO₂ solution (3.75 mL, 1.88 mmol) following procedure 1 (step 3) to give the final product (450 mg, 61% yield); MS (ESI) m/z 329.

### Example 178

Preparation of 4-[3-(2,2-dimethoxyethyl)-7-morpholin-4-yl-3H-[1,2,3]triazolo[4,5-d]pyrimidin-5-yl]aniline was prepared from 5-chloro-3-(2,2-dimethoxy-ethyl)-7-morpholin-4-yl-3H-[1,2,3]triazolo[4,5-d]pyrimidine (600 mg, 1.82 mmol) 4-(4,4,5,5-tetramethyl-[1,3,2]dioxaborolan-2-yl)-phenylamine (650 mg, 2.9 mmol) following procedure 2 to give the titled product (580 mg, 82% yield). MS (ESI) m/z 386.

### Example 179

Preparation of 1-{4-[3-(2,2-dimethoxyethyl)-7-morpholin-4-yl-3H-[1,2,3]triazolo[4,5-d]pyrimidin-5-yl]phenyl}-3-(4-methylphenyl)urea. To a stirred solution of 4-[3-(2,2-dimethoxyethyl)-7-morpholin-4-yl-3H-[1,2,3]triazolo[4,5-d]pyrimidin-5-yl]aniline (100 mg, 0.36 mmol) in anhydrous CH₂Cl₂ (15 mL) was added a solution of 4-methyphenylisocyanate (72 mg, 0.54 mmol) in CH₂Cl₂ (15 mL) and a catalytic amount of DMAP (5 mg). The mixture was stirred for 16 hours. The solvents were removed in a nitrogen stream and the crude mixture was dissolved in DMSO (2 mL) and was purified by semi-prep-HPLC (TFA-method) to give the product as a white solid (30 mg, 16%). MS (ESI) m/z 519.3.

### Example 180

Preparation of 1-{4-[3-(2,2-dimethoxyethyl)-7-morpholin-4-yl-3H-[1,2,3]triazolo[4,5-d]pyrimidin-5-yl]phenyl}-3-(4-fluorophenyl)urea. To a stirred solution of 4-[3-(2,2-dimethoxyethyl)-7-morpholin-4-yl-3H-[1,2,3]triazolo[4,5-d]pyrimidin-5-yl]aniline (100 mg, 0.36 mmol) in anhydrous CH₂Cl₂ (15 mL) was added a solution of 4-fluorophenylisocyanate (72 mg, 0.54 mmol) in CH₂Cl₂ (15 mL) and a catalytic amount of DMAP (5 mg). The mixture was stirred for 16 hours. The solvents were removed in a nitrogen stream and the crude mixture was dissolved in DMSO (2 mL) and was purified by semi-prep-HPLC (TFA-method) to give the product as a white solid (11 mg, 11%). MS (ESI) m/z 523.

### Example 181

Preparation of 1-{4-[3-(2,2-dimethoxyethyl)-7-morpholin-4-yl-3H-[1,2,3]triazolo[4,5-d]pyrimidin-5-yl]phenyl}-3-pyridin-3-ylurea. To a stirred solution of 4-[3-(2,2-dimethoxyethyl)-7-morpholin-4-yl-3H-[1,2,3]triazolo[4,5-d]pyrimidin-5-yl]aniline (132 mg, 0.47 mmol) in anhydrous CH₂Cl₂ (15 mL) was added a solution of 3-isocyanatopyridine (82 mg, 0.68 mmol) in CH₂Cl₂ (15 mL) and NEt₃ (3 mL, 21.7 mmol)). The mixture was stirred for 16 hours. The solvents were removed in a nitrogen stream and the crude mixture was dissolved in DMSO (2 mL) and was purified by semi-prep-HPLC (TFA-method) to give the product as a white solid (60 mg, 45%). MS (ESI) m/z 506.2.

### Example 182

Preparation of 4-[({4-[3-(2,2-dimethoxyethyl)-7-morpholin-4-yl-3H-[1,2,3]triazolo[4,5-d]pyrimidin-5-yl]phenyl}carbamoyl)amino]benzamide. To a stirred solution of triphosgene (239 mg, 0.8 mmol) in CH₂Cl₂/THF (1:1, 10 mL) was added 4-(3-(2,2-dimethoxyethyl)l-7-morpholin-4-yl-3H-[1,2,3]triazolo[4,5-d]pyrimidin-5-yl)-phenylamine (385 mg, 1.0 mmol) at 25° C. The reaction mixture was stirred for 15 min and 4-aminobenzamide (272 mg, 2 mmol) and NEt₃ (664 μL, 4.8 mmol) was added and the reaction mixture was stirred for additional 1 hr. The solvents were removed on a rotary evaporator and the crude mixture was purified by semi-prep-HPLC (TFA-method) to give 4-[({4-[3-(2,2-dimethoxyethyl)-7-morpholin-4-yl-3H-[1,2,3]triazolo[4,5-d]pyrimidin-5-yl]phenyl}carbamoyl)amino]benzamide (20 mg, 4% yield) MS (ESI) m/z 548.

Preparation of 1-{4-[7-morpholin-4-yl-3-(2-oxo-ethyl)-3H-[1,2,3]triazolo[4,5-d]pyrimidin-5-yl]-phenyl}-3-pyridin-4-yl-urea. To a stirred solution of triphosgene (93 mg, 0.31 mmol) in CH₂Cl₂ (3 mL) was added 4-(3-(2,2-dimethoxyethyl)1-7-morpholin-4-yl-3H-[1,2,3]triazolo[4,5-d]pyrimidin-5-yl)-phenylamine (240 mg, 62 mmol) at 25° C. The reaction mixture was stirred for 15 min and 4-aminopyridine (113 mg, 1.2 mmol) and NEt₃ (166 μL, 1.2 mmol) was added and the reaction mixture was stirred for additional 1 hr. The solvents were removed on a rotary evaporator and the crude compound was purified by silica gel chromatography (CH₂Cl₂/MeOH/NH₃) to give the intermediate 1-{4-[3-(2,2-dimethoxyethyl)-7-morpholin-4-yl-3H-[1,2,3]triazolo[4,5-d]pyrimidin-5-yl]phenyl}-3-pyridin-4-ylurea (160 mg, 50% yield) MS (ESI) m/z 506.

In a one-neck flask equipped with a reflux condenser were dissolved 1-{4-[3-(2,2-dimethoxyethyl)-7-morpholin-4-yl-3H-[1,2,3]triazolo[4,5-d]pyrimidin-5-yl]phenyl}-3-pyridin-4-ylurea (160 mg, 0.32 mmol) in dioxane (2 mL) and HCl (6N, 2 mL). The mixture was heated to 80° C. for 2 hours. The solvents were removed to give the crude 1-{4-[7-morpholin-4-yl-3-(2-oxo-ethyl)-3H-[1,2,3]triazolo[4,5-d]pyrimidin-5-yl]-phenyl}-3-pyridin-4-yl-urea (150 mg, 100% yield) MS (ESI) m/z 460.3.

### Example 183

Preparation of 1-{4-[3-(2-hydroxyethyl)-7-morpholin-4-yl-3H-[1,2,3]triazolo[4,5-d]pyrimidin-5-yl]phenyl}-3-pyridin-4-ylurea. 1-{4-[7-morpholin-4-yl-3-(2-oxo-ethyl)-3H-[1,2,3]triazolo[4,5-d]pyrimidin-5-yl]-phenyl}-3-pyridin-4-yl-urea (150 mg, 0.32 mmol) was dissolved in methanol (1 mL) and Me₂NH (2M solution in THF (320 μL, 0.64 mmol), NaBH₃CN (40 mg, 0.64 mmol) and ZnCl₂ (40 mg, 0.32 mmol) was added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions and solvent removal, the product was obtained as a white solid. (24 mg, 15%). MS (ESI) m/z 489.4.

### Example 184

Preparation of 1-{4-[3-(2-hydroxyethyl)-7-morpholin-4-yl-3H-[1,2,3]triazolo[4,5-d]pyrimidin-5-yl]phenyl}-3-pyridin-3-ylurea. In a one-neck flask equipped with reflux condenser was dissolved 1-{4-[3-(2,2-dimethoxyethyl)-7-morpholin-4-yl-3H-[1,2,3]triazolo[4,5-d]pyrimidin-5-yl]phenyl}-3-pyridin-3-ylurea (60 mg, 0.11 mmol) in dioxane (2 mL) and HCl (6N, 2 mL). The mixture was heated to 80° C. for 2 hours. The solvents were removed to give the crude 1-{4-[7-morpholin-4-yl-3-(2-oxo-ethyl)-3H-[1,2,3]triazolo[4,5-d]pyrimidin-5-yl]-phenyl}-3-pyridin-3-yl-urea, which was dissolved in methanol (2 mL) and added to a stirred solution of NaBH₄ (100 mg, 1.58 mmol) in methanol (5 mL). The reaction mixture was stirred for 15 minutes at 25° C. and water (0.05 mL) was added and stirring was continued for another 30 minutes. The solvents were evaporated and the residue was dissolved in DMSO and purified by preparative HPLC using ACN/water/NH₃-gradients as eluent to give the 1-{4-[3-(2-hydroxyethyl)-7-morpholin-4-yl-3H-[1,2,3]triazolo[4,5-d]pyrimidin-5-yl]phenyl}-3-pyridin-3-ylurea as white solid (40 mg, 74%), MS (ESI) m/z 462.

### Example 185

Preparation of 1-{4-[3-(2-hydroxyethyl)-7-morpholin-4-yl-3H-[1,2,3]triazolo[4,5-d]pyrimidin-5-yl]phenyl}-3-(4-methoxyphenyl)urea. To a stirred solution of 4-[3-(2,2-dimethoxyethyl)-7-morpholin-4-yl-3H-[1,2,3]triazolo[4,5-d]pyrimidin-5-yl]aniline (100 mg, 0.36 mmol) in anhydrous CH₂Cl₂ (15 mL) was added a solution of 4-methoxyphenylisocyanate (60 mg, 0.42 mmol) in CH₂Cl₂ (15 mL) and a catalytic amount of DMAP (1 mg). The mixture was stirred for 16 hours. The solvents were removed in a nitrogen stream and the crude mixture was dissolved in dioxane (2 mL) and HCl (6N, 2 mL). The mixture was heated to 80° C. for 2 hours. The solvents were removed to give the crude 1-{4-[7-morpholin-4-yl-3-(2-oxo-ethyl)-3H-[1,2,3]triazolo[4,5-d]pyrimidin-5-yl]-phenyl}-3-(4-methoxyphenyl)-urea, which was dissolved in methanol (2 mL) and added to a stirred solution of NaBH₄ (100 mg, 1.58 mmol) in methanol (5 mL). The reaction mixture was stirred for 15 minutes at 25° C. and water (0.05 mL) was added and stirring was continued for another 30 minutes. The solvents were evaporated and the residue was dissolved in DMSO and purified by preparative HPLC using ACN/water/TFA-gradients as eluent to give the 1-{4-[3-(2-hydroxyethyl)-7-morpholin-4-yl-3H-[1,2,3]triazolo[4,5-d]pyrimidin-5-yl]phenyl}-3-(4-methoxyphenyl)urea as white solid (28 mg, 24%), MS (ESI) m/z 491.

### Example 186

Preparation of 1-{4-[3-(2-hydroxyethyl)-7-morpholin-4-yl-3H-[1,2,3]triazolo[4,5-d]pyrimidin-5-yl]phenyl}-3-phenylurea. To a stirred solution of 4-[3-(2,2-dimethoxyethyl)-7-morpholin-4-yl-3H-[1,2,3]triazolo[4,5-d]pyrimidin-5-yl]aniline (100 mg, 0.36 mmol) in anhydrous CH₂Cl₂ (15 mL) was added a solution of phenylisocyanate (50 mg, 0.42 mmol) in CH₂Cl₂ (15 mL) and a catalytic amount of DMAP (1 mg). The mixture was stirred for 16 hours. The solvents were removed in a nitrogen stream and the crude mixture was dissolved in dioxane (2 mL) and HCl (6N, 2 mL). The mixture was heated to 80° C. for 2 hours. The solvents were removed to give the crude 1-{4-[7-morpholin-4-yl-3-(2-oxo-ethyl)-3H-[1,2,3]triazolo[4,5-d]pyrimidin-5-yl]-phenyl}-3-phenylurea, which was dissolved in methanol (2 mL) and added to a stirred solution of NaBH₄ (100 mg, 1.58 mmol) in methanol (5 mL). The reaction mixture was stirred for 15 minutes at 25° C. and water (0.05 mL) was added and stirring was continued for another 30 minutes. The solvents were evaporated and the residue was dissolved in DMSO and purified by preparative HPLC using ACN/water/TFA-gradients as eluent to give the 1-{4-[3-(2-hydroxyethyl)-7-morpholin-4-yl-3H-[1,2,3]triazolo[4,5-d]pyrimidin-5-yl]phenyl}-3-phenylurea as white solid (9 mg, 5%), MS (ESI) m/z 461.

### 3-(5-Chloro-7-morpholin-4-yl-[1,2,3]triazolo[4,5-d]pyrimidin-3-ylmethyl)-azetidine-1-carboxylic Acid Tert-Butyl Ester

### Step 1

3-[(2-Chloro-6-morpholin-4-yl-5-nitro-pyrimidin-4-ylamino)-methyl]-azetidine-1-carboxylic acid tert-butyl ester was prepared from 2,6-dichloro-5-nitro-4-morpholino-pyrimidine (1.75 g, 4.5 mmol) and 3-aminomethyl-azetidine-1-carboxylic acid tert-butyl ester-HCl (1 g, 4.5 mmol) following procedure 1 (step 1) to give the yellow product (1.36 g, 70% yield); MS (ESI) m/z 429.

### Step 2

3-[(5-Amino-2-chloro-6-morpholin-4-yl-pyrimidin-4-ylamino)-methyl]-azetidine-1-carboxylic acid tert-butyl ester was prepared by the reduction of 3-[(2-chloro-6-morpholin-4-yl-5-nitro-pyrimidin-4-ylamino)-methyl]-azetidine-1-carboxylic acid tert-butyl ester (1.3 g, 3.03 mmol) following procedure 1 (step 2) to give the final product (1.11 g, 93% yield); MS (ESI) m/z 389

### Step 3

3-(5-Chloro-7-morpholin-4-yl-[1,2,3]triazolo[4,5-d]pyrimidin-3-ylmethyl)-azetidine-1-carboxylic acid tert-butyl ester was prepared from 3-[(5-amino-2-chloro-6-morpholin-4-yl-pyrimidin-4-ylamino)-methyl]-azetidine-1-carboxylic acid tert-butyl ester (1.1 g, 2.76 mmol) and aqueous (0.5N) NaNO₂ solution (11 mL, 13 mmol) following procedure 1 (step 3) to give the final product (934 mg, 82% yield); MS (ESI) m/z 410.3.

### Example 187

Preparation of tert-Butyl 3-{[5-(4-aminophenyl)-7-morpholin-4-yl-3H-[1,2,3]triazolo[4,5-d]pyrimidin-3-yl]methyl}azetidine-1-carboxylate. was prepared from 3-(5-chloro-7-morpholin-4-yl-[1,2,3]triazolo[4,5-d]pyrimidin-3-ylmethyl)-azetidine-1-carboxylic acid tert-butyl ester (900 mg, 2.19 mmol) 4-(4,4,5,5-tetramethyl-[1,3,2]dioxaborolan-2-yl)-phenylamine (721 mg, 3.29 mmol) following procedure 2 to give the titled product (703 mg, 82% yield). MS (ESI) m/z 467.3.

### Example 188

Preparation of tert-butyl 3-[(7-morpholin-4-yl-5-{4-[(phenylcarbamoyl)amino]phenyl}-3H-[1,2,3]triazolo[4,5-d]pyrimidin-3-yl)methyl]azetidine-1-carboxylate. To a stirred solution of tert-butyl 3-{[5-(4-aminophenyl)-7-morpholin-4-yl-3H-[1,2,3]triazolo[4,5-d]pyrimidin-3-yl]methyl}azetidine-1-carboxylate (680 mg, 1.46 mmol) in anhydrous CHCl₃ (6 mL) was added phenylisocyanate (261 mg, 2.2 mmol) and NEt₃ (305 μL, 2.2 mmol). The mixture was stirred for 18 hours and the solvents were removed in vacuo to obtain the crude product, which was purified flash chromatography eluting with Hex/EtOAc to give tert-butyl 3-[(7-morpholin-4-yl-5-{4-[(phenylcarbamoyl)amino]phenyl}-3H-[1,2,3]triazolo[4,5-d]pyrimidin-3-yl)methyl]azetidine-1-carboxylate (724 mg, 51% yield MS (ESI) m/z 586.4.

### Example 189

Preparation of 1-{4-[3-(azetidin-3-ylmethyl)-7-morpholin-4-yl-3H-[1,2,3]triazolo[4,5-d]pyrimidin-5-yl]phenyl}-3-phenylurea. tert-Butyl 3-[(7-morpholin-4-yl-5-{4-[(phenylcarbamoyl)amino]phenyl}-3H-[1,2,3]triazolo[4,5-d]pyrimidin-3-yl)methyl]azetidine-1-carboxylate (700 mg, 1.19 mmol). was dissolved CH₂Cl₂ (2 mL) and TFA (917 μL, 11.9 mmol) was added and the mixture was stirred for 4 hours at 25° C., then the mixture was made basic with NaOH (1N). The product precipitated as white solid, which was collected by filtration. The filter cake was washed with CHCl₃ (1 mL) and the solid was dried in a vacuum oven to give. (554 mg, 96% yield). MS (ESI) m/z 486.3.

### Example 190

Preparation of 1-(4-{3-[(1-benzoylazetidin-3-yl)methyl]-7-morpholin-4-yl-3H-[1,2,3]triazolo[4,5-d]pyrimidin-5-yl}phenyl)-3-phenylurea. To a stirred solution of 1-{4-[3-(azetidin-3-ylmethyl)-7-morpholin-4-yl-3H-[1,2,3]triazolo[4,5-d]pyrimidin-5-yl]phenyl}-3-phenylurea (60 mg, 0.12 mmol) and NEt₃ (26 μL, 0.19 mmol) in THF (1 mL) was added benzoyl chloride (26 mg, 0.19 mmol). The solvents were removed in a nitrogen stream and the crude mixture was purified by semi-prep-HPLC (NH₃-method) to give the product a white solid (38 mg, 54%). MS (ESI) m/z 590.4.

### Example 191

Preparation of 1-(4-{3-[(1-benzylazetidin-3-yl)methyl]-7-morpholin-4-yl-3H-[1,2,3]triazolo[4,5-d]pyrimidin-5-yl}phenyl)-3-phenylurea. 1-(4-{3-[(1-Benzoylazetidin-3-yl)methyl]-7-morpholin-4-yl-3H-[1,2,3]triazolo[4,5-d]pyrimidin-5-yl}phenyl)-3-phenylurea (40 mg, 0.08 mmol) was dissolved in methanol (1 mL) and benzaldehyde (70 μL, 0.7 mmol), NaBH₃CN (40 mg, 0.63 mmol) and ZnCl₂ (40 mg, 0.29 mmol) was added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions and solvent removal, the product was obtained as a white solid. (27 mg, 47%). MS (ESI) m/z 576.4.

### Example 192

1-[4-(3-{[1-(4-fluorobenzyl)azetidin-3-yl]methyl}-7-morpholin-4-yl-3H-[1,2,3]triazolo[4,5-d]pyrimidin-5-yl)phenyl]-3-phenylurea was prepared from 1-(4-{3-[(1-benzoylazetidin-3-yl)methyl]-7-morpholin-4-yl-3H-[1,2,3]triazolo[4,5-d]pyrimidin-5-yl}phenyl)-3-phenylurea (40 mg, 0.08 mmol), 4-fluorobenzaldehyde (81 mg, 0.7 mmol), NaBH₃CN (40 mg, 0.63 mmol), and ZnCl₂ (40 mg, 0.29 mmol) as described in the example above to give the product (24 mg, 42% yield); MS (ESI) m/z 594.

### Example 193

1-[4-(7-Morpholin-4-yl-3-{[1-(4-pyridin-4-ylbenzyl)azetidin-3-yl]methyl}-3H-[1,2,3]triazolo[4,5-d]pyrimidin-5-yl)phenyl]-3-phenylurea was prepared from 1-(4-{3-[(1-benzoylazetidin-3-yl)methyl]-7-morpholin-4-yl-3H-[1,2,3]triazolo[4,5-d]pyrimidin-5-yl}phenyl)-3-phenylurea (40 mg, 0.08 mmol), 4-(4-formylphenyl)-pyridine (120 mg, 0.7 mmol), NaBH₃CN (40 mg, 0.63 mmol), and ZnCl₂ (40 mg, 0.29 mmol) as described in the example above to give the product (26 mg, 36% yield); MS (ESI) m/z 653.

### Example 194

1-(4-{3-[(1-{4-[3-(Dimethylamino)propoxy]benzyl}azetidin-3-yl)methyl]-7-morpholin-4-yl-3H-[1,2,3]triazolo[4,5-d]pyrimidin-5-yl}phenyl)-3-phenylurea was prepared from 1-(4-{3-[(1-benzoylazetidin-3-yl)methyl]-7-morpholin-4-yl-3H-[1,2,3]triazolo[4,5-d]pyrimidin-5-yl}phenyl)-3-phenylurea (40 mg, 0.08 mmol), 4-(3-dimethylaminopropoxy)-benzaldehyde (136 mg, 0.7 mmol), NaBH₃CN (40 mg, 0.63 mmol), and ZnCl₂ (40 mg, 0.29 mmol) as described in the example above to give the product (40 mg, 54% yield). MS (ESI) m/z 677.

### Example 195

3-[7-Morpholin-4-yl-3-(2-piperidin-1-ylethyl)-3H-[1,2,3]triazolo[4,5-d]pyrimidin-5-yl]phenol was prepared from 5-chloro-7-morpholin-4-yl-3-(2-piperidin-1-yl-ethyl)-3H-[1,2,3]triazolo[4,5-d]pyrimidine (150 mg, 0.42 mmol) and (3-hydroxyphenyl)-boronic acid (89 mg, 0.64 mmol) following procedure 2 to give the off-white product (43 mg, 24% yield); MS (ESI) m/z 410.4.

### Example 196

3-[7-Morpholin-4-yl-3-(2-pyridin-2-yl-ethyl)-3H-[1,2,3]triazolo[4,5-d]pyrimidin-5-yl]-phenol was prepared from 5-chloro-7-morpholin-4-yl-3-(2-pyridin-2-yl-ethyl)-3H-[1,2,3]triazolo[4,5-d]pyrimidine (80 mg, 0.23 mmol) and (3-hydroxyphenyl)-boronic acid (48 mg, 0.35 mmol) following procedure 2 to give the off-white product (52 mg, 56% yield); MS (ESI) m/z 404.4.

N-9-Benzyl Series.

### Example 197

4-Chloro-N-(4-{[5-(3-hydroxyphenyl)-7-morpholin-4-yl-3H-[1,2,3]triazolo[4,5-d]pyrimidin-3-yl]methyl}phenyl)benzamide was prepared from 4-chloro-N-[4-(5-chloro-7-morpholin-4-yl-[1,2,3]triazolo[4,5-d]pyrimidin-3-ylmethyl)-phenyl]-benzamide (150 mg, 0.31 mmol) and (3-hydroxyphenyl)-boronic acid (64 mg, 0.46 mmol) following procedure 2 to give the off-white product (30 mg, 18% yield); MS (ESI) m/z 542.3.

### Example 198

### Step 1

Preparation of 1-{4-[7-morpholin-4-yl-3-(tetrahydro-2H-pyran-4-yl)-3H-[1,2,3]triazolo[4,5-d]pyrimidin-5-yl]phenyl}-3-pyridin-4-ylurea. Starting from 2,6-dichloro-5-nitro-4-morpholino-pyrimidine (1.127 g, 4.1 mmol) and 4-amino-tetrahydropyran (500 mg, 4.1 mmol) and following Procedure 1 (Step 1), 5-chloro-7-morpholin-4-yl-3-(tetrahydro-2H-pyran-4-yl)-3H-[1,2,3]triazolo[4,5-d]pyrimidine was isolated as yellow solid. The product was found to be pure enough for further transformations. Yield: 700 mg, 52%; mp 142° C.; MS (ESI) m/z 325.2.

### Step 2

Starting from 5-chloro-7-morpholin-4-yl-3-(tetrahydro-2H-pyran-4-yl)-3H-[1,2,3]triazolo[4,5-d]pyrimidine (648 mg, 2 mmol) and 4-aminophenyl boronic acid (301.4 mg, 2.2 mmol) and following Procedure 2,5-(4-amino-phenyl)-7-morpholin-4-yl-3-(tetrahydro-2H-pyran-4-yl)-3H-[1,2,3]triazolo[4,5-d]pyrimidine was isolated as tan colored solid. Yield: 450 mg 59%; (M+H) 382.

### Step 3

Starting from 5-(4-amino-phenyl)-7-morpholin-4-yl-3-(tetrahydro-2H-pyran-4-yl)-3H-[1,2,3]triazolo[4,5-d]pyrimidine and following the procedure as outlined in Example 182, 1-{4-[7-morpholin-4-yl-3-(tetrahydro-2H-pyran-4-yl)-3H-[1,2,3]triazolo[4,5-d]pyrimidin-5-yl]phenyl}-3-pyridin-4-ylurea was isolated. The product was purified by silica gel column chromatography by eluting with 10% MeOH, 90% ethyl acetate, and NH₄OH (10 ml/l). The white solid isolated was suspended in MeOH/HCl and the HCl salt of the product was isolated. Yield. 180 mg, 80%; mp 332° C.; m/z 502.4.

### Example 199

### Step 1

Preparation of 1-[4-(3-methyl-7-morpholin-4-yl-3H-[1,2,3]triazolo[4,5-d]pyrimidin-5-yl)phenyl]-3-pyridin-3-ylurea. Starting from 2,6-dichloro-5-nitro-4-morpholino-pyrimidine (2.75 g, 10 mmol) and methylamine in THF solution (2.5 ml, 10 mmol) and following the Procedure 1 (step 1), 5-chloro-3-methyl-7-morpholin-4-yl-3H-[1,2,3]triazolo[4,5-d]pyrimidine was isolated as yellow solid. The product was found to be pure enough for further transformations. Yield: 1.3 g, 51%; mp 168° C.; MS (APCI) m/z 255.2.

### Step 2

Starting from 5-chloro-3-methyl-7-morpholin-4-yl-3H-[1,2,3]triazolo[4,5-d]pyrimidine (1.3 g, 5.1 mmol) and 4-aminophenylpinacolyl borane (2.2 g, 10 mmol) following the procedure as outlined in Scheme 2,4-(3-methyl-7-morpholin-4-yl-3H-[1,2,3]triazolo[4,5-d]pyrimidin-5-yl)aniline was isolated as brown solid after purifying the crude material by SiO₂ column chromatography eluting it with 75% EtOAc:Hex. Yield: 900 mg, 56%; mp 153° C.; MS (ESI) m/z 312.3.

### Step 3

Starting from 4-(3-methyl-7-morpholin-4-yl-3H-[1,2,3]triazolo[4,5-d]pyrimidin-5-yl)aniline (60 mg, 0.19 mmol) and 3-pyridylisocyanate (25 mg, 0.20 mmol) and following the procedure as outlined in Example 39, 1-[4-(3-methyl-7-morpholin-4-yl-3H-[1,2,3]triazolo[4,5-d]pyrimidin-5-yl)phenyl]-3-pyridin-3-ylurea was isolated as solid. The solid was suspended in diethyl ether and filtered. It was found to be pure enough. Yield: 60 mg, 72%; mp 272° C.; m/z 432.46.

### Example 200

Preparation of 1-[4-(3-methyl-7-morpholin-4-yl-3H-[1,2,3]triazolo[4,5-d]pyrimidin-5-yl)phenyl]-3-(2-thienyl)urea. Starting from 4-(3-methyl-7-morpholin-4-yl-3H-[1,2,3]triazolo[4,5-d]pyrimidin-5-yl)aniline (60 mg, 0.19 mmol) and 2-thienylisocyanate (20 mg, 0.20 mmol) and following the procedure as outlined in Example 40, 1-[4-(3-methyl-7-morpholin-4-yl-3H-[1,2,3]triazolo[4,5-d]pyrimidin-5-yl)phenyl]-3-(2-thienyl)urea was isolated as white solid. Yield: 62 mg, 72%; mp 182° C.; m/z 437.5.

### Example 201

Preparation of 1-[4-(3-methyl-7-morpholin-4-yl-3H-[1,2,3]triazolo[4,5-d]pyrimidin-5-yl)phenyl]-3-(3-thienyl)urea. Starting from 4-(3-methyl-7-morpholin-4-yl-3H-[1,2,3]triazolo[4,5-d]pyrimidin-5-yl)aniline (40 mg, 0.12 mmol) and 3-thienylisocyanate (20 mg, 0.20 mmol) and following the procedure as outlined in Example 142, 1-[4-(3-methyl-7-morpholin-4-yl-3H-[1,2,3]triazolo[4,5-d]pyrimidin-5-yl)phenyl]-3-(3-thienyl)urea was isolated as white solid. Yield: 20 mg, 38%; mp 272° C.; m/z 437.5.

### Example 202

### Preparation of 3-{3-[4-(dimethylamino)butyl]-7-morpholin-4-yl-3H-[1,2,3]triazolo[4,5-d]pyrimidin-5-yl}phenol

### Step 1

2-Chloro-N-(4,4-diethoxybutyl)-6-morpholino-5-nitropyrimidin-4-amine was prepared from 2,6-dichloro-5-nitro-4-morpholino-pyrimidine (397 mg, 1.43 mmol) and 4,4-diethoxybutan-1-amine (322 mg, 2 mmol) following procedure 1 (step 1) to give the final product (513 mg, 89% yield); MS (ESI) m/z 404.3.

### Step 2

2-Chloro-N⁴-(4,4-diethoxybutyl)-6-morpholino-pyrimidine-4,5-diamine was prepared by the reduction of 2-chloro-N-(4,4-diethoxybutyl)-6-morpholino-5-nitropyrimidin-4-amine (513 mg, 1.3 mmol) following procedure 1 (step 2) to give the final product (354 mg, 75% yield); MS (ESI) m/z 374.6.

### Step 3

4-(5-Chloro-3-(4,4-diethoxybutyl)-3H-[1,2,3]triazolo[4,5-d]pyrimidin-7-yl)morpholine was prepared from 2-chloro-N⁴-(4,4-diethoxybutyl)-6-morpholino-pyrimidine-4,5-diamine (396 mg, 1.1 mmol) and aqueous (0.5N) NaNO₂ solution (4 mL, 2 mmol) following procedure 1 (step 3) to give the final product (270 mg, 64% yield); MS (ESI) m/z 385.2.

### Step 4

3-(3-(4,4-diethoxybutyl)-7-morpholino-3H-[1,2,3]triazolo[4,5-d]pyrimidin-5-yl)-phenol was prepared from 4-(5-chloro-3-(4,4-diethoxybutyl)-3H-[1,2,3]triazolo[4,5-d]pyrimidin-7-yl)morpholine (270 mg, 0.7 mmol) and 3-hydroxyphenyl boronic acid (193 mg, 1.4 mmol) following procedure 2 to give the final product (285 mg, 92% yield). MS (ESI) m/z 443.3.

### Step 5

To a solution of 3-(3-(4,4-diethoxybutyl)-7-morpholino-3H-[1,2,3]triazolo[4,5-d]pyrimidin-5-yl)-phenol (339 mg, 0.77 mmol) in EtOH (10 mL) was added 6N HCl (5 mL), and the resulting mixture was heated at 70° C. for 6 h. The mixture was cooled to room temperature, and extracted with EtOAc. Removal of solvent gave the product 4-(5-(3-hydroxyphenyl)-7-morpholino-3H-[1,2,3]triazolo[4,5-d]pyrimidin-3-yl)butanal (255 mg, 90% yield). MS (ESI) m/z 369.5.

### Step 6

4-(5-(3-hydroxyphenyl)-7-morpholino-3H-[1,2,3]triazolo[4,5-d]pyrimidin-3-yl)butanal (26 mg, 0.07 mmol) was dissolved in methanol (1 mL) and dimethylamine (2M in THF, 0.14 mL, 0.28 mmol), NaBH₃CN (9 mg, 0.14 mmol) and ZnCl₂ (19 mg, 0.14 mmol) was added. The suspension was stirred for 24 hours and the solvents were removed in vacuo. The crude product was dissolved in DMSO (2 mL), filtered and purified by semi-prep-HPLC using ACN/water/NH₃ as mobile phase. After combining the product fractions and solvent removal, the product 3-{3-[4-(dimethylamino)butyl]-7-morpholin-4-yl-3H-[1,2,3]triazolo[4,5-d]pyrimidin-5-yl}phenol was obtained as a white solid (9 mg, 37% yield). MS (ESI) m/z 398.3.

### Example 203

3-{3-[4-(Methylamino)butyl]-7-morpholin-4-yl-3H-[1,2,3]triazolo[4,5-d]pyrimidin-5-yl}phenol was prepared from 4-(5-(3-hydroxyphenyl)-7-morpholino-3H-[1,2,3]triazolo[4,5-d]pyrimidin-3-yl)butanal (26 mg, 0.07 mmol), methylamine (2M in THF, 0.14 mL, 0.28 mmol), NaBH₃CN (9 mg, 0.14 mmol) and ZnCl₂ (19 mg, 0.14 mmol) as described in the example above to give the product (8 mg, 35% yield); MS (ESI) m/z 384.3.

### Example 204

3-[3-(4-Aminobutyl)-7-morpholin-4-yl-3H-[1,2,3]triazolo[4,5-d]pyrimidin-5-yl]phenol was prepared from 4-(5-(3-hydroxyphenyl)-7-morpholino-3H-[1,2,3]triazolo[4,5-d]pyrimidin-3-yl)butanal (26 mg, 0.07 mmol), ammonium hydroxide (30%, 0.2 mL), NaBH₃CN (9 mg, 0.14 mmol) and ZnCl₂ (19 mg, 0.14 mmol) as described in the example above to give the product (4 mg, 10% yield); MS (ESI) m/z 370.3.

### Example 205

3-[7-Morpholin-4-yl-3-(4-pyrrolidin-1-ylbutyl)-3H-[1,2,3]triazolo[4,5-d]pyrimidin-5-yl]phenol was prepared from 4-(5-(3-hydroxyphenyl)-7-morpholino-3H-[1,2,3]triazolo[4,5-d]pyrimidin-3-yl)butanal (26 mg, 0.07 mmol), pyrrolidine (13 mg, 0.28 mmol), NaBH₃CN (9 mg, 0.14 mmol) and ZnCl₂ (19 mg, 0.14 mmol) as described in the example above to give the product (12 mg, 48% yield); MS (ESI) m/z 424.4.

### Example 206

3-{3-[4-(4-Benzylpiperazin-1-yl)butyl]-7-morpholin-4-yl-3H-[1,2,3]triazolo[4,5-d]pyrimidin-5-yl}phenol was prepared from 4-(5-(3-hydroxyphenyl)-7-morpholino-3H-[1,2,3]triazolo[4,5-d]pyrimidin-3-yl)butanal (26 mg, 0.07 mmol), 1-benzylpiperazine (45 mg, 0.28 mmol), NaBH₃CN (9 mg, 0.14 mmol) and ZnCl₂ (19 mg, 0.14 mmol) as described in the example above to give the product (15 mg, 47% yield); MS (ESI) m/z 515.4.

### Example 207

Preparation of 4-{[5-(3-hydroxyphenyl)-7-morpholin-4-yl-3H-[1,2,3]triazolo[4,5-d]pyrimidin-3-yl]methyl}-N-methylbenzamide. To a suspension of 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) in CH₂Cl₂ (2 mL) was added oxalyl chloride (2M in CH₂Cl₂, 0.3 μL, 0.6 mmol), followed by addition of 1 drop of DMF. The resulting mixture was stirred at room temperature for 1 h, then concentrated in vacuo. The resulting residue (acid chloride) was then dissolved in 2 mL THF, and triethylamine (18 mg, 0.18 mmol), then methylamine (2M in THF, 0.3 mL, 0.6 mmol) were added. The resulting mixture was stirred at room temperature overnight and the solvents were removed in vacuo. The crude product was dissolved in DMSO (2 mL), filtered and purified by semi-prep-HPLC using ACN/water/NH₃ as mobile phase. After combining the product fractions and solvent removal, the title compound was obtained as a white solid (18 mg, 54% yield). MS (ESI) m/z 446.2.

### Example 208

Preparation of tert-butyl 4-[(4-{[5-(3-hydroxyphenyl)-7-morpholin-4-yl-3H-[1,2,3]triazolo[4,5-d]pyrimidin-3-yl]methyl}benzoyl)amino]piperidine-1-carboxylate was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 4-amino-1-Boc-piperidine (36 mg, 0.18 mmol) as described in the example above to give the product (11.7 mg, 27% yield); MS (ESI) m/z 615.3.

### Example 209

Preparation of tert-butyl [1-(4-{[5-(3-hydroxyphenyl)-7-morpholin-4-yl-3H-[1,2,3]triazolo[4,5-d]pyrimidin-3-yl]methyl}benzoyl)piperidin-4-yl]carbamate was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 4-amino-1-Boc-piperidine (36 mg, 0.18 mmol) as described in the example above to give the product (16.3 mg, 37% yield); MS (ESI) m/z 615.5.

### Example 210

Preparation of N-(2-acetamidoethyl)-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and N-acetylethylene-diamine (19 mg, 0.18 mmol) as described in the example above to give the product (14.3 mg, 38% yield); MS (ESI) m/z 517.3.

### Example 211

Preparation of 4-{[5-(3-hydroxyphenyl)-7-morpholin-4-yl-3H-[1,2,3]triazolo[4,5-d]pyrimidin-3-yl]methyl}-N-(3-pyrrolidin-1-ylpropyl)benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 1-(3-aminopropyl)pyrrolidine (23 mg, 0.18 mmol) as described in the example above to give the product (23.2 mg, 59% yield); MS (ESI) m/z 543.4.

### Example 212

Preparation of N-benzyl-4-{[5-(3-hydroxyphenyl)-7-morpholin-4-yl-3H-[1,2,3]triazolo[4,5-d]pyrimidin-3-yl]methyl}benzamide. was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and benzylamine (19 mg, 0.18 mmol) as described in the example above to give the product (12 mg, 31% yield); MS (ESI) m/z 522.3.

### Example 213

Preparation of 4-{[5-(3-hydroxyphenyl)-7-morpholin-4-yl-3H-[1,2,3]triazolo[4,5-d]pyrimidin-3-yl]methyl}-N-(2-pyrrolidin-1-ylethyl)benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 1-(2-aminoethyl)pyrrolidine (21 mg, 0.18 mmol) as described in the example above to give the product (17.5 mg, 45% yield); MS (ESI) m/z 529.5.

### Example 214

Preparation of N-[2-(dimethylamino)ethyl]-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and N,N-dimethylethylenediamine (15 mg, 0.18 mmol) as described in the example above to give the product (13.7 mg, 37% yield); MS (ESI) m/z 503.3.

### Example 215

Preparation of N-[3-(dimethylamino)propyl]-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 3-(dimethylamino)-1-propylamine (18 mg, 0.18 mmol) as described in the example above to give the product (18.8 mg, 50% yield); MS (ESI) m/z 517.3.

### Example 216

Preparation of 4-{[5-(3-hydroxyphenyl)-7-morpholin-4-yl-3H-[1,2,3]triazolo[4,5-d]pyrimidin-3-yl]methyl}-N-pyridin-3-ylbenzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 3-aminopyridine (17 mg, 0.18 mmol) as described in the example above to give the product (18.4 mg, 49% yield); MS (ESI) m/z 509.3.

### Example 217

Preparation of N-(4-fluorophenyl)-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 4-fluoroaniline (19 mg, 0.18 mmol) as described in the example above to give the product (18.2 mg, 58% yield); MS (ESI) m/z 526.5.

### Example 218

tert-Butyl 4-{4-[(4-{[5-(3-hydroxyphenyl)-7-morpholin-4-yl-3H-[1,2,3]triazolo[4,5-d]pyrimidin-3-yl]methyl}benzoyl)amino]phenyl}piperazine-1-carboxylate was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and tert-butyl 4-(4-aminophenyl)piperazine-1-carboxylate (50 mg, 0.18 mmol) as described in the example above to give the product (24.1 mg, 50% yield); MS (ESI) m/z 692.7.

### Example 219

Preparation of N-ethyl-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and ethylamine (2M in THF, 0.09 mL, 0.18 mmol) as described in the example above to give the product (11.9 mg, 43% yield); MS (ESI) m/z 460.4.

### Example 220

Preparation of N,N-diethyl-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and diethylamine (2M in THF, 0.09 mL, 0.18 mmol) as described in the example above to give the product (15.1 mg, 52% yield); MS (ESI) m/z 488.5.

### Example 221

Preparation of N-cyclopropyl-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and cyclopropylamine (10 mg, 0.18 mmol) as described in the example above to give the product (7.7 mg, 27% yield); MS (ESI) m/z 472.5.

### Example 222

Preparation of N-tert-butyl-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and t-butylamine (13 mg, 0.18 mmol) as described in the example above to give the product (17.7 mg, 61% yield); MS (ESI) m/z 488.5.

### Example 223

Preparation of 4-{[5-(3-hydroxyphenyl)-7-morpholin-4-yl-3H-[1,2,3]triazolo[4,5-d]pyrimidin-3-yl]methyl}-N-(2-phenylethyl)benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and phenethylamine (22 mg, 0.18 mmol) as described in the example above to give the product (19.7 mg, 61% yield); MS (ESI) m/z 536.5.

### Example 224

Preparation of 4-{[5-(3-hydroxyphenyl)-7-morpholin-4-yl-3H-[1,2,3]triazolo[4,5-d]pyrimidin-3-yl]methyl}-N-[(1S)-1-phenylethyl]benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and (s)-(−)-α-methylbenzylamine (22 mg, 0.18 mmol) as described in the example above to give the product (19.8 mg, 62% yield); MS (ESI) m/z 536.5.

### Example 225

Preparation of 4-{[5-(3-hydroxyphenyl)-7-morpholin-4-yl-3H-[1,2,3]triazolo[4,5-d]pyrimidin-3-yl]methyl}-N-[2-(1H-indol-3-yl)ethyl]benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and tryptamine (29 mg, 0.18 mmol) as described in the example above to give the product (15.3 mg, 37% yield); MS (ESI) m/z 575.5.

### Example 226

Preparation of N-(2-hydroxy-2-phenylethyl)-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 2-amino-1-phenylethanol (25 mg, 0.18 mmol) as described in the example above to give the product (16.9 mg, 51% yield); MS (ESI) m/z 552.5.

### Example 227

Preparation of 3-{7-morpholin-4-yl-3-[4-(piperidin-1-ylcarbonyl)benzyl]-3H-[1,2,3]triazolo[4,5-d]pyrimidin-5-yl}phenol was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and piperidine (15 mg, 0.18 mmol) as described in the example above to give the product (17.2 mg, 57% yield); MS (ESI) m/z 500.5.

### Example 228

Preparation of 3-{7-morpholin-4-yl-3-[4-(pyrrolidin-1-ylcarbonyl)benzyl]-3H-[1,2,3]triazolo[4,5-d]pyrimidin-5-yl}phenol was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and pyrrolidine (13 mg, 0.18 mmol) as described in the example above to give the product (15.4 mg, 53% yield); MS (ESI) m/z 486.5.

### Example 229

Preparation of 3-(7-morpholin-4-yl-3-{4-[(4-phenylpiperazin-1-yl)carbonyl]benzyl}-3H-[1,2,3]triazolo[4,5-d]pyrimidin-5-yl)phenol was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and 1-phenylpiperazine (29 mg, 0.18 mmol) as described in the example above to give the product (25.8 mg, 62% yield); MS (ESI) m/z 577.5.

### Example 230

Preparation of N-(2-furylmethyl)-4-{[5-(3-hydroxyphenyl)-7-morpholin-4-yl-3H-[1,2,3]triazolo[4,5-d]pyrimidin-3-yl]methyl}benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and furfurylamine (17 mg, 0.18 mmol) as described in the example above to give the product (17.6 mg, 57% yield); MS (ESI) m/z 512.5.

### Example 231

Preparation of 4-{[5-(3-hydroxyphenyl)-7-morpholin-4-yl-3H-[1,2,3]triazolo[4,5-d]pyrimidin-3-yl]methyl}-N-[2-(1H-imidazol-5-yl)ethyl]benzamide was prepared from 4-{[5-(3-hydroxyphenyl)-7-morpholin-4-yl-3H-[1,2,3]triazolo[4,5-d]pyrimidin-3-yl]methyl}benzoic acid (26 mg, 0.06 mmol) and histamine (20 mg, 0.18 mmol) as described in the example above to give the product (6.4 mg, 17% yield); MS (ESI) m/z 526.5.

### Example 232

Preparation of tert-butyl {5-[3-(hydroxymethyl)phenyl]-7-morpholin-4-yl-3H-[1,2,3]triazolo[4,5-d]pyrimidin-3-yl}acetate was prepared from tert-butyl {5-chloro-7-morpholin-4-yl-3H-[1,2,3]triazolo[4,5-d]pyrimidin-3-yl}acetate (268 mg, 0.76 mmol) and 3-hydroxymethylphenyl boronic acid (173 mg, 1.14 mmol) following procedure 2 to give the product as off-white solid (208 mg, 64% yield). MS (ESI) m/z 427.4.

### Example 233

Preparation of tert-butyl [5-(3-hydroxyphenyl)-7-morpholin-4-yl-3H-[1,2,3]triazolo[4,5-d]pyrimidin-3-yl]acetate was prepared from tert-butyl {5-chloro-7-morpholin-4-yl-3H-[1,2,3]triazolo[4,5-d]pyrimidin-3-yl}acetate (268 mg, 0.76 mmol) and 3-hydroxyphenyl boronic acid (157 mg, 1.14 mmol) following procedure 2 to give the product as off-white solid (98 mg, 32% yield). MS (ESI) m/z 413.4.

### Example 234

Preparation of tert-butyl (7-morpholin-4-yl-5-{4-[(pyridin-4-ylcarbamoyl)amino]phenyl}-3H-[1,2,3]triazolo[4,5-d]pyrimidin-3-yl)acetate. A mixture of 4-aminopyridine (120 mg, 1.28 mmol), 4-isocyanatophenylboronic acid pinacol ester (245 mg, 1 mmol) and triethylamine (0.2 mL, 1.28 mmol) in DME (2 mL) was stirred at room temperature for 2 h. To the mixture were then added tert-butyl {5-chloro-7-morpholin-4-yl-3H-[1,2,3]triazolo[4,5-d]pyrimidin-3-yl}acetate (230 mg, 0.64 mmol), Pd(PPh₃)₄ (37 mg) and 2M Na₂CO₃ (1.3 mL). The resulting mixture was heated at 130° C. for 30 min in the microwave, and then cooled to room temperature. Work-up and purification according procedure 2 to give the title product (98 mg, 30% yield). MS (ESI) m/z 532.1.

### Example 235

Preparation of 2-{5-[3-(hydroxymethyl)phenyl]-7-morpholin-4-yl-3H-[1,2,3]triazolo[4,5-d]pyrimidin-3-yl}-N-pyridin-3-ylacetamide. A mixture of {5-[3-(hydroxymethyl)phenyl]-7-morpholin-4-yl-3H-[1,2,3]triazolo[4,5-d]pyrimidin-3-yl}acetic acid (22 mg, 0.06 mmol), EDCI (23 mg, 0.12 mmol) and 3-aminopyridine (11 mg, 0.12 mmol) in acetonitrile (2 mL) was stirred at room temperature overnight. Solvent was removed in vacuum, and the residue was subjected to HPLC separation to give the product as off-white solid (17.6 mg, 52% yield). MS (ESI) m/z 447.1.

### Example 236

Preparation of 2-{5-[3-(hydroxymethyl)phenyl]-7-morpholin-4-yl-3H-[1,2,3]triazolo[4,5-d]pyrimidin-3-yl}-N-methylacetamide was prepared from {5-[3-(hydroxymethyl)phenyl]-7-morpholin-4-yl-3H-[1,2,3]triazolo[4,5-d]pyrimidin-3-yl}acetic acid (22 mg, 0.06 mmol) and methylamine (8 mg, 0.12 mmol) as described in the example above to give the product as off-white solid (4 mg, 12% yield). MS (ESI) m/z 384.2.

### Example 237

Preparation of 2-{5-[3-(hydroxymethyl)phenyl]-7-morpholin-4-yl-3H-[1,2,3]triazolo[4,5-d]pyrimidin-3-yl}acetamide was prepared from {5-[3-(hydroxymethyl)phenyl]-7-morpholin-4-yl-3H-[1,2,3]triazolo[4,5-d]pyrimidin-3-yl}acetic acid (22 mg, 0.06 mmol) and ammonium hydrochloride (7 mg, 0.12 mmol) as described in the example above to give the product as off-white solid (3 mg, 10% yield). MS (ESI) m/z 370.2.

### Example 238

Preparation of N-(4-fluorophenyl)-2-{5-[3-(hydroxymethyl)phenyl]-7-morpholin-4-yl-3H-[1,2,3]triazolo[4,5-d]pyrimidin-3-yl}acetamide was prepared from {5-[3-(hydroxymethyl)phenyl]-7-morpholin-4-yl-3H-[1,2,3]triazolo[4,5-d]pyrimidin-3-yl}acetic acid (22 mg, 0.06 mmol) and 4-fluoroaniline (13 mg, 0.12 mmol) as described in the example above to give the product as off-white solid (10.2 mg, 29% yield). MS (ESI) m/z 464.1.

### Example 239

Preparation of N-[2-(dimethylamino)ethyl]-2-{5-[3-(hydroxymethyl)phenyl]-7-morpholin-4-yl-3H-[1,2,3]triazolo[4,5-d]pyrimidin-3-yl}acetamide was prepared from {5-[3-(hydroxymethyl)phenyl]-7-morpholin-4-yl-3H-[1,2,3]triazolo[4,5-d]pyrimidin-3-yl}acetic acid (22 mg, 0.06 mmol) and N,N-dimethylethylenediamine (11 mg, 0.12 mmol) as described in the example above to give the product as off-white solid (5.6 mg, 17% yield). MS (ESI) m/z 441.2.

### Example 240

Preparation of {5-[3-(hydroxymethyl)phenyl]-7-morpholin-4-yl-3H-[1,2,3]triazolo[4,5-d]pyrimidin-3-yl}acetic acid. To a solution of tert-butyl {5-[3-(hydroxymethyl)phenyl]-7-morpholin-4-yl-3H-[1,2,3]triazolo[4,5-d]pyrimidin-3-yl}acetate (180 mg, 0.42 mmol) in CH₂Cl₂ (5 mL) was added TFA (2 mL), and the resulting mixture was stirred at room temperature for 5 h. The solvent was removed under reduced pressure, and the residue was subjected to HPLC separation to give the title product as off-white solid (136 mg, 87% yield), MS (ESI) m/z 371.1.

### Example 241

Preparation of methyl 4-{[5-(3-hydroxyphenyl)-7-morpholin-4-yl-3H-[1,2,3]triazolo[4,5-d]pyrimidin-3-yl]methyl}benzoate. A mixture of methyl 4-[(5-chloro-7-morpholin-4-yl-3H-[1,2,3]triazolo[4,5-d]pyrimidin-3-yl)methyl]benzoate (748 mg, 1.9 mmol), 3-hydroxyphenylboronic acid (400 mg, 2.9 mmol), Pd(PPh₃)₄ (112 mg), DME (6 mL) and 2M Na₂CO₃ (3 mL) was heated at 140° C. for 30 min in the microwave, and then cooled to room temperature. Work-up and purification according procedure 2 to give the title product as off-white solid (722 mg, 84% yield). MS (ESI) m/z 447.3. HRMS: calculated for C₂₃H₂₂N₆O₄+H+, 447.17753; found (ESI, [M+H]+ Observed), 447.1769.

### Example 242

Preparation of 4-{[5-(3-hydroxyphenyl)-7-morpholin-4-yl-3H-[1,2,3]triazolo[4,5-d]pyrimidin-3-yl]methyl}benzoic acid. To a solution of methyl 4-{[5-(3-hydroxyphenyl)-7-morpholin-4-yl-3H-[1,2,3]triazolo[4,5-d]pyrimidin-3-yl]methyl}benzoate (641 mg, 1.44 mmol) in THF (10 mL) and MeOH (10 mL) was added 1N NaOH (4.3 mL), and the resulting mixture was heated at 70° C. for 5 h. The reaction mixture was cooled to room temperature, and adjusted pH to 2-3 by addition of 1N HCl. The mixture was concentrated under reduced pressure, and the residue was treated with water. The resulting solid was collected by filtration to give the title compound as off-white solid (616 mg, 99% yield). MS (ESI) m/z 433.3; HRMS: calculated for C₂₂H₂₀N₆O₄+H⁺, 433.16188; found (ESI, [M+H]⁺ Observed), 433.1612.

### Example 243

Preparation of methyl 4-({5-[3-(hydroxymethyl)phenyl]-7-morpholin-4-yl-3H-[1,2,3]triazolo[4,5-d]pyrimidin-3-yl}methyl)benzoate. A mixture of methyl 4-[(5-chloro-7-morpholin-4-yl-3H-[1,2,3]triazolo[4,5-d]pyrimidin-3-yl)methyl]benzoate (450 mg, 1.2 mmol), 3-hydroxymethylphenylboronic acid (264 mg, 1.7 mmol), Pd(PPh₃)₄ (67 mg), DME (6 mL) and 2M Na₂CO₃ (2.5 mL) was heated at 140° C. for 30 min in the microwave, and then cooled to room temperature. Work-up and purification according procedure 2 to give the title product as off-white solid (168 mg, 31% yield). MS (ESI) m/z 461.5; HRMS: calculated for C₂₄H₂₄N₆O₄+H+, 461.19318; found (ESI, [M+H]+ Observed), 461.1932.

### Example 244

Preparation of methyl 4-{[5-(3-fluoro-5-hydroxyphenyl)-7-morpholin-4-yl-3H-[1,2,3]triazolo[4,5-d]pyrimidin-3-yl]methyl}benzoate was prepared from methyl 4-[(5-chloro-7-morpholin-4-yl-3H-[1,2,3]triazolo[4,5-d]pyrimidin-3-yl)methyl]benzoate (200 mg, 0.52 mmol) and 3-fluoro-5-hydroxyphenylboronic acid (120 mg, 0.77 mmol) as described in the example above to give the product as off-white solid (124 mg, 52% yield). MS (ESI) m/z 465.1. HRMS: calculated for C₂₃H₂₁FN₆O₄+H⁺, 465.16811; found (ESI, [M+H]+ Observed), 465.1679.

### Example 245

Preparation of [5-(3-hydroxyphenyl)-7-morpholin-4-yl-3H-[1,2,3]triazolo[4,5-d]pyrimidin-3-yl]acetic acid. To a solution of tert-butyl [5-(3-hydroxyphenyl)-7-morpholin-4-yl-3H-[1,2,3]triazolo[4,5-d]pyrimidin-3-yl] (50 mg, 0.12 mmol) in CH₂Cl₂ (5 mL) was added TFA (2 mL), and the resulting mixture was stirred at room temperature for 5 h. The solvent was removed under reduced pressure, and the residue was subjected to HPLC separation to give the title product as off-white solid (27 mg, 62% yield). MS (ESI) m/z 357.2; HRMS: calculated for C₁₆H₁₆N₆O₄+H⁺, 357.13058; found (ESI, [M+H]⁺ Observed), 357.1308.

### Example 246

Preparation of 1-{4-[(2,2-dimethylhydrazino)carbonyl]phenyl}-3-[4-(3-ethyl-7-morpholin-4-yl-3H-[1,2,3]triazolo[4,5-d]pyrimidin-5-yl)phenyl]urea. The compound was prepared as described in examples above using 4-(3-(4-(3-ethyl-7-morpholino-3H-[1,2,3]triazolo[4,5-d]pyrimidin-5-yl)phenyl)ureido)benzoic acid (100 mg, 0.2 mmol), N,N-dimethylhydrazine (40 μL, 0.52 mmol) and NEt₃ (60 μL, 0.40 mmol), HOBT (54 mg, 0.40 mmol) and EDCI (80 mg, 0.40 mmol) in anhydrous THF (2 mL). The solvents were removed in a N²-stream and the crude mixture was purified by semi-prep-HPLC (NH₃-method) to give 1-{4-[(2,2-dimethylhydrazino)carbonyl]phenyl}-3-[4-(3-ethyl-7-morpholin-4-yl-3H-[1,2,3]triazolo[4,5-d]pyrimidin-5-yl)phenyl]urea (10 mg, 10% yield), MS (ESI) m/z 531.2.

### Example 247

Preparation of 1-[4-(3-ethyl-7-morpholin-4-yl-3H-[1,2,3]triazolo[4,5-d]pyrimidin-5-yl)phenyl]-3-(4-nitrophenyl)urea. To the 4-(3-ethyl-7-morpholino-3H-[1,2,3]triazolo[4,5-d]pyrimidin-5-yl)aniline (300 mg, 0.917 mmol) in CHCl₃ (8 mL) was added Et₃N, stirred for 15 min. and added 1-isocyanato-4-nitrobenzene (227 mg, 1.38 mmol). The mixture was stirred overnight then filtered and purified by silica gel chromatography using EtOAc:Hex (1:1) to give 1-(4-(3-ethyl-7-morpholino-3H-[1,2,3]triazolo[4,5-d]pyrimidin-5-yl)phenyl)-3-(4-nitrophenyl)urea (280 mg, 62% yield) as a beige solid, MS (ESI) m/z=490.2.

### Example 248

Preparation of 1-(4-aminophenyl)-3-[4-(3-ethyl-7-morpholin-4-yl-3H-[1,2,3]triazolo[4,5-d]pyrimidin-5-yl)phenyl]urea. To the mixture 1-(4-(3-ethyl-7-morpholino-3H-[1,2,3]triazolo[4,5-d]pyrimidin-5-yl)phenyl)-3-(4-nitrophenyl)urea (950 mg, 1.94 mmol), MeOH (30 mL), THF (10 mL), and CH₂C12 (10 mL) was added Raney nickel (2.38 g.) then Hydrazine.H₂O (475 mg, 9.48 mmol). The mixture was stirred for 15 min. then filtered, evaporated the solvents and purified by silica gel chromatography using 10% MeOH in CHCl₃ to give 1-(4-aminophenyl)-3-(4-(3-ethyl-7-morpholino-3H-[1,2,3]triazolo[4,5-d]pyrimidin-5-yl)phenyl)urea (634 mg, 71% yield) as an off-white solid, MS (ESI) m/z=460.3.

### Example 249

Preparation of N-[4-({[4-(3-ethyl-7-morpholin-4-yl-3H-[1,2,3]triazolo[4,5-d]pyrimidin-5-yl)phenyl]carbamoyl}amino)phenyl]-N²,N²-dimethylglycinamide. To the 1-(4-aminophenyl)-3-(4-(3-ethyl-7-morpholino-3H-[1,2,3]triazolo[4,5-d]pyrimidin-5-yl)phenyl)urea (95 mg, 0.207 mmol) and CHCl₃ (1.3 mL) was added Et₃N (87 μL, 0.622 mmol) stirred for 15 min. and added 2-(dimethylamino)acetyl chloride. HCl (49 mg, 0.311 mmol) followed by DMAP (5 mg). The mixture was stirred overnight and purified by silica gel chromatography using CH₂Cl₂, MeOH, 7N NH₃ in MeOH (10:1:0.22) method to give N-[4-({[4-(3-ethyl-7-morpholin-4-yl-3H-[1,2,3]triazolo[4,5-d]pyrimidin-5-yl)phenyl]carbamoyl}amino)phenyl]-N²,N²-dimethylglycinamide (70 mg, 62% yield) as a beige solid, MS (ESI) m/z=574.4.

### Example 250

Preparation of 3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoic acid. To a stirred solution of triphosgene (126 mg, 0.42 mmol) in THF (4 mL) was added methyl 3-(5-(4-aminophenyl)-7-morpholino-3H-[1,2,3]triazolo[4,5-d]pyrimidin-3-yl)benzoate (200 mg, 0.53 mmol) at 25° C. The reaction mixture was stirred for 15 min and NEt₃ (73 μL, 0.53 mmol) was added. The mixture was stirred for 1 h and 4-amino-N-(2-(dimethylamino)ethyl)benzamide (331 mg, 1.6 mmol) and NEt₃ (733 μL, 5.3 mmol) were added and the reaction mixture was stirred for additional 1 hr. The solvents were distilled on a rotary evaporator and the crude mixture was purified by semi-prep-HPLC (NH₃-method) to give methyl 3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoate (230 mg, 65% yield), MS (ESI) m/z 665.

To a stirred suspension of methyl 3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoate (230 mg, 0.34 mmol) in THF (5 mL) and MeOH (2 mL) was added NaOH aqueous (5N) (1 mL, 5 mmol) and the mixture was stirred over night. The solvents were removed on rotary evaporator and water was added and the mixture was made acidic with 6N HCl. Upon acidification the product precipitated, which was collected by filtration to obtain as off white solid (130 mg, 59% yield), MS (ESI) m/z 651.3.

### Example 251

Preparation of 4-[({4-[3-(3-carbamoylphenyl)-7-morpholin-4-yl-3H-[1,2,3]triazolo[4,5-d]pyrimidin-5-yl]phenyl}carbamoyl)amino]-N-[2-(dimethylamino)ethyl]benzamide. The compound was prepared as described in examples above using 3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoic acid (70 mg, 0.11 mmol), NH₃ (0.5 M solution in dioxan) (440 μL, 0.22 mmol) and NEt₃ (30 μL, 0.22 mmol), HOBT (30 mg, 0.22 mmol) and EDCI (42 mg, 0.22 mmol) in anhydrous THF (2 mL) and DMF (1 mL). The solvents were removed in a N²-stream and the crude mixture was purified by semi-prep-HPLC (NH₃-method) to give 4-[{4-[3-(3-carbamoylphenyl)-7-morpholin-4-yl-3H-[1,2,3]triazolo[4,5-d]pyrimidin-5-yl]phenyl}carbamoyl)amino]-N-[2-(dimethylamino)ethyl]benzamide (12 mg, 16% yield), MS (ESI) m/z 650.

### Example 252

Preparation of 1-[4-(3-ethyl-7-morpholin-4-yl-3H-[1,2,3]triazolo[4,5-d]pyrimidin-5-yl)phenyl]-3-{4-[(pyridin-4-ylmethyl)amino]phenyl}urea. To the 1-(4-aminophenyl)-3-(4-(3-ethyl-7-morpholino-3H-[1,2,3]triazolo[4,5-d]pyrimidin-5-yl)phenyl)urea (50 mg, 0.109 mmol) and MeOH (0.5 mL) was added isonicotinaldehyde (93 mg, 0.872 mmol) and stirred for 30 minutes then added the mixture of ZnCl₂ (50 mg), NaHBCN (50 mg) and MeOH (0.5 mL) then stirred overnight. The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-{4-[(pyridin-4-ylmethyl)amino]phenyl}urea a as a TFA salt (45.6 mg, 54% yield), MS (ESI) m/z=551.5.

### Example 253

Preparation of 1-[4-(3-ethyl-7-morpholin-4-yl-3H-[1,2,3]triazolo[4,5-d]pyrimidin-5-yl)phenyl]-3-{4-[(pyridin-3-ylmethyl)amino]phenyl}urea. The compound was prepared as described in the example above using -(4-aminophenyl)-3-(4-(3-ethyl-7-morpholino-3H-[1,2,3]triazolo[4,5-d]pyrimidin-5-yl)phenyl)urea (50 mg, 0.109 mmol), nicotinaldehyde (93 mg, 0.872 mmol), ZnCl₂ (50 mg), NaHBCN (50 mg) and MeOH (1 mL). The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-{4-[(pyridin-3-ylmethyl)amino]phenyl}urea as a TFA salt (49.8 mg, 59% yield), MS (ESI) m/z=551.5.

### Example 254

Preparation of 1-[4-(3-ethyl-7-morpholin-4-yl-3H-[1,2,3]triazolo[4,5-d]pyrimidin-5-yl)phenyl]-3-(4-{[(6-fluoropyridin-3-yl)methyl]amino}phenyl)urea. The compound was prepared as described in the example above using -(4-aminophenyl)-3-(4-(3-ethyl-7-morpholino-3H-[1,2,3]triazolo[4,5-d]pyrimidin-5-yl)phenyl)urea (50 mg, 0.109 mmol), 6-fluoronicotinaldehyde (109 mg, 0.872 mmol), ZnCl₂ (50 mg), NaHBCN (50 mg) and MeOH (1 mL). The solvent was removed in an N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-(4-{[(6-fluoropyridin-3-yl)methyl]amino}phenyl)urea as a TFA salt (10.2 mg, 14% yield), MS (ESI) m/z=569.2.

### Example 255

Preparation of 1-[4-(3-ethyl-7-morpholin-4-yl-3H-[1,2,3]triazolo[4,5-d]pyrimidin-5-yl)phenyl]-3-(4-{[(6-methoxypyridin-3-yl)methyl]amino}phenyl)urea. The compound was prepared as described in the example above using -(4-aminophenyl)-3-(4-(3-ethyl-7-morpholino-3H-[1,2,3]triazolo[4,5-d]pyrimidin-5-yl)phenyl)urea (50 mg, 0.109 mmol), 6-methoxynicotinaldehyde (120 mg, 0.872 mmol), ZnCl₂ (50 mg), NaHBCN (50 mg) and MeOH (1 mL). The solvent was removed in an N₂-stream and the crude product was purified by HPLC (TFA-method) to give 1-(4-(3-ethyl-7-morpholino-3H-[1,2,3]triazolo[4,5-d]pyrimidin-5-yl)phenyl)-3-(4-((6-methoxypyridin-3-yl)methylamino)phenyl)urea as a TFA salt (31.6 mg, 36% yield), MS (ESI) m/z=581.3.

### Example 256

Preparation of N-[2-(dimethylamino)ethyl]-4-[({4-[3-(1-methylethyl)-7-morpholin-4-yl-3H-[1,2,3]triazolo[4,5-d]pyrimidin-5-yl]phenyl}carbamoyl)amino]benzamide. To a stirred solution of triphosgene (35 mg, 0.12 mmol) in CH₂Cl₂ (4 mL) was added 4-(3-isopropyl-7-morpholino-3H-[1,2,3]triazolo[4,5-d]pyrimidin-5-yl)aniline (50 mg, 0.14 mmol) at 25° C. The reaction mixture was stirred for 15 min and NEt₃ (20 μL, 0.14 mmol) was added. Stirring was continued for 1 h and 4-amino-N-(2-(dimethylamino)ethyl)benzamide (90 mg, 0.43 mmol) and NEt₃ (200 μL, 1.4 mmol) were added and the reaction mixture was stirred for additional 1 hr. The solvents were removed in a N₂ stream and the crude mixture was purified by semi-prep-HPLC (NH₃-method) to give methyl N-[2-(dimethylamino)ethyl]-4-[({4-[3-(1-methylethyl)-7-morpholin-4-yl-3H-[1,2,3]triazolo[4,5-d]pyrimidin-5-yl]phenyl}carbamoyl)amino]benzamide (27 mg, 39% yield), MS (ESI) m/z 572.

### Example 257

Preparation of 1-{4-[3-(1-methylethyl)-7-morpholin-4-yl-3H-[1,2,3]triazolo[4,5-d]pyrimidin-5-yl]phenyl}-3-{4-[(4-methylpiperazin-1-yl)carbonyl]phenyl}urea. To a stirred solution of triphosgene (35 mg, 0.12 mmol) in CH₂Cl₂ (4 mL) was added 4-(3-isopropyl-7-morpholino-3H-[1,2,3]triazolo[4,5-d]pyrimidin-5-yl)aniline (50 mg, 0.14 mmol) at 25° C. The reaction mixture was stirred for 15 min and NEt₃ (20 μL, 0.14 mmol) was added. Stirring was continued for 1 h and (4-aminophenyl)(4-methylpiperazin-1-yl)methanone (103 mg, 0.43 mmol) and NEt₃ (200 μL, 1.4 mmol) were added and the reaction mixture was stirred for additional 1 hr. The solvents were removed in a N₂ stream and the crude mixture was purified by semi-prep-HPLC (NH₃-method) to give 1-{4-[3-(1-methylethyl)-7-morpholin-4-yl-3H-[1,2,3]triazolo[4,5-d]pyrimidin-5-yl]phenyl}-3-{4-[(4-methylpiperazin-1-yl)carbonyl]phenyl}urea (43 mg, 39% yield), MS (ESI) m/z 585.4.

### Example 258

Preparation of 1-[4-(3-ethyl-7-morpholin-4-yl-3H-[1,2,3]triazolo[4,5-d]pyrimidin-5-yl)phenyl]-3-{4-[(4-methylpiperazin-1-yl)methyl]phenyl}urea. To a stirred solution of triphosgene (35 mg, 0.12 mmol) in CH₂Cl₂ (4 mL) was added 4-(3-isopropyl-7-morpholino-3H-[1,2,3]triazolo[4,5-d]pyrimidin-5-yl)aniline (50 mg, 0.14 mmol) at 25° C. The reaction mixture was stirred for 15 min and NEt₃ (20 μL, 0.14 mmol) was added. Stirring was continued for 1 h and 4-((4-methylpiperazin-1-yl)methyl)aniline (90 mg, 0.43 mmol) and NEt₃ (200 μL, 1.4 mmol) were added and the reaction mixture was stirred for additional 1 hr. The solvents were removed in a N₂ stream and the crude mixture was purified by semi-prep-HPLC (TFA-method) to give 1-[4-(3-ethyl-7-morpholin-4-yl-3H-[1,2,3]triazolo[4,5-d]pyrimidin-5-yl)phenyl]-3-{4-[(4-methylpiperazin-1-yl)methyl]phenyl}urea as TFA salt (14 mg, 15% yield), MS (ESI) m/z 557.

### Example 259

Preparation of 1-[4-(3-ethyl-7-morpholin-4-yl-3H-[1,2,3]triazolo[4,5-d]pyrimidin-5-yl)phenyl]-3-[4-(4-methylpiperazin-1-yl)phenyl]urea. To a stirred solution of triphosgene (109 mg, 0.37 mmol) in CH₂Cl₂ (4 mL) was added 4-(3-ethyl-7-morpholino-3H-[1,2,3]triazolo[4,5-d]pyrimidin-5-yl)aniline (150 mg, 0.45 mmol) at 25° C. The reaction mixture was stirred for 15 min and NEt₃ (62 μL, 0.45 mmol) was added. Stirring was continued for 1 h and 4-(4-methylpiperazin-1-yl)aniline (258 mg, 0.43 mmol) and NEt₃ (622 μL, 4.5 mmol) were added and the reaction mixture was stirred for additional 1 hr. The solvents were removed in a N₂ stream and the crude mixture was purified by semi-prep-HPLC (TFA-method) to give 1-[4-(3-ethyl-7-morpholin-4-yl-3H-[1,2,3]triazolo[4,5-d]pyrimidin-5-yl)phenyl]-3-[4-(4-methylpiperazin-1-yl)phenyl]urea as bis-TFA salt (92 mg, 27% yield), MS (ESI) m/z 543.3.

### Example 260

Preparation of 4-({[4-(3-ethyl-7-morpholin-4-yl-3H-[1,2,3]triazolo[4,5-d]pyrimidin-5-yl)phenyl]carbamoyl}amino)-N-pyridin-3-ylbenzamide. To a stirred solution of triphosgene (45 mg, 0.15 mmol) in CH₂Cl₂ (4 mL) was added 4-(3-ethyl-7-morpholino-3H-[1,2,3]triazolo[4,5-d]pyrimidin-5-yl)aniline (100 mg, 0.3 mmol) at 25° C. The reaction mixture was stirred for 15 min and NEt₃ (42 μL, 0.3 mmol) was added. Stirring was continued for 1 h and 4-amino-N-(pyridin-3-yl)benzamide (191 mg, 0.9 mmol) and NEt₃ (420 μL, 3.0 mmol) were added and the reaction mixture was stirred for additional 1 hr. The solvents were removed in a N₂ stream and the crude mixture was purified by semi-prep-HPLC (NH₃-method) to give 4-({[4-(3-ethyl-7-morpholin-4-yl-3H-[1,2,3]triazolo[4,5-d]pyrimidin-5-yl)phenyl]carbamoyl}amino)-N-pyridin-3-ylbenzamide (63 mg, 37% yield), MS (ESI) m/z 565.

### Example 261

Preparation of N-[4-({[4-(3-ethyl-7-morpholin-4-yl-3H-[1,2,3]triazolo[4,5-d]pyrimidin-5-yl)phenyl]carbamoyl}amino)phenyl]-4-methylpiperazine-1-carboxamide. To the 1-(4-aminophenyl)-3-(4-(3-ethyl-7-morpholino-3H-[1,2,3]triazolo[4,5-d]pyrimidin-5-yl)phenyl)urea (40 mg, 0.087 mmol) and THF (1 mL) was added Et₃N (36 μL, 0.262 mmol) stirred for 15 min. and added 4-methylpiperazine-1-carbonyl chloride (42 mg, 0.262 mmol) followed by catalytic amount of DMAP then stirred overnight. The solvent was removed in a N₂-stream and the crude product was purified by HPLC (TFA-method) to give N-[4-({[4-(3-ethyl-7-morpholin-4-yl-3H-[1,2,3]triazolo[4,5-d]pyrimidin-5-yl)phenyl]carbamoyl}amino)phenyl]-4-methylpiperazine-1-carboxamide as a TFA salt (38.1 mg, 63% yield), MS (ESI) m/z=586.3.

### Example 262

N-[4-({[4-(3-ethyl-7-morpholin-4-yl-3H-[1,2,3]triazolo[4,5-d]pyrimidin-5-yl)phenyl]carbamoyl}amino)phenyl]pyridine-4-carboxamide. The compound was prepared as described in the example above using 1-(4-aminophenyl)-3-(4-(3-ethyl-7-morpholino-3H-[1,2,3]triazolo[4,5-d]pyrimidin-5-yl)phenyl)urea (40 mg, 0.087 mmol), isonicotinoyl chloride (46 mg, 0.261 mmol), Et₃N (36 μL, 0.262 mmol), DMAP (cat.) and THF (1 mL). The solvent was removed in a N₂-stream and the crude product was purified by HPLC (TFA-method) to give N-[4-({[4-(3-ethyl-7-morpholin-4-yl-3H-[1,2,3]triazolo[4,5-d]pyrimidin-5-yl)phenyl]carbamoyl}amino)phenyl]pyridine-4-carboxamide a TFA salt (34.2 mg, 70% yield), MS (ESI) m/z=565.2.

### Example 263

Preparation of N-[4-({[4-(3-ethyl-7-morpholin-4-yl-3H-[1,2,3]triazolo[4,5-d]pyrimidin-5-yl)phenyl]carbamoyl}amino)phenyl]morpholine-4-carboxamide. The compound was prepared as described in the example above using 1-(4-aminophenyl)-3-(4-(3-ethyl-7-morpholino-3H-[1,2,3]triazolo[4,5-d]pyrimidin-5-yl)phenyl)urea (40 mg, 0.087 mmol), morpholine-4-carbonyl chloride (39 mg, 0.261 mmol), Et₃N (36 μL, 0.262 mmol), DMAP (cat.) and THF (1 mL). The solvent was removed in a N₂-stream and the crude product was purified by HPLC (TFA-method) to give N-(4-(3-(4-(3-ethyl-7-morpholino-3H-[1,2,3]triazolo[4,5-d]pyrimidin-5-yl)phenyl)ureido)phenyl)morpholine-4-carboxamide as a TFA salt (8.7 mg, 17% yield), MS (ESI) m/z=573.3.

### Example 264

Preparation of 3-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]benzamide. The compound was prepared as described in the example above using 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 3-(dimethylamino)benzoyl chloride (58 mg, 0.261 mmol), Et₃N (36 μL, 0.262 mmol), DMAP (cat.) and THF (1 mL). The solvent was removed in a N₂-stream and the crude product was purified by HPLC (TFA-method) to give N-(4-(3-(4-(3-ethyl-7-morpholino-3H-[1,2,3]triazolo[4,5-d]pyrimidin-5-yl)phenyl)ureido)phenyl)morpholine-4-carboxamide as a TFA salt (11.7 mg, 19% yield), MS (ESI) m/z=607.3.

### Example 265

1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]urea. To a stirred solution of triphosgene (21 mg, 0.070 mmol) in CHCl₂ (1.5 mL) was added 4-1-(4-aminophenyl)-3-(4-(3-ethyl-7-morpholino-3H-[1,2,3]triazolo[4,5-d]pyrimidin-5-yl)phenyl)urea (40 mg, 0.087 mmol) at 25° C. The mixture was stirred for 15 min. and added Et₃N (18 μL, 0.131 mmol) and stirred for 1 hr. then N,N-dimethylethylenediamine (23 mg, 0.262 mmol) and Et₃N (103 μL, 0.736 mmol) was added and the reaction mixture was stirred for additional 1 hr. The solvents were distilled on a rotary evaporator and the crude mixture was purified by semi-prep-HPLC (TFA-method) to give 1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]urea as a TFA salt (35 mg, 58% yield), MS (ESI) m/z 574.4.

### Example 266

### 4-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]piperidine-1-carboxamide

To a stirred solution of triphosgene (21 mg, 0.070 mmol) in CHCl₂ (1.5 mL) was added 4-1-(4-aminophenyl)-3-(4-(3-ethyl-7-morpholino-3H-[1,2,3]triazolo[4,5-d]pyrimidin-5-yl)phenyl)urea (40 mg, 0.087 mmol) at 25° C. The mixture was stirred for 15 min. and added Et₃N (18 μL, 0.131 mmol) and stirred for 1 hr. then N,N-dimethylpiperidin-4-amine (34 mg, 0.262 mmol) and Et₃N (103 μL, 0.736 mmol) was added and the reaction mixture was stirred for additional 1 hr. The solvents were distilled on a rotary evaporator and the crude mixture was purified by semi-prep-HPLC (TFA-method) to give 4-(dimethylamino)-N-(4-(3-(4-(3-ethyl-7-morpholino-3H-[1,2,3]triazolo[4,5-d]pyrimidin-5-yl)phenyl)ureido)phenyl)piperidine-1-carboxamide as a TFA salt (48 mg, 75% yield), MS (ESI) m/z=614.4.

### Example 267

1-[4-(3-ethyl-7-morpholin-4-yl-3H-[1,2,3]triazolo[4,5-d]pyrimidin-5-yl)phenyl]-3-(4-{[(1-methylpiperidin-4-yl)carbamoyl]amino}phenyl)urea. To a stirred solution of triphosgene (21 mg, 0.0.069 mmol) in CH₂Cl₂ (1.5 mL) was added 1-(4-aminophenyl)-3-(4-(3-ethyl-7-morpholino-3H-[1,2,3]triazolo[4,5-d]pyrimidin-5-yl)phenyl)urea (40 mg, 0.087 mmol) at 25° C. The reaction mixture was stirred for 30 min then NEt₃ (121 μL, 0.87 mmol) and 1-methylpiperidin-4-amine (30 mg, 0.262 mmol) were added. Stirred for 2.5 hrs. and the solvent was removed in a N₂ stream and the crude mixture was purified by HPLC (TFA-method) to give 1-[4-(3-ethyl-7-morpholin-4-yl-3H-[1,2,3]triazolo[4,5-d]pyrimidin-5-yl)phenyl]-3-(4-{[(1-methylpiperidin-4-yl)carbamoyl]amino}phenyl)urea as a TFA salt (4.5 mg, 7% yield), MS (ESI) m/z=600.7

### Example 268

1-[4-(3-ethyl-7-morpholin-4-yl-3H-[1,2,3]triazolo[4,5-d]pyrimidin-5-yl)phenyl]-3-[4-({[2-(4-methylpiperazin-1-yl)ethyl]carbamoyl}amino)phenyl]urea. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 2-(4-methylpiperazin-1-yl)ethanamine (38 mg, 0.262 mmol), triethylamine (121 μL, 0.87 mmol), and methylene chloride (1.5 mL). The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-[4-({[2-(4-methylpiperazin-1-yl)ethyl]carbamoyl}amino)phenyl]urea as a TFA salt (35.5 mg; 48% yield), MS (ESI) m/z 629.3

### Example 269

N-[4-({[4-(3-ethyl-7-morpholin-4-yl-3H-[1,2,3]triazolo[4,5-d]pyrimidin-5-yl)phenyl]carbamoyl}amino)phenyl]-4-methyl-1,4-diazepane-1-carboxamide. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 1-methyl-1,4-diazepane (30 mg, 0.262 mmol), triethylamine (121 μL, 0.87 mmol) and methylene chloride (1.5 mL). The solvent was removed in a N₂-stream and the crude product was purified by HPLC (TFA-method) to give N-(4-(3-(4-(3-ethyl-7-morpholino-3H-[1,2,3]triazolo[4,5-d]pyrimidin-5-yl)phenyl)ureido)phenyl)-4-methyl-1,4-diazepane-1-carboxamide as a TFA salt (26.6 mg; 43% yield), MS (ESI) m/z=600.3.

### Example 270

### 1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]-1-methylurea

The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), N1,N1,N2-trimethylethane-1,2-diamine (27 mg, 0.262 mmol), triethylamine (121 μL, 0.87 mmol) and methylene chloride (1.5 mL). The solvent was removed in a N₂-stream and the crude product was purified by HPLC (TFA-method) to give 1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]-1-methylurea as a TFA salt (29.8 mg; 49% yield), MS (ESI) m/z=588.3.

### Example 271

1-[4-(3-ethyl-7-morpholin-4-yl-3H-[1,2,3]triazolo[4,5-d]pyrimidin-5-yl)phenyl]-3-(4-{[(2-pyrrolidin-1-ylethyl)carbamoyl]amino}phenyl)urea. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 2-(pyrrolidin-1-yl)ethanamine (30 mg, 0.262 mmol), triethylamine (121 μL, 0.87 mmol), and methylene chloride (1.5 mL). The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-(4-{[(2-pyrrolidin-1-ylethyl)carbamoyl]amino}phenyl)urea as a TFA salt (27.9 mg; 45% yield), MS (ESI) m/z 600.7

### Example 272

N-[4-({[4-(3-ethyl-7-morpholin-4-yl-3H-[1,2,3]triazolo[4,5-d]pyrimidin-5-yl)phenyl]carbamoyl}amino)phenyl]-4-pyrrolidin-1-ylpiperidine-1-carboxamide. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 4-(pyrrolidin-1-yl)piperidine (40 mg, 0.262 mmol), triethylamine (121 μL, 0.87 mmol), and methylene chloride (1.5 mL). The solvent was removed in a N₂-stream and the crude product was purified by HPLC (TFA-method) to give N-(4-(3-(4-(3-ethyl-7-morpholino-3H-[1,2,3]triazolo[4,5-d]pyrimidin-5-yl)phenyl)ureido)phenyl)-4-(pyrrolidin-1-yl)piperidine-1-carboxamide as a TFA salt (27.9 mg; 45% yield), MS (ESI) m/z 640.3.

### Example 273

1-[4-(3-ethyl-7-morpholin-4-yl-3H-[1,2,3]triazolo[4,5-d]pyrimidin-5-yl)phenyl]-3-(4-{[(pyridin-2-ylmethyl)carbamoyl]amino}phenyl)urea. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), pyridin-2-ylmethanamine (30 mg, 0.262 mmol), triethylamine (121 μL, 0.87 mmol), and methylene chloride (1.5 mL). The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-(4-{[(pyridin-2-ylmethyl)carbamoyl]amino}phenyl)urea as a TFA salt (22.8 mg; 37% yield), MS (ESI) m/z 594.3

### Example 274

N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), piperazine (23 mg, 0.262 mmol), triethylamine (121 μL, 0.87 mmol), and methylene chloride (1.5 mL). The solvent was removed in a N₂-stream and the crude product was purified by HPLC (TFA-method) to give N-(4-(3-(4-(3-ethyl-7-morpholino-3H-[1,2,3]triazolo[4,5-d]pyrimidin-5-yl)phenyl)ureido)phenyl)piperazine-1-carboxamide as a TFA salt (3 mg; 5% yield), MS (ESI) m/z=572.6

### Example 275

4-ethyl-N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 1-ethylpiperazine (30 mg, 0.262 mmol), triethylamine (121 μL, 0.87 mmol) and methylene chloride (1.5 mL). The solvent was removed in a N₂-stream and the crude product was purified by HPLC (TFA-method) to give 4-ethyl-N-(4-(3-(4-(3-ethyl-7-morpholino-3H-[1,2,3]triazolo[4,5-d]pyrimidin-5-yl)phenyl)ureido)phenyl)piperazine-1-carboxamide as a TFA salt (27.6 mg; 44% yield), MS (ESI) m/z=600.3.

### Example 276

1-[4-(3-ethyl-7-morpholin-4-yl-3H-[,2,3]triazolo[4,5-d]pyrimidin-5-yl)phenyl]-3-(4-{[(2-methoxyethyl)carbamoyl]amino}phenyl)urea. The compound was prepared as described in the example above using triphosgene (21 mg, 0.70 mmol), 1-(4-aminophenyl)-3-(4-(3-ethyl-7-morpholino-3H-[1,2,3]triazolo[4,5-d]pyrimidin-5-yl)phenyl)urea (40 mg, 0.087 mmol), 2-methoxyethanamine (20 mg, 0.262 mmol), triethylamine (121 μL, 0.87 mmol) and methylene chloride (1.5 mL). The solvent was removed in a N₂-stream and the crude product was purified by HPLC (TFA-method) to give 1-[4-(3-ethyl-7-morpholin-4-yl-3H-[1,2,3]triazolo[4,5-d]pyrimidin-5-yl)phenyl]-3-(4-{[(2-methoxyethyl)carbamoyl]amino}phenyl)urea as a TFA salt (5.4 mg; 11% yield), MS (ESI) m/z=561.3.

### Example 277

Preparation of 1-{4-[3-(1-methylethyl)-7-morpholin-4-yl-3H-[1,2,3]triazolo[4,5-d]pyrimidin-5-yl]phenyl}-3-[4-(4-methylpiperazin-1-yl)phenyl]urea. To a stirred solution of triphosgene (109 mg, 0.37 mmol) in CH₂Cl₂ (4 mL) was added 4-(3-isopropyl-7-morpholino-3H-[1,2,3]triazolo[4,5-d]pyrimidin-5-yl)aniline (150 mg, 0.44 mmol) at 25° C. The reaction mixture was stirred for 15 min and NEt₃ (62 μL, 0.45 mmol) was added. Stirring was continued for 1 h and 4-(4-methylpiperazin-1-yl)aniline (258 mg, 0.43 mmol) and NEt₃ (622 μL, 4.5 mmol) were added and the reaction mixture was stirred for additional 1 hr. The solvents were removed in a N₂ stream and the crude mixture was purified by semi-prep-HPLC (TFA-method) to give 1-[4-(3-ethyl-7-morpholin-4-yl-3H-[1,2,3]triazolo[4,5-d]pyrimidin-5-yl)phenyl]-3-[4-(4-methylpiperazin-1-yl)phenyl]urea (86 mg, 35% yield), MS (ESI) m/z 557.6.

### Example 278

Preparation of 1-{4-[3-(1-methylethyl)-7-morpholin-4-yl-3H-[1,2,3]triazolo[4,5-d]pyrimidin-5-yl]phenyl}-3-(4-nitrophenyl)urea. To a stirred solution of 4-(3-isopropyl-7-morpholino-3H-[1,2,3]triazolo[4,5-d]pyrimidin-5-yl)aniline (200 mg, 0.6 mmol) in anhydrous THF (4 mL) was added a solution of 4-nitrophenylisocyanat (118 mg, 0.72 mmol) in THF (1 mL) The mixture was stirred for 8 hours and the yellow solid was collected by filtration. The filter cake was washed with hexane (1 mL) and dried in a vacuum oven to give the product 1-{4-[3-(1-methylethyl)-7-morpholin-4-yl-3H-[1,2,3]triazolo[4,5-d]pyrimidin-5-yl]phenyl}-3-(4-nitrophenyl)urea as yellow solid (140 mg, 46% yield), MS (ESI) m/z 504.4.

### Example 279

Preparation of N-[4-({[4-(3-isopropyl-7-morpholin-4-yl-3H-[1,2,3]triazolo[4,5-d]pyrimidin-5-yl)phenyl]carbamoyl}amino)phenyl]methanesulfonamide. To a stirred solution of 1-(4-aminophenyl)-3-[4-(3-isopropyl-7-morpholin-4-yl-3H-[1,2,3]triazolo[4,5-d]pyrimidin-5-yl)phenyl]urea (100 mg, 0.21 mmol) and NaOH aqueous (2.5N) (200 μL, 0.5 mmol) in THF (1 mL) was added MeSO₃Cl (20 μL, 0.253 mmol) and the mixture was stirred for 2 hours. The formed precipitate was collected by filtration and washed with water and allowed to dry on the filter to give N-[4-({[4-(3-isopropyl-7-morpholin-4-yl-3H-[1,2,3]triazolo[4,5-d]pyrimidin-5-yl)phenyl]carbamoyl}amino)phenyl]methanesulfonamide as off white solid (92 mg, 79% yield)MS (ESI) m/z 552.2.

### Example 280

Preparation of 1-(4-aminophenyl)-3-[4-(3-isopropyl-7-morpholin-4-yl-3H-[1,2,3]triazolo[4,5-d]pyrimidin-5-yl)phenyl]urea. In a three-necked flask was suspended under nitrogen atmosphere 1-{4-[3-(1-methylethyl)-7-morpholin-4-yl-3H-[1,2,3]triazolo[4,5-d]pyrimidin-5-yl]phenyl}-3-(4-nitrophenyl)urea (200 mg, 0.4 mmol) and Pd/C (10% wet) (200 mg) in methanol (150 mL) and CH₂Cl₂ (50 mL). The mixture was hydrogenated at 1 atm pressure using a H₂-ballon. After 1 hr the reaction was completed and the mixture was filtered over Celite and the filtrate was evaporated to dryness to give the product as brown solid 1-(4-aminophenyl)-3-[4-(3-isopropyl-7-morpholin-4-yl-3H-[1,2,3]triazolo[4,5-d]pyrimidin-5-yl)phenyl]urea (180 mg, 95% yield). MS (ESI) m/z 473.

### Example 281

Preparation of 1-(4-{[4-(dimethylamino)piperidin-1-yl]carbonyl}phenyl)-3-(4-{3-ethyl-7-[(3S)-3-methylmorpholin-4-yl]-3H-[1,2,3]triazolo[4,5-d]pyrimidin-5-yl}phenyl). To a stirred solution of triphosgene (21 mg, 0.70 mmol) in CHCl₃ (1.5 mL) was added (S)-4-(3-ethyl-7-(3-methylmorpholino)-3H-[1,2,3]triazolo[4,5-d]pyrimidin-5-yl)aniline (30 mg, 0.088 mmol) at 25° C. The reaction mixture was stirred for 15 min and added triethylamine (18 μL, 0.132 mmol) stirred for 60 min. then added (4-aminophenyl)(4-(dimethylamino)piperidin-1-yl)methanone (65 mg, 0.264 mmol). Stirred for additional 30 min. and added triethylamine (104 μL, 0.748 mmol) then stirred overnight. The solvent was removed in a N₂-stream and the crude product was purified by HPLC (TFA-method) to give (S)-1-(4-(4-(dimethylamino)piperidine-1-carbonyl)phenyl)-3-(4-(3-ethyl-7-(3-methylmorpholino)-3H-[1,2,3]triazolo[4,5-d]pyrimidin-5-yl)phenyl)urea as a TFA salt (31.2 mg, 49% yield). MS (ES) m/z=613.3

### Example 282

Preparation of 1-(4-{3-ethyl-7-[(3S)-3-methylmorpholin-4-yl]-3H-[1,2,3]triazolo[4,5-d]pyrimidin-5-yl}phenyl)-3-{4-[(4-methylpiperazin-1-yl)carbonyl]phenyl}urea. The compound was prepared as described in the example above using triphosgene (21 mg, 0.70 mmol), (S)-4-(3-ethyl-7-(3-methylmorpholino)-3H-[1,2,3]triazolo[4,5-d]pyrimidin-5-yl)aniline (30 mg, 0.088 mmol), (4-aminophenyl)(4-methylpiperazin-1-yl)methanone (58 mg, 0.264 mmol) and triethylamine (123 μL, 0.88 mmol) in methylene chloride (1.5 mL). The solvent was removed in a N₂-stream and the crude product was purified by HPLC (TFA-method) to give 1-(4-{3-ethyl-7-[(3S)-3-methylmorpholin-4-yl]-3H-[1,2,3]triazolo[4,5-d]pyrimidin-5-yl}phenyl)-3-{4-[(4-methylpiperazin-1-yl)carbonyl]phenyl}urea as a TFA salt (18.4 mg; 30% yield), MS (ESI) m/z=585.3.

### Example 283

Preparation of 4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-pyrrolidin-1-ylethyl)benzamide . The compound was prepared as described in the example above using triphosgene (21 mg, 0.70 mmol), (S)-4-(3-ethyl-7-(3-methylmorpholino)-3H-[1,2,3]triazolo[4,5-d]pyrimidin-5-yl)aniline (30 mg, 0.088 mmol), 4-amino-N-(2-(pyrrolidin-1-yl)ethyl)benzamide (62 mg, 0.264 mmol) and triethylamine (123 μL, 0.88 mmol) in methylene chloride (1.5 mL). The solvent was removed in a N₂-stream and the crude product was purified by HPLC (TFA-method) to give 4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-pyrrolidin-1-ylethyl)benzamide as a TFA salt (24.8 mg, 40% yield), MS (ESI) m/z=599.3

### Example 284

Preparation of 4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-piperidin-1-ylethyl)benzamide. The compound was prepared as described in the example above using triphosgene (21 mg, 0.70 mmol), (S)-4-(3-ethyl-7-(3-methylmorpholino)-3H-[1,2,3]triazolo[4,5-d]pyrimidin-5-yl)aniline (30 mg, 0.088 mmol), 4-amino-N-(2-(piperidin-1-yl)ethyl)benzamide 65 mg, 0.264 mmol) and triethylamine (123 μL, 0.88 mmol) in methylene chloride (1.5 mL) The solvent was removed in a N₂-stream and the crude product was purified by HPLC (TFA-method) to give 4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-piperidin-1-ylethyl)benzamide as a TFA salt (8.7 mg, 14% yield), MS (ESI) m/z=613.3.

### Example 285

Preparation of N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-methylbenzamide. The compound was prepared as described in the example above using triphosgene (21 mg, 0.70 mmol), (S)-4-(3-ethyl-7-(3-methylmorpholino)-3H-[1,2,3]triazolo[4,5-d]pyrimidin-5-yl)aniline (30 mg, 0.088 mmol), 4-amino-N-(2-(dimethylamino)ethyl)-N-methylbenzamide (58 mg, 0.264 mmol) and triethylamine (123 μL, 0.88 mmol) in methylene chloride (1.5 mL) The solvent was removed in a N₂-stream and the crude product was purified by HPLC (TFA-method) to give N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-methylbenzamide as a TFA salt (8.5 mg, 14% yield), MS (ESI) m/z=587.3.

### Example 286

Preparation of N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}benzamide. The compound was prepared as described in the example above using triphosgene (21 mg, 0.70 mmol), (S)-4-(3-ethyl-7-(3-methylmorpholino)-3H-[1,2,3]triazolo[4,5-d]pyrimidin-5-yl)aniline (30 mg, 0.088 mmol), 4-amino-N-(2-(dimethylamino)ethyl)benzamide (55 mg, 0.264 mmol) and triethylamine (123 μL, 0.88 mmol) in methylene chloride (1.5 mL) The solvent was removed in a N₂-stream and the crude product was purified by HPLC (TFA-method) to give N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}benzamide as a TFA salt (27 mg, 45% yield), MS (ESI) m/z 573.3.

### Example 287

### Preparation of Methyl 5-({[4-(3-ethyl-7-morpholin-4-yl-3H-[1,2,3]triazolo[4,5-d]pyrimidin-5-yl)phenyl]carbamoyl}amino)pyridine-2-carboxylate WYE-132810-1

To a stirred solution of triphosgene (274 mg, 0.92 mmol) in THF (10 mL) was added 4-(3-ethyl-7-morpholino-3H-[1,2,3]triazolo[4,5-d]pyrimidin-5-yl)aniline (500 mg, 1.54 mmol) at 25° C. The reaction mixture was stirred for 15 min and NEt₃ (213 μL, 1.54 mmol) was added. The mixture was stirred for 1 h and methyl 5-aminopicolinate (703 mg, 462 mmol) and NEt₃ (2130 μL, 15.4 mmol) were added and the reaction mixture was stirred for additional 12 hr than CHCl₃ (100 mL) were added and the organic layer were extracted with sat NH₄Cl-sol (10 mL) and brine (10 mL) and the combined organic layers were dried over MgSO₄. Filtration and solvent removal on a rotary evaporator gave the off-white product to give methyl 5-({[4-(3-ethyl-7-morpholin-4-yl-3H-[1,2,3]triazolo[4,5-d]pyrimidin-5-yl)phenyl]carbamoyl}amino)pyridine-2-carboxylate (530 mg, 68% yield), MS (ESI) m/z 504.2.

### Example 288

Preparation of 5-({[4-(3-ethyl-7-morpholin-4-yl-3H-[1,2,3]triazolo[4,5-d]pyrimidin-5-yl)phenyl]carbamoyl}amino)pyridine-2-carboxylic acid. To a stirred suspension of methyl 5-({[4-(3-ethyl-7-morpholin-4-yl-3H-[1,2,3]triazolo[4,5-d]pyrimidin-5-yl)phenyl]carbamoyl}amino)pyridine-2-carboxylate (530 mg, 1.04 mmol) in IPA (5 mL) was added NaOH aqueous (2N) (2 mL, 4 mmol) and the mixture was heated at reflux for 2 hours. The mixture was made acidic with 6N HCl. Upon acidification the product precipitated, which was collected by filtration to obtain as off white solid (100 mg, 19% yield), MS (ESI) m/z 490.

### Example 289

Preparation of 1-[4-(3-ethyl-7-morpholin-4-yl-3H-[1,2,3]triazolo[4,5-d]pyrimidin-5-yl)phenyl]-3-{6-[(4-methylpiperazin-1-yl)carbonyl]pyridin-3-yl}urea. The compound was prepared as described in examples above using 5-({[4-(3-ethyl-7-morpholin-4-yl-3H-[1,2,3]triazolo[4,5-d]pyrimidin-5-yl)phenyl]carbamoyl}amino)pyridine-2-carboxylic acid (50 mg, 0.1 mmol), N-methylpiperazine (20 μL, 0.2 mmol) and NEt₃ (50 μL, 0.4 mmol), HOBT (27 mg, 0.2 mmol) and EDCI (38 mg, 0.2 mmol) in anhydrous DMF (1 mL). The solvents were removed in a N2-stream and the crude mixture was purified by semi-prep-HPLC (NH3-method) to give N-[2-(dimethylamino)ethyl]-5-({[4-(3-ethyl-7-morpholin-4-yl-3H-[1,2,3]triazolo[4,5-d]pyrimidin-5-yl)phenyl]carbamoyl}amino)-N-methylpyridine-2-carboxamide (20 mg, 34% yield)MS (ESI) m/z 572.2.

### Example 290

Preparation of N-[2-(dimethylamino)ethyl]-5-({[4-(3-ethyl-7-morpholin-4-yl-3H-[1,2,3]triazolo[4,5-d]pyrimidin-5-yl)phenyl]carbamoyl}amino)-N-methylpyridine-2-carboxamide. The compound was prepared as described in examples above using 5-({[4-(3-ethyl-7-morpholin-4-yl-3H-[1,2,3]triazolo[4,5-d]pyrimidin-5-yl)phenyl]carbamoyl}amino)pyridine-2-carboxylic acid (50 mg, 0.1 mmol), N,N-dimethylethylenediamine

(18 μL, 0.2 mmol) and NEt₃ (50 μL, 0.4 mmol), HOBT (27 mg, 0.2 mmol) and EDCI (38 mg, 0.2 mmol) in anhydrous DMF (1 mL). The solvents were removed in a N2-stream and the crude mixture was purified by semi-prep-HPLC (NH₃-method) to give N-[2-(dimethylamino)ethyl]-5-({[4-(3-ethyl-7-morpholin-4-yl-3H-[1,2,3]triazolo[4,5-d]pyrimidin-5-yl)phenyl]carbamoyl}amino)-N-methylpyridine-2-carboxamide (14 mg, 18% yield), MS (ESI) m/z 560.

Biological Evaluation—

mTOR Kinase Assay Methods

The routine human TOR assays with purified enzyme are performed in 96-well plates by DELFIA format as follows. Enzyme is first diluted in kinase assay buffer (10 mM HEPES (pH 7.4), 50 mM NaCl, 50 mM β-glycerophosphate, 10 mM MnCl₂, 0.5 mM DTT, 0.25 mM microcystin LR, and 100 μg/mL BSA). To each well, 12 μL of the diluted enzyme is mixed briefly with 0.5 μL test inhibitor or the control vehicle dimethylsulfoxide (DMSO). The kinase reaction is initiated by adding 12.5 μL kinase assay buffer containing ATP and His6-S6K (substrate) to give a final reaction volume of 25 μL containing 800 ng/mL FLAG-TOR, 100 μM ATP and 1.25 μM His6-S6K. The reaction plate is incubated for 2 hours (linear at 1-6 hours) at room temperature with gentle shaking and then terminated by adding 25 μL Stop buffer (20 mM HEPES, pH 7.4), 20 mM EDTA, 20 mM EGTA). The DELFIA detection of the phosphorylated His6-S6K (Thr-389) is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution are purchased from PerkinElmer. The terminated kinase reaction mixture (45 μL) is transferred to a MaxiSorp plate (Nunc) containing 55 μL PBS. The His6-S6K is allowed to attach for 2 hours after which the wells are aspirated and washed once with PBS. DELFIA Assay buffer (100 μL) with 40 ng/mL Eu—P(T389)-S6K antibody is added. The antibody binding is continued for 1 hour with gentle agitation. The wells are then aspirated and washed 4 times with PBS containing 0.05% Tween-20 (PBST). DELFIA Enhancement solution (100 μL) is added to each well and the plates are read in a PerkinElmer Victor model plate reader.

Fluorescence Polarization Assay for PI3K

Materials

Reaction Buffer: 20 mM HEPES, pH 7.5, 2 mM MgCl₂, 0.05% CHAPS; and 0.01% BME (added fresh) Stop/Detection Buffer: 100 mM HEPES, pH 7.5, 4 mM EDTA, 0.05% CHAPS; ATP 20 mM in water; PIP2 (diC8, Echelon, Salt Lake City Utah, cat# P-4508) 1 mM in water (MW=856.5); GST-GRP 1.75 mg/mL or 1.4 mg/mL in 10% glycerol; Red detector (TAMRA) 2.5 μM; Plate: Nunc 384 well black polypropylene fluorescence plate.

Methods

PI3-Kinase reactions were performed in 5 μM HEPES, pH 7, 2.5 μM MgCl₂, and 25 μM ATP, with diC8-PI(4,5)P2 (Echelon, Salt Lake City Utah) as substrate. Nunc 384 well black polypropylene fluorescent plates were used for PI3K assays. Reactions were quenched by the addition of EDTA to a final concentration of 10 μM. Final reaction volumes were 10 ml. For evaluation of PI 3-K inhibitors, 5 ng of enzyme and 2.5 μM of substrate was used per 10 ml reaction volume, and inhibitor concentrations ranged from 100 μM to 20 μM; the final level of DMSO in reactions never exceeded 2%. Reactions were allowed to proceed for one hour at 25° C. After I hour, GST-tagged GRP1 (general receptor for phosphoinositides) PH domain fusion protein was added to a final concentration of 100 nM, and BODIPY-TMRI(1,3,4,5)P4 (Echelon) was also added to a final concentration of 5 nM. Final sample volumes were 25 μl with a final DMSO concentration of 0.8%. Assay Plates were read on PerkinElmer Envision plate readers with appropriate filters for Tamra [BODIPY-TMRI(1,3,4,5)P4]. Data obtained were used to calculate enzymatic activity and enzyme inhibition by inhibitor compounds.

In Vitro Cell Culture Growth Assay Methods:

Cell Lines used are human pancreatic (PC3) and ovarian (OVCAR3) tumor cell lines. PC3 and OVCAR3 are plated in 96-well culture plates at approximately 3000 cells per well. One day following plating, various concentrations of PI3K inhibitors in DMSO are added to cells (final DMSO concentration in cell assays is 0.25%). Three days after drug treatment, viable cell densities are determined by cell mediated metabolic conversion of the dye MTS, a well-established indicator of cell proliferation in vitro. Cell growth assays are performed using kits purchased from Promega Corporation (Madison, Wis.), following the protocol provided by the vendor. Measuring absorbance at 490 nm generates MTS assay results. Compound effect on cell proliferation is assessed relative to untreated control cell growth. The drug concentration that conferred 50% inhibition of growth is determined as IC₅₀ (μM).

Table 1 shows the results of the described biological assays.

TABLE 1

| | PI3 Kinase α | PI3 Kinase γ | TOR Kinase |
|---|---|---|---|
| Example | IC₅₀ (nM) | IC₅₀ (nM) | IC₅₀ (μM)) |
| 1 | 121 | 667 | 228.25 |
| 2 | 1820 | 4329 | 272.5 |
| 3 | 69 | 133 | 180 |
| 4 | 66 | 204 | 305 |
| 5 | 68 | 133 | 180 |
| 6 | 100 | 620 | 91.75 |
| 7 | 171 | 406 | 245 |
| 8 | 86 | 196 | 80.5 |
| 9 | 57 | 242 | 108.5 |
| 10 | 132 | 154 | 715 |
| 11 | 83 | 132 | 115.5 |
| 12 | 80 | 168 | 135 |
| 13 | 31 | 111 | 44.5 |
| 14 | 60 | 94 | 2150 |
| 15 | 16 | 106 | 1210 |
| 16 | 61 | 161 | 395 |
| 17 | 217 | 370 | 705 |
| 18 | 75 | 261 | 82.5 |
| 19 | 83 | 277 | 210 |
| 20 | 541 | 454 | 51.5 |
| 21 | 66 | 240 | 140 |
| 22 | 304 | 318 | 1300 |
| 23 | 68 | 44 | 305 |
| 24 | 86 | 219 | 272.5 |
| 25 | 143 | 407 | 146.75 |
| 26 | 16 | 108 | 40.25 |
| 27 | 385 | 2528 | 1625 |
| 28 | 998 | 8833 | 10350 |
| 29 | 1137 | 3109 | 11000 |
| 30 | 231 | 2856 | 625 |
| 31 | 298 | 2282 | 4550 |
| 32 | 554 | 4073 | 310 |
| 33 | 3 | 19 | 0.7 |
| 34 | 131.5 | 309 | 180 |
| 35 | 8 | 37 | 21 |
| 36 | 179 | 895 | 43.5 |
| 37 | 73.5 | 156.5 | 1.87 |
| 38 | 340.8 | 7443.7 | 460 |
| 39 | 9.5 | 25 | 0.89 |
| 40 | 2.5 | 11.5 | 3 |
| 41 | 23.3 | 84.5 | 2.55 |
| 42 | 10.5 | 35.3 | 0.7 |
| 43 | 1 | 18 | 0.34 |
| 44 | 1.2 | 16 | 1.65 |
| 45 | <2.2 | 11.3 | 1.2 |
| 46 | 2.8 | 14.5 | 1.08 |
| 47 | <2.7 | 9 | 2.15 |
| 48 | 5.5 | 31 | 1.1 |
| 49 | <3.1 | 25 | 1.1 |
| 50 | 1.8 | 18 | 0.84 |
| 51 | 3.6 | 31 | 1.85 |
| 52 | <1.9 | 11.5 | 0.51 |
| 53 | 3.5 | 29 | 1.25 |
| 54 | <1.8 | 7 | 0.78 |
| 55 | 1.5 | 16.5 | 1.3 |
| 56 | 1.7 | 11 | 1.05 |
| 57 | <1.7 | 7 | 1.25 |
| 58 | 2.5 | 14 | 3.7 |
| 59 | 2.5 | 26 | 1.18 |
| 60 | <2.6 | 16.5 | 0.52 |
| 61 | 1.6 | 12 | 1.15 |
| 62 | 6 | 33 | 2.7 |
| 63 | 4.5 | 30 | 1.4 |
| 64 | 3.3 | 15.5 | 0.64 |
| 65 | 5630 | 1798 | 420 |
| 66 | 400 | 2773 | 125 |
| 67 | 2140 | >10000.0 | 225 |
| 68 | 9 | 70.3 | 0.51 |
| 69 | 8.5 | 97.5 | 2.2 |
| 70 | 5.5 | 102.5 | 0.85 |
| 71 | 7 | 55.5 | 0.84 |
| 72 | 8 | 88.5 | 0.55 |
| 73 | 15.5 | 92.5 | 3.1 |
| 74 | 9.5 | 97 | 3.2 |
| 75 | 13.5 | 92.5 | 6.1 |
| 76 | 5 | 32 | 1.25 |
| 77 | 1836 | 8000 | 300 |
| 78 | 6 | 13.5 | 0.84 |
| 79 | 7 | 34 | 150 |
| 80 | 3 | 19.5 | 3.05 |
| 81 | 1028.3 | 4633 | 255 |
| 82 | 5 | 21.5 | 0.51 |
| 83 | 10.5 | 48.5 | 0.56 |
| 84 | 3 | 34.5 | 0.44 |
| 85 | 25 | 78 | 0.35 |
| 86 | 23.5 | 69 | 1.6 |
| 87 | 13.5 | 69.5 | 2.55 |
| 88 | 77.5 | 197 | 90 |
| 89 | 15.5 | 40 | 1.9 |
| 90 | 34.5 | 100 | 2.7 |
| 91 | 41.5 | 409 | 1.65 |
| 92 | 149 | 645 | 255 |
| 93 | 2.5 | 18 | 0.4 |
| 94 | 34.5 | 132 | 3.3 |
| 95 | 33.7 | 91 | 2.7 |
| 96 | 49.7 | 619.7 | 5 |
| 97 | 1018 | 4358 | 595 |
| 98 | 54 | 595 | 705 |
| 99 | 924 | 5752 | 960 |
| 100 | 1656 | 3145 | 1000 |
| 101 | 537.5 | 3546 | 810 |
| 102 | 1255 | 1922 | 1145 |
| 103 | 987 | 5048 | 1300 |
| 104 | 1168 | 3030 | 905 |
| 105 | 1384.5 | 2955 | 1550 |
| 106 | 556 | 2143 | 515 |
| 107 | 1040 | 2487 | 3650 |
| 108 | 941 | 2772 | 6150 |
| 109 | 241.5 | 900.5 | 1850 |
| 110 | 200.5 | 401.5 | 3500 |
| 111 | 439 | 2285.5 | 1650 |
| 112 | 154 | 1024 | 4150 |
| 113 | 726 | 3351 | 5750 |
| 114 | 255 | 982.5 | 2125 |
| 115 | 240.5 | 3632.5 | 465 |
| 116 | >10000.0 | >10000.0 | 4000 |
| 117 | 38.3 | 143.7 | 210 |
| 118 | 27.5 | 101.5 | 18 |
| 119 | 22.5 | 314.5 | 24 |
| 120 | 15.5 | 290.5 | 140 |
| 121 | 36 | 551 | 51 |
| 122 | 56.5 | 260.5 | 220 |
| 123 | 4 | 8.5 | 1.2 |
| 124 | 108 | 889.5 | 410 |
| 125 | 130.5 | 1962 | 1450 |
| 126 | 60.5 | 873 | 265 |
| 127 | 133.5 | 1239 | 170 |
| 128 | 164 | 1100 | 121 |
| 129 | 30 | 156 | 19000 |
| 130 | 34 | 165 | 26000 |
| 131 | 58 | 790.5 | 48.5 |
| 132 | 21 | 1426.5 | 39 |
| 133 | 370 | 1379 | 65.5 |
| 134 | 2 | 25.5 | 0.38 |
| 135 | 9.5 | 30 | 0 |
| 136 | 38 | 88 | 1.35 |
| 137 | 13 | 30.5 | 2.3 |
| 138 | 3 | 12.5 | 0.28 |
| 139 | 318 | 1504 | 320 |
| 140 | 26.5 | 118.5 | 12 |
| 141 | 6.5 | 30 | 2.35 |
| 142 | 4 | 20 | 1.65 |
| 143 | 189 | 3794 | 59.5 |
| 144 | 81.5 | 406 | 505 |
| 145 | 62 | 426.5 | 135 |
| 146 | 13 | 280 | 535 |
| 147 | 59 | 734.5 | 225 |
| 148 | 111 | 1402 | 135 |
| 149 | 67 | 735 | 34000 |
| 150 | 21.5 | 223 | 4.3 |
| 151 | 295.5 | 747.5 | 72 |
| 152 | 104.5 | 392 | 930 |
| 153 | 146.5 | 176 | 109 |
| 154 | 205.5 | 58 | 82.5 |
| 155 | 48 | 176.5 | 77.5 |
| 156 | 170 | 557 | 285 |
| 157 | 61 | 144 | 20.5 |
| 158 | 74.5 | 342 | 115 |
| 159 | 166 | 685 | 1550 |
| 160 | 13 | 108.5 | 41 |
| 161 | 80 | 285.5 | 320 |
| 162 | 11.5 | 103.5 | 210 |
| 163 | 13.5 | 49.5 | 5.7 |
| 164 | 22 | 147 | 4.25 |
| 165 | 151 | 3578 | 35.5 |
| 166 | 36.5 | 494.5 | 3.4 |
| 167 | 9 | 91.5 | 17 |
| 168 | 200 | 3241 | 4.05 |
| 169 | >10000.0 | >10000.0 | 270 |
| 170 | 5626 | 10376 | 5750 |
| 171 | 76.5 | 144.5 | 205 |
| 172 | 203.5 | 1226.5 | 1550 |
| 173 | 570 | 1850.5 | 945 |
| 174 | 285 | 955.5 | 6900 |
| 175 | 1413 | 9107 | 1600 |
| 176 | 23.7 | 163.7 | 1450 |
| 177 | 83.5 | 435 | 250 |
| 178 | 1341 | >10000.0 | 1700 |
| 179 | 141.7 | 342 | 29 |
| 180 | 67.5 | 152 | 7.1 |
| 181 | 58.5 | 185.5 | 4.15 |
| 182 | 4 | 27 | 3.4 |
| 183 | 5.3 | 20 | 3.45 |
| 184 | 20 | 47 | 3.95 |
| 185 | 14 | 44.7 | 7.6 |
| 186 | 4.7 | 22 | 4.25 |
| 187 | 2076 | 12000 | 970 |
| 188 | 19.5 | 517.5 | 28.5 |
| 189 | 8 | 28 | 20 |
| 190 | 19 | 346.5 | 38.5 |
| 191 | 91 | 329 | 87 |
| 192 | 148 | 549 | 135 |
| 193 | 68 | 407.5 | 175 |
| 194 | 31 | 210 | 120 |
| 195 | 689.3 | 5207.3 | 5050 |
| 196 | 75.5 | 1058 | 20000 |
| 197 | 66 | 220 | 320 |
| 198 | 10 | 43 | 1.09 |
| 199 | 10.5 | 49 | 2.95 |
| 200 | 6.5 | 22 | 3.7 |
| 201 | 4.5 | 25.5 | 4.85 |
| 202 | 224 | 780 | 2100 |
| 203 | 100 | 618 | 1750 |
| 204 | 132 | 108 | 2300 |
| 205 | 332 | 1206 | 3500 |
| 206 | 116 | 216 | 945 |
| 207 | 342 | 2645 | 520 |
| 208 | 122 | 1362 | 9600 |
| 209 | 447 | 1669 | 1300 |
| 210 | 692 | 782 | 575 |
| 211 | 289 | 2726 | 1040 |
| 212 | 78 | 766 | 535 |
| 213 | 282 | 1378 | 820 |
| 214 | 386 | 1545 | 1550 |
| 215 | 335 | 4033 | 1500 |
| 216 | 58 | 361 | 605 |
| 217 | 240 | 240 | 63 |
| 218 | 71 | 8078 | 20000 |
| 219 | 260 | 1836 | 435 |
| 220 | 1153 | 2421 | 1350 |
| 221 | 303 | 1178 | 940 |
| 222 | 420 | 1400 | 1050 |
| 223 | 175 | 2509 | 5700 |
| 224 | 90 | 1912 | 3050 |
| 225 | 43 | 135 | 7 |
| 226 | 126 | 958 | 495 |
| 227 | 1128 | 950 | 645 |
| 228 | 911 | 544 | 605 |
| 229 | 286 | 10250 | 12800 |
| 230 | 118 | 938 | 480 |
| 231 | 60 | 2300 | 2200 |
| 232 | 370 | 1948 | 4000 |
| 233 | 501 | 2718 | 2000 |
| 234 | 74 | 173 | 49 |
| 235 | 637 | 2227 | 4000 |
| 236 | 560 | 1876 | 4000 |
| 237 | 460 | 2590 | 4000 |
| 238 | 245 | 2074 | 4000 |
| 239 | 1129 | 5064 | 4000 |
| 240 | 658 | 1698 | 3850 |
| 241 | 630 | 4906 | 1600 |
| 242 | 509 | 842 | 230 |
| 243 | 765 | 9587 | 20000 |
| 244 | 1511 | 1606 | 3500 |
| 245 | 298 | 1939 | 390 |
| 246 | 531 | 8.00 | 20.75 |
| 247 | 490 | 15.00 | 32.50 |
| 248 | 460 | 7.33 | 24.33 |
| 249 | 574 | 1.33 | 6.00 |
| 250 | 651 | 0.95 | 5.50 |
| 251 | 650 | 0.80 | 9.50 |
| 252 | 551.5 | 3.83 | 22.00 |
| 253 | 551.5 | 3.30 | 15.00 |
| 254 | 569.2 | 7.50 | 33.50 |
| 255 | 581.3 | 5.00 | 22.50 |
| 256 | 572 | 2.00 | 12.00 |
| 257 | 585.4 | 10.00 | 79.00 |
| 258 | 557 | 1.35 | 17.50 |
| 259 | 543.3 | 4.00 | 8.50 |
| 260 | 565 | 1.85 | 21.50 |
| 261 | 586 | 1.85 | 9.50 |
| 262 | 565.2 | 1.85 | 10.00 |
| 263 | 573.3 | 3.10 | 20.00 |
| 264 | 607.3 | 1.83 | 22.00 |
| 265 | 574.4 | 1.65 | 5.00 |
| 266 | 614.4 | 1.45 | 6.00 |
| 267 | 600.7 | 1.70 | 8.00 |
| 268 | 629.3 | 1.80 | 10.50 |
| 269 | 600.3 | 1.65 | 5.50 |
| 270 | 588.3 | 1.65 | 6.50 |
| 271 | 600.7 | 1.70 | 6.50 |
| 272 | 640.3 | 0.75 | 6.50 |
| 273 | 594.3 | 3.30 | 11.00 |
| 274 | 572.6 | 1.70 | 4.00 |
| 275 | 600.3 | 0.90 | 6.00 |
| 276 | 561.3 | 4.45 | 17.50 |
| 277 | 557.6 | 5.50 | 21.50 |
| 278 | 504.4 | 21.50 | 154.50 |
| 279 | 552.2 | 12.00 | 101.50 |
| 280 | 473 | 7.50 | 36.50 |
| 281 | 613.3 | 2.80 | 35.00 |
| 282 | 585.3 | 2.15 | 30.00 |
| 283 | 599.3 | 3.35 | 47.50 |
| 284 | 613.3 | 4.35 | 49.50 |
| 285 | 587.3 | 2.20 | 23.50 |
| 286 | 573.3 | 1.85 | 31.00 |
| 287 | 504 | 91.50 | 202.50 |
| 288 | 490 | 3.30 | 14.50 |
| 289 | 572 | 6.00 | 29.00 |
| 290 | 560 | 1.85 | 41.00 |

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

It is intended that each of the patents, applications, and printed publications, including books, mentioned in this patent document be hereby incorporated by reference in their entirety.

What is claimed is:

1. A compound of the Formula 1: 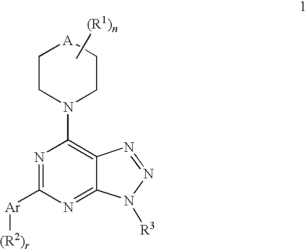
or a pharmaceutically acceptable salt thereof, wherein
A is —O—, —CH₂O—, or —S(O)ₘ—;
m is 0, 1, or 2;
Ar is phenyl, naphthyl, or a nitrogen-containing mono- or bicyclic heteroaryl;
R¹ is independently C₁-C₆alkyl, C₆-C₁₄aryl, C₁-C₉heteroaryl, C₂-C₆alkenyl, C₂-C₆alkynyl, or C₃-C₈cycloalkyl;
or two R¹ groups on the same carbon atom, when taken together with the carbon to which they are attached, optionally form a carbonyl (C═O) group;
n is 0, 1, 2, or 3;
R² is independently halogen; C₁-C₆alkyl; C₂-C₆alkenyl; C₁-C₆alkoxy; C₂-C₆alkynyl; C₃-C₈cycloalkyl; C₆-C₁₄aryl; C₁-C₉heteroaryl; hydroxyl; C₁-C₆hydroxylalkyl-; —NR⁴R⁵; —NO₂₂; —CHO; —CN; —C(O)NR⁴R⁵; R⁶C(O)NH—; —CO₂H; —CF₃; —OCF₃; R⁴R⁵NC(O)NH—; or R⁶OC(O)NH—;
r is 0, 1, 2, 3, 4, or 5;
R⁴ and R⁵ are each independently H; (C₁-C₆alkoxy)carbonyl; C₁-C₆alkyl; C₆-C₁₄aryl, optionally substituted with R⁷R⁸NC(O)—, R⁷R⁸NC(O)NH—, CO₂H, —CONH₂, —CN, —NO₂, R⁷R⁸N—, R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, C₁-C₉heterocyclyl-O—, C₁-C₆alkyl, C₁-C₆alkoxy, C₁-C₆hydroxylalkyl-, C₁-C₉heterocycle, wherein the ring portion of the C₁-C₉heterocycle group is optionally substituted by C₁-C₆alkyl, halogen, NH₂—C₁-C₆alkylene-, (C₁-C₆alkyl)-NH—C₁-C₆alkylene-, (C₁-C₆alkyl)(C₁-C₆alkyl)N—C₁-C₆alkylene-, or (C₁-C₆alkoxy)carbonyl; C₁-C₉heteroaryl, optionally substituted by R⁷R⁸NC(O)—, R⁷R⁸NC(O)NH—, CO₂H, —CONH₂, —CN, —NO₂, R⁷R⁸N, R⁷R⁸N—C₁-C₆alkylene, R⁷R⁸N—C₁-C₆alkylene-O—, R⁷R⁸N—C₁-C₆alkylene-NH—, R⁷R⁸N—NH—, C₁-C₉heteroaryl, C₁-C₉heteroaryl-O—, C₁-C₉heterocyclyl-O—, C₁-C₆alkyl, C₁-C₆alkoxy, C₁-C₆hydroxylalkyl-, C₁-C₉heterocycle, wherein the ring portion of the C₁-C₉heterocycle group is optionally substituted by C₁-C₆alkyl, halogen, NH₂—C₁-C₆alkylene-, (C₁-C₆alkyl)-NH—C₁-C₆alkylene-, (C₁-C₆alkyl)(C₁-C₆alkyl)N—C₁-C₆alkylene-, or (C₁-C₆alkoxy)carbonyl-; C₁-C₉heterocycle optionally substituted by (C₆-C₁₄aryl)alkyl-OC(O)— or C₁-C₆alkyl; C₃-C₈cycloalkyl; heterocyclyl(C₁-C₆alkyl) optionally substituted with C₁-C₆alkyl; C₁-C₆alkyl-OC(O)N(C₁-C₃alkyl)C₁-C₆alkylene; NH₂—C₁-C₆alkylene-; (C₁-C₆alkyl)-NH—C₁-C₆alkylene-; or (C₁-C₆alkyl) (C₁-C₆alkyl)N—C₁-C₆alkylene-;
or R⁴ and R⁵ when taken together with the nitrogen to which they are attached optionally form a 3- to 7-membered nitrogen containing heterocycle wherein up to two of the carbon atoms of the heterocycle is optionally replaced with —N(H)—, —O—, or —S(O)ₚ—;
p is 0, 1 or 2;
R⁶ is C₁-C₆alkyl; C₆-C₁₄aryl; (C₆-C₁₄aryl)alkyl, optionally substituted by NH₂; or C₁-C₆ perfluoroalkyl-;
R⁷ and R⁸ are each independently H; C₁-C₆alkyl optionally substituted with C₁-C₆alkoxy; C₁-C₈acyl optionally substituted with NH₂, (C₁-C₆alkyl)amino, or di(C₁-C₆alkyl)amino; (C₁-C₆alkyl)SO₂— optionally substituted with NH₂, (C₁-C₆alkyl)amino, or di(C₁-C₆alkyl)amino; (C₁-C₆alkyl)SO— optionally substituted with NH₂, (C₁-C₆alkyl)amino, or di(C₁-C₆alkyl)amino; C₆-C₁₄aryl-; (C₆-C₁₄aryl)SO₂—; (C₆-C₁₄aryl)SO—; aryl(C₁-C₆alkyl) optionally substituted with C₁-C₆alkoxy, C₁-C₆alkyl, or halo; C₁-C₉heteroaryl; (C₁-C₉heteroaryl)SO₂—; (C₁-C₉heteroaryl)SO—; heterocyclylSO₂—; heterocyclylSO—; C₁-C₆hydroxylalkyl; heteroaryl(C₁-C₆alkyl) optionally substituted with C₁-C₆alkoxy, C₁-C₆alkyl, or halo; heterocyclyl(C₁-C₆alkyl) optionally substituted with C₁-C₆alkyl; C₁-C₉heterocycle optionally substituted by (C₆-C₁₄aryl)alkyl-OC(O)—; NH₂—C₁-C₆alkylene-; (C₁-C₆alkyl)-NH—C₁-C₆alkylene-; or (C₁-C₆alkyl)(C₁-C₆alkyl)N—C₁-C₆alkylene-;
or R⁷ and R⁸ when taken together with the nitrogen to which they are attached optionally form a 3- to 7-membered nitrogen containing heterocycle wherein up to two of the carbon atoms of the heterocycle are optionally replaced with —N(R⁹)—, —O—, or —S(O)_{q}—, and wherein the heterocycle is optionally substituted with from 1 to 3 substituents independently selected from C₁-C₆alkyl; (C₁-C₆alkyl)amino-, C₆-C₁₄aryl, di(C₁-C₆alkyl)amino-, H₂N—, C₁-C₉heteroaryl, and C₁-C₉heterocycle;
q is 0, 1 or 2;
R⁹ is H or C₁-C₆alkyl;
R³ is:
(a) hydrogen;
(b) C₁-C₆alkyl optionally substituted with from 1 to 3 substituents independently selected from:
(i) C₁-C₆alkoxy,
(ii) NH₂,
(iii) (C₁-C₆alkyl)amino,
(iv) di(C₁-C₆alkyl)amino,
(v) CO₂H,
(vi) and (C₁-C₆alkoxy)carbonyl;
(c) carboxyamidoalkyl optionally substituted with a substituent selected from:
(i) halogen,
(ii) and di(C₁-C₆alkyl)amino;
(d) C₁-C₆ perfluoroalkyl-;
(e) C₃-C₈cycloalkyl;
(f) C₆-C₁₄aryl optionally substituted with a substituent selected from:
(i) —O—C 1-C₆alkylene-NH₂,
(ii) —COOH,
(iii) C₁-C₆hydroxylalkyl,
(iv) R¹⁰R¹¹NC(O)—,
(v) and (C₁-C₆alkoxy)carbonyl;
(g) monocyclic C₁-C₆heterocycle optionally substituted with from 1 to 3 substituents independently selected from:
(i) C₁-C₈acyl, wherein the C₁-C₈acyl is optionally substituted with a NH₂,
(ii) C₁-C₆alkyl,
(iii) heteroaryl(C₁-C₆alkyl) wherein the ring portion of the heteroaryl(C₁-C₆alkyl) group is optionally substituted with from 1 to 3 substituents independently selected from:
A) C₁-C₆alkylC(O)NH—,
B) halogen,
C) NH₂,
D) and C₁-C₆alkyl,
(iv) heterocyclyl(C₁-C₆alkyl), wherein the ring portion of the heterocyclyl(C₁-C₆alkyl) group is optionally substituted by a (C₆-C₁₄aryl)alkyl,
(v) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) di(C₁-C₆alkyl)amino-(C₁-C₆alkylene)-O—,
D) and C₁-C₉heteroaryl;
(vi) and (C₁-C₆alkoxy)carbonyl;
(h) heterocyclyl(C₁-C₆alkyl) optionally substituted with a substituent selected from:
(i) C₁-C₆alkyl,
(ii) C₃-C₈cycloalkyl,
(iii) (C₁-C₆alkoxy)carbonyl,
(iv) C₁-C₆alkylcarboxy,
(v) (C₆-C₁₄aryl)alkyl wherein the ring portion of the (C₆-C₁₄aryl)alkyl group is optionally substituted by a:
A) halogen,
B) C₁-C₉heteroaryl,
C) or di(C₁-C₆alkyl)amino-(C₁-C₆alkylene)-O—,
(vi) heteroaryl(C₁-C₆alkyl) wherein the ring portion of the heteroaryl(C₁-C₆alkyl) group is optionally substituted by a halogen,
(vii) and C₁-C₈acyl, wherein the C₁-C₈acyl is optionally substituted with from 1 to 3 independently selected halogens,
(i) (C₁-C₆alkyl)-C(O)—NH—(C₁-C₆alkylene)-;
(j) heteroaryl(C₁-C₆alkyl);
(k) (C₆-C₁₄aryl)alkyl wherein the ring portion of the (C₆-C₁₄aryl)alkyl group is optionally substituted by a:
(i) ClC₆H₄C(O)NH—,
(ii) (C₁-C₆alkoxy)carbonyl,
(iii) CO₂H,
(iv) or R¹⁰R¹¹NC(O);
(l) C₁-C₆hydroxylalkyl;
(m) or C₁-C₉heteroaryl;
R¹⁰ and R¹¹ are each independently:
(a) H;
(b) C₁-C₆alkyl optionally substituted with a substituent selected from:
(i) C₁-C₆alkylC(O)NH—,
(ii) NH₂,
(iii) (C₁-C₆alkyl)amino,
(iv) or di(C₁-C₆alkyl)amino,
(c) C₃-C₈cycloalkyl;
(d) C₆-C₁₄aryl optionally substituted with a substituent selected from:
(i) halogen,
(ii) and monocyclic C₁-C₆heterocycle wherein the monocyclic C₁-C₆heterocycle is optionally substituted with (C₁-C₆alkoxy)carbonyl;
(e) C₁-C₉heteroaryl;
(f) heteroaryl(C₁-C₆alkyl);
(g) heterocyclyl(C₁-C₆alkyl);
(h) (C₆-C₁₄aryl)alkyl, wherein the chain portion of the (C₆-C₁₄aryl)alkyl group is optionally substituted by a hydroxyl;
(i) or monocyclic C₁-C₆heterocycle optionally substituted with a (C₁-C₆alkoxy)carbonyl;
or R¹⁰ and R¹¹ when taken together with the nitrogen to which they are attached optionally form a 3- to 7-membered nitrogen-containing heterocycle wherein up to two of the carbon atoms of the heterocycle are optionally replaced with —N(H)—, —N(C₁-C₆alkyl)-, —N(C₆-C₁₄aryl)-, or —O—, and wherein the nitrogen-containing heterocycle is optionally substituted by a C₁-C₆alkyl; C₆-C₁₄aryl, (C₁-C₆alkoxy)C(O)NH—, or C₁-C₉heterocycle.

2. The compound of claim 1, wherein n is 0.

3. The compound of claim 1, wherein A is —O—.

4. The compound of claim 1, wherein r is 1.

5. The compound of claim 1, wherein Ar represents phenyl.

6. The compound of claim 5, wherein Ar represents phenyl substituted in the 4-position by R².

7. The compound of claim 1, wherein R² is —NHC(O)NR⁴R⁵.

8. The compound of claim 7, wherein R⁵ is H.

9. The compound of claim 1, wherein R⁴ is C₆-C₁₄aryl, optionally substituted with R⁷R⁸NC(O)—.

10. The compound of claim 9, wherein R⁴ is phenyl, substituted with R⁷R⁸NC(O)—.

11. The compound of claim 10, wherein R⁴ is phenyl, substituted in the 4-position with R⁷R⁸NC(O)—.

12. The compound of claim 11, wherein R⁷ is (C₁-C₆alkyl)(C₁-C₆alkyl)N—C₁-C₆alkylene-.

13. The compound of claim 11, wherein R⁸ is H.

14. The compound of claim 11, wherein R⁷ and R⁸ taken together with the nitrogen to which they are attached form a 3- to 7-membered nitrogen containing heterocycle wherein up to two of the carbon atoms of the heterocycle are optionally replaced with —N(R⁹)—, —O—, or —S(O)_{q}—.

15. The compound of claim 14, wherein R⁷ and R⁸ taken together with the nitrogen to which they are attached form a 6-membered nitrogen containing heterocycle wherein one of the carbon atoms of the heterocycle is replaced with —N(R⁹)—.

16. The compound of claim 15, wherein R⁹ is C₁-C₆alkyl.

17. The compound of claim 1, wherein R³ is C₁-C₆alkyl.

18. The compound of claim 17, wherein R³ is ethyl.

19. The compound of claim 1, wherein n is 0, A is —O—, r is 1, Ar is phenyl, R² is —NHC(O)NR⁴R⁵, R⁴ is C₆-C₁₄aryl, optionally substituted with R⁷R⁸NC(O)—, and R³ is C₁-C₆alkyl.

20. The compound of claim 1, wherein n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁵ is H, and R³ is ethyl.

21. The compound of claim 1, wherein n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ is (C₁-C₆alkyl)(C₁-C₆alkyl)N—C₁-C₆alkylene-, R⁸ is H, R⁵ is H, and R³ is ethyl.

22. The compound of claim 1, wherein n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 3- to 7-membered nitrogen containing heterocycle wherein up to two of the carbon atoms of the heterocycle are optionally replaced with —N(R⁹)—, —O—, or —S(O)_{q}—, R⁵ is H, and R³ is ethyl.

23. The compound of claim 1, wherein n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 6-membered nitrogen containing heterocycle wherein one of the carbon atoms of the heterocycle is replaced with —N(R⁹)—, R⁵ is H, and R³ is ethyl.

24. The compound of claim 1, wherein n is 0, A is —O—, r is 1, Ar is phenyl, substituted in the 4-position, R² is —NHC(O)NR⁴R⁵, R⁴ is phenyl, substituted in the 4-position with R⁷R⁸NC(O)—, R⁷ and R⁸ taken together with the nitrogen to which they are attached form a 6-membered nitrogen containing heterocycle wherein one of the carbon atoms of the heterocycle is replaced with —N(R⁹)—, R⁹ is C₁-C₆alkyl, R⁵ is H, and R³ is ethyl.

25. A compound selected from the group consisting of:
3-[3-(1-benzylpiperidin-4-yl)-7-morpholin-4-yl-3H-[1,2,3]triazolo[4,5-d]pyrimidin-5-yl]phenol;
5-[3-(1-benzylpiperidin-4-yl)-7-morpholin-4-yl-3H-[1,2,3]triazolo[4,5-d]pyrimidin-5-yl]pyrimidin-2-amine;
5-[3-(1-benzylpiperidin-4-yl)-7-morpholin-4-yl-3H-[1,2,3]triazolo[4,5-d]pyrimidin-5-yl]pyridin-3-ol;
1-{4-[3-(1-benzylpiperidin-4-yl)-7-morpholin-4-yl-3H-[1,2,3]triazolo[4,5-d]pyrimidin-5-yl]phenyl}-3-[2-(dimethylamino)ethyl]urea;
N-{4-[3-(1-benzylpiperidin-4-yl)-7-morpholin-4-yl-3H-[1,2,3]triazolo[4,5-d]pyrimidin-5-yl]phenyl}-2,2,2-trifluoroacetamide;
1-{4-[3-(1-benzylpiperidin-4-yl)-7-morpholin-4-yl-3H-[1,2,3]triazolo[4,5-d]pyrimidin-5-yl]phenyl}-3-methylurea;
N-{2-[5-(3-hydroxyphenyl)-7-morpholin-4-yl-3H-[1,2,3]triazolo[4,5-d]pyrimidin-3-yl]ethyl}acetamide;
N-(2-{5-[3-(hydroxymethyl)phenyl]-7-morpholin-4-yl-3H-[1,2,3]triazolo[4,5-d]pyrimidin-3-yl}ethyl)acetamide;
3-[7-morpholin-4-yl-3-(3-pyrrolidin-1-ylpropyl)-3H-[1,2,3]triazolo[4,5-d]pyrimidin-5-yl]phenol;
{3-[7-morpholin-4-yl-3-(3-pyrrolidin-1-ylpropyl)-3H-[1,2,3]triazolo[4,5-d]pyrimidin-5-yl]phenyl}methanol;
5-(1H-indazol-4-yl)-7-morpholin-4-yl-3-(3-pyrrolidin-1-ylpropyl)-3H-[1,2,3]triazolo[4,5-d]pyrimidine;
5-(7-morpholin-4-yl-3-piperidin-4-yl-3H-[1,2,3]triazolo[4,5-d]pyrimidin-5-yl)pyridin-3-ol;
5-{3-[1-(2-furylmethyl)piperidin-4-yl]-7-morpholin-4-yl-3H-[1,2,3]triazolo[4,5-d]pyrimidin-5-yl}pyridin-3-ol;
5-{3-[1-(4-fluorobenzyl)piperidin-4-yl]-7-morpholin-4-yl-3H-[1,2,3]triazolo[4,5-d]pyrimidin-5-yl}pyridin-3-ol;
5-(3-{1-[(6-bromopyridin-3-yl)methyl]piperidin-4-yl}-7-morpholin-4-yl-3H-[1,2,3]triazolo[4,5-d]pyrimidin-5-yl)pyridin-3-ol;
5-(3-{1-[(5-bromopyridin-3-yl)methyl]piperidin-4-yl}-7-morpholin-4-yl-3H-[1,2,3]triazolo[4,5-d]pyrimidin-5-yl)pyridin-3-ol;
5-[3-(1-{4-[3-(dimethylamino)propoxy]benzyl}piperidin-4-yl)-7-morpholin-4-yl-3H-[1,2,3]triazolo[4,5-d]pyrimidin-5-yl]pyridin-3-ol;
5-{3-[1-(3,4-difluorobenzyl)piperidin-4-yl]-7-morpholin-4-yl-3H-[1,2,3]triazolo[4,5-d]pyrimidin-5-yl}pyridin-3-ol;
5-(3-{1-[(1-methyl-1H-pyrrol-2-yl)methyl]piperidin-4-yl}-7-morpholin-4-yl-3H-[1,2,3]triazolo[4,5-d]pyrimidin-5-yl)pyridin-3-ol;
5-(3-{1-[(6-chloropyridin-3-yl)methyl]piperidin-4-yl}-7-morpholin-4-yl-3H-[1,2,3]triazolo[4,5-d]pyrimidin-5-yl)pyridin-3-ol;
5-(3-{1-[(5-methyl-2-thienyl)methyl]piperidin-4-yl}-7-morpholin-4-yl-3H-[1,2,3]triazolo[4,5-d]pyrimidin-5-yl)pyridin-3-ol;
5-[3-(1-methylpiperidin-4-yl)-7-morpholin-4-yl-3H-[1,2,3]triazolo[4,5-d]pyrimidin-5-yl]pyridin-3-ol;
5-{3-[1-(2,4-difluorobenzyl)piperidin-4-yl]-7-morpholin-4-yl-3H-[1,2,3]triazolo[4,5-d]pyrimidin-5-yl}pyridin-3-ol;
5-(3-{1-[(1-methyl-1H-imidazol-5-yl)methyl]piperidin-4-yl}-7-morpholin-4-yl-3H-[1,2,3]triazolo[4,5-d]pyrimidin-5-yl)pyridin-3-ol;
N-[3-({4-[5-(5-hydroxypyridin-3-yl)-7-morpholin-4-yl-3H-[1,2,3]triazolo[4,5-d]pyrimidin-3-yl]piperidin-1-yl}methyl)pyridin-2-yl]-2,2-dimethylpropanamide;
5-(3-{1-[(4,5-dimethyl-2-thienyl)methyl]piperidin-4-yl}-7-morpholin-4-yl-3H-[1,2,3]triazolo[4,5-d]pyrimidin-5-yl)pyridin-3-ol;
5-[3-(1-butylpiperidin-4-yl)-7-morpholin-4-yl-3H-[1,2,3]triazolo[4,5-d]pyrimidin-5-yl]pyridin-3-ol;
5-(3-{1-[(4-benzylpiperazin-1-yl)methyl]piperidin-4-yl}-7-morpholin-4-yl-3H-[1,2,3]triazolo[4,5-d]pyrimidin-5-yl)pyridin-3-ol;
5-{7-morpholin-4-yl-3-[1-(1H-pyrrol-2-ylmethyl)piperidin-4-yl]-3H-[1,2,3]triazolo[4,5-d]pyrimidin-5-yl}pyridin-3-ol;
5-(3-{1-[(1-methyl-1H-pyrazol-5-yl)methyl]piperidin-4-yl}-7-morpholin-4-yl-3H-[1,2,3]triazolo[4,5-d]pyrimidin-5-yl)pyridin-3-ol;
5-{7-morpholin-4-yl-3-[1-(4-pyridin-4-ylbenzyl)piperidin-4-yl]-3H-[1,2,3]triazolo[4,5-d]pyrimidin-5-yl}pyridin-3-ol;
4-(3-ethyl-7-morpholin-4-yl-3H-[1,2,3]triazolo[4,5-d]pyrimidin-5-yl)aniline;
1-[4-(3-ethyl-7-morpholin-4-yl-3H-[1,2,3]triazolo[4,5-d]pyrimidin-5-yl)phenyl]-3-pyridin-4-ylurea;
1-[2-(dimethylamino)ethyl]-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-(2-methylpyridin-4-yl)urea;
1-[4-(3-ethyl-7-morpholin-4-yl-3H-[1,2,3]triazolo[4,5-d]pyrimidin-5-yl)phenyl]-3-(4H-1,2,4-triazol-4-yl)urea;
1-[4-(3-ethyl-7-morpholin-4-yl-3H-[1,2,3]triazolo[4,5-d]pyrimidin-5-yl)phenyl]-3-(1,3-thiazol-2-yl)urea;
2-(4-aminophenyl)ethyl [4-(3-ethyl-7-morpholin-4-yl-3H-[1,2,3]triazolo[4,5-d]pyrimidin-5-yl)phenyl]carbamate;
1-[4-(3-ethyl-7-morpholin-4-yl-3H-[1,2,3]triazolo[4,5-d]pyrimidin-5-yl)phenyl]-3-pyridin-3-ylurea;
1-[4-(3-ethyl-7-morpholin-4-yl-3H-[1,2,3]triazolo[4,5-d]pyrimidin-5-yl)phenyl]-3-(2-thienyl)urea;
methyl 4-({[4-(3-ethyl-7-morpholin-4-yl-3H-[1,2,3]triazolo[4,5-d]pyrimidin-5-yl)phenyl]carbamoyl}amino)benzoate;
4-({[4-(3-ethyl-7-morpholin-4-yl-3H-[1,2,3]triazolo[4,5-d]pyrimidin-5-yl)phenyl]carbamoyl}amino)benzoic acid;
N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)benzamide;
1-[4-(3-ethyl-7-morpholin-4-yl-3H-[1,2,3]triazolo[4,5-d]pyrimidin-5-yl)phenyl]-3-{4-[(4-methylpiperazin-1-yl)carbonyl]phenyl}urea;
N-[2-(dimethylamino)ethyl]-4-({[4-(3-ethyl-7-morpholin-4-yl-3H-[1,2,3]triazolo[4,5-d]pyrimidin-5-yl)phenyl]carbamoyl}amino)-N-methylbenzamide;
4-({[4-(3-ethyl-7-morpholin-4-yl-3H-[1,2,3]triazolo[4,5-d]pyrimidin-5-yl)phenyl]carbamoyl}amino)-N-(2-hydroxyethyl)benzamide;
N-[3-(dimethylamino)propyl]-4-({[4-(3-ethyl-7-morpholin-4-yl-3H-[1,2,3]triazolo[4,5-d]pyrimidin-5-yl)phenyl]carbamoyl}amino)benzamide;
1-[4-(3-ethyl-7-morpholin-4-yl-3H-[1,2,3]triazolo[4,5-d]pyrimidin-5-yl)phenyl]-3-{4-[(4-morpholin-4-ylpiperidin-1-yl)carbonyl]phenyl}urea;
4-({[4-(3-ethyl-7-morpholin-4-yl-3H-[1,2,3]triazolo[4,5-d]pyrimidin-5-yl)phenyl]carbamoyl}amino)-N-[2-(4-methylpiperazin-1-yl)ethyl]benzamide;
1-[4-(1,4′-bipiperidin-1′-ylcarbonyl)phenyl]-3-[4-(3-ethyl-7-morpholin-4-yl-3H-[1,2,3]triazolo[4,5-d]pyrimidin-5-yl)phenyl]urea;
4-({[4-(3-ethyl-7-morpholin-4-yl-3H-[1,2,3]triazolo[4,5-d]pyrimidin-5-yl)phenyl]carbamoyl}amino)-N-(pyridin-4-ylmethyl)benzamide;
4-({[4-(3-ethyl-7-morpholin-4-yl-3H-[1,2,3]triazolo[4,5-d]pyrimidin-5-yl)phenyl]carbamoyl}amino)-N-methyl-N-[2-(methylamino)ethyl]benzamide;
4-({[4-(3-ethyl-7-morpholin-4-yl-3H-[1,2,3]triazolo[4,5-d]pyrimidin-5-yl)phenyl]carbamoyl}amino)-N-(2-morpholin-4-ylethyl)benzamide;
1-[4-(3-ethyl-7-morpholin-4-yl-3H-[1,2,3]triazolo[4,5-d]pyrimidin-5-yl)phenyl]-3-(4-{[(3R)-3-methylpiperazin-1-yl]carbonyl}phenyl)urea;
4-({[4-(3-ethyl-7-morpholin-4-yl-3H-[1,2,3]triazolo[4,5-d]pyrimidin-5-yl)phenyl]carbamoyl}amino)-N-[3-(4-methylpiperazin-1-yl)propyl]benzamide;
4-({[4-(3-ethyl-7-morpholin-4-yl-3H-[1,2,3]triazolo[4,5-d]pyrimidin-5-yl)phenyl]carbamoyl}amino)-N-(2-piperidin-1-ylethyl)benzamide;
1-{4-[(3,3-dimethylpiperazin-1-yl)carbonyl]phenyl}-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-{4-[(4-pyridin-2-ylpiperazin-1-yl)carbonyl]phenyl}urea;
4-({[4-(3-ethyl-7-morpholin-4-yl-3H-[1,2,3]triazolo[4,5-d]pyrimidin-5-yl)phenyl]carbamoyl}amino)-N-[(1-ethylpyrrolidin-2-yl)methyl]benzamide;
4-({[4-(3-ethyl-7-morpholin-4-yl-3H-[1,2,3]triazolo[4,5-d]pyrimidin-5-yl)phenyl]carbamoyl}amino)benzamide;
4-({[4-(3-ethyl-7-morpholin-4-yl-3H-[1,2,3]triazolo[4,5-d]pyrimidin-5-yl)phenyl]carbamoyl}amino)-N,N-dimethylbenzamide;
N-butyl-4-({[4-(3-ethyl-7-morpholin-4-yl-3H-[1,2,3]triazolo[4,5-d]pyrimidin-5-yl)phenyl]carbamoyl}amino)benzamide;
4-({[4-(3-ethyl-7-morpholin-4-yl-3H-[1,2,3]triazolo[4,5-d]pyrimidin-5-yl)phenyl]carbamoyl}amino)-N-(2-pyridin-2-ylethyl)benzamide;
N-ethyl-4-({[4-(3-ethyl-7-morpholin-4-yl-3H-[1,2,3]triazolo[4,5-d]pyrimidin-5-yl)phenyl]carbamoyl}amino)benzamide;
benzyl 4-({[4-(3-ethyl-7-morpholin-4-yl-3H-[1,2,3]triazolo[4,5-d]pyrimidin-5-yl)phenyl]carbamoyl}amino)piperidine-1-carboxylate;
1-[4-(3-ethyl-7-morpholin-4-yl-3H-[1,2,3]triazolo[4,5-d]pyrimidin-5-yl)phenyl]-3-piperidin-4-ylurea;
4-{3-ethyl-7-[(3S)-3-methylmorpholin-4-yl]-3H-[1,2,3]triazolo[4,5-d]pyrimidin-5-yl}aniline;
1-(4-{3-ethyl-7-[(3S)-3-methylmorpholin-4-yl]-3H-[1,2,3]triazolo[4,5-d]pyrimidin-5-yl}phenyl)-3-[4-(2-hydroxyethyl)phenyl]urea;
1-(4-{3-ethyl-7-[(3S)-3-methylmorpholin-4-yl]-3H-[1,2,3]triazolo[4,5-d]pyrimidin-5-yl}phenyl)-3-(2-thienyl)urea;
1-(4-{3-ethyl-7-[(3S)-3-methylmorpholin-4-yl]-3H-[1,2,3]triazolo[4,5-d]pyrimidin-5-yl}phenyl)-3-[4-(hydroxymethyl)phenyl]urea;
1-(4-{3-ethyl-7-[(3S)-3-methylmorpholin-4-yl]-3H-[1,2,3]triazolo[4,5-d]pyrimidin-5-yl}phenyl)-3-pyridin-4-ylurea;
1-(4-{3-ethyl-7-[(3S)-3-methylmorpholin-4-yl]-3H-[1,2,3]triazolo[4,5-d]pyrimidin-5-yl}phenyl)-3-pyridin-3-ylurea;
1-(4-{3-ethyl-7-[(3S)-3-methylmorpholin-4-yl]-3H-[1,2,3]triazolo[4,5-d]pyrimidin-5-yl}phenyl)-3-(4-methoxyphenyl)urea;
1-(4-{3-ethyl-7-[(3S)-3-methylmorpholin-4-yl]-3H-[1,2,3]triazolo[4,5-d]pyrimidin-5-yl}phenyl)-3-(4-fluorophenyl)urea;
1-(4-cyanophenyl)-3-(4-{3-ethyl-7-[(3S)-3-methylmorpholin-4-yl]-3H-[1,2,3]triazolo[4,5-d]pyrimidin-5-yl}phenyl)urea;
1-(4-{3-ethyl-7-[(3S)-3-methylmorpholin-4-yl]-3H-[1,2,3]triazolo[4,5-d]pyrimidin-5-yl}phenyl)-3-[4-(4-methylpiperazin-1-yl)phenyl]urea;
4-(3-cyclopropyl-7-morpholin-4-yl-3H-[1,2,3]triazolo[4,5-d]pyrimidin-5-yl)aniline;
1-[4-(3-cyclopropyl-7-morpholin-4-yl-3H-[1,2,3]triazolo[4,5-d]pyrimidin-5-yl)phenyl]-3-pyridin-4-ylurea;
1-[4-(3-cyclopropyl-7-morpholin-4-yl-3H-[1,2,3]triazolo[4,5-d]pyrimidin-5-yl)phenyl]-3-pyridin-4-ylurea;
1-[4-(3-cyclopropyl-7-morpholin-4-yl-3H-[1,2,3]triazolo[4,5-d]pyrimidin-5-yl)phenyl]-3-(2-thienyl)urea;
4-(3-isopropyl-7-morpholin-4-yl-3H-[1,2,3]triazolo[4,5-d]pyrimidin-5-yl)aniline;
1-[4-(3-isopropyl-7-morpholin-4-yl-3H-[1,2,3]triazolo[4,5-d]pyrimidin-5-yl)phenyl]-3-pyridin-4-ylurea;
1-[4-(3-isopropyl-7-morpholin-4-yl-3H-[1,2,3]triazolo[4,5-d]pyrimidin-5-yl)phenyl]-3-pyridin-3-ylurea;
1-[4-(hydroxymethyl)phenyl]-3-[4-(3-isopropyl-7-morpholin-4-yl-3H-[1,2,3]triazolo[4,5-d]pyrimidin-5-yl)phenyl]urea;
1-[4-(3-isopropyl-7-morpholin-4-yl-3H-[1,2,3]triazolo[4,5-d]pyrimidin-5-yl)phenyl]-3-(4-morpholin-4-ylphenyl)urea;
1-[4-(dimethylamino)phenyl]-3-[4-(3-isopropyl-7-morpholin-4-yl-3H-[1,2,3]triazolo[4,5-d]pyrimidin-5-yl)phenyl]urea;
1-(4-fluorophenyl)-3-[4-(3-isopropyl-7-morpholin-4-yl-3H-[1,2,3]triazolo[4,5-d]pyrimidin-5-yl)phenyl]urea;
1-[2-(dimethylamino)ethyl]-3-[4-(3-isopropyl-7-morpholin-4-yl-3H-[1,2,3]triazolo[4,5-d]pyrimidin-5-yl)phenyl]urea;
1-[4-(3-isopropyl-7-morpholin-4-yl-3H-[1,2,3]triazolo[4,5-d]pyrimidin-5-yl)phenyl]-3-(4-methoxyphenyl)urea;
1-[4-(3-isopropyl-7-morpholin-4-yl-3H-[1,2,3]triazolo[4,5-d]pyrimidin-5-yl)phenyl]-3-(4-methylphenyl)urea;
1-[4-(3-isopropyl-7-morpholin-4-yl-3H-[1,2,3]triazolo[4,5-d]pyrimidin-5-yl)phenyl]-3-methylurea;
1-[(1-ethylpyrrolidin-2-yl)methyl]-3-[4-(3-isopropyl-7-morpholin-4-yl-3H-[1,2,3]triazolo[4,5-d]pyrimidin-5-yl)phenyl]urea;
4-({[4-(3-isopropyl-7-morpholin-4-yl-3H-[1,2,3]triazolo[4,5-d]pyrimidin-5-yl)phenyl]carbamoyl}amino)benzamide;
1-[4-(3-isopropyl-7-morpholin-4-yl-3H-[1,2,3]triazolo[4,5-d]pyrimidin-5-yl)phenyl]-3-isoxazol-4-ylurea;
1-[4-(3-isopropyl-7-morpholin-4-yl-3H-[1,2,3]triazolo[4,5-d]pyrimidin-5-yl)phenyl]-3-(1H-pyrrol-3-yl)urea;
1-[4-(3-isopropyl-7-morpholin-4-yl-3H-[1,2,3]triazolo[4,5-d]pyrimidin-5-yl)phenyl]urea;
tert-butyl 4-{2-[5-(3-hydroxyphenyl)-7-morpholin-4-yl-3H-[1,2,3]triazolo[4,5-d]pyrimidin-3-yl]ethyl}piperazine-1-carboxylate;
3-[7-morpholin-4-yl-3-(2-piperazin-1-ylethyl)-3H-[1,2,3]triazolo[4,5-d]pyrimidin-5-yl]phenol;
3-{3-[2-(4-benzoylpiperazin-1-yl)ethyl]-7-morpholin-4-yl-3H-[1,2,3]triazolo[4,5-d]pyrimidin-5-yl}phenol;
3-{7-morpholin-4-yl-3-[2-(4-propionylpiperazin-1-yl)ethyl]-3H-[1,2,3]triazolo[4,5-d]pyrimidin-5-yl}phenol;
3-(3-{2-[4-(4-fluorobenzoyl)piperazin-1-yl]ethyl}-7-morpholin-4-yl-3H-[1,2,3]triazolo[4,5-d]pyrimidin-5-yl)phenol;
3-(3-{2-[4-(3,4-difluorobenzoyl)piperazin-1-yl]ethyl}-7-morpholin-4-yl-3H-[1,2,3]triazolo[4,5-d]pyrimidin-5-yl)phenol;
3-{3-[2-(4-isonicotinoylpiperazin-1-yl)ethyl]-7-morpholin-4-yl-3H-[1,2,3]triazolo[4,5-d]pyrimidin-5-yl}phenol;
3-(7-morpholin-4-yl-3-{2-[4-(phenylacetyl)piperazin-1-yl]ethyl}-3H-[1,2,3]triazolo[4,5-d]pyrimidin-5-yl)phenol;
3-{3-[2-(4-acetylpiperazin-1-yl)ethyl]-7-morpholin-4-yl-3H-[1,2,3]triazolo[4,5-d]pyrimidin-5-yl}phenol;
3-{3-[2-(4-cyclohexylpiperazin-1-yl)ethyl]-7-morpholin-4-yl-3H-[1,2,3]triazolo[4,5-d]pyrimidin-5-yl}phenol;
3-{3-[2-(4-butylpiperazin-1-yl)ethyl]-7-morpholin-4-yl-3H-[1,2,3]triazolo[4,5-d]pyrimidin-5-yl}phenol;
3-{3-[2-(4-isobutylpiperazin-1-yl)ethyl]-7-morpholin-4-yl-3H-[1,2,3]triazolo[4,5-d]pyrimidin-5-yl}phenol;
3-(3-{2-[4-(3-fluorobenzyl)piperazin-1-yl]ethyl}-7-morpholin-4-yl-3H-[1,2,3]triazolo[4,5-d]pyrimidin-5-yl)phenol;
3-{3-[2-(4-{4-[3-(dimethylamino)propoxy]benzyl}piperazin-1-yl)ethyl]-7-morpholin-4-yl-3H-[1,2,3]triazolo[4,5-d]pyrimidin-5-yl}phenol;
3-(7-morpholin-4-yl-3-{2-[4-(pyridin-3-ylmethyl)piperazin-1-yl]ethyl}-3H-[1,2,3]triazolo[4,5-d]pyrimidin-5-yl)phenol;
3-(7-morpholin-4-yl-3-{2-[4-(1H-pyrrol-2-ylmethyl)piperazin-1-yl]ethyl}-3H-[1,2,3]triazolo[4,5-d]pyrimidin-5-yl)phenol;
3-(3-{2-[4-(2-furylmethyl)piperazin-1-yl]ethyl}-7-morpholin-4-yl-3H-[1,2,3]triazolo[4,5-d]pyrimidin-5-yl)phenol;
3-{3-[2-(4-benzylpiperazin-1-yl)ethyl]-7-morpholin-4-yl-3H-[1,2,3]triazolo[4,5-d]pyrimidin-5-yl}phenol;
methyl 3-[5-(3-hydroxyphenyl)-7-morpholin-4-yl-3H-[1,2,3]triazolo[4,5-d]pyrimidin-3-yl]benzoate;
methyl 3-[5-(3-formylphenyl)-7-morpholin-4-yl-3H-[1,2,3]triazolo[4,5-d]pyrimidin-3-yl]benzoate;
[(7-morpholin-4-yl-3H-[1,2,3]triazolo[4,5-d]pyrimidine-3,5-diyl)di-3,1-phenylene]dimethanol;
3-[5-(3-hydroxyphenyl)-7-morpholin-4-yl-3H-[1,2,3]triazolo[4,5-d]pyrimidin-3-yl]benzoic acid;
3-[5-(3-hydroxyphenyl)-7-morpholin-4-yl-3H-[1,2,3]triazolo[4,5-d]pyrimidin-3-yl]benzamide;
3-(7-morpholin-4-yl-3-{3-[(4-pyrrolidin-1-ylpiperidin-1-yl)carbonyl]phenyl}-3H-[1,2,3]triazolo[4,5-d]pyrimidin-5-yl)phenol;
3-[5-(3-hydroxyphenyl)-7-morpholin-4-yl-3H-[1,2,3]triazolo[4,5-d]pyrimidin-3-yl]-N-methylbenzamide;
N-[2-(dimethylamino)ethyl]-3-[5-(3-hydroxyphenyl)-7-morpholin-4-yl-3H-[1,2,3]triazolo[4,5-d]pyrimidin-3-yl]benzamide;
3-(7-morpholin-4-yl-5-{4-[(phenylcarbamoyl)amino]phenyl}-3H-[1,2,3]triazolo[4,5-d]pyrimidin-3-yl)benzoic acid;
tert-butyl 3-[5-(3-hydroxyphenyl)-7-morpholin-4-yl-3H-[1,2,3]triazolo[4,5-d]pyrimidin-3-yl]azetidine-1-carboxylate;
3-(3-azetidin-3-yl-7-morpholin-4-yl-3H-[1,2,3]triazolo[4,5-d]pyrimidin-5-yl)phenol;
3-{3-[1-(2-aminobenzoyl)azetidin-3-yl]-7-morpholin-4-yl-3H-[1,2,3]triazolo[4,5-d]pyrimidin-5-yl}phenol;
3-[3-(1-benzylazetidin-3-yl)-7-morpholin-4-yl-3H-[1,2,3]triazolo[4,5-d]pyrimidin-5-yl]phenol;
3-(3-{1-[(6-fluoropyridin-3-yl)methyl]azetidin-3-yl}-7-morpholin-4-yl-3H-[1,2,3]triazolo[4,5-d]pyrimidin-5-yl)phenol;
(11bS)-11,11b-dimethyl-2,3,5,6,11,11b-hexahydro-1H-indolizino[8,7-b]indol-8-ol;
diethyl 8-ethynyl-7-hydroxydibenzo[b,d]furan-3,4-dicarboxylate;
tert-butyl 3-(7-morpholin-4-yl-5-{4-[(phenylcarbamoyl)amino]phenyl}-3H-[1,2,3]triazolo[4,5-d]pyrimidin-3-yl)azetidine-1-carboxylate;
tert-butyl 3-(7-morpholin-4-yl-5-{4-[(2-thienylcarbamoyl)amino]phenyl}-3H-[1,2,3]triazolo[4,5-d]pyrimidin-3-yl)azetidine-1-carboxylate;
4-[7-morpholin-4-yl-3-(2,2,2-trifluoroethyl)-3H-[1,2,3]triazolo[4,5-d]pyrimidin-5-yl]aniline;
1-{4-[7-morpholin-4-yl-3-(2,2,2-trifluoroethyl)-3H-[1,2,3]triazolo[4,5-d]pyrimidin-5-yl]phenyl}-3-pyridin-4-ylurea;
1-{4-[7-morpholin-4-yl-3-(2,2,2-trifluoroethyl)-3H-[1,2,3]triazolo[4,5-d]pyrimidin-5-yl]phenyl}-3-pyridin-3-ylurea;
1-{4-[7-morpholin-4-yl-3-(2,2,2-trifluoroethyl)-3H-[1,2,3]triazolo[4,5-d]pyrimidin-5-yl]phenyl}-3-pyrimidin-5-ylurea;
1-[4-(dimethylamino)phenyl]-3-{4-[7-morpholin-4-yl-3-(2,2,2-trifluoroethyl)-3H-[1,2,3]triazolo[4,5-d]pyrimidin-5-yl]phenyl}urea;
1-[4-(2-hydroxyethyl)phenyl]-3-{4-[7-morpholin-4-yl-3-(2,2,2-trifluoroethyl)-3H-[1,2,3]triazolo[4,5-d]pyrimidin-5-yl]phenyl}urea;
tert-butyl methyl {2-[({4-[7-morpholin-4-yl-3-(2,2,2-trifluoroethyl)-3H-[1,2,3]triazolo[4,5-d]pyrimidin-5-yl]phenyl}carbamoyl)amino]ethyl}carbamate;
1-[2-(methylamino)ethyl]-3-{4-[7-morpholin-4-yl-3-(2,2,2-trifluoroethyl)-3H-[1,2,3]triazolo[4,5-d]pyrimidin-5-yl]phenyl}urea;
1-{4-[7-morpholin-4-yl-3-(2,2,2-trifluoroethyl)-3H-[1,2,3]triazolo[4,5-d]pyrimidin-5-yl]phenyl}-3-(2-thienyl)urea;
1-{4-[7-morpholin-4-yl-3-(2,2,2-trifluoroethyl)-3H-[1,2,3]triazolo[4,5-d]pyrimidin-5-yl]phenyl}-3-(3-thienyl)urea;
tert-butyl 4-[5-(3-hydroxyphenyl)-7-morpholin-4-yl-3H-[1,2,3]triazolo[4,5-d]pyrimidin-3-yl]piperidine-1-carboxylate;
3-(7-morpholin-4-yl-3-piperidin-4-yl-3H-[1,2,3]triazolo[4,5-d]pyrimidin-5-yl)phenol;
3-{7-morpholin-4-yl-3-[1-(1H-pyrrol-2-ylmethyl)piperidin-4-yl]-3H-[1,2,3]triazolo[4,5-d]pyrimidin-5-yl}phenol;
3-[3-(1-{4-[3-(dimethylamino)propoxy]benzyl}piperidin-4-yl)-7-morpholin-4-yl-3H-[1,2,3]triazolo[4,5-d]pyrimidin-5-yl]phenol;
3-{3-[1-(4-fluorobenzyl)piperidin-4-yl]-7-morpholin-4-yl-3H-[1,2,3]triazolo[4,5-d]pyrimidin-5-yl}phenol;
tert-butyl 4-[5-(2-aminopyrimidin-5-yl)-7-morpholin-4-yl-3H-[1,2,3]triazolo[4,5-d]pyrimidin-3-yl]piperidine-1-carboxylate;
3-{7-morpholin-4-yl-3-[1-(pyridin-2-ylmethyl)piperidin-4-yl]-3H-[1,2,3]triazolo[4,5-d]pyrimidin-5-yl}phenol;
tert-butyl 4-(7-morpholin-4-yl-5-{4-[(pyridin-3-ylcarbamoyl)amino]phenyl}-3H-[1,2,3]triazolo[4,5-d]pyrimidin-3-yl)piperidine-1-carboxylate;
tert-butyl 4-{5-[4-({[2-(dimethylamino)ethyl]carbamoyl}amino)phenyl]-7-morpholin-4-yl-3H-[1,2,3]triazolo[4,5-d]pyrimidin-3-yl}piperidine-1-carboxylate;
1-[2-(dimethylamino)ethyl]-3-[4-(7-morpholin-4-yl-3-piperidin-4-yl-3H-[1,2,3]triazolo[4,5-d]pyrimidin-5-yl)phenyl]urea;
1-[2-(dimethylamino)ethyl]-3-(4-{3-[1-(4-fluorobenzyl)piperidin-4-yl]-7-morpholin-4-yl-3H-[1,2,3]triazolo[4,5-d]pyrimidin-5-yl}phenyl)urea;
1-[2-(dimethylamino)ethyl]-3-(4-{7-morpholin-4-yl-3-[1-(pyridin-3-ylmethyl)piperidin-4-yl]-3H-[1,2,3]triazolo[4,5-d]pyrimidin-5-yl}phenyl)urea;
1-[4-(3-{1-[(6-bromopyridin-3-yl)methyl]piperidin-4-yl}-7-morpholin-4-yl-3H-[1,2,3]triazolo[4,5-d]pyrimidin-5-yl)phenyl]-3-[2-(dimethylamino)ethyl]urea;
1-(4-{3-[1-(4-chloro-2-fluorobenzyl)piperidin-4-yl]-7-morpholin-4-yl-3H-[1,2,3]triazolo[4,5-d]pyrimidin-5-yl}phenyl)-3-[2-(dimethylamino)ethyl]urea;
1-[2-(dimethylamino)ethyl]-3-[4-(3-{1-[(6-fluoropyridin-3-yl)methyl]piperidin-4-yl}-7-morpholin-4-yl-3H-[1,2,3]triazolo[4,5-d]pyrimidin-5-yl)phenyl]urea;
1-[2-(dimethylamino)ethyl]-3-[4-(3-{1-[(5-methyl-2-thienyl)methyl]piperidin-4-yl}-7-morpholin-4-yl-3H-[1,2,3]triazolo[4,5-d]pyrimidin-5-yl)phenyl]urea;
1-{4-[3-(1-butylpiperidin-4-yl)-7-morpholin-4-yl-3H-[1,2,3]triazolo[4,5-d]pyrimidin-5-yl]phenyl}-3-[2-(dimethylamino)ethyl]urea;
1-[2-(dimethylamino)ethyl]-3-(4-{7-morpholin-4-yl-3-[1-(4-pyridin-4-ylbenzyl)piperidin-4-yl]-3H-[1,2,3]triazolo[4,5-d]pyrimidin-5-yl}phenyl)urea;
1-[2-(dimethylamino)ethyl]-3-(4-{7-morpholin-4-yl-3-[1-(1H-pyrrol-2-ylmethyl)piperidin-4-yl]-3H-[1,2,3]triazolo[4,5-d]pyrimidin-5-yl}phenyl)urea;
1-[2-(dimethylamino)ethyl]-3-{4-[3-(1-{4-[3-(dimethylamino)propoxy]benzyl}piperidin-4-yl)-7-morpholin-4-yl-3H-[1,2,3]triazolo[4,5-d]pyrimidin-5-yl]phenyl}urea;
1-[4-(7-morpholin-4-yl-3-piperidin-4-yl-3H-[1,2,3]triazolo[4,5-d]pyrimidin-5-yl)phenyl]-3-pyridin-3-ylurea;
1-{4-[3-(1-methylpiperidin-4-yl)-7-morpholin-4-yl-3H-[1,2,3]triazolo[4,5-d]pyrimidin-5-yl]phenyl}-3-pyridin-3-ylurea;
tert-butyl 4-[5-(4-{[(4-fluorophenyl)carbamoyl]amino}phenyl)-7-morpholin-4-yl-3H-[1,2,3]triazolo[4,5-d]pyrimidin-3-yl]piperidine-1-carboxylate;
tert-butyl 4-(7-morpholin-4-yl-5-{4-[(pyridin-4-ylcarbamoyl)amino]phenyl}-3H-[1,2,3]triazolo[4,5-d]pyrimidin-3-yl)piperidine-1-carboxylate;
1-[4-(7-morpholin-4-yl-3-piperidin-4-yl-3H-[1,2,3]triazolo[4,5-d]pyrimidin-5-yl)phenyl]-3-pyridin-4-ylurea;
tert-butyl 4-(5-{4-[(methylcarbamoyl)amino]phenyl}-7-morpholin-4-yl-3H-[1,2,3]triazolo[4,5-d]pyrimidin-3-yl)piperidine-1-carboxylate;
tert-butyl 4-[5-(4-{[(methoxycarbonyl)carbamoyl]amino}phenyl)-7-morpholin-4-yl-3H-[1,2,3]triazolo[4,5-d]pyrimidin-3-yl]piperidine-1-carboxylate;
1-{4-[3-(1-benzylpiperidin-4-yl)-7-morpholin-4-yl-3H-[1,2,3]triazolo[4,5-d]pyrimidin-5-yl]phenyl}-3-(3-chlorophenyl)urea;
5-(3-{1-[(2-amino-1,3-thiazol-5-yl)methyl]piperidin-4-yl}-7-morpholin-4-yl-3H-[1,2,3]triazolo[4,5-d]pyrimidin-5-yl)pyridin-3-ol;
3-{3-[(1-ethylpyrrolidin-2-yl)methyl]-7-morpholin-4-yl-3H-[1,2,3]triazolo[4,5-d]pyrimidin-5-yl}phenol;
{5-[3-(1-benzylpiperidin-4-yl)-7-morpholin-4-yl-3H-[1,2,3]triazolo[4,5-d]pyrimidin-5-yl]pyridin-3-yl}methanol;
[5-(7-morpholin-4-yl-3-piperidin-4-yl-3H-[1,2,3]triazolo[4,5-d]pyrimidin-5-yl)pyridin-3-yl]methanol;
4-(3-ethyl-7-morpholin-4-yl-3H-[1,2,3]triazolo[4,5-d]pyrimidin-5-yl)-2-methoxyaniline;
[3-(7-morpholin-4-yl-3-piperidin-4-yl-3H-[1,2,3]triazolo[4,5-d]pyrimidin-5-yl)phenyl]methanol;
{3-[3-(1-benzylpiperidin-4-yl)-7-morpholin-4-yl-3H-[1,2,3]triazolo[4,5-d]pyrimidin-5-yl]phenyl}methanol;
4-[3-(2,2-dimethoxyethyl)-7-morpholin-4-yl-3H-[1,2,3]triazolo[4,5-d]pyrimidin-5-yl]aniline;
1-{4-[3-(2,2-dimethoxyethyl)-7-morpholin-4-yl-3H-[1,2,3]triazolo[4,5-d]pyrimidin-5-yl]phenyl}-3-(4-methylphenyl)urea;
1-{4-[3-(2,2-dimethoxyethyl)-7-morpholin-4-yl-3H-[1,2,3]triazolo[4,5-d]pyrimidin-5-yl]phenyl}-3-(4-fluorophenyl)urea;
1-{4-[3-(2,2-dimethoxyethyl)-7-morpholin-4-yl-3H-[1,2,3]triazolo[4,5-d]pyrimidin-5-yl]phenyl}-3-pyridin-3-ylurea;
4-[({4-[3-(2,2-dimethoxyethyl)-7-morpholin-4-yl-3H-[1,2,3]triazolo[4,5-d]pyrimidin-5-yl]phenyl}carbamoyl)amino]benzamide;
1-{4-[3-(2-hydroxyethyl)-7-morpholin-4-yl-3H-[1,2,3]triazolo[4,5-d]pyrimidin-5-yl]phenyl}-3-pyridin-4-ylurea;
1-{4-[3-(2-hydroxyethyl)-7-morpholin-4-yl-3H-[1,2,3]triazolo[4,5-d]pyrimidin-5-yl]phenyl}-3-pyridin-3-ylurea;
1-{4-[3-(2-hydroxyethyl)-7-morpholin-4-yl-3H-[1,2,3]triazolo[4,5-d]pyrimidin-5-yl]phenyl}-3-(4-methoxyphenyl)urea;
1-{4-[3-(2-hydroxyethyl)-7-morpholin-4-yl-3H-[1,2,3]triazolo[4,5-d]pyrimidin-5-yl]phenyl}-3-phenylurea;
tert-butyl 3-{[5-(4-aminophenyl)-7-morpholin-4-yl-3H-[1,2,3]triazolo[4,5-d]pyrimidin-3-yl]methyl}azetidine-1-carboxylate;
tert-butyl 3-[(7-morpholin-4-yl-5-{4-[(phenylcarbamoyl)amino]phenyl}-3H-[1,2,3]triazolo[4,5-d]pyrimidin-3-yl)methyl]azetidine-1-carboxylate;
1-{4-[3-(azetidin-3-ylmethyl)-7-morpholin-4-yl-3H-[1,2,3]triazolo[4,5-d]pyrimidin-5-yl]phenyl}-3-phenylurea;
1-(4-{3-[(1-benzoylazetidin-3-yl)methyl]-7-morpholin-4-yl-3H-[1,2,3]triazolo[4,5-d]pyrimidin-5-yl}phenyl)-3-phenylurea;
1-(4-{3-[(1-benzylazetidin-3-yl)methyl]-7-morpholin-4-yl-3H-[1,2,3]triazolo[4,5-d]pyrimidin-5-yl}phenyl)-3-phenylurea;
1-[4-(3-{[1-(4-fluorobenzyl)azetidin-3-yl]methyl}-7-morpholin-4-yl-3H-[1,2,3]triazolo[4,5-d]pyrimidin-5-yl)phenyl]-3-phenylurea;
1-[4-(7-morpholin-4-yl-3-{[1-(4-pyridin-4-ylbenzyl)azetidin-3-yl]methyl}-3H-[1,2,3]triazolo[4,5-d]pyrimidin-5-yl)phenyl]-3-phenylurea;
1-(4-{3-[(1-{4-[3-(dimethylamino)propoxy]benzyl}azetidin-3-yl)methyl]-7-morpholin-4-yl-3H-[1,2,3]triazolo[4,5-d]pyrimidin-5-yl}phenyl)-3-phenylurea;
3-[7-morpholin-4-yl-3-(2-piperidin-1-ylethyl)-3H-[1,2,3]triazolo[4,5-d]pyrimidin-5-yl]phenol;
3-[7-morpholin-4-yl-3-(2-pyridin-2-ylethyl)-3H-[1,2,3]triazolo[4,5-d]pyrimidin-5-yl]phenol;
4-chloro-N-(4-{[5-(3-hydroxyphenyl)-7-morpholin-4-yl-3H-[1,2,3]triazolo[4,5-d]pyrimidin-3-yl]methyl}phenyl)benzamide;
1-{4-[7-morpholin-4-yl-3-(tetrahydro-2H-pyran-4-yl)-3H-[1,2,3]triazolo[4,5-d]pyrimidin-5-yl]phenyl}-3-pyridin-4-ylurea;
1-[4-(3-methyl-7-morpholin-4-yl-3H-[1,2,3]triazolo[4,5-d]pyrimidin-5-yl)phenyl]-3-pyridin-3-ylurea;
1-[4-(3-methyl-7-morpholin-4-yl-3H-[1,2,3]triazolo[4,5-d]pyrimidin-5-yl)phenyl]-3-(2-thienyl)urea;
1-[4-(3-methyl-7-morpholin-4-yl-3H-[1,2,3]triazolo[4,5-d]pyrimidin-5-yl)phenyl]-3-(3-thienyl)urea;
3-{3-[4-(dimethylamino)butyl]-7-morpholin-4-yl-3H-[1,2,3]triazolo[4,5-d]pyrimidin-5-yl}phenol;
3-{3-[4-(methylamino)butyl]-7-morpholin-4-yl-3H-[1,2,3]triazolo[4,5-d]pyrimidin-5-yl}phenol;
3-[3-(4-aminobutyl)-7-morpholin-4-yl-3H-[1,2,3]triazolo[4,5-d]pyrimidin-5-yl]phenol;
3-[7-morpholin-4-yl-3-(4-pyrrolidin-1-ylbutyl)-3H-[1,2,3]triazolo[4,5-d]pyrimidin-5-yl]phenol;
3-{3-[4-(4-benzylpiperazin-1-yl)butyl]-7-morpholin-4-yl-3H-[1,2,3]triazolo[4,5-d]pyrimidin-5-yl}phenol;
4-{[5-(3-hydroxyphenyl)-7-morpholin-4-yl-3H-[1,2,3]triazolo[4,5-d]pyrimidin-3-yl]methyl}-N-methylbenzamide;
tert-butyl 4-[(4-{[5-(3-hydroxyphenyl)-7-morpholin-4-yl-3H-[1,2,3]triazolo[4,5-d]pyrimidin-3-yl]methyl}benzoyl)amino]piperidine-1-carboxylate;
tert-butyl [1-(4-{[5-(3-hydroxyphenyl)-7-morpholin-4-yl-3H-[1,2,3]triazolo[4,5-d]pyrimidin-3-yl]methyl}benzoyl)piperidin-4-yl]carbamate;
N-(2-acetamidoethyl)-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(3-pyrrolidin-1-ylpropyl)benzamide;
N-benzyl-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(2-pyrrolidin-1-ylethyl)benzamide;
N-[2-(dimethylamino)ethyl]-4-{[5-(3-hydroxyphenyl)-7-morpholin-4-yl-3H-[1,2,3]triazolo[4,5-d]pyrimidin-3-yl]methyl}benzamide;
N-[3-(dimethylamino)propyl]-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-pyridin-3-ylbenzamide;
N-(4-fluorophenyl)-4-{[5-(3-hydroxyphenyl)-7-morpholin-4-yl-3H-[1,2,3]triazolo[4,5-d]pyrimidin-3-yl]methyl}benzamide;
tert-butyl 4-{4-[(4-{[5-(3-hydroxyphenyl)-7-morpholin-4-yl-3H-[1,2,3]triazolo[4,5-d]pyrimidin-3-yl]methyl}benzoyl)amino]phenyl}piperazine-1-carboxylate;
N-ethyl-4-{[5-(3-hydroxyphenyl)-7-morpholin-4-yl-3H-[1,2,3]triazolo[4,5-d]pyrimidin-3-yl]methyl}benzamide;
N,N-diethyl-4-{[5-(3-hydroxyphenyl)-7-morpholin-4-yl-3H-[1,2,3]triazolo[4,5-d]pyrimidin-3-yl]methyl}benzamide;
N-cyclopropyl-4-{[5-(3-hydroxyphenyl)-7-morpholin-4-yl-3H-[1,2,3]triazolo[4,5-d]pyrimidin-3-yl]methyl}benzamide;
N-tert-butyl-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-(2-phenylethyl)benzamide;
4-{[5-(3-hydroxyphenyl)-7-morpholin-4-yl-3H-[1,2,3]triazolo[4,5-d]pyrimidin-3-yl]methyl}-N-[(1S)-1-phenylethyl]benzamide;
4-{[5-(3-hydroxyphenyl)-7-morpholin-4-yl-3H-[1,2,3]triazolo[4,5-d]pyrimidin-3-yl]methyl}-N-[2-(1H-indol-3-yl)ethyl]benzamide;
N-(2-hydroxy-2-phenylethyl)-4-{[5-(3-hydroxyphenyl)-7-morpholin-4-yl-3H-[1,2,3]triazolo[4,5-d]pyrimidin-3-yl]methyl}benzamide;
3-{7-morpholin-4-yl-3-[4-(piperidin-1-ylcarbonyl)benzyl]-3H-[1,2,3]triazolo[4,5-d]pyrimidin-5-yl}phenol;
3-{7-morpholin-4-yl-3-[4-(pyrrolidin-1-ylcarbonyl)benzyl]-3H-[1,2,3]triazolo[4,5-d]pyrimidin-5-yl}phenol;
3-(7-morpholin-4-yl-3-{4-[(4-phenylpiperazin-1-yl)carbonyl]benzyl}-3H-[1,2,3]triazolo[4,5-d]pyrimidin-5-yl)phenol;
N-(2-furylmethyl)-4-{[5-(3-hydroxyphenyl)-7-morpholin-4-yl-3H-[1,2,3]triazolo[4,5-d]pyrimidin-3-yl]methyl}benzamide;
4-{[5-(3-hydroxyphenyl)-7-morpholin-4-yl-3H-[1,2,3]triazolo[4,5-d]pyrimidin-3-yl]methyl}-N-[2-(1H-imidazol-5-yl)ethyl]benzamide;
tert-butyl {5-[3-(hydroxymethyl)phenyl]-7-morpholin-4-yl-3H-[1,2,3]triazolo[4,5-d]pyrimidin-3-yl}acetate;
tert-butyl [5-(3-hydroxyphenyl)-7-morpholin-4-yl-3H-[1,2,3]triazolo[4,5-d]pyrimidin-3-yl]acetate;
tert-butyl (7-morpholin-4-yl-5-{4-[(pyridin-4-ylcarbamoyl)amino]phenyl}-3H-[1,2,3]triazolo[4,5-d]pyrimidin-3-yl)acetate;
2-{5-[3-(hydroxymethyl)phenyl]-7-morpholin-4-yl-3H-[1,2,3]triazolo[4,5-d]pyrimidin-3-yl}-N-pyridin-3-ylacetamide;
2-{5-[3-(hydroxymethyl)phenyl]-7-morpholin-4-yl-3H-[1,2,3]triazolo[4,5-d]pyrimidin-3-yl}-N-methylacetamide;
2-{5-[3-(hydroxymethyl)phenyl]-7-morpholin-4-yl-3H-[1,2,3]triazolo[4,5-d]pyrimidin-3-yl}acetamide;
N-(4-fluorophenyl)-2-{5-[3-(hydroxymethyl)phenyl]-7-morpholin-4-yl-3H-[1,2,3]triazolo[4,5-d]pyrimidin-3-yl}acetamide;
N-[2-(dimethylamino)ethyl]-2-{5-[3-(hydroxymethyl)phenyl]-7-morpholin-4-yl-3H-[1,2,3]triazolo[4,5-d]pyrimidin-3-yl}acetamide;
{-[3-(hydroxymethyl)phenyl]-7-morpholin-4-yl-3H-[1,2,3]triazolo[4,5-d]pyrimidin-3-yl}acetic acid;
methyl 4-{[5-(3-hydroxyphenyl)-7-morpholin-4-yl-3H-[1,2,3]triazolo[4,5-d]pyrimidin-3-yl]methyl}benzoate;
4-{[5-(3-hydroxyphenyl)-7-morpholin-4-yl-3H-[1,2,3]triazolo[4,5-d]pyrimidin-3-yl]methyl}benzoic acid;
methyl 4-({5-[3-(hydroxymethyl)phenyl]-7-morpholin-4-yl-3H-[1,2,3]triazolo[4,5-d]pyrimidin-3-yl}methyl)benzoate;
methyl 4-{[5-(3-fluoro-5-hydroxyphenyl)-7-morpholin-4-yl-3H-[1,2,3]triazolo[4,5-d]pyrimidin-3-yl]methyl}benzoate;
and [5-(3-hydroxyphenyl)-7-morpholin-4-yl-3H-[1,2,3]triazolo[4,5-d]pyrimidin-3-yl]acetic acid.

26. A compound selected from the group consisting of:
1-{4-[(2,2-dimethylhydrazino)carbonyl]phenyl}-3-[4-(3-ethyl-7-morpholin-4-yl-3H-[1,2,3]triazolo[4,5-d]pyrimidin-5-yl)phenyl]urea;
1-[4-(3-ethyl-7-morpholin-4-yl-3H-[1,2,3]triazolo[4,5-d]pyrimidin-5-yl)phenyl]-3-(4-nitrophenyl)urea;
1-(4-aminophenyl)-3-[4-(3-ethyl-7-morpholin-4-yl-3H-[1,2,3]triazolo[4,5-d]pyrimidin-5-yl)phenyl]urea;
N-[4-({[4-(3-ethyl-7-morpholin-4-yl-3H-[1,2,3]triazolo[4,5-d]pyrimidin-5-yl)phenyl]carbamoyl}amino)phenyl]-N2,N2-dimethylglycinamide;
3-[5-(4-{[(4-{[2-(dimethylamino)ethyl]carbamoyl}phenyl)carbamoyl]amino}phenyl)-7-morpholin-4-yl-3H-[1,2,3]triazolo[4,5-d]pyrimidin-3-yl]benzoic acid;
4-[({4-[3-(3-carbamoylphenyl)-7-morpholin-4-yl-3H-[1,2,3]triazolo[4,5-d]pyrimidin-5-yl]phenyl}carbamoyl)amino]-N-[2-(dimethylamino)ethyl]benzamide;
1-[4-(3-ethyl-7-morpholin-4-yl-3H-[1,2,3]triazolo[4,5-d]pyrimidin-5-yl)phenyl]-3-{4-[(pyridin-4-ylmethyl)amino]phenyl}urea;
1-[4-(3-ethyl-7-morpholin-4-yl-3H-[1,2,3]triazolo[4,5-d]pyrimidin-5-yl)phenyl]-3-{4-[(pyridin-3-ylmethyl)amino]phenyl}urea;
1-[4-(3-ethyl-7-morpholin-4-yl-3H-[1,2,3]triazolo[4,5-d]pyrimidin-5-yl)phenyl]-3-(4-{[(6-fluoropyridin-3-yl)methyl]amino}phenyl)urea;
1-[4-(3-ethyl-7-morpholin-4-yl-3H-[1,2,3]triazolo[4,5-d]pyrimidin-5-yl)phenyl]-3-(4-{[(6-methoxypyridin-3-yl)methyl]amino}phenyl)urea;
N-[2-(dimethylamino)ethyl]-4-[({4-[3-(1-methylethyl)-7-morpholin-4-yl-3H-[1,2,3]triazolo[4,5-d]pyrimidin-5-yl]phenyl}carbamoyl)amino]benzamide;
1-{4-[3-(1-methylethyl)-7-morpholin-4-yl-3H-[1,2,3]triazolo[4,5-d]pyrimidin-5-yl]phenyl}-3-{4-[(4-methylpiperazin-1-yl)carbonyl]phenyl}urea;
1-[4-(3-ethyl-7-morpholin-4-yl-3H-[1,2,3]triazolo[4,5-d]pyrimidin-5-yl)phenyl]-3-{4-[(4-methylpiperazin-1-yl)methyl]phenyl}urea;
1-[4-(3-ethyl-7-morpholin-4-yl-3H-[1,2,3]triazolo[4,5-d]pyrimidin-5-yl)phenyl]-3-[4-(4-methylpiperazin-1-yl)phenyl]urea;
4-({[4-(3-ethyl-7-morpholin-4-yl-3H-[1,2,3]triazolo[4,5-d]pyrimidin-5-yl)phenyl]carbamoyl}amino)-N-pyridin-3-ylbenzamide;
N-[4-({[4-(3-ethyl-7-morpholin-4-yl-3H-[1,2,3]triazolo[4,5-d]pyrimidin-5-yl)phenyl]carbamoyl}amino)phenyl]-4-methylpiperazine-1-carboxamide;
N-[4-({[4-(3-ethyl-7-morpholin-4-yl-3H-[1,2,3]triazolo[4,5-d]pyrimidin-5-yl)phenyl]carbamoyl}amino)phenyl]pyridine-4-carboxamide;
N-[4-({[4-(3-ethyl-7-morpholin-4-yl-3H-[1,2,3]triazolo[4,5-d]pyrimidin-5-yl)phenyl]carbamoyl}amino)phenyl]morpholine-4-carboxamide;
3-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]benzamide;
1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]urea;
4-(dimethylamino)-N-[4-({[4-(3-ethyl-7-morpholin-4-yl-3H-[1,2,3]triazolo[4,5-d]pyrimidin-5-yl)phenyl]carbamoyl}amino)phenyl]piperidine-1-carboxamide;
1-[4-(3-ethyl-7-morpholin-4-yl-3H-[1,2,3]triazolo[4,5-d]pyrimidin-5-yl)phenyl]-3-(4-{[(1-methylpiperidin-4-yl)carbamoyl]amino}phenyl)urea;
1-[4-(3-ethyl-7-morpholin-4-yl-3H-[1,2,3]triazolo[4,5-d]pyrimidin-5-yl)phenyl]-3-[4-({[2-(4-methylpiperazin-1-yl)ethyl]carbamoyl}amino)phenyl]urea;
N-[4-({[4-(3-ethyl-7-morpholin-4-yl-3H-[1,2,3]triazolo[4,5-d]pyrimidin-5-yl)phenyl]carbamoyl}amino)phenyl]-4-methyl-1,4-diazepane-1-carboxamide;
1-[2-(dimethylamino)ethyl]-3-[4-({[4-(3-ethyl-7-morpholin-4-yl-3H-[1,2,3]triazolo[4,5-d]pyrimidin-5-yl)phenyl]carbamoyl}amino)phenyl]-1-methylurea;
1-[4-(3-ethyl-7-morpholin-4-yl-3H-[1,2,3]triazolo[4,5-d]pyrimidin-5-yl)phenyl]-3-(4-{[(2-pyrrolidin-1-ylethyl)carbamoyl]amino}phenyl)urea;
N-[4-({[4-(3-ethyl-7-morpholin-4-yl-3H-[1,2,3]triazolo[4,5-d]pyrimidin-5-yl)phenyl]carbamoyl}amino)phenyl]-4-pyrrolidin-1-ylpiperidine-1-carboxamide;
1-[4-(3-ethyl-7-morpholin-4-yl-3H-[1,2,3]triazolo[4,5-d]pyrimidin-5-yl)phenyl]-3-(4-{[(pyridin-2-ylmethyl)carbamoyl]amino}phenyl)urea;
N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide;
4-ethyl-N-[4-({[4-(3-ethyl-7-morpholin-4-yl-3H-[1,2,3]triazolo[4,5-d]pyrimidin-5-yl)phenyl]carbamoyl}amino)phenyl]piperazine-1-carboxamide;
1-[4-(3-ethyl-7-morpholin-4-yl-3H-[1,2,3]triazolo[4,5-d]pyrimidin-5-yl)phenyl]-3-(4-{[(2-methoxyethyl)carbamoyl]amino}phenyl)urea;
1-{4-[3-(1-methylethyl)-7-morpholin-4-yl-3H-[1,2,3]triazolo[4,5-d]pyrimidin-5-yl]phenyl}-3-[4-(4-methylpiperazin-1-yl)phenyl]urea;
1-{4-[3-(1-methylethyl)-7-morpholin-4-yl-3H-[1,2,3]triazolo[4,5-d]pyrimidin-5-yl]phenyl}-3-(4-nitrophenyl)urea;
N-[4-({[4-(3-isopropyl-7-morpholin-4-yl-3H-[1,2,3]triazolo[4,5-d]pyrimidin-5-yl)phenyl]carbamoyl}amino)phenyl]methanesulfonamide;
1-(4-aminophenyl)-3-[4-(3-isopropyl-7-morpholin-4-yl-3H-[1,2,3]triazolo[4,5-d]pyrimidin-5-yl)phenyl]urea;
1-(4-{[4-(dimethylamino)piperidin-1-yl]carbonyl}phenyl)-3-(4-{3-ethyl-7-[(3S)-3-methylmorpholin-4-yl]-3H-[1,2,3]triazolo[4,5-d]pyrimidin-5-yl}phenyl)urea;
1-(4-{3-ethyl-7-[(3S)-3-methylmorpholin-4-yl]-3H-[1,2,3]triazolo[4,5-d]pyrimidin-5-yl}phenyl)-3-{4-[(4-methylpiperazin-1-yl)carbonyl]phenyl}urea;
4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-pyrrolidin-1-ylethyl)benzamide;
4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-(2-piperidin-1-ylethyl)benzamide;
N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}-N-methylbenzamide;
N-[2-(dimethylamino)ethyl]-4-{[(4-{3-ethyl-7-[(3S)-3-methylmorpholin-4-yl]-3H-[1,2,3]triazolo[4,5-d]pyrimidin-5-yl}phenyl)carbamoyl]amino}benzamide;
methyl 5-({[4-(3-ethyl-7-morpholin-4-yl-3H-[1,2,3]triazolo[4,5-d]pyrimidin-5-yl)phenyl]carbamoyl}amino)pyridine-2-carboxylate;
5-({[4-(3-ethyl-7-morpholin-4-yl-3H-[1,2,3]triazolo[4,5-d]pyrimidin-5-yl)phenyl]carbamoyl}amino)pyridine-2-carboxylic acid;
1-[4-(3-ethyl-7-morpholin-4-yl-3H-[1,2,3]triazolo[4,5-d]pyrimidin-5-yl)phenyl]-3-{6-[(4-methylpiperazin-1-yl)carbonyl]pyridin-3-yl}urea;
and N-[2-(dimethylamino)ethyl]-5-({[4-(3-ethyl-7-morpholin-4-yl-3H-[1,2,3]triazolo[4,5-d]pyrimidin-5-yl)phenyl]carbamoyl}amino)-N-methylpyridine-2-carboxamide.

27. A compound selected from the group consisting of:
N-(2-(dimethylamino)ethyl)-N-methyl-4-(3-(4-(3-methyl-7-morpholino-3H-[1,2,3]triazolo[4,5-d]pyrimidin-5-yl)phenyl)ureido)benzamide;
N-(2-(dimethylamino)ethyl)-4-(3-(4-(3-methyl-7-morpholino-3H-[1,2,3]triazolo[4,5-d]pyrimidin-5-yl)phenyl)ureido)benzamide;
1-(4-(3-methyl-7-morpholino-3H-[1,2,3]triazolo[4,5-d]pyrimidin-5-yl)phenyl)-3-(4-(4-methylpiperazine-1-carbonyl)phenyl)urea;
1-(4-(4-(dimethylamino)piperidine-1-carbonyl)phenyl)-3-(4-(3-methyl-7-morpholino-3H-[1,2,3]triazolo[4,5-d]pyrimidin-5-yl)phenyl)urea;
N-(2-(dimethylamino)ethyl)-N-methyl-4-(3-(4-(7-morpholino-3-(2,2,2-trifluoroethyl)-3H-[1,2,3]triazolo[4,5-d]pyrimidin-5-yl)phenyl)ureido)benzamide;
N-(2-(dimethylamino)ethyl)-4-(3-(4-(7-morpholino-3-(2,2,2-trifluoroethyl)-3H-[1,2,3]triazolo[4,5-d]pyrimidin-5-yl)phenyl)ureido)benzamide;
1-(4-(4-methylpiperazine-1-carbonyl)phenyl)-3-(4-(7-morpholino-3-(2,2,2-trifluoroethyl)-3H-[1,2,3]triazolo[4,5-d]pyrimidin-5-yl)phenyl)urea;
and 1-(4-(4-(dimethylamino)piperidine-1-carbonyl)phenyl)-3-(4-(7-morpholino-3-(2,2,2-trifluoroethyl)-3H-[1,2,3]triazolo[4,5-d]pyrimidin-5-yl)phenyl)urea.

28. A composition comprising the compound of claim 1 and a pharmaceutically acceptable carrier.

29. The composition of claim 28 wherein the pharmaceutically acceptable carrier is suitable for oral administration and the composition comprises an oral dosage form.

30. A method of inhibiting PI3K, comprising administering to a mammal the compound or a pharmaceutically acceptable salt of the compound of claim 1, in an amount effective to inhibit PI3K.

31. A method of inhibiting mTOR, comprising administering to a mammal the compound or a pharmaceutically acceptable salt of the compound of claim 1, in an amount effective to inhibit mTOR.

32. A method of treating advanced renal cell carcinoma, comprising administering to a mammal in need thereof the compound or a pharmaceutically acceptable salt of the compound of claim 1, in an amount effective to treat advanced renal cell carcinoma.

33. A method of treating acute lymphoblastic leukemia, comprising administering to a mammal in need thereof the compound or a pharmaceutically acceptable salt of the compound of claim 1, in an amount effective to treat acute lymphoblastic leukemia.

34. A method of treating malignant melanoma, comprising administering to a mammal in need thereof the compound or a pharmaceutically acceptable salt of the compound of claim 1, in an amount effective to treat malignant melanoma.

35. A method of treating soft-tissue or bone sarcoma, comprising administering to a mammal in need thereof the compound or a pharmaceutically acceptable salt of the compound of claim 1, in an amount effective to treat soft-tissue or bone sarcoma.

36. A method of synthesizing a compound of claim 1 comprising reacting a boronic acid of the formula (R²)ᵣ—Ar—B(OH)₂ with the 5-chloro-3H-[1,2,3]triazolo[4,5-d]pyrimidine 2: 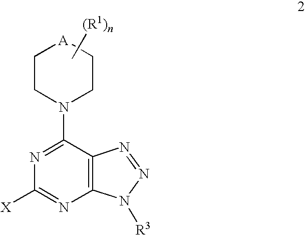
wherein X is halo and A, Ar, R¹, n, R², r, and R³, are as defined in claim 1; 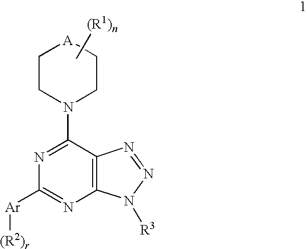
thereby producing the 3H-[1,2,3]triazolo[4,5-d]pyrimidine 1.

37. The method of claim 36 further comprising further comprising: (a) reacting the 2,4,6-trihalo-5-nitropyrimidine of Formula 3 with an amine 4 to substitute the halogen 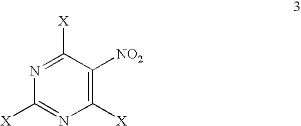
atom at position 4 of the pyrimidine: 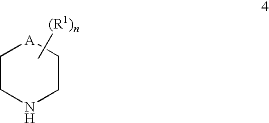
thereby producing 5: 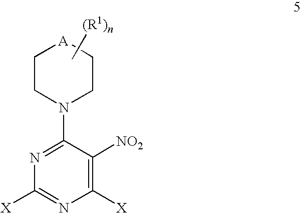
(b) reacting dihalo pyrimidine 5 with amine R³—NH₂ replacing the halogen atom at position 6 of the pyrimidine ring with radical R³—NH—;
c) reducing the product of the proceeding reaction to convert the nitro group at position 5 of the pyrimidine ring to an amino group without removing the halogen atom at position 2 of the pyrimidine;
d) diazotizing and cyclizing the diaminopyrimidine; 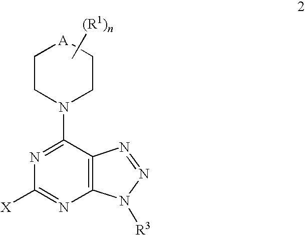
thereby producing 3H-[1,2,3]triazolo[4,5-d]pyrimidine 2.
